(19) United States
(12) Patent Application Publication
(10) Pub. No.: US 2025/0228953 A1
(43) Pub. Date: Jul. 17, 2025

(54) INTRACELLULAR TARGETING OF OLIGONUCLEOTIDES

(71) Applicant: Entrada Therapeutics, Inc., Boston, MA (US)

(72) Inventors: Ziqing Qian, Wellesley, MA (US); Mahboubeh Kheirabadi, Natick, MA (US); Matthew Streeter, Weymouth, MA (US)

(21) Appl. No.: 18/698,647

(22) PCT Filed: Nov. 7, 2022
(86) PCT No.: PCT/US2022/079409

§ 371 Date: Apr. 4, 2024

Related U.S. Application Data
(60) Provisional application No. 63/277,139, filed on Nov. 8, 2021.
(60) Provisional application No. 63/290,813, filed on Dec. 17, 2021.

(51) Int. Cl. A61K 47/64; A61K 47/54; C12N 15/113

(52) U.S. Cl. CPC A61K 47/64; A61K 47/549; C12N 15/113; C12N 2310/11; C12N 2310/3233; C12N 2310/351; C12N 2310/3513; C12N 2320/32

(57) ABSTRACT

Compounds are provided include a cell penetrating peptide, a therapeutic oligonucleotide, and a carbohydrate targeting moiety. The compounds may also include an exocyclic peptide. The compounds may be targeted to liver cells. The therapeutic oligonucleotide may be an oligonucleotide for treating a disease or disorder associated with a liver cell.

### CROSS-REFERENCE TO RELATED APPLICATIONS

This application claims priority to U.S. provisional patent application No. 63/277,139, which was filed on Nov. 8, 2021, and U.S. provisional patent application No. 63/290,813, which was filed on Dec. 17, 2021, the disclosures of each of which are hereby incorporated by reference in their entireties.

### INTRODUCTION

Nucleic acids and their synthetic analogs hold enormous potential as therapeutic agents, especially against targets that are challenging for conventional drug modalities (e.g., missing/defective proteins caused by genetic mutations).

However, a major problem in bringing the potential of such therapies to the clinic is their limited ability to gain access to the intracellular compartment when administered systemically. Carrier systems, such as polymers, cationic liposomes or chemical modifications, for example by the covalent attachment of cholesterol molecules, have been used facilitate intracellular delivery of nucleic acid therapeutics. Still, intracellular delivery efficiency by these approaches is often low and improved delivery systems to increase efficacy of intracellular delivery have remained elusive.

N-acetylgalactosamine (GalNAc) has shown promise in targeting therapeutic oligonucleotides to liver cells. However, further improvement in intracellular targeting, such as targeting to liver cells, such as hepatocytes would be desired.

The present disclosure addresses this and other issues.

### SUMMARY

The present disclosure describes, among other things, compounds comprising a cell penetrating peptide (CPP), a therapeutic oligonucleotide (TO), and a carbohydrate targeting moiety (CTM). In embodiments, the compound may further comprise an exocyclic peptide (EP). In embodiments, the compound comprises a cyclic cell penetrating peptide (CPP); an exocyclic peptide (EP); a therapeutic oligonucleotide (TO); a carbohydrate targeting moiety (CTM); and one or more linkers linking the CPP, the EP, the TO, and the CTM. In embodiments, the compounds enhance delivery to a target cell relative to compounds that do not comprise the CPP. In embodiments, the compounds enhance delivery to liver cells, such as hepatocytes, relative to compounds that do not comprise the CPP. In embodiments, the compounds enhance delivery to a target cell relative to compounds that do not comprise the CPP and the EP. In embodiments, the compounds may enhance delivery to liver cells, such as hepatocytes, relative to compounds that do not comprise the CPP, the EP and the CTM. In embodiments, the CPP is a cyclic CPP, for example, a cyclic CPP disclosed in International Patent Application No. PCT/US2022/071489, filed Mar. 31, 2022, Publication No. WO 2022/213118, entitled “CYCLIC CELL PENETRATING PEPTIDES,” the disclosure of which is hereby incorporated by reference in its entirety. In embodiments, the compounds may enhance delivery to liver cells, beyond hepatocytes such as kupffer cells (macrophages), endothelial cells, relative to compounds that do not comprise the CPP and EP.

In embodiments, the compounds may have a structure according to any one of Formulas A-M, as follows:

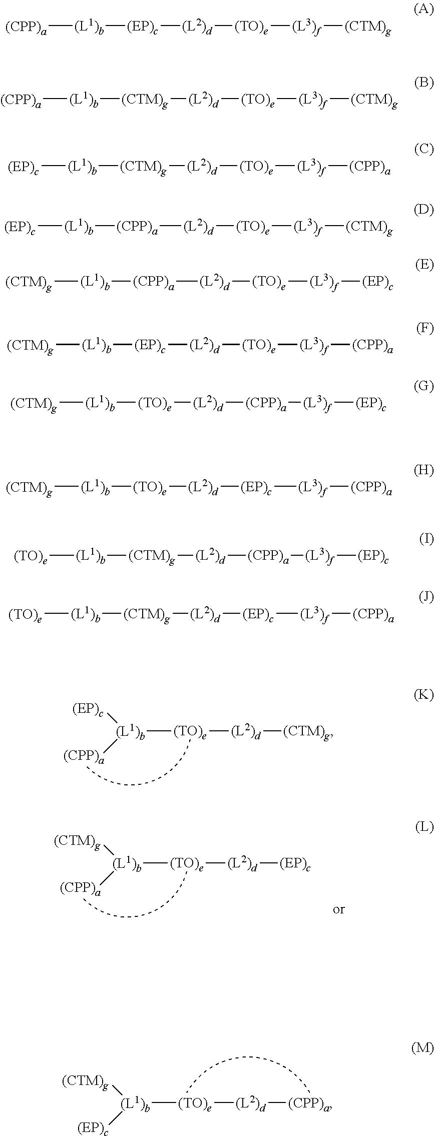

wherein:
CPP is a cell penetrating peptide moiety,
EP is an exocyclic peptide,
CTM is a carbohydrate targeting moiety,
TO is a therapeutic oligonucleotide,
each L¹, L², and L³ are independently a linker,
a, e, and g are each independently an integer from 1 to 10, and
b, c, d, and f are each independently an integer from 0 to 10.

In embodiments, one or more CTM comprises a GalNAc moiety.

In embodiments, when c is greater than d, when a is greater than b, or when g is greater than f, one or more linker (L¹, L², L³) may be branched to accommodate more than one of CPP, CTM, or EP.

In embodiments, the therapeutic oligonucleotide (TO) includes, but is not limited to, a small interfering RNA (siRNA), a microRNA (miRNA), a ribozyme, an immune stimulating nucleic acid, an antisense oligonucleotide, an antagomir, an antimir, a microRNA a mimic, a supermir, a Ul adaptor, an aptamer, or a guide RNA. In embodiments, the therapeutic oligonucleotide includes an antisense oligonucleotide (ASO). In embodiments, the ASO includes a nucleotide sequence complementary to a target nucleotide sequence. In embodiments, the therapeutic oligonucleotide includes at least one modified nucleotide that includes a phosphorothioate (PS) nucleotide, a phosphorodiamidate morpholino (PMO) nucleotide, a locked nucleic acid (LNA), a peptide nucleic acid (PNA), a nucleotide that includes a 2′-O-methyl (2′-OMe) modified backbone, a 2′O-methoxy-ethyl (2′-MOE) nucleotide, a 2′,4′ constrained ethyl (cEt) nucleotide, a 2′-deoxy-2′-fluoro-beta-D-arabinonucleic acid (2′F-ANA), or a combination thereof. In embodiments, the therapeutic oligonucleotide includes one or more phosphorodiamidate morpholino (PMO) nucleosides, 2′-O-methylated nucleosides, locked nucleic acids (LNAs), or a combination thereof. In embodiments, the therapeutic oligonucleotide is from about 5 to about 1000, about 5 to about 500, about 5 to about 100, about 5 to about 50, about 5 to about 30, about 10 to about 30, about 15 to about 30, about 20 to about 30, about 5 to about 25, about 10 to about 25, about 15 to about 25, about 20 to about 25, about 5 to about 20, about 10 to about 20, or about 15 to about 20 nucleotides in length.

In embodiments, the therapeutic oligonucleotide (TO) includes an antisense oligonucleotide (ASO). In embodiments, the ASO includes a nucleotide sequence complementary to a target nucleotide sequence. In embodiments, the target nucleotide sequence may encode a polypeptide or protein, or portion thereof. In embodiments, the target nucleotide sequence may encode a mutant polypeptide or protein, or portion thereof. The mutant polypeptide or protein, or portion thereof may be associated with a disease.

In embodiments, at least a portion of the compound of Formula A-M is cyclic. In embodiments, one or more CPP is a cyclic CPP (cCPP). In embodiments, one or more of the CCPs and one or more of the cargos together form a cyclic or bicyclic ring. In embodiments, a linker may form a part of the cyclic or bicyclic ring with the CPP and the cargo. In embodiments, a compound of Formula A-J may comprise a CCP-Cargo ring structure as shown in Formula Z-I or Z-II:

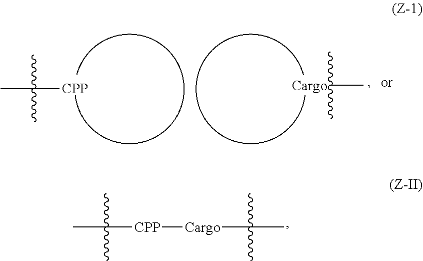

where a linker may or may not form a portion of a ring. When a linker does not form a part of a ring, a bond may be formed between a group of the CPP and a group of the cargo.

In embodiments, L¹ or L² may accommodate more than one of CPP, CTM, or EP. In embodiments, the compounds may have a structure according to Formula N or O, as follows:

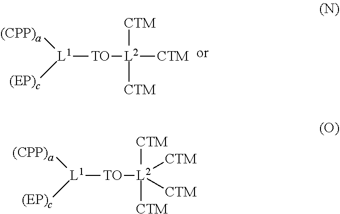

wherein
L¹ and L² are each independently a linker;
a is an integer from 1 to 10; and
c is an integer from 0 to 10.

In embodiments, the compounds may have a structure according to Formula P, as follows:

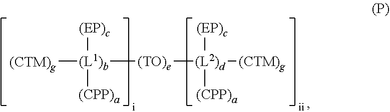

wherein
each L¹ and each L² are each independently a linker,
i and ii are each independently 0 to 10, provided that at least one of i or ii is 1 or greater; each a, b, c, d, f, and g are each independently an integer from 0 to 10, provided that at least one a is 1 or greater and at least one g is 1 or greater; and
e is an integer from 1 to 10.

In embodiments, the compound has a structure of Formula Q, Q¹, Q² or Q³

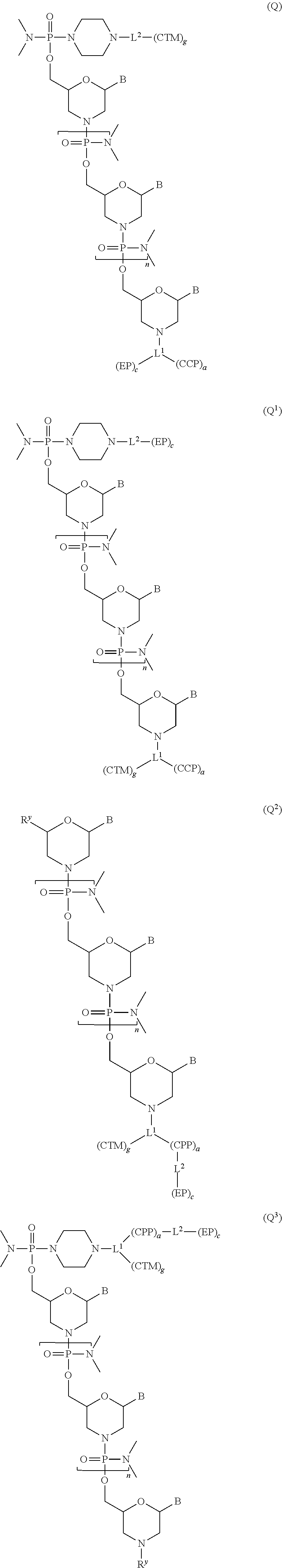

wherein:
CPP is a cell penetrating peptide;
EP is an exocyclic peptide,
CTM is a carbohydrate targeting moiety;
a is an integer from 1 to 10;
c is an integer from 0 to 10;
g is an integer from 1 to 10;
L¹ is a linker;
L² is a linker;
L³ is a linker;
R^{y} is H or —CH₂OR^{z};
R^{z} is a capping group;
B is each independently a nucleobase of the therapeutic oligonucleotide; and
n is an integer from 1 to 1000.

It is understood that other permutation of the CPP, CTM and/or EP can be envisioned and synthesized in a similar fashion.

L¹ or L² may be branched to may be branched to accommodate more than one cargo. L¹ or L² may be branched to accommodate more than one of CPP, CTM, or EP.

In embodiments, the compound is of the formula:

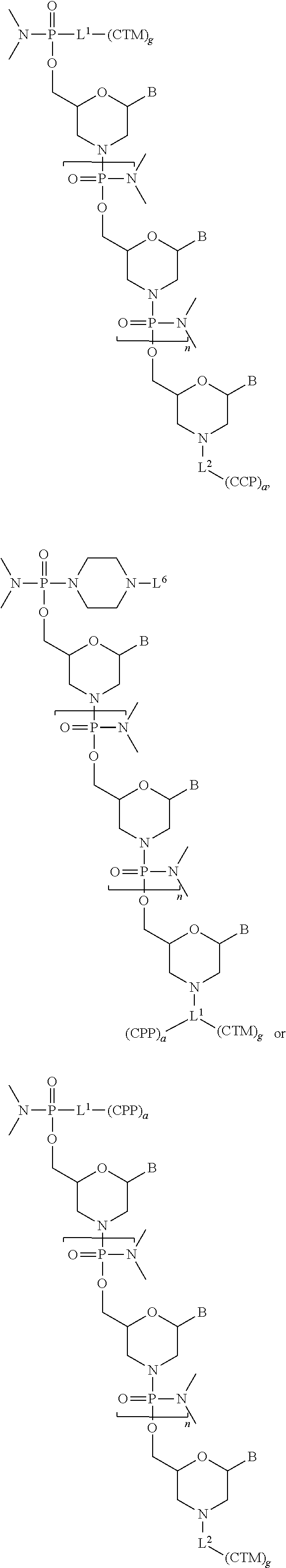

wherein L¹ or L² comprises a 1,2,3-triazolyl group.

In embodiments, the triazolyl group is a group of the formula:

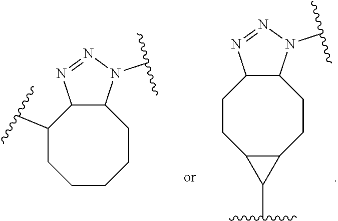 [SMILES: *C1CCCCCC2C1N=NN2* ; *C1C2CCC3N=NN(*)C3CCC12]

In embodiments, a pharmaceutical composition is provided that includes a compound described herein and a pharmaceutically acceptable carrier.

In embodiments, a cell is provided that includes a compound described herein.

In embodiments, methods of making and using the compound are provided.

In embodiments, methods of treating a disease or disorder are provided. In embodiments, the disease or disorder may include, but is not limited to, one or more of Pompe disease, Wilson disease, amyloidotic cardiomyopathy, hypercholesterolemia, hemophilia or rare bleeding disorders (including, for example, hemophilia A or hemophilia B), paroxysmal nocturnal hemoglobinuria, alpha-1-antitrypsin deficiency, primary hyperoxaluria type 1, hepatitis (including, for example, hepatitis A, hepatitis B, hepatitis C, hepatitis D, hepatitis E, hepatitis F, hepatitis G, or hepatitis H), hepatic porphyrias, beta-thalassemia or iron overload disorders, angioedema (including, for example, hereditary angioedema), thromboprophylaxis, hypertriglyceridemia, hyperlipidemia, hypertension (including, for example, treatment resistant hypertension), hereditary hemochromatosis (HH), pre-eclampsia, chronic liver infection, thrombosis, orphan genetic disease, cardiovascular disease, fibrotic liver diseases, Non-alcoholic Fatty Liver Disease (NAFLD) (including, for example, non-alcoholic steatohepatitis (NASH)), diabetes (including, for example, type 1 diabetes, type 2 diabetes, and pre-diabetes), high lipoprotein(a), dislipidemias, acromegaly, ornithine transcarbamylase deficiency, obesity, liver cancer (including, for example, hepatocellular carcinoma (HCC), fibrolamellar HCC, hepatoblastoma, chloangriocarcinoma, angiosarcoma, hemangiosarcoma, or liver metastasis, mucopolysaccharidosis type 1, mucopolysaccharidosis type 2, methylmalonic acidemia, autoimmune hepatitis, and phenylketonuria.

In embodiments, the disease or disorder to be treated includes liver diseases or disorders characterized by unwanted cell proliferation, hematological disorders, metabolic disorders, or disorders characterized by inflammation. A proliferation disorder of the liver can be, for example, a benign or malignant disorder, e.g., a cancer, e.g., a hepatocellular carcinoma (HCC), hepatic metastasis, or hepatoblastoma. A hepatic hematology or inflammation disorder can be a disorder involving clotting factors, a complement-mediated inflammation or a fibrosis, for example. Metabolic diseases of the liver include dyslipidemias and irregularities in glucose regulation. In embodiments, the disease or disorder to be treated includes a genetic liver disease or disorder. The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE FIGURES

FIG. 1 shows modified nucleotides that can be used in therapeutic oligonucleotides described herein.

FIGS. 2A-2D provide structures for morpholino subunit monomers that can be used in synthesizing phosphorodiamidate-linked morpholino oligomers. FIG. 2A provides the structure for adenine morpholino monomer. FIG. 2B provides the structure for cytosine morpholino monomer. FIG. 2C provides the structure for guanine morpholino monomer. FIG. 2D provides the structure for thymine morpholino monomer.

FIGS. 3A-3D illustrate conjugation chemistries for connecting an oligonucleotide (such as a therapeutic oligonucleotide) to a peptide (such as a cyclic cell penetrating peptide). FIG. 3A shows the amide bond formation between peptides with carboxylic acid group or with TFP activated ester and primary amine residues at the 5′ end of oligonucleotide. FIG. 3B shows the conjugation of secondary amine or primary amine modified oligonucleotide at 3′ and peptide-TFP ester through amide bond formation. FIG. 3C shows the conjugation of peptide-azide to the 5′ cyclooctyne modified oligonucleotide via copper-free azide-alkyne cycloaddition. FIG. 3D demonstrates another exemplary conjugation between 3′ modified cyclooctyne oligonucleotides or 3′ modified azide oligonucleotides and CPP containing linker-azide or linker-alkyne/cyclooctyne moiety, via a copper-free azide-alkyne cycloaddition or cupper catalyzed azide-alkyne cycloaddition, respectively (click reaction).

FIG. 4 shows conjugation chemistry for connecting an oligonucleotide (such as therapeutic oligonucleotide moiety) and CPP with an additional linker modality containing a polyethylene glycol (PEG) moiety.

FIG. 5 shows a synthetic scheme for PMO1-EEV1 (FIG. 5).

FIG. 6 shows the structure of PMO1-EEV1.

FIG. 7 shows a scheme for synthesizing GalNAc-PMO2 a compound used in studies described in the Examples herein

FIG. 8 shows the structure of GalNAc-PMO2.

FIG. 9 shows a scheme for synthesizing GalNAc-PMO2-EEV1, a compound used in studies described in the Examples herein

FIG. 10 shows the structure of GalNAc-PMO2-EEV1.

FIG. 11A is a scheme for synthesizing PMO3.

FIG. 11B is the structure of PMO3.

FIG. 12 is a scheme for synthesizing PMO3-GalNAc-NHAc.

FIG. 13 is the structure of PMO3-GalNAc-NHAc.

FIG. 14 is a scheme for synthesizing PMO3-GalNAc-EEV1.

FIG. 15 is the structure of PMO3-GalNAc-EEV1.

FIG. 16 is an overview of a study design for administrating and evaluating pharmacodynamic and biodistribution effects of compounds illustrative of those described herein.

FIGS. 17A-17B show results illustrating exon skipping percentage, eGFP (pg/μg).

FIG. 18 shows compound concentration in liver tissue.

FIG. 19 shows representative images of liver sections.

FIG. 20 shows strong eGFP colocalization with arginase-1 (hepatocyte marker) for GalNAc-PMO2-EEV1.

FIG. 21 shows significant co-localization of eGFP and CD31 stain for PMO1-EEV1 and GalNAc-PMO2-EEV1.

FIG. 22 shows co-localization of eGFP and F4/80 stain for PMO1 and PMO1-EEV1.

FIG. 23 is an overview of a second study design for administrating and evaluating duration of action for pharmacodynamic effects of compounds illustrative of those described herein.

FIGS. 24A-24B, show percent splice correction and eGFP (pg/μg).

FIG. 25 shows a third study design evaluating different EEV amino acid composition needed to act synergistically with GalNAc liver targeting.

FIG. 26 illustrates eGFP protein level in liver after 1 week.

FIG. 27 shows a fourth study design evaluating the site of CTM site of conjugation (5′ vs 3′) to act synergistically with EEV for liver targeting and efficacy.

FIG. 28 shows eGFP (pg/μg) for PMO1, PMO1-EEV1, GalNAc-PMO2, GalNAc-PMO2-EEV1, PMO3-GalNAc-NHAc and PMO3-GalNAc-EEV1 via both IV and SC.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

### Definitions

As used in the description and the appended claims, the singular forms “a,” “an,” and “the” include plural referents unless the context clearly dictates otherwise. Thus, for example, reference to “a composition” includes mixtures of two or more such compositions, reference to “a compound” includes mixtures of two or more such compounds, reference to “the moiety” includes compounds having two or more such moieties, and the like.

The term “about” when immediately preceding a numerical value means a range (e.g., plus or minus 20% of that value, for example, within 10%). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10 to 30”) refers, respectively to all values in the series, or the endpoints of the range.

“Amino acid” refers to an organic compound that includes an amino group and a carboxylic acid group and has the general formula

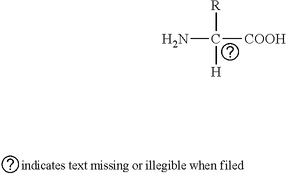

where R can be any organic group. An amino acid may be a naturally occurring amino acid or non-naturally occurring amino acid. An amino acid may be a proteogenic amino acid or a non-proteogenic amino acid. An amino acid can be chiral or achiral. An amino acid can be an L-amino acid or a D-amino acid. The term “amino acid side chain” or “side chain” refers to the characterizing substituent (“R”) bound to the α-carbon of a natural or non-natural α-amino acid.

“2-[2-[2-aminoethoxy]ethoxy]acetic acid” is also referred to as AEEA or miniPEG.

As used herein, the term “cell penetrating peptide” or “CPP” refers to a peptide that facilitates the delivery of a cargo, e.g., a therapeutic oligonucleotide, into a cell. In embodiments, the CPP is cyclic, and is represented as “cCPP”. In embodiments, he cCPP is capable of directing cargo, such as a therapeutic oligonucleotide, to penetrate the membrane of a cell. In embodiments, the cCPP delivers the cargo, such as a therapeutic oligonucleotide, to the cytosol of the cell. In embodiments, the cCPP delivers the cargo, such as a therapeutic oligonucleotide, to a cellular location where a translation of mRNA to form a polypeptide occurs. Cyclic CPPs are disclosed, for example, in International Patent Application No. PCT/US2022/071489, filed Mar. 31, 2022, Publication No. WO 2022/213118, entitled “CYCLIC CELL PENETRATING PEPTIDES,” the disclosure of which is hereby incorporated by reference in its entirety.

As used herein, the term “endosomal escape vehicle” (EEV) refers to a cCPP that is conjugated by a chemical linkage (i.e., a covalent bond or non-covalent interaction) to a moiety such as a linker as defined herein, an exocyclic peptide (EP) as defined herein, a cell targeting moiety (CTM) as defined herein, or a combination thereof. In embodiments, an EEV comprises a cCPP linked to an exocyclic peptide (EP) as defined herein.

As used herein, the term “EEV-conjugate” refers to an endosomal escape vehicle defined herein conjugated by a chemical linkage (i.e., a covalent bond or non-covalent interaction) to a cargo. In embodiments, the cargo is a therapeutic oligonucleotide that is delivered into a cell by the EEV.

As used herein, the term “exocyclic peptide” (EP) and “modulatory peptide” (MP) may be used interchangeably to refer to two or more amino acid residues linked by a peptide bond that is conjugated to a cyclic peptide disclosed herein. In embodiments, the EP, when conjugated to a cyclic peptide disclosed herein, alters the tissue distribution and/or retention of the compound. Typically, the EP comprises at least one positively charged amino acid residue, e.g., at least one lysine residue and/or at least one arginine residue. Non-limiting examples of EP are described herein. In embodiments, the EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV, the nucleoplasmin bipartite NLS with the sequence NLSKRPAAIKKAGQAKKKK, the c-myc nuclear localization sequence having the amino acid sequence PAAKRVKLD or RQRRNELKRSF, the sequence RMRKFKNKGKDTAELRRRRVEVSVELRKAKKDEQILKR RNV of the IBB domain from importin-alpha, the sequences VSRKRPRP and PPKKARED of the myoma T protein, the sequence PQPKKKPL of human p53, the sequence SALIKKKKKMAP of mouse c-abl IV, the sequences DRLRR and PKQKKRK of the influenza virus NSI, the sequence RKLKKKIKKL of the Hepatitis virus delta antigen and the sequence REKKKFLKRR of the mouse Mxl protein, the sequence KRKGDEVDGVDEVAKKKSKK of the human poly(ADP-ribose) polymerase and the sequence RKCLQAGMNLEARKTKK of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., a CPP and a cargo, e.g., a therapeutic oligonucleotide (TO), or a CTM and a cargo, e.g., a therapeutic oligonucleotide (TO)). In embodiments, the linker comprises one or more natural or non-natural amino acids or a polypeptide. In other embodiments, the linker comprises a synthetic compound containing two or more appropriate functional groups suitable to bind a CPP or CTM to a cargo moiety, to thereby form a compound disclosed herein. In another embodiment, the linker includes a bonding moiety (M) to thereby conjugate the CPP to the cargo, e.g., a therapeutic oligonucleotide. In other embodiments, a linker includes comprising a PEG group, an aromatic group or a alkyl group. In embodiments, the linker conjugates la CTM to a TO. The term linker will have to be understood to conjugate two or more groups as appropriate throughout the specification, and as one of ordinary skill in the art would understand.

As used herein, “cell targeting moiety” refers to a molecule or macromolecule that specifically binds to a molecule, such as a receptor, on the surface of a target cell. In embodiments, the cell surface molecule is expressed only on the surface of a target cell. In embodiments, the cell surface molecule is also present on the surface of one or more non-target cells, but the amount of cell surface molecule expression is higher on the surface of the target cells. Examples of a cell targeting moiety include, but are not limited to, an antibody, a peptide, a protein, an aptamer or a small molecule.

As used herein, a “carbohydrate targeting moiety” or “CTM” refers to a cell targeting moiety that includes a carbohydrate moiety. The CTM may be a liver cell targeting moiety. As used herein, “carbohydrate” refers to a compound which is either a carbohydrate moiety made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include sugars (mono-, di-, tri- and oligosaccharides containing from about 4-9 monosaccharide units), and polysaccharides such as starches, glycogen, cellulose and polysaccharide gums. Specific monosaccharides include C₅ and above (e.g., C₅-C₈) sugars; di- and trisaccharides include sugars having two or three monosaccharide units (e.g., C₅-C₈).

The term “monosaccharide” includes, but is not limited to, allose, altrose, arabinose, cladinose, erythrose, erythrulose, fructose, D-fucitol, L-fucitol, fucosamine, fucose, fuculose, galactosamine, D-galactosaminitol, N-acetyl-galactosamine, galactose, glucosamine, N-acetyl-glucosamine, glucosaminitol, glucose, glucose-6-phosphate, gulose glyceraldehyde, L-glycero-D-mannos-heptose, glycerol, glycerone, gulose, idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tartaric acid, threose, xylose and xylulose. The monosaccharide can be in D- or L configuration. The monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include amino monosaccharides. In embodiments, an amino monosaccharide is galactosamine, glucosamine, mannosamine, fucosamine, quinovosamine, neuraminic acid, muramic acid, lactosediamine, acosamine, bacillosamine, daunosamine, desosamine, forosamine, garosamine, kanosamine, kansosamine, mycaminose, mycosamine, perosamine, pneumosamine, purpurosamine, or rhodosamine. It is understood that the monosaccharide and the like can be further substituted.

The terms “disaccharide”, “trisaccharide” and “polysaccharide” includes, but is not limited to, abequose, acrabose, anucetose, amylopectin, amylose, apiose, arcanose, ascarylose, ascorbic acid, boivinose, cellobiose, cellobiose, cellulose, chacotriose, chalcose, chitin, colitose, cyclodextrin, cymarose, dextrin, 2-deoxyribose, 2deoxyglucose, diginose, digitalose, digitoxose, evalose, evemitrose, fructooligosachharide, galto-oligosaccharide, gentianose, gentiobiose, glucan, glucogen, glycogen, hamamelose, heparin, inulin, isolevoglucosenone, isomaltose, isomaltotriose, isopanose, kojibiose, lactose, lactosamine, lactosediamine, laminarabiose, levoglucosan, levoglucosenone, β-maltose, maltriose, mannan-oligosaccharide, manninotnose, melezitose, melibiose, muramic acid, mycarose, mycinose, neuraminic acid, nigerose, nojirimycin, noviose, oleandrose, panose, paratose, planteose, pnmeverose, raffinose, rhodinose, rutinose, sarmentose, sedoheptulose, sedoheptulosan, solatriose, sophorose, stachyose, streptose, sucrose, am-trehalose, trehalosamine, turanose, tyvelose, xylobiose, umbelliferose and the like. Further, it is understood that the “disaccharide”, “trisaccharide” and “polysaccharide” and the like can be further substituted. Disaccharide also includes amino sugars and their derivatives, particularly, a mycaminose, derivatized at the C-4′ position or a 4 deoxy-3-amino-glucose derivatized at the C-6′ position.

The terms “peptide,” “protein,” and “polypeptide” are used interchangeably to refer to a natural or synthetic molecule comprising two or more amino acids. In embodiments, two or more amino acid residues are linked by the carboxyl group of one amino acid to the alpha amino group. In embodiments, two or more amino acids of the polypeptide are joined by a peptide bond. In embodiments, the polypeptide includes a peptide backbone modification in which two or more amino acids are covalently attached by a bond other than a peptide bond. In embodiments, the polypeptide includes one or more non-natural amino acids, amino acid analogs, or other synthetic molecules that are capable of integrating into a polypeptide. The term polypeptide includes naturally occurring and artificial amino acids. The term polypeptide includes peptides, for example, that include from about 2 to about 100 amino acid residues as well as proteins, that include more than about 100 amino acid residues, or more than about 1000 amino acid residues.

As used herein, the term “contiguous,” as it relates to amino acids, refers to two amino acids, which are connected by a covalent bond. For example, in the context of a representative cyclic peptide such as

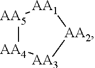

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic peptides described herein have amino acids (e.g., arginine) incorporated therein through formation of one or more peptide bonds. The amino acids incorporated into the cyclic peptide may be referred to residues, or simply as an amino acid. Thus, arginine or an arginine residue refers to

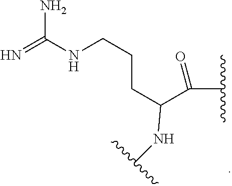 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

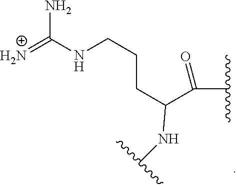 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. In embodiments, the chiral molecule is an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁n alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkynyl groups include all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to the group —C(O)R, where R is hydrogen, alkyl, alkenyl, alkynyl, carbocyclyl, or heterocyclyl, as defined herein. Unless stated otherwise specifically in the specification, an acyl group can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system that includes hydrogen, 6 to 40 carbon atoms and at least one aromatic ring. For purposes of this disclosure, the aryl can be a monocyclic, bicyclic, tricyclic or tetracyclic ring system, which can include fused or bridged ring systems. Aryls include, but are not limited to, aryl divalent radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In embodiments, the aryl divalent and is attached, directly or indirectly, to the CPP through a single bond and, directly or indirectly, to the cargo through a single bond. Unless stated otherwise specifically in the specification, an aryl group can be optionally substituted.

“Heteroaryl” refers to a 5- to 22-membered ring system radical comprising hydrogen atoms, one to fourteen carbon atoms, one to eight heteroatoms selected from nitrogen, oxygen and sulfur, and at least one aromatic ring. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). In embodiments, the heteroaryl is divalent and is attached, directly or indirectly, to the CPP through a single bond and, directly or indirectly, to the cargo through a single bond. Unless stated otherwise specifically in the specification, a heteroaryl group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can include from 3 to 20 carbon atoms in the ring. Unless stated otherwise specifically in the specification, the carbocyclyl can be a monocyclic, bicyclic, tricyclic or tetracyclic ring system, which can include fused or bridged ring systems Carbocyclic rings include aryls and cycloalkyl, cycloalkenyl, and cycloalkynyl as defined herein. Unless stated otherwise specifically in the specification, a carbocyclyl group can be optionally substituted. In embodiments, the carbocyclyl divalent, and is attached, directly or indirectly, to the CPP through a single bond and, directly or indirectly, to the cargo through a single bond. Unless stated otherwise specifically in the specification, a carbocyclyl group can be optionally substituted.

“Cycloalkyl” refers to a stable non-aromatic monocyclic or polycyclic fully saturated hydrocarbon having from 3 to 40 carbon atoms and at least one ring, wherein the ring consists solely of carbon and hydrogen atoms, which can include fused or bridged ring systems. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. In embodiments, the cycloalkyl divalent and is attached, directly or indirectly, to the CPP through a single bond and, directly or indirectly, to the cargo through a single bond. Unless otherwise stated specifically in the specification, a cycloalkyl group can be optionally substituted.

“Cycloalkenyl” refers to a stable non-aromatic monocyclic or polycyclic hydrocarbon having from 3 to 40 carbon atoms, at least one ring having, and one or more carbon-carbon double bonds, wherein the ring consists solely of carbon and hydrogen atoms, which can include fused or bridged ring systems. Monocyclic cycloalkenyls include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. In embodiments, cycloalkenyl is divalent and is attached, directly or indirectly, to the CPP through a single bond and, directly or indirectly, to the cargo through a single bond. Unless otherwise stated specifically in the specification, a cycloalkenyl group can be optionally substituted.

“Cycloalkynyl” refers to a stable non-aromatic monocyclic or polycyclic hydrocarbon having from 3 to 40 carbon atoms, at least one ring having, and one or more carbon-carbon triple bonds, wherein the ring consists solely of carbon and hydrogen atoms, which can include fused or bridged ring systems. Monocyclic cycloalkynyls include, for example, cycloheptynyl, cyclooctynyl, and the like. The cycloalkynyl is attached, directly or indirectly, to the CPP through a single bond and, directly or indirectly, to the cargo through a single bond. Unless otherwise stated specifically in the specification, a cycloalkynyl group can be optionally substituted.

“Heterocyclyl,” “heterocyclic ring” or “heterocycle” refers to a stable 3- to 22-membered ring system which consists of two to fourteen carbon atoms and from one to eight heteroatoms selected from nitrogen, oxygen and sulfur. Heterocyclyl or heterocyclic rings include heteroaryls as defined below. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, succinimidyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In embodiments, the heterocyclyl is divalent and is attached, directly or indirectly, to the CPP through a single bond and, directly or indirectly, to the cargo through a single bond. Unless stated otherwise specifically in the specification, a heterocyclyl group can be optionally substituted.

The term “ether” used herein refers to a divalent moiety having a formula —[(R₁)ₘ—O—(R₂)ₙ]_{z}— wherein each of m, n, and z are independently an integer from 1 to 40, and R₁ and R₂ are independently an alkylene. Examples include polyethylene glycol. The ether is attached, directly or indirectly, to the CPP through a single bond and, directly or indirectly, to the cargo through a single bond. Unless stated otherwise specifically in the specification, the ether can be optionally substituted.

The term “capping group” refers to any group that does not substantially interfere with the biological function of the molecule such as but not limited to: optionally substituted alkyl; (optionally substituted alkenyl; optionally substituted alkynyl; optionally substituted carbocyclyl; optionally substituted heterocyclyl; —(R1-J-R2) wherein R1 alkylene, alkenylene, alkynylene, carbocyclyl, or heterocyclyl, R2 is independently selected from H, alkyl, alkenyl, alkynyl, carbocyclyl, and heterocyclyl, J is independently C, NR3, —NR3C(O)—, S, and O; optionally substituted alkoxy; H; OSO2(alkyl); OSO2(aryl); or methyl -PEG (m-PEG).

The term “substituted” used herein means any of the above groups (i.e., alkylene, alkenylene, alkynylene, aryl, carbocyclyl, cycloalkyl, cycloalkenyl, cycloalkynyl, heterocyclyl, heteroaryl, and/or ether) wherein at least one hydrogen atom is replaced by a bond to a non-hydrogen atoms such as, but not limited to: a deuterium atom; a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{R}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents. Further, those skilled in the art will recognize that “substituted” also encompasses instances in which one or more hydrogen atoms on any of the above groups are replaced by a substituent listed in this paragraph, and the substituent then forms a covalent bond with the CPP or cargo. The resulting bonding group can be considered a “substituent.” For example, In embodiments, any of the above groups can be substituted at a first position with a carboxylic acid (i.e., —C(═O)OH) which forms an amide bond with an appropriate amino acid CPP (e.g., lysine), and also substituted at a second position with either an electrophilic group (e.g., —C(═O)H, —CO₂R_{g}, -halide, etc.) or a nucleophilic group (—NH₂, —NHR_{g}, —OH, etc.) which forms a bond with the 5′ end of a nucleotide cargo, e.g., a therapeutic oligonucleotide (TO), or alternatively which forms a bond with the 3′ end of the oligonucleotide cargo, e.g., a therapeutic oligonucleotide (TO). The resulting bond, e.g., amide bond, can be considered a “substituent.” In embodiments, the second position is substituted with a thiol group which forms a disulfide bond with a thiol group attached to the cargo. The resulting disulfide is encompassed by the term substituent.

As used herein, the symbol

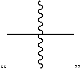

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

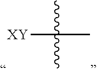

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

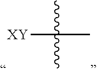

infers that when R³ is “XY”, the point of attachment bond is the same bond as the bond by which R³ is depicted as being bonded to CH₃.

As used herein, the term “sequence identity” refers to the percentage of nucleic acids or amino acids between two oligonucleotide or polypeptide sequences, respectively, that are the same and in the same relative position. As such, one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Tends Genet. 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows: (Identical Residues×100)/(Length of Alignment−Total Number of Gaps in Alignment).

In other embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP, gapped BLAST, and PSI-BLAST, in existence as of the date of filing. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. A “subject” may be a mammal, such as a primate or a human. Thus, the subject can be a human or veterinary patient. In embodiments, the term “patient” refers to a subject under the treatment of a clinician, e.g., a physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can include a 10%, 20%, 30%, 40%, 50%, 60%, 70%, 80%, 90%, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

As used herein, “treat,” “treating,” “treatment” and variants thereof, refers to any administration of one or more disclosed compounds that partially or completely alleviates, ameliorates, relieves, prevents, inhibits, delays onset of, reduces severity of, and/or reduces incidence of one or more symptoms or features of a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to reducing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” means that the amount of the composition used is of sufficient quantity to ameliorate one or more causes or symptoms of a disease or disorder. Such amelioration only requires a reduction or alteration, not necessarily elimination.

The term “pharmaceutically acceptable” refers to compounds, materials, compositions, and/or dosage forms which are, within the scope of sound medical judgment, suitable for use in contact with the tissues of human beings or animals without excessive toxicity, irritation, allergic response, or other problems or complications commensurate with a reasonable benefit/risk ratio.

The term “pharmaceutically acceptable salts” include compounds obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

The term “carrier” refers to a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to reduce degradation of the active ingredient or to reduce one or more adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In such embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized active agent. In embodiments, a dosage unit is a vial that includes reconstituted active agent. In embodiments, the active agent comprises a compound disclosed herein.

As used herein, the term “expression” refers to the functions and steps by which information encoded in an oligonucleotide, such as a gene, is converted into a polypeptide in a cell, including, but not limited to, transcription, translation and assembly of the encoded polypeptide.

As used herein, an “expression construct” is an oligonucleotide comprising a sequence that is capable of being expressed in a cell. The sequence capable of being expressed may be a coding sequence. In embodiments, the coding sequence comprises or encodes one or more introns. In embodiments, the coding sequence comprises or encodes no introns. In embodiments, the expression construct comprises regulatory sequences that result in efficient transcription of the coding sequence. In embodiments, the regulatory sequences include one or more of a promotor and an enhancer. The expression construct may be an expression vector.

As used herein, the terms “antisense oligonucleotide” and “ASO” are used interchangeably to refer to a polymeric nucleic acid structure which is at least partially complementary to a target nucleic acid molecule to which it (the ASO) hybridizes. The ASO may be a short (in embodiments, less than 50 consecutive bases) polynucleotide or polynucleotide homologue that includes a sequence complimentary to a target sequence. In embodiments, the ASO is a polynucleotide or polynucleotide homologue that includes a sequence complimentary to a target sequence in a target pre-mRNA strand. The ASO may be formed of natural nucleotides, nucleosides, or nucleobases; synthetic nucleotides, nucleosides, or nucleobases; nucleotide, nucleoside, or nucleobase homologues; or any combination thereof. In embodiments, the ASO includes an oligonucleoside. In embodiments, the ASO includes an antisense oligonucleotide. In embodiments, the ASO includes a conjugate group. Nonlimiting examples of ASOs include, but are not limited to, primers, probes, antisense oligonucleotides, external guide sequence (EGS) oligonucleotides, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound. In embodiments, an ASO modulates (increases, decreases, or changes) expression of a target nucleic acid.

As used herein, the terms “targeting” or “targeted to” refer to the association of a therapeutic oligonucleotide, for example, an ASO with a target nucleic acid molecule or a region of a target nucleic acid molecule. In embodiments, the therapeutic oligonucleotide includes an ASO that is capable of hybridizing to a target nucleic acid under physiological conditions. In embodiments, the ASO targets a specific portion or site within the target nucleic acid, for example, a portion of the target nucleic acid having at least one identifiable structure, function, or characteristic such as a particular exon or intron, or selected nucleobases or motifs within an exon or intron.

As used herein, the terms “target nucleic acid” refers to a nucleic acid molecule having a nucleic acid sequence to which the ASO binds or hybridizes. Target nucleic acids include, but are not limited to, RNA (including, but not limited to pre-mRNA and mRNA or portions thereof), cDNA derived from such RNA, as well as non-translated RNA, such as miRNA. For example, in embodiments, a target nucleic acid can be a cellular gene (or mRNA transcribed from such gene) whose expression is associated with a particular disorder or disease state, or a nucleic acid molecule from an infectious agent. The term “portion” refers to a defined number of contiguous (i.e., linked) nucleobases of a nucleic acid. In some embodiments, the target nucleic acid is a target RNA.

The term “target RNA” refers to an RNA molecule to which a therapeutic oligonucleotide binds. For example, an ASO may hybridize to the target RNA. In one embodiment, the target RNA is mRNA. In one embodiment, the target RNA is pre-mRNA. In one embodiment, the target RNA includes a splice site. In one embodiment, the target RNA includes a polyadenylation site or a portion thereof.

The “target pre-mRNA” is the pre-mRNA that includes the target sequence to which the ASO hybridizes.

The “target mRNA” is the mRNA sequence resulting from splicing of the target pre-mRNA sequence. In some embodiments, the target mRNA does not encode a functional protein. In some embodiments, the target mRNA retains one or more intron sequences.

The “target gene” of the present disclosure refers to the gene that encodes the target mRNA or pre-mRNA.

The “target protein” refers to a polypeptide having the amino acid sequence encoded by the target mRNA. In embodiments, the target protein may not be a functional protein.

“Wild type target protein” refers to a native, functional protein isomer produced by a wild type, normal, or unmutated version of the target gene. The wild type target protein also refers to a protein resulting from a target pre-mRNA that has been re-spliced.

A “re-spliced target protein”, as used herein, refers to the protein encoded by the mRNA resulting from the splicing of the target pre-mRNA to which the ASO hybridizes. Re-spliced target protein may be identical to a wild type target protein, may be homologous to a wild type target protein, may be a functional variant of a wild type target protein, may be an isoform of a wild type target protein, or may be an active fragment of a wild type target protein.

As used herein, the term “messenger RNA” or “mRNA” refers to an RNA molecule that encodes a protein and includes pre-mRNA and mature mRNA. “Pre-mRNA” refers to a newly synthesized eukaryotic mRNA molecule directly after DNA transcription. In embodiments, a pre-mRNA is capped with a 5′ cap, modified with a 3′ poly-A tail, and/or spliced to produce a mature mRNA sequence. In embodiments, pre-mRNA includes one or more introns. In one embodiment, the pre-mRNA undergoes a process known as splicing to remove one or more introns and join exons. In embodiments, pre-mRNA includes a polyadenylation site.

As used herein, the term “codon” refers to set sequences of oligonucleotides that cells use to translate information encoded in an mRNA into polypeptides. A codon typically includes a sequence of three contiguous oligonucleotides. To encode the 20 natural amino acids used to assemble proteins, cells rely on 64 triplets of RNA bases (G, C, A, or U), called codons. Each codon uniquely specifies an amino acid. For example, the codon TCA specifies the amino acid serine. Three of the 64 codons are reserved for signaling the end of a protein chain. These three codons are called stop codons and have one of the following sequences: UAG (sometimes referred to as the “amber” stop codon), UAA (sometimes referred to as the “ochre” stop codon), and UGA (sometimes referred to as the “opal” stop codon).

As used herein, the term “gene” refers to a nucleic acid molecule having a nucleic acid sequence that encompasses a 5′ promoter region associated with the expression of the gene product, any intron and exon regions, and 3′ untranslated regions (“UTR”) associated with the expression of the gene product.

As used herein, the term “transcript” refers an RNA molecule transcribed from DNA and includes, but is not limited to mRNA, mature mRNA, pre-mRNA, and partially processed RNA.

As used herein, the term “nucleoside” refers to glycosylamine that includes a nucleobase and a sugar. Nucleosides include, but are not limited to, natural nucleosides, abasic nucleosides, modified nucleosides, and nucleosides having mimetic bases and/or sugar groups. A “natural nucleoside” or “unmodified nucleoside” is a nucleoside that includes a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside that includes a phosphate group covalently linked to the sugar. Nucleotides may be modified with any of a variety of substituents. A modified nucleotide is considered a “nucleotide” for purposes of the present disclosure.

As used herein, the term “nucleobase” refers to the base portion of a nucleoside or nucleotide. A nucleobase may include any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase that includes a heterocycle.

As used herein, the term “oligonucleotide” refers to an oligomeric compound that includes a plurality of linked nucleotides or nucleosides. In certain embodiment, one or more nucleotides of an oligonucleotide is modified. In embodiments, an oligonucleotide includes ribonucleic acid (RNA) or deoxyribonucleic acid (DNA). In embodiments, oligonucleotides are composed of natural and/or modified nucleobases, sugars and covalent internucleoside linkages, and may further include non-nucleic acid conjugates.

As used herein, “therapeutic oligonucleotide” is an oligonucleotide that may be administered to a subject to treat a disease or disorder.

As used herein, “therapeutic oligonucleotide (TO) moiety” refers to a therapeutic oligonucleotide within a compound as described herein. The compound may comprise any suitable therapeutic oligonucleotide (TO). In embodiments, the TO includes, but is not limited to, a small interfering RNA (siRNA), a microRNA (miRNA), a ribozyme, an immune stimulating nucleic acid, an antisense oligonucleotide, an antagomir, an antimir, a microRNA a mimic, a supermir, a Ul adaptor, an aptamer, or a guide RNA. In embodiments, the therapeutic oligonucleotide includes an antisense oligonucleotide (ASO). In embodiments, the ASO includes a nucleotide sequence complementary to a target nucleotide sequence.

As used herein “internucleoside linkage” refers to a covalent linkage between adjacent nucleosides.

As used herein “natural internucleotide linkage” refers to a 3′ to 5′ phosphodiester linkage.

As used herein, the term “modified internucleoside linkage” refers to any linkage between nucleosides or nucleotides other than a naturally occurring internucleoside linkage.

As used herein “oligonucleoside” refers to an oligomeric compound that includes a plurality of linked nucleotides or nucleosides, similar to an oligonucleotide except that the internucleoside linkages do not contain a phosphorus atom.

As used herein the term “chimeric therapeutic oligonucleotide” refers to an therapeutic oligonucleotide, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In embodiments, a chimeric oligomeric compound comprises modified nucleosides that can be in isolated positions or grouped together in regions that will define a particular motif. Any combination of modifications and or mimetic groups can include a chimeric oligomeric compound as described herein.

As used herein, the term “mixed-backbone therapeutic oligonucleotide” refers to a therapeutic oligonucleotide wherein at least one internucleoside linkage of the therapeutic oligonucleotide is different from at least one other internucleoside linkage of the therapeutic oligonucleotide.

As used herein, the term “nucleobase complementarity” refers to a nucleobase that is capable of base pairing with another nucleobase. For example, in DNA, adenine (A) is complementary to thymine (T) and in RNA, adenine (A) is complementary to uracil (U). In embodiments, complementary nucleobase refers to a nucleobase of an ASO that is capable of base pairing with a nucleobase of its target nucleic acid. For example, if a nucleobase at a certain position of an ASO is capable of hydrogen bonding with a nucleobase at a certain position of a target nucleic acid, then the position of hydrogen bonding between the ASO and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an ASO and its target are complementary to each other when a sufficient number of corresponding positions in each molecule are occupied by nucleobases that can bond with each other to allow stable association between the ASO and the target. One skilled in the art recognizes that the inclusion of mismatches is possible without eliminating the ability of the oligomeric compounds to remain in association. Therefore, described herein are ASOs that may include up to about 20% nucleotides that are mismatched (i.e., are not nucleobase complementary to the corresponding nucleotides of the target). In embodiments, the ASOs contain no more than about 15%, for example, not more than about 10%, for example, not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” refers to the pairing of complementary oligomeric compounds (e.g., a nucleobase of an ASO and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

“Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

As used herein, the term “specifically hybridizes” refers to the ability of an oligomeric compound to hybridize to one nucleic acid site with greater affinity than it hybridizes to another nucleic acid site. In embodiments, an ASO specifically hybridizes to more than one target site. In embodiments, an oligomeric compound specifically hybridizes with its target under stringent hybridization conditions.

As used herein, the term “2-modified” or “2′-substituted” refers to a sugar that includes a substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl.

As used herein, the term “MOE” or “2′-MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of a nucleobase of an oligonucleotide for another nucleobase. High-affinity modifications include, but are not limited to, BNAs, locked nucleic acids (LNAs) and 2′-MOE. In embodiments, modifications are made to nucleobases of a therapeutic oligonucleotide that increase affinity of the modified nucleobase for another nucleobase.

As used herein the term “mimetic” refers to groups that are substituted for a sugar, a nucleobase, and/or internucleoside linkage in an therapeutic oligonucleotide. Generally, a mimetic is used in place of the sugar or sugar-internucleoside linkage combination. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH₃)—CH₂-4′) and Aminooxy BNA (2′-N(CH)—O—CH₂-4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene group, thereby forming a 2′-C,4′-C-oxymethylene linkage. LNAs include, but are not limited to, α-L-LNA, and β-D-LNA.

As used herein, the term “cap structure” or “terminal cap moiety” refers to chemical modifications, which have been incorporated at either end of a therapeutic oligonucleotide.

Several terms are used interchangeably thought the present disclosure. The terms GalNAc and GalNac are used interchangeably herein. The terms GalNAc-PMO2 and GalNac PMO2 are used interchangeably herein. The terms GalNAc-PMO2EEV1, GalNAc-PMO2-EEV1, GalNac PMO2-EEV1, and GalNAC PMO-EEV1 are used interchangeably herein.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### Compounds

Disclosed herein, in various embodiments, are compounds comprising a cell penetrating peptide (CPP), a therapeutic oligonucleotide, and a carbohydrate targeting moiety (CTM). In embodiments, the compound may further comprise an exocyclic peptide (EP). In embodiments, the EP comprises a nuclear localization sequence (NLS). In embodiments, the compounds enhance delivery to a target cell relative to compounds that do not comprise the CPP. In embodiments, the compounds enhance delivery to a target cell relative to compounds that do not comprise the CPP and the EP. In embodiments, the compounds may enhance delivery to liver cells, such as hepatocytes, relative to compounds that do not comprise the CPP. In embodiments, the compounds may enhance delivery to liver cells, such as hepatocytes, relative to compounds that do not comprise the CPP and the EP.

In embodiments, the compounds may have a structure according to any one of Formulas A-M, as follows:

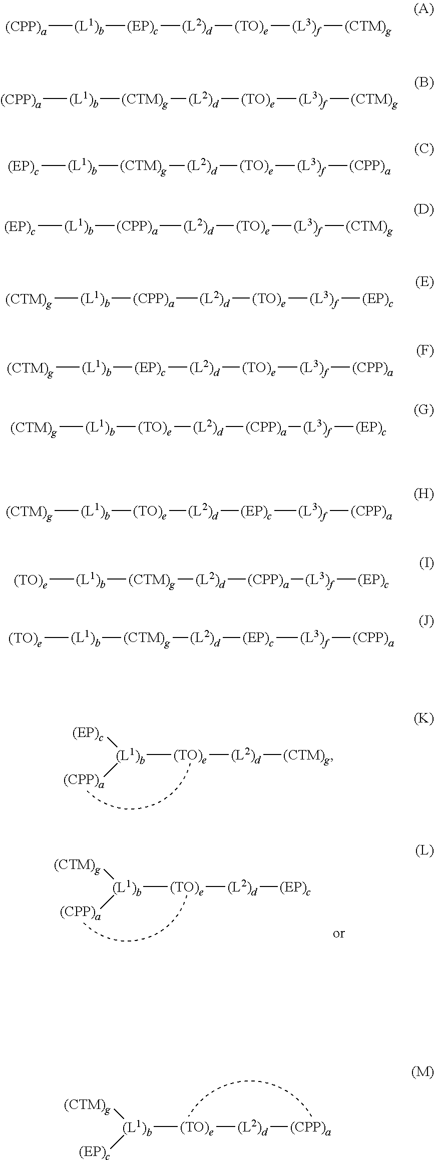

wherein:
the dashed line represents an optional connection between TO and CPP;
each L¹, L², and L³ are independently a linker;
a, e, and g are each independently an integer from 1 to 10; and
b, c, d, and f are each independently an integer from 0 to 10.

In embodiments, a is 1, 2, 3, 4, 5, 6, 7, 8, 9, or 10, inclusive of all subranges therebetween. In embodiments, a is 1, 2, 3, or 4. In embodiments, a is 1. When a is greater than 1 (e.g., 2, 3, 4, 5, 6, 7, 8, 9, or 10), each CPP may independently be selected from any suitable CPP. In embodiments, when a is greater than 1, each CPP is the same CPP.

In embodiments, b is 0, 1, 2, 3, 4, 5, 6, 7, 8, 9, or 10, inclusive of all subranges therebetween. In embodiments, b is equal to a. In embodiments, a is greater than b. When a is greater than b, the linker (e.g., L¹, L², or L³) may be branched to accommodate more than one CPP. In embodiments, b is 1 and a is an integer from 1 to 3. In embodiment, b is 1 and a is 1.

In embodiments, c is 0, 1, 2, 3, 4, 5, 6, 7, 8, 9, or 10, inclusive of all subranges therebetween. In embodiments, c is 0, 1, 2, 3, or 4. In embodiments, c is 1. When c is greater than 1 (e.g., 2, 3, 4, 5, 6, 7, 8, 9, or 10), each EP may independently be selected from any suitable EP. In embodiments, when c is greater than 1, each EP is the same EP.

In embodiments, d is 0, 1, 2, 3, 4, 5, 6, 7, 8, 9, or 10, inclusive of all subranges therebetween. In embodiments, d is equal to c. In embodiments, c is greater than d. When c is greater than d, the linker (e.g., L¹, L², or L³) may be branched to accommodate more than one EP.

In embodiments, e is 1, 2, 3, 4, 5, 6, 7, 8, 9, or 10, inclusive of all subranges therebetween. In embodiments, e is 1, 2, 3, 4, 5, 6, 7, or 8. In embodiments, e is 1, 2, 3, or 4. In embodiments, e is 1. In embodiments, d is 0 and e is 0. In embodiments, d is 0 and e is 1. In embodiments, d is 1 and e is 1.

In embodiments, f is 0, 1, 2, 3, 4, 5, 6, 7, 8, 9, or 10, inclusive of all subranges therebetween. In embodiments, f is equal to g. In embodiments, g is greater than f. When g is greater than f, the linker (e.g., L¹, L², or L³) may be branched to accommodate more than one CTM. In embodiments, f is 1 and g is an integer from 1 to 4. In embodiments, f is 1 and g is 3 or 4. In embodiments, f is 1, and g is 3. In embodiments, f is 1, and g is 4.

In embodiments, g is 1, 2, 3, 4, 5, 6, 7, 8, 9, or 10, inclusive of all subranges therebetween. In embodiments, g is 1, 2, 3, or 4. In embodiments, g is 3. In embodiments, g is 4. When g is greater than 1 (e.g., 2, 3, 4, 5, 6, 7, 8, 9, or 10), each CTM may independently be selected from any suitable CTM. In embodiments, when g is greater than 1, each CTM is the same CTM. In embodiments, each CTM is GalNAc.

In embodiments, one or more CTM comprises a GalNAc moiety.

In embodiments, at least a portion of the compound of Formula A-M is cyclic. In embodiments, one or more CPP is a cyclic CPP (cCPP). In embodiments, one or more of the CCPs and one or more of the cargos together form a cyclic or bicyclic ring. A linker may form a part of the cyclic or bicyclic ring with the CPP and the cargo. In embodiments, a compound of Formula A-M may comprise a CCP-Cargo ring structure as shown in Formula Z-I or Z-II:

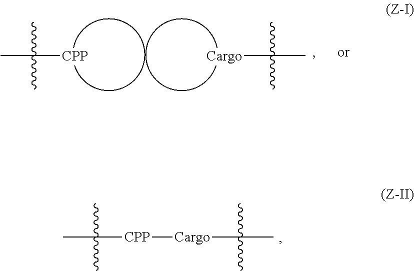

where a linker may or may not form a portion of a ring. When a linker does not form a part of a ring, a bond may be formed between a group of the CPP and a group of the cargo.

In embodiments, a is 1, 2, 3, 4, 5, 6, 7, 8, 9, or 10, inclusive of all subranges therebetween. In embodiments, a is 1, 2, 3, or 4. In embodiments, a is 1. When a is greater than 1 (e.g., 2, 3, 4, 5, 6, 7, 8, 9, or 10), each CPP may independently be selected from any suitable CPP. In embodiments, when a is greater than 1, each CPP is the same CPP.

In embodiments, c is 0, 1, 2, 3, 4, 5, 6, 7, 8, 9, or 10, inclusive of all subranges therebetween. In embodiments, c is 0, 1, 2, 3, or 4. In embodiments, c is 1. When c is greater than 1 (e.g., 2, 3, 4, 5, 6, 7, 8, 9, or 10), each EP may independently be selected from any suitable EP. In embodiments, when c is greater than 1, each EP is the same EP.

In embodiments, g is 1, 2, 3, 4, 5, 6, 7, 8, 9, or 10, inclusive of all subranges therebetween. In embodiments, g is 1, 2, 3, or 4. In embodiments, g is 3. In embodiments, g is 4. When g is greater than 1 (e.g., 2, 3, 4, 5, 6, 7, 8, 9, or 10), each CTM may independently be selected from any suitable CTM. In embodiments, when g is greater than 1, each CTM is the same CTM. In embodiments, each CTM is GalNAc.

In embodiments, b is 0, 1, 2, 3, 4, 5, 6, 7, 8, 9, or 10, inclusive of all subranges therebetween. In embodiments, b is equal to a. In embodiments, a is greater than b. When a is greater than b, the linker (e.g., L¹ or L²) may be branched to accommodate more than one CPP. In embodiments, b is 1.

In embodiments, d is 0, 1, 2, 3, 4, 5, 6, 7, 8, 9, or 10, inclusive of all subranges therebetween. In embodiments, d is equal to c. In embodiments, c is greater than d. When c is greater than d, the linker (e.g., L¹ or L²) may be branched to accommodate more than one EP. In embodiments, b is 1.

In embodiments, f is 0, 1, 2, 3, 4, 5, 6, 7, 8, 9, or 10, inclusive of all subranges therebetween. In embodiments, f is equal to g. In embodiments, g is greater than f. When g is greater than f, the linker (e.g., L¹ or L²) may be branched to accommodate more than one CTM. In embodiments, f is 1. In embodiments, f is 1, and g is 3. In embodiments, f is 1, and g is 4.

In embodiments, e is 1, 2, 3, 4, 5, 6, 7, 8, 9, or 10, inclusive of all subranges therebetween. In embodiments, e is 1, 2, 3, 4, 5, 6, 7, or 8. In embodiments, e is 1, 2, 3, or 4. In embodiments, e is 1.

In embodiments, at least a portion of the compound is cyclic. In embodiments, the CPP is cyclic. In embodiments, one or more of the CPPs and one or more of the cargos together form a ring, e.g., as indicated by the dashed lines in Formulas K, L, or M above. A linker may or may not form a portion of the ring structure (e.g., b may be 0 or 1 within the ring structure). In embodiments, a bond is formed between a group of the CPP and a group of the cargo. The cyclic portion may be monocyclic or bicyclic (e.g., as indicated in Formula Z-I or Z-II).

In embodiments, the compounds may have a structure according to any one of Formulas N or O, as follows:

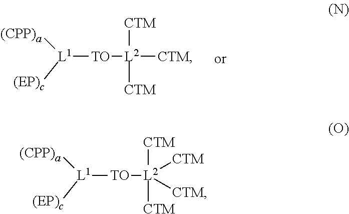

wherein
each CTM is independently a carbohydrate targeting moiety, and
CPP, EP, cargo, L¹, L², a, and c are as defined above.

In embodiments, a is an integer from 1 to 3. In embodiments, a is 1. In embodiments, c is 0. In embodiments, c is 1.

In embodiments, the compounds may have a structure according to Formula P, as follows:

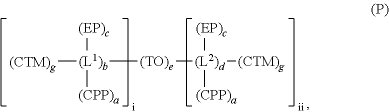

wherein
i and ii are each independently 0 to 10, provided that at least one of i or ii is 1 or greater,
each a, b, c, d, f, and g are each independently an integer from 0 to 10, provided that at least one a is 1 or greater and at least one g is 1 or greater,
e is an integer from 1 to 10,
each L¹ and each L² are each independently a linker,
each CPP is independently a cell penetrating peptide moiety; e.g., as defined above,
each EP is independently an exocyclic peptide; e.g., as defined above,
each cargo is independently a therapeutic oligonucleotide (TO); e.g., as defined above, and
each CTM is independently a carbohydrate targeting moiety; e.g., as defined above.

In embodiments, i is 1 and ii is 1. In embodiments, a is 1, 2, or 3. In embodiments, a is 1. In embodiments, b is 1 and f is 1. In embodiments, c is 0 or 1. In embodiments, c is 1. In embodiments, g is 3 or 4. In embodiments, g is 3.

L¹ or L² may be branched to may be branched to accommodate more than one cargo. L¹ or L² may be branched to accommodate collectively more than one of CPP, CTM, or EP.

In embodiments, one or more CPP is a cyclic CPP (cCPP).

In embodiments, one or more CTM comprises a GalNAc moiety.

In embodiments, the therapeutic oligonucleotide (TO) includes a small interfering RNA (siRNA), a microRNA (miRNA), a ribozyme, an immune stimulating nucleic acid, an antisense oligonucleotide (ASO), an antagomir, an antimir, a microRNA a mimic, a supermir, a Ul adaptor, an aptamer, or a guide RNA. In embodiments, the therapeutic oligonucleotide includes an antisense oligonucleotide (ASO). In embodiments, the ASO includes a nucleotide sequence complementary to a target nucleotide sequence.

In embodiments, the therapeutic oligonucleotide (TO) includes at least one modified nucleotide that includes a phosphorothioate (PS) nucleotide, a phosphorodiamidate morpholino nucleotide, a locked nucleic acid (LNA), a peptide nucleic acid (PNA), a nucleotide that includes a 2′-O-methyl (2′-OMe) modified backbone, a 2′O-methoxy-ethyl (2′-MOE) nucleotide, a 2′,4′ constrained ethyl (cEt) nucleotide, a 2′-deoxy-2′-fluoro-beta-D-arabinonucleic acid (2′F-ANA), or a combination thereof. In embodiments, the therapeutic oligonucleotide includes one or more phosphorodiamidate morpholino nucleosides, 2′-O-methylated nucleosides, locked nucleic acids (LNAs), or a combination thereof. In embodiments, the therapeutic oligonucleotide includes one or more phosphorodiamidate morpholino nucleosides.

In embodiments, the therapeutic oligonucleotide (TO) is from about 5 to about 1000, about 5 to about 500, about 5 to about 100, about 5 to about 50, about 5 to about 30, about 10 to about 30, about 15 to about 30, about 20 to about 30, about 5 to about 25, about 10 to about 25, about 15 to about 25, about 20 to about 25, about 5 to about 20, about 10 to about 20, or about 15 to about 20 nucleotides in length. In embodiments, the therapeutic oligonucleotide (TO) is 10, 11, 12, 13, 14, 15, 16, 17, 18, 19, 20, 21, 22, 23, 24, 25, 26, 27, 28, 29 or 30 nucleotides in length.

In embodiments, the therapeutic oligonucleotide (TO) includes an antisense oligonucleotide (ASO). In embodiments, the ASO includes a nucleotide sequence complementary to a target nucleotide sequence. The target nucleotide sequence may encode a mutant polypeptide or protein, or portion thereof. The mutant polypeptide or protein, or portion thereof may be associated with a disease.

In embodiments, the compound includes at least one CPP (e.g., 1, 2, 3, 4, 5, 6, 7, 8, 9, 10, or more CPPs), at least one therapeutic oligonucleotide (e.g., 1, 2, 3, 4, 5, 6, 7, 8, 9, 10, or more therapeutic oligonucleotides), and at least one CTM (e.g., 1, 2, 3, 4, 5, 6, 7, 8, 9, 10, or more CTMs). In embodiments, the compound may further comprise at least one EP (e.g., 1, 2, 3, 4, 5, 6, 7, 8, 9, 10, or more EPs).

In embodiments, the CPP may be coupled directly to one or more of the therapeutic oligonucleotide (TO), the EP, and the CTM. In embodiments, the CPP may be coupled to one or more of the TO, the EP, and the CTM via a linker.

In embodiments, the therapeutic oligonucleotide may be directly coupled to one or more of the CPP, the EP, and the CTM. In embodiments, the therapeutic oligonucleotide may be coupled to one or more of the CPP, the EP, and the CTM via a linker.

In embodiments, the EP may be directly coupled to one or more of the CPP, the therapeutic oligonucleotide, or the CTM. In embodiments, the EP may be coupled to one or more of the CPP, the therapeutic oligonucleotide, and the CTM via a linker.

In embodiments, the CTM may be directly coupled to one or more of the CPP, the therapeutic oligonucleotide, and the EP. In embodiments, the CTM may be coupled to one or more of the CPP, the therapeutic oligonucleotide, and the EP via a linker.

In embodiments, one or more CTMs (e.g., 1, 2, 3, 4, 5, 6, 7, 8, 9, or 10 CTMs) are coupled to the therapeutic oligonucleotide via a linker. In embodiments, two or more CTMs (e.g., 2, 3, 4, 5, 6, 7, 8, 9, or 10 CTMs) are coupled to the therapeutic oligonucleotide via a linker. In embodiments, three or more CTMs (e.g., 3, 4, 5, 6, 7, 8, 9, or 10 CTMs) are coupled to the therapeutic oligonucleotide via a linker. In embodiments, three CTMs are coupled to the therapeutic oligonucleotide via a linker. In embodiments, four CTMs are coupled to the therapeutic oligonucleotide via a linker.

In embodiments, one or more CTMs (e.g., 1, 2, 3, 4, 5, 6, 7, 8, 9, or 10 CTMs) are coupled to the therapeutic oligonucleotide via a first linker, and one or more CPPs (e.g., 1, 2, 3, 4, 5, 6, 7, 8, 9, or 10 CPPs) are coupled to the therapeutic oligonucleotide via a second linker. In embodiments, one or more CTMS (e.g., 1, 2, 3, 4, 5, 6, 7, 8, 9, or 10 CTMs) are coupled to the therapeutic oligonucleotide via a first linker, and one or more CPPs (e.g., 1, 2, 3, 4, 5, 6, 7, 8, 9, or 10 CPPs) and one or more EPs (e.g., 1, 2, 3, 4, 5, 6, 7, 8, 9, or 10 EPs) are coupled to the therapeutic oligonucleotide via a second linker. In embodiments, three CTMs are coupled to the therapeutic oligonucleotide via a first linker, and one CPP is coupled to the therapeutic oligonucleotide via a second linker. In embodiments, four CTMs are coupled to the therapeutic oligonucleotide via a first linker, and one CPP is coupled to the therapeutic oligonucleotide via a second linker. In embodiments, three CTMs are coupled to the therapeutic oligonucleotide via a first linker, and one CPP and one EP are coupled to the therapeutic oligonucleotide via a second linker. In embodiments, four CTMs are coupled to the therapeutic oligonucleotide via a first linker, and one CPP and one EP are coupled to the therapeutic oligonucleotide via a second linker.

As used herein, “coupled” refers to a covalent or non-covalent association between moieties of the compound, including fusion of the moieties and chemical conjugation of the moieties. A non-limiting example of a means to non-covalently attach the moieties is through the interaction of streptavidin/biotin, e.g., by conjugating biotin to one moiety and fusing another moiety to streptavidin. In the resulting compound, the one moiety is coupled to the other moiety via a non-covalent association between biotin and streptavidin. The moieties may be coupled to one another, directly or indirectly, through any appropriate site on either of these moieties.

In embodiments, one or more moieties of the compound may be conjugated, directly or indirectly, to a chemically reactive side chain of an amino acid of the CPP or the EP. Any amino acid side chain on the CPP or EP that is capable of forming a covalent bond, or which may be so modified, can be used to directly or indirectly couple the therapeutic oligonucleotide (TO), the CTM to the CPP or the EP. The amino acid on the CPP or the EP can be a natural or non-natural amino acid. In embodiments, the chemically reactive side chain includes an amine group, a carboxylic acid group, an amide group, a hydroxyl group, a sulfhydryl group, a guanidinyl group, a phenolic group, a thioether group, an imidazolyl group, or an indolyl group. In embodiments, the amino acid of the CPP or EP to which a moiety is directly or indirectly coupled includes lysine, arginine, aspartic acid, glutamic acid, asparagine, glutamine, serine, threonine, tyrosine, cysteine, arginine, tyrosine, methionine, histidine, tryptophan or analogs thereof. In embodiments, the amino acid on the CPP or EP used to directly or indirectly couple the moiety is ornithine, 2,3-diaminopropionic acid, or analogs thereof. In embodiments, the amino acid is lysine, or an analog thereof. In embodiments, the amino acid is glutamic acid, or an analog thereof. In embodiments, the amino acid is aspartic acid, or an analog thereof. In embodiments, the amino acid on the CPP or EP used to directly or indirectly couple the therapeutic oligonucleotide (TO) is glutamine. In embodiments, the side chain is substituted with a bond to the moiety or a linker.

In embodiments, the compounds disclosed herein have a structure according to Formula Q, Q¹ or Q²

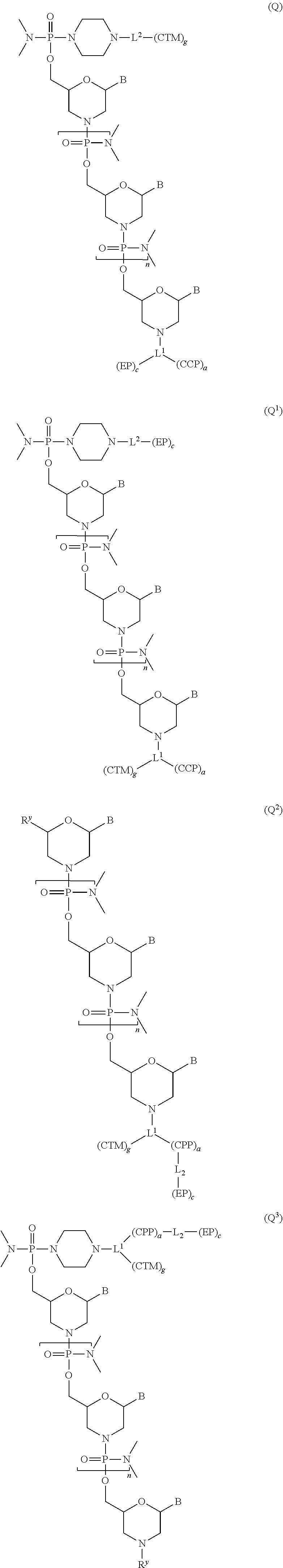

wherein:
CPP is a cell penetrating peptide;
EP is an exocyclic peptide,
CTM is a carbohydrate targeting moiety,
a is an integer from 1 to 10;
c is an integer from 0 to 10;
g is an integer from 1 to 10;
L¹ is a linker;
L² is a linker;
L³ is a linker;
R^{y} is H or —CH₂OR^{z};
R^{y} is a capping group;
B is each independently a nucleobase of the therapeutic oligonucleotide; and
n is an integer from 1 to 1000.

In embodiments, n is an integer from 5 to 1000, 5 to 500, 5 to 100, 5 to 50, 5 to 30, 10 to 30, 15 to 30, 20 to 30, 5 to 25, 10 to 25, 15 to 25, 20 to 25, 5 to 20, 10 to 20, or 15 to 20. In embodiments, n is an integer from 5 to 500. In embodiments, n is an integer from 5 to 50. In embodiments, n is an integer from 15 to 30. In embodiments, n is 10, 11, 12, 13, 14, 15, 16, 17, 18, 19, 20, 21, 22, 23, 24, 25, 26, 27, 28, 29 or 30.

In embodiments of the compound of Formula Q, Q¹ or Q², g is an integer from 1 to 9 (e.g., 1, 2, 3, 4, 5, 6, 7, 8, or 9, inclusive or all subranges therebetween). In embodiments, g is an integer from 1 to 4. In embodiments, g is 3. In embodiments, g is 4.

In embodiments of the compound of Formula Q, Q¹ or Q², a is an integer from 1 to 9 (e.g., 1, 2, 3, 4, 5, 6, 7, 8, or 9, inclusive or all subranges therebetween). In embodiments, a is an integer from 1 to 3. In embodiments, a is 1.

In embodiments of the compound of Formula Q, Q¹ or Q², C is an integer from 0 to 9 (e.g., 0, 1, 2, 3, 4, 5, 6, 7, 8, or 9, inclusive or all subranges therebetween). In embodiments, c is 0 or 1. In embodiments, c is 1.

In embodiments of the compound of Formula Q, Q¹ or Q², g is 3, a is 1, c is 0, and n is about 5 to about 500. In embodiments, g is 3, a is 1, c is 1, and n is about 5 to about 500. In embodiments, g is 3, a is 1, c is 0, and n is about 5 to about 50. In embodiments, g is 3, a is 1, c is 1, and n is about 5 to about 50.

In embodiments, CPP is a cCPP.

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a therapeutic moiety (e.g., an oligonucleotide, peptide or small molecule) to penetrate the membrane of a cell. The cCPP can deliver the therapeutic moiety to the cytosol of the cell. The cCPP can deliver the cargo to a cellular location where a target (e.g., pre-mRNA) is located. To conjugate the cCPP to a therapeutic moiety (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

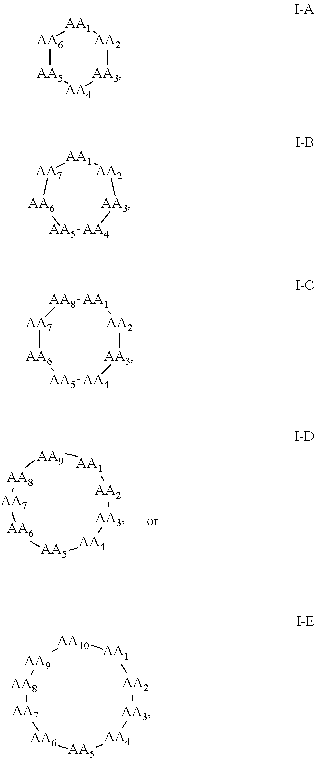

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1

| | Abbreviations* | Abbreviations* |
|---|---|---|
| Amino Acid | L-amino acid | D-amino acid |
| Alanine | Ala (A) | ala (a) |
| Allo-isoleucine | Aile | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| 3-(2-naphthyl)-alanine | Nal (Φ) | nal (ϕ) |
| 3-(1-naphthyl)-alanine | 1-Nal | 1-nal |
| Norleucine | Nle (Ω) | nle |
| Phenylalanine | Phe (F) | phe (f) |
| Phenylglycine | Phg (Ψ) | phg |
| 4-(phosphonodifluoromethyl) | F₂Pmp (Λ) | f₂pmp |
| phenylalanine | | |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: capital letters indicate the L-amino acid form, lower case letter indicate the D-amino acid form.

The cCPP can comprise 4 to 20 amino acids, wherein: (i) at least one amino acid has a side chain comprising a guanidine group, or a protonated form thereof; (ii) at least one amino acid has no side chain or a side chain comprising

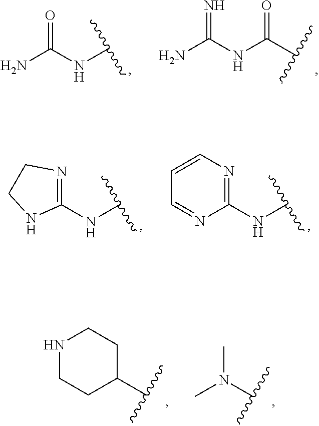

or a protonated form thereof; and (iii) at least two amino acids independently have a side chain comprising an aromatic or heteroaromatic group.

At least two amino acids can have no side chain or a side chain comprising

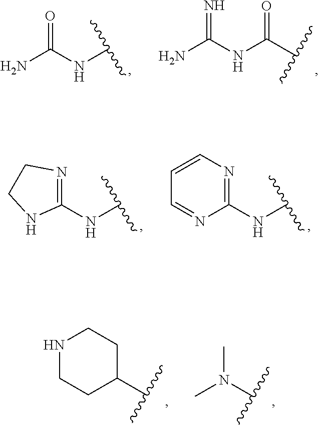

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

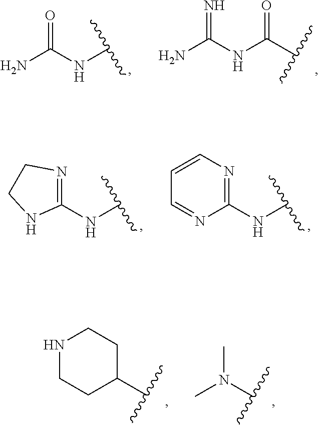

or a protonated form thereof.

The cCPP can comprise from 6 to 20 amino acid residues which form the cCPP, wherein: (i) at least two amino acid can independently beglycine, β-alanine, or 4-aminobutyric acid residues; (ii) at least one amino acid can have a side chain comprising an aryl or heteroaryl group; and (iii) at least one amino acid has a side chain comprising a guanidine group,

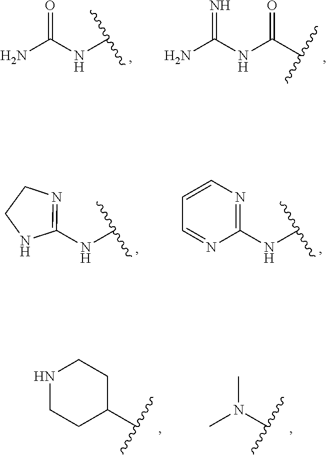

or a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

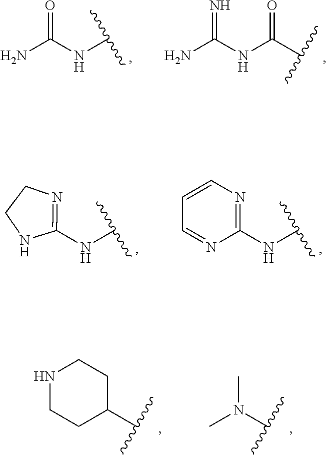

or a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, 3-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous.

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

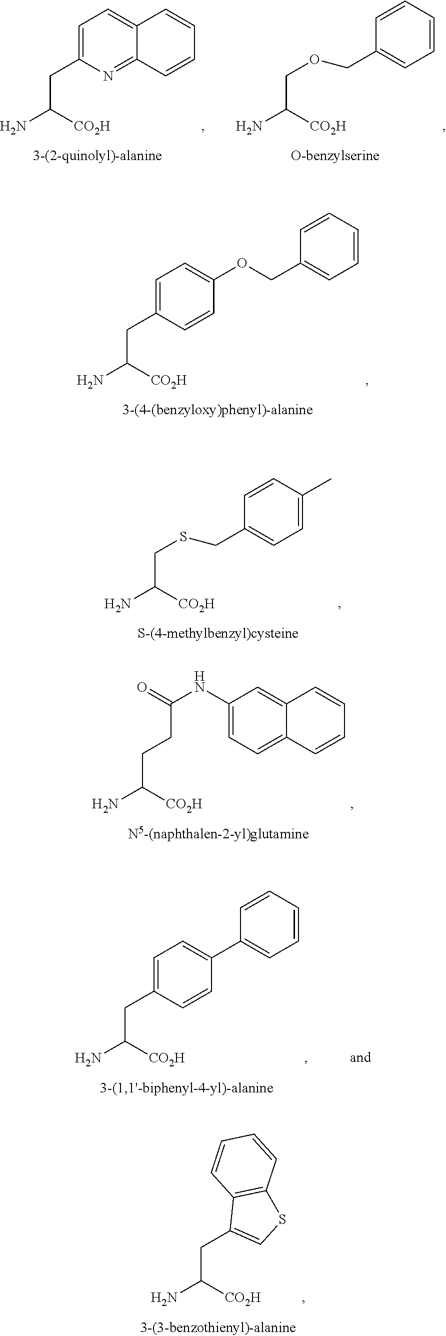

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, R-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 2 lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 2
Amino Acid Hydrophobicity

| | | Eisenberg | Engleman | Kyrie and | Hoop and | |
|---|---|---|---|---|---|---|
| Amino Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif; a cCPP having a Phe-Nal-Arg motif may exhibit improved cytosolic delivery efficiency compared to an otherwise identical cCPP having a Nal-Phe-Arg motif; and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Ångstroms; Å²) of an amino acid side chain that is accessible to a solvent, SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. The SASA values described herein are based on the theoretical values listed in Table 3 below, as reported by Tien, et al. (PLOS ONE 8(11): e80635 avlaible at doi.org/10.1371/journal.pone.0080635), which is herein incorporated by reference in its entirety for all purposes.

TABLE 3
Amino Acid SASA Values

| | | | Miller | Rose |
|---|---|---|---|---|
| Residue | Theoretical | Empirical | et al. (1987) | et al. (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

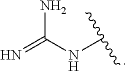 [SMILES: *NC(=N)N]

As used herein, a protonated form of guanidine refers to the structure:

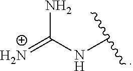 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

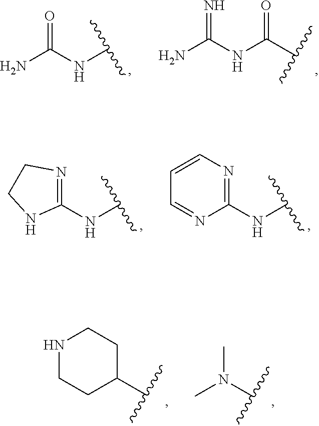

or a protonated form thereof.

The disclosure relates to a cCPP comprising from 4 to 20 amino acids residues, wherein: (i) at least one amino acid has a side chain comprising a guanidine group, or a protonated form thereof; (ii) at least one amino acid residue has no side chain or a side chain comprising

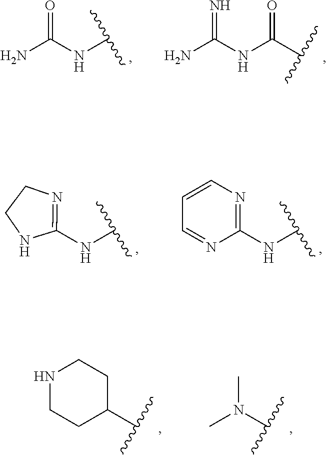

or a protonated form thereof, and (iii) at least two amino acids residues independently have a side chain comprising an aromatic or heteroaromatic group.

At least two amino acids residues can have no side chain or a side chain comprising

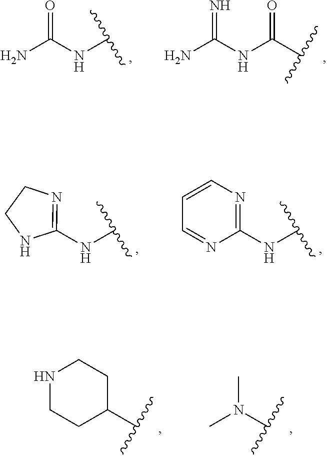

or a protonated form thereof. As used herein, when no side chain is present, the amino acid residues have two hydrogen atoms on the carbon atom(s) (e.g., —CH₂—) linking the amine and carboxylic acid.

The cCPP can comprise at least one amino acid having a side chain comprising one of the following moieties:

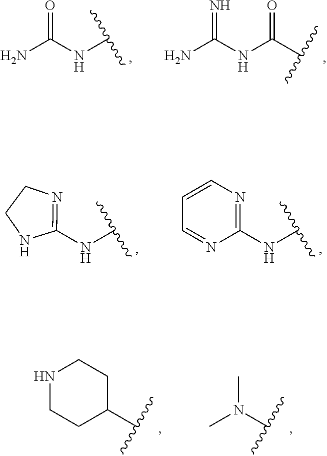

or a protonated form thereof.

The cCPP can comprise at least two amino acids each independently having one of the following moieties

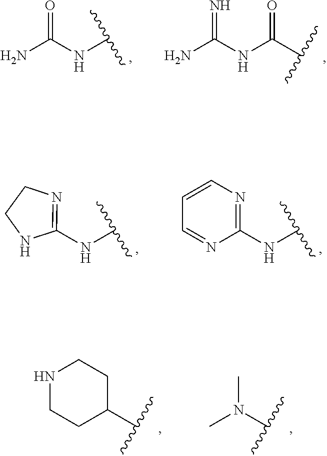

or a protonated form thereof. At least two amino acids can have a side chain comprising the same moiety selected from:

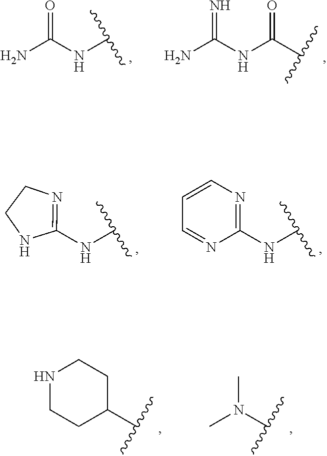

or a protonated form thereof. At least one amino acid can have a side chain comprising

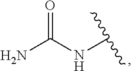 [SMILES: *NC(N)=O]

or a protonated form thereof. At least two amino acids can have a side chain comprising

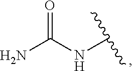 [SMILES: *NC(N)=O]

or a protonated form thereof. One, two, three, or four amino acids can have a side chain comprising

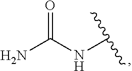 [SMILES: *NC(N)=O]

or a protonated form thereof. One amino acid can have a side chain comprising

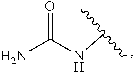 [SMILES: *NC(N)=O]

or a protonated form thereof. Two amino acids can have a side chain comprising

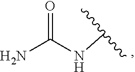 [SMILES: *NC(N)=O]

or a protonated form thereof.

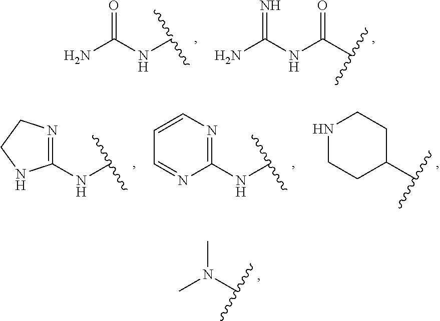

or a protonated form thereof, can be attached to the terminus of the amino acid side chain.

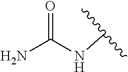 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

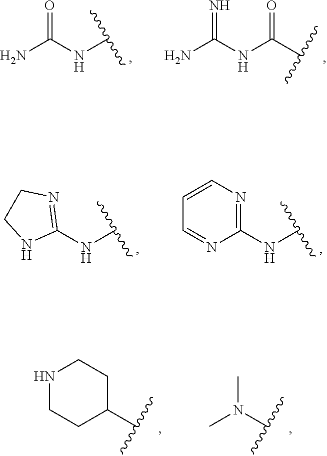

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamine acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

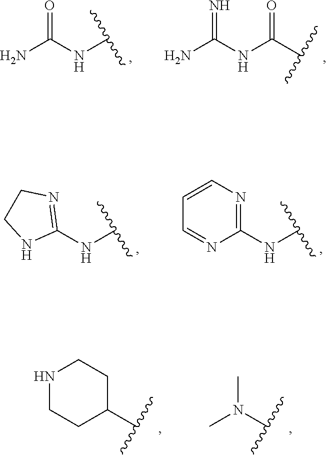

or a protonated form thereof, can be adjacent to an amino acid having a side chain comprising an aromatic or heteroaromatic group. An amino acid having a side chain comprising:

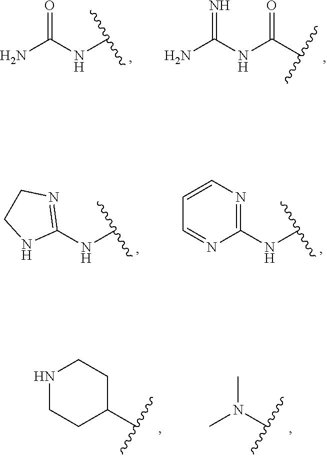

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

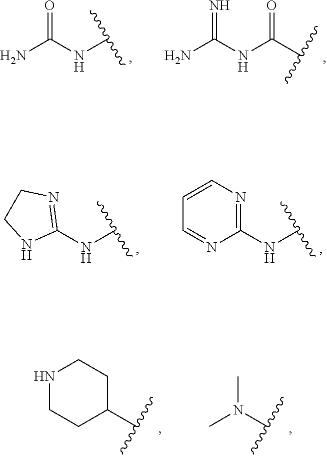

or protonated forms there, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

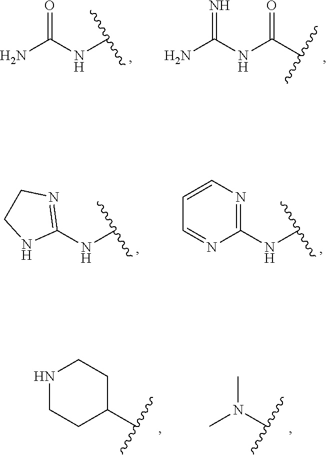

or a protonated form thereof. The cCPPs can comprise at least two contiguous amino acids having a side chain comprising an aromatic or heteroaromatic group and at least two non-adjacent amino acids having a side chain comprising

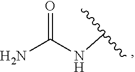 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

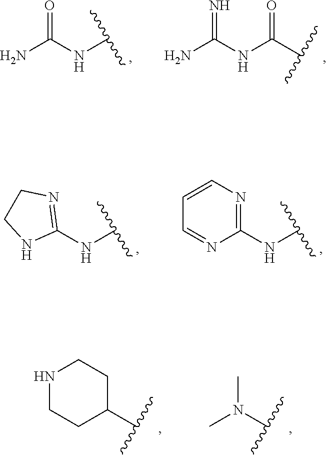

protonated form thereof, are alternating with at least two amino acids having a side chain comprising a guanidine group or protonated form thereof.

The cCPP can comprise the structure of Formula (Q):

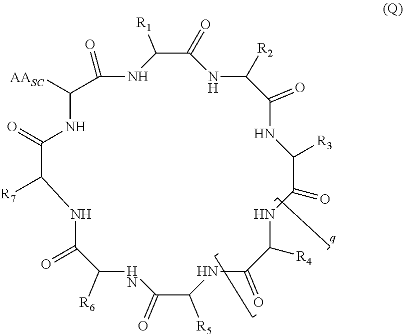

or a protonated form thereof,
wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, si-homoarginine, 3-(1-piperidinyl)alanine;
AA_{SC} is an amino acid side chain; and
q is 1, 2, 3 or 4.

In embodiments, at least one of R₄, R₅, R₆, R₇ are independently a uncharged, non-aromatic side chain of an amino acid. In embodiments, at least one of R₄, R₅, R₆, R₇ are independently H or a side chain of citrulline.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine, naphtha alanine (3-Naphth-2-yl-alanine) or a combination thereof. In embodiments, at least two uncharged, non-aromatic amino acids of the cyclic peptide are citrulline, glycine or a combination thereof. In embodiments, the compound is a cyclic peptide having 6 to 12 amino acids wherein two amino acids of the cyclic peptide are arginine, at least two amino acids are aromatic, hydrophobic amino acids selected from phenylalanine, naphtha alanine and combinations thereof, and at least two amino acids are uncharged, non-aromatic amino acids selected from citrulline, glycine and combinations thereof.

In embodiments, the cyclic peptide of Formula (Q) is not a cyclic peptide having a sequence of:

CPP sequences

| | | |
|---|---|---|
| FΦRRRQ | RRFRΦRQ | FΦRRRRQK |
| FΦRRRC | FRRRRΦQ | FΦRRRRQC |
| FΦRRRU | rRFRΦRQ | fΦRrRrRQ |
| RRRΦFQ | RRΦFRRQ | FΦRRRRRQ |
| RRRRΦF | CRRRRFWQ | RRRRΦFDΩC |
| FΦRRRR | FfΦRrRrQ | FΦRRR |
| FϕrRrRq | FFΦRRRRQ | FWRRR |
| FϕrRrRQ | RFRFRΦRQ | RRRΦF |
| FΦRRRRQ | URRRRFWQ | RRRWF |
| fΦRrRrQ | CRRRRFWQ | |

where F is L-phenylalanine, f is D-phenylalanine, Φ is L-3-(2-naphthyl)-alanine, Φ is D-3-(2-naphthyl)-alanine, R is L-arginine, r is D-arginine, Q is L-glutamine, q is D-glutamine, C is L-cysteine, U is L-selenocysteine, W is L-tryptophan, K is L-lysine, D is L-aspartic acid, and Ω is L-norleucine.

The cCPP can comprise the structure of Formula (I):

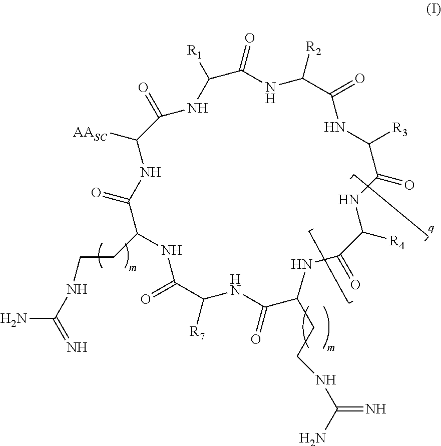

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₇ are independently H or an amino acid side chain;
AA_{SC} is an amino acid side chain;
q is 1, 2, 3 or 4; and
each m is independently an integer 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₁ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₁ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 1, Table 2 or Table 3. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃ alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₁ can be H or the side chain of an amino acid in Table 1, Table 2 or Table 3. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃ alkylene-heteroaryl. R_{f} can be H or -alkylene-aryl. R, can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 1, Table 2 or Table 3. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R, can be H or the side chain of an amino acid in Table 1, Table 2 or Table 3. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R, can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (Q) can comprise the structure of Formula (I)

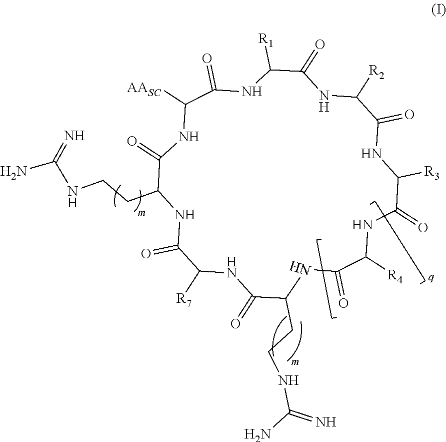

or protonated form thereof, wherein AA_{SC}, R₁, R₂, R₃, R₄, R₇, m and q are as defined herein

The cCPP of Formula (Q) can comprise the structure of Formula (I-a) or Formula (I-b):

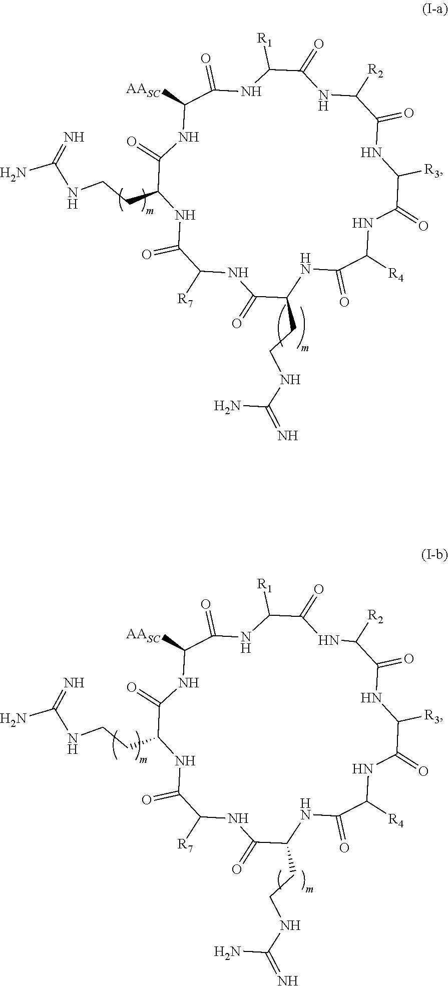

or protonated form thereof, wherein AA_{SC}, R₁, R₂, R₃, R₄, and m are as defined herein.

The cCPP of Formula (Q) can comprise the structure of Formula (I-1), (I-2), (I-3) or (I-4):

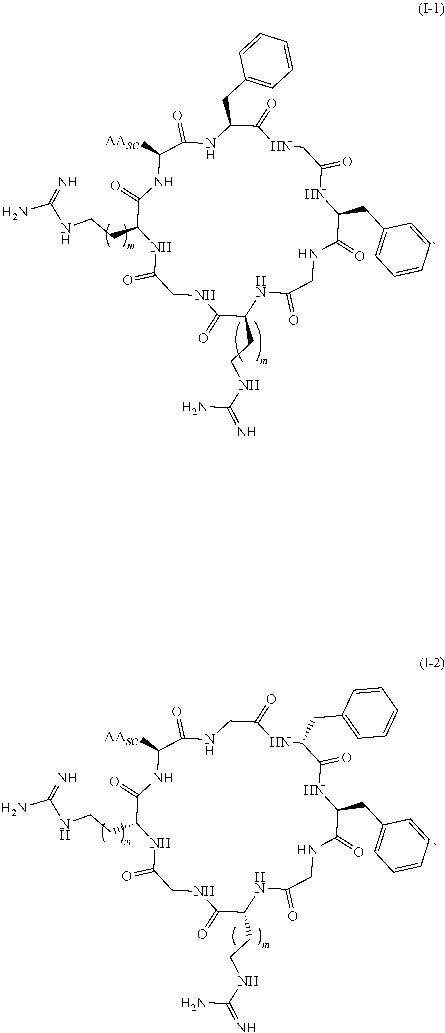

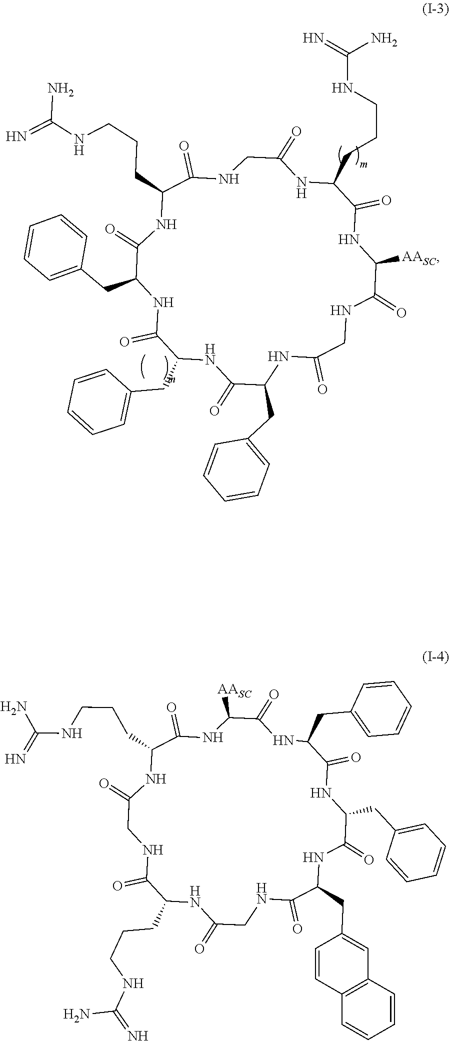

or protonated form thereof, wherein AA_{SC} and m are as defined herein.

The cCPP of Formula (Q) can comprise the structure of Formula (I-5) or (I-6):

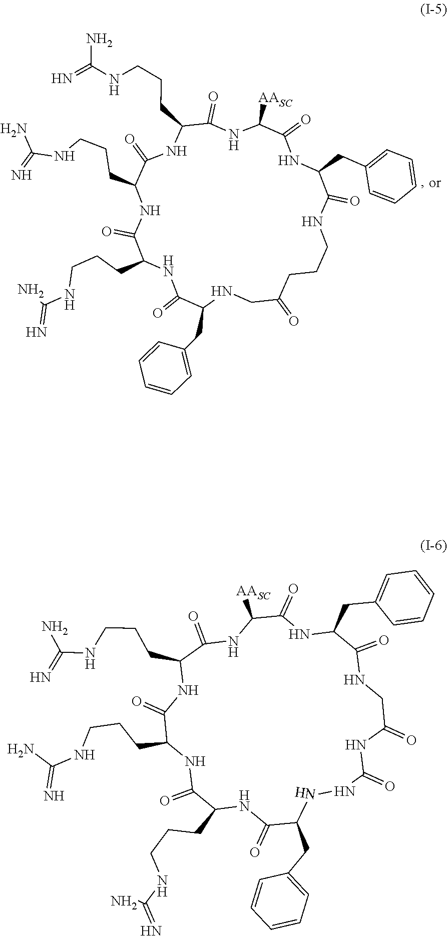

or protonated form thereof, wherein AA_{SC} is as defined herein.

The cCPP can comprise one of the following sequences: FGFGRGR; GfFGrGr, FfΦGRGR; FfFGRGR; or FfΦGrGr. The cCPP can have one of the following sequences: FGFGRGRQ; GfFGrGrQ, FfΦGRGRQ; FfFGRGRQ; or FfΦGrGrQ.

The disclosure also relates to a cCPP having the structure of Formula (II):

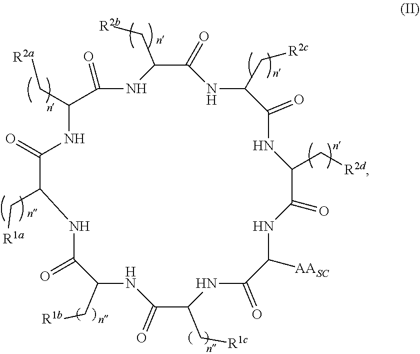

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

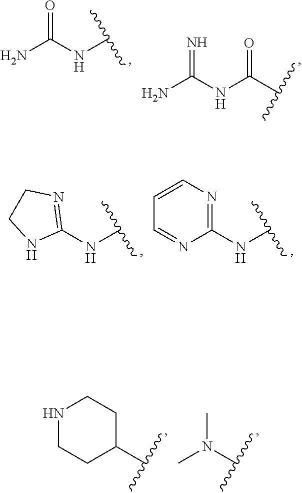

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2b} or R^{2d} is absent.

At least two of R^{2a}, R^{2b}, R^{2c} and R^{2d} can be

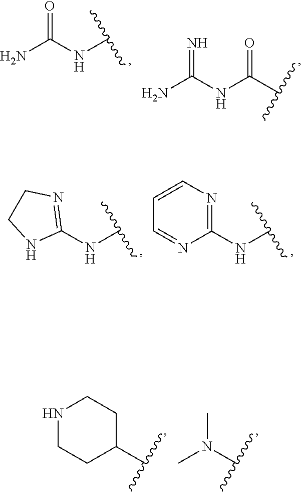

or a protonated form thereof. Two or three of R^{2a}, R^{2b}, R^{2c} and R^{2d} can be

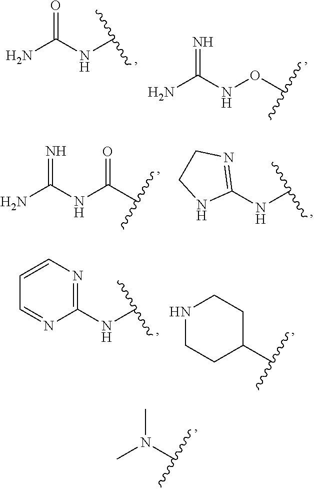

or a protonated form thereof. One of R^{2a}, R^{2b}, R^{2c} and R^{2d} can be

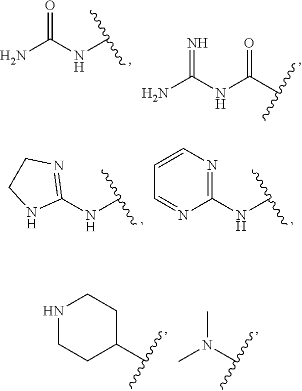

or a protonated form thereof. At least one of R^{2a}, R^{2b}, R^{2c} and R^{2d} can be

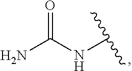 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

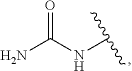 [SMILES: *NC(N)=O]

a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

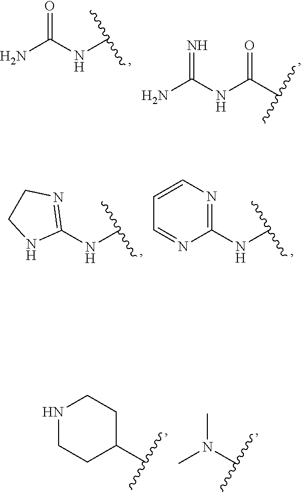

or a protonated form thereof. At least or R^{2a}, R^{2b}, R^{2c} and R^{2d} can be

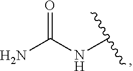 [SMILES: *NC(N)=O]

or a protonated form thereof, and the remaining of R^{2a}, R^{2b}, R^{2c} and R^{2d} can be guaninide or a protonated form thereof. At least two R^{2a}, R^{2b}, R^{2c} and R^{2d} groups can be

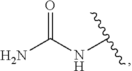 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

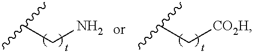

wherein t can be an integer from 0 to 5. AA_{SC} can be

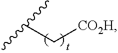

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

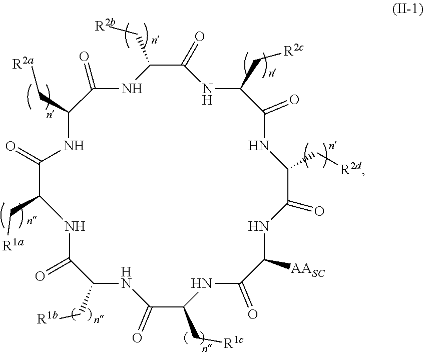

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

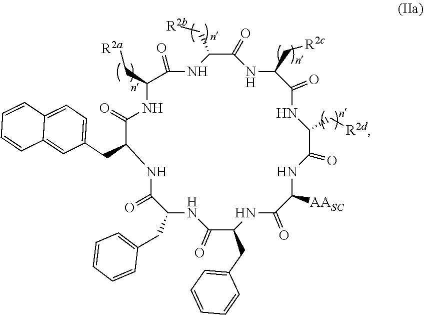

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

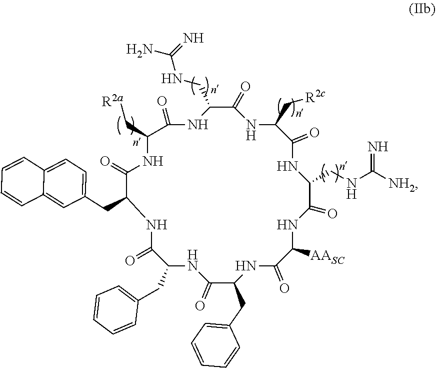

wherein R^{2a}, R^{2b}, AA_{SC}, and n′ are as defined herein.

The cCPP can have the structure of Formula (IIc):

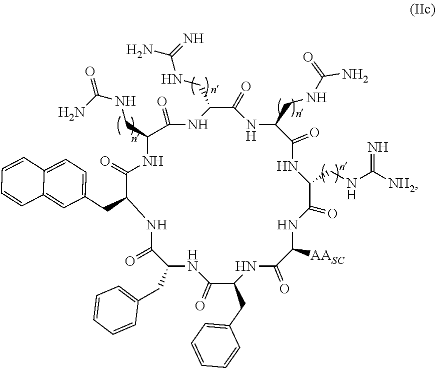

or a protonated form thereof, wherein:
AA_{SC} and n′ are as defined herein.

The cCPP can have the structure of Formula (I):

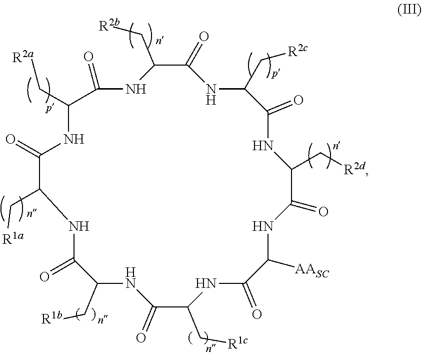

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

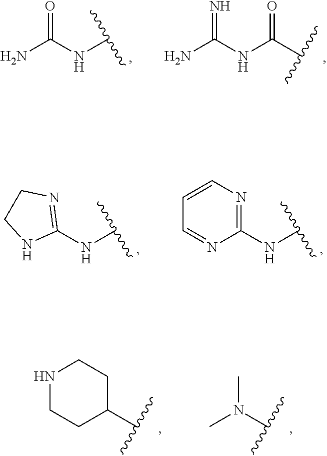

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

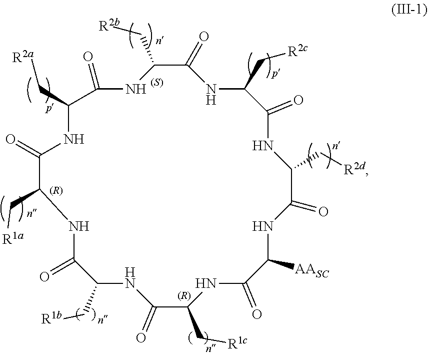

wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

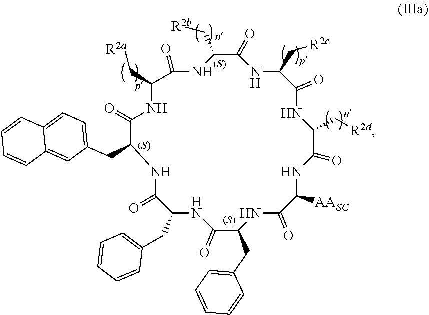

wherein:
AA_{SC}, R^{2a}, R^{2c}, R^{2b}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. W can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

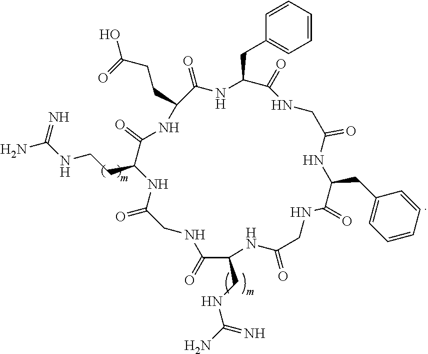

The cCPP of Formula (Q) can be selected from:

CPP Sequence

(FfΦRrRrQ)
(FfΦCit-r-Cit-rQ)
(FfΦGrGrQ)
(FfFGRGRQ)
(FGFGRGRQ)
(GfFGrGrQ)
(FGFGRRRQ)
(FGFRRRRQ)

The cCPP of Formula (Q) can be selected from:

CPP Sequence

FΦRRRRQ
fΦRrRrQ
FfΦRrRrQ
FfΦCit-r-Cit-rQ
FfΦGrGrQ
FfΦRGRGQ
FfFGRGRQ
FGFGRGRQ
GfFGrGrQ
FGFGRRRQ
FGFRRRRQ

In embodiments the cCPP is selected from:

| CPP sequence | CPP sequence | CPP sequence |
|---|---|---|
| FΦRRRQ | FRRRRΦQ | CRRRRFWQ |
| FΦRRRC | rRFRΦRQ | FΦRRRRQK |
| FΦRRRU | RRΦFRRQ | FΦRRRRQC |
| RRRΦFQ | CRRRRFWQ | fΦRrRrRQ |
| RRRRΦF | FfΦRrRrQ | FΦRRRRRQ |
| FΦRRRR | FFΦRRRRQ | RRRRΦFDΩC |
| FϕrRrRq | RFRFRΦRQ | FΦRRR |
| FϕrRrRQ | URRRRFWQ | FWRRR |
| FΦRRRRO | RRFRΦRQ | RRRΦF |
| fΦRrRrQ | FRRRRΦQ | RRRWF |
| RRFRΦRQ | | |

Where Φ=L-naphthylalanine; ϕ=D-naphthylalanine; Ω=L-norleucine

In embodiments, the cCPP is not selected from:

| CPP sequence | CPP sequence | CPP sequence |
|---|---|---|
| FΦRRRQ | FRRRRΦQ | CRRRRFWQ |
| FΦRRRC | rRFRΦRQ | FΦRRRRQK |
| FΦRRRU | RRΦFRRQ | FΦRRRRQC |
| RRRΦFQ | CRRRRFWQ | fΦRrRrRQ |
| RRRRΦF | FfΦRrRrQ | FΦRRRRRQ |
| FΦRRRR | FFΦRRRRQ | RRRRΦFDΩC |
| FϕrRrRq | RFRFRΦRQ | FΦRRR |
| FϕrRrRQ | URRRRFWQ | FWRRR |
| FΦRRRRQ | RRFRΦRQ | RRRΦF |
| fΦRrRrQ | FRRRRΦQ | RRRWF |
| RRFRΦRQ | | |

Where Φ=L-naphthylalanine; ϕ=D-naphthylalanine; Ω=L-norleucine

The cCPP can comprise the structure of Formula (R)

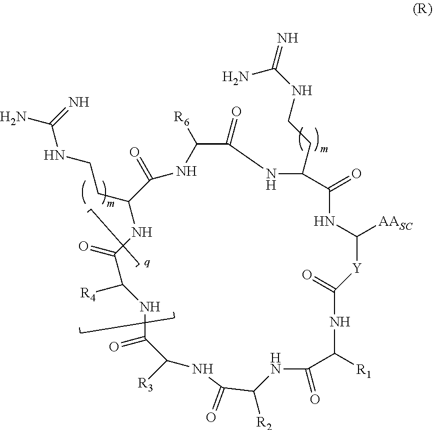

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
Y is

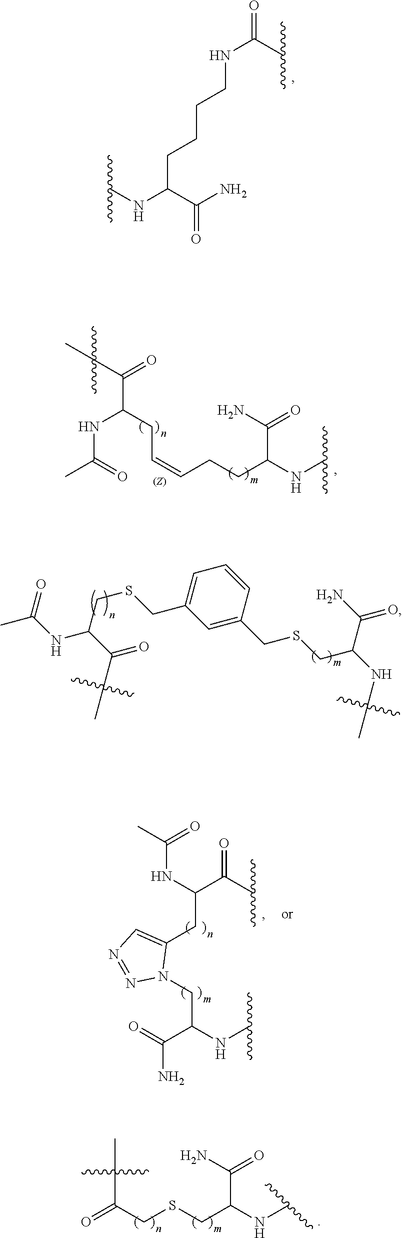

q is 1, 2, 3 or 4;
each m is independently an integer 0, 1, 2, or 3, and
each n is independently an integer 0, 1, 2, or 3.

The cCPP of Formula (R), wherein Y is

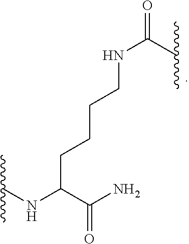 [SMILES: *NC(CCCCNC(*)=O)C(N)=O]

The cCPP of Formula (R), wherein Y is:

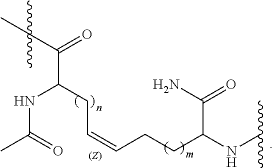

The cCPP of Formula (R), wherein Y is:

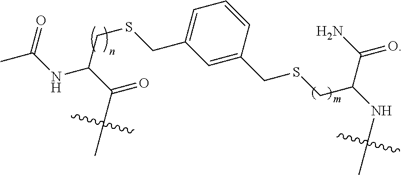

The cCPP of Formula (R), wherein Y is:

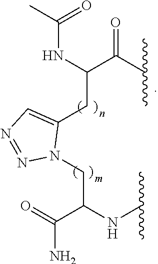

The cCPP of Formula (R), wherein Y is

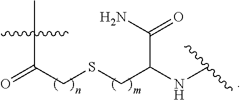

In embodiments, AA_{SC} can be conjugated to a linker.

Additionally, the cCPP used in the compounds and methods described herein can include any sequence disclosed in: U.S. Pat. Nos. 10,626,147; 10,815,276; International PCT Application Publication No. WO/2018/089648 (including the corresponding US publication), and International PCT Application Publication No. WO 2018/098231, each of which is incorporated by reference in its entirety for all purposes.

### Linker

The cCPP of the disclosure can be conjugated to a linker. The linker can link a therapeutic moiety to the cCPP. The linker can be attached to the side chain of an amino acid of the cCPP, and the therapeutic oligonucleotide can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the therapeutic moiety is an oligonucleotide, the linker can be covalently bound to the 5′ end of the cargo or the 3′ end of the cargo. The linker can be covalently bound to the 5′ end of the therapeutic moiety. The linker can be covalently bound to the 3′ end of the therapeutic moiety. If the cargo is a peptide, the linker can be covalently bound to the N-terminus or the C-terminus of the therapeutic moiety. The linker can be covalently bound to the backbone of the oligonucleotide or peptide therapeutic moiety. The linker can be any appropriate moiety which conjugates a cCPP described herein to a therapeutic moiety such as an oligonucleotide, peptide or small molecule.

In embodiments, the 5′ end, the 3′ end, the backbone, or a nucleobase of the TO moiety is directly or indirectly (e.g., through a linker) to a chemically reactive side chain of an amino acid of the CPP. In embodiments, the therapeutic oligonucleotide (TO) is chemically conjugated to the CPP or to a linker through a moiety on the 5′ or 3′ end of the therapeutic oligonucleotide (TO).

In embodiments, the TO moiety is covalently linked to the CPP. Such conjugates may alternatively be described as having a cell penetrating moiety and a TO moiety. A covalently-linked TO moiety-CPP conjugate, in accordance with certain embodiments, includes the TO moiety component and a cyclic or linear CPP component associated with one another by a linker (L). The linker (L) may include a bonding group (M).

In embodiments where the compounds include a linker (L), the linker (L) conjugates the CPP to the TO moiety. In embodiments, the linker (L) conjugates the TO moiety to an amino acid side chain of the CPP. In embodiments, the linker (L) conjugates the CPP to the 5′ end, the 3′ end, or a nucleobase of the TO moiety.

In embodiments, compounds that include a TO moiety and CPP may also include an exocyclic peptide (EP), for example, a nuclear localization sequence (NLS). In embodiments, the EP is coupled to the TO moiety. In embodiments, the EP is coupled to the CPP. In embodiments, the EP is coupled to the TO moiety and the CPP. Coupling between the EP, TO moiety, CPP, or combinations thereof, may be non-covalent or covalent. In embodiments, the EP is attached through a peptide bond to the N-terminus of the CPP. In embodiments, the EP is attached through a peptide bond to the C-terminus of the CPP. In embodiments, the EP is attached to the CPP through a side chain of an amino acid in the CPP. In embodiments, the EP is attached to the CPP through a side chain of a lysine which is conjugated to the side chain of a glutamine in the CPP. In embodiments, the EP is conjugated to the 5′ end, 3′ end, or a nucleobase of the TO moiety. In embodiments, the EP is coupled to the TO moiety or the CPP via a linker. In embodiments, the C-terminus of the EP is coupled to the CPP or TO moiety through an amino acid side chain on the CPP or EP. For example, an EP may include a terminal lysine which is then coupled to a CPP containing a glutamine through an amide bond. When the EP contains a terminal lysine, and the side chain of the lysine is used to attach the CPP, the C- or N-terminus of the EP may be attached to the linker coupled to the TO moiety.

L may be any appropriate moiety which conjugates CPP (e.g., as described herein) to a TO moiety. Thus, prior to conjugation to the CPP and TO, the linker may have two or more functional groups, each of which are independently capable of forming a covalent bond to the CPP moiety and the TO moiety, or alternatively one or both of the CPP and the TO moiety are modified to include functional groups that are capable of forming a bond to the linker. In embodiments, L is covalently bound to the 5′ end, the 3′ end, or a nucleobase of the TO moiety. In embodiments, L is covalently bound to the 5′ end of the TO or the 3′ end of the TO moiety. In embodiments, L is covalently bound to the 5′ end of the TO moiety. In other embodiments, L is covalently bound to the 3′ end of the TO moiety. In still other embodiments, L is covalently bound to a nucleobase of the TO moiety.

In embodiments, L is covalently bound to a nucleophilic moiety on the therapeutic oligonucleotide (TO). In embodiments, the nucleophilic moiety is conjugated to the TO moiety so that the therapeutic oligonucleotide (TO) can be attached to the CPP through L. In embodiments, L is covalently bound to a piperazine moiety on the TO moiety. In embodiments, L is covalently bound to a side chain or terminus of an amino acid on the CPP. In certain embodiments, L is covalently bound to the side chain of an amino acid on the CPP.

The linker can comprise hydrocarbon linker.

The linker can comprise a cleavage site. The cleavage site can be a disulfide, or caspase-cleavage site (e.g, Val-Cit-PABC).

The linker may be any appropriate moiety which conjugates a cyclic peptide described herein to one or more additional moieties, e.g., an exocyclic cyclic sequence, a CTM, a TO moiety, or one or more of an exocyclic cyclic sequence, a CTM, and a TO moiety. Thus, prior to conjugation to the cyclic peptide and additional moiety or moieties, the linker has two or more functional groups, each of which are independently capable of forming a covalent bond to the cyclic peptide and one or more additional moieties. In various embodiments, the linker is covalently bound to the 5′ end, the 3′ end, a nucleobase, or a backbone of the TO moiety. For example, the linker may be covalently bound to the 5′ end or the 3′ end of the TO moiety. In embodiments, the linker is covalently bound to the 5′ end of the TO moiety. In other embodiments, the linker is covalently bound to the 3′ end of the TO moiety. In still other embodiments, the linker is covalently bound to the backbone of the TO moiety. In other embodiments, the linker is covalently bound to a nucleobase of the TO moiety. In embodiments, the linker is any appropriate moiety which conjugates a cyclic peptide described herein to a TO moiety.

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹⁻J-R²)z″-subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R₃ is independently selected from H, alkyl, alkenyl, alkynyl, carbocyclyl, and heterocyclyl, each of which is optionally substituted, and z″ is an integer from 1 to 50; (viii) —(R¹⁻J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R₃ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹⁻J-R₂)z″-, wherein each of R₁ and R₂, at each instance, are independently alkylene, each J is independently C, NR³, —NR³C(O)—, S, and O, wherein R; is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R₃ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹⁻-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

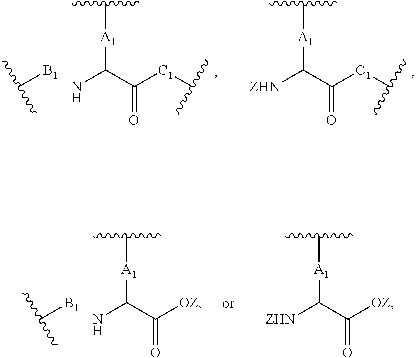

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

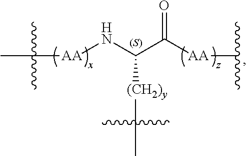

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

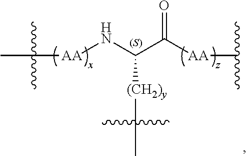

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

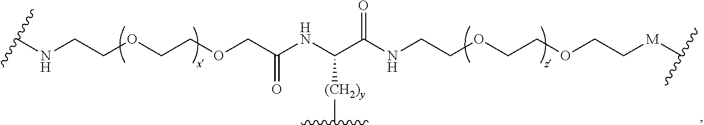

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

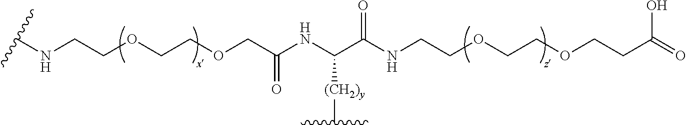

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

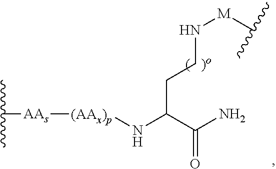

wherein
M is a group that conjugates L to a cargo, for example, an oligonucleotide;
AAₛ is a side chain or terminus of an amino acid on the cCPP;
each AAₛ is independently an amino acid residue;
is an integer from 0 to 10; and
p is an integer from 0 to 5.

The linker can have a structure:

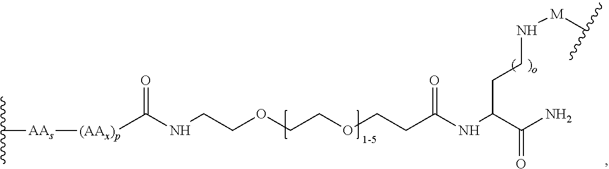

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

M may be covalently bound to the TO moiety at any suitable location on the TO moiety. In embodiments, M is covalently bound to a nucleophilic moiety on the TO moiety. In embodiments, the nucleophilic moiety is a nitrogen-containing moiety. In embodiments, M is covalently bound to a piperazine moiety of the TO moiety.

M can comprise an alkylene, alkenylene, alkynylene, carbocyclyl, or heterocyclyl, each of which is optionally substituted. M can be selected from:

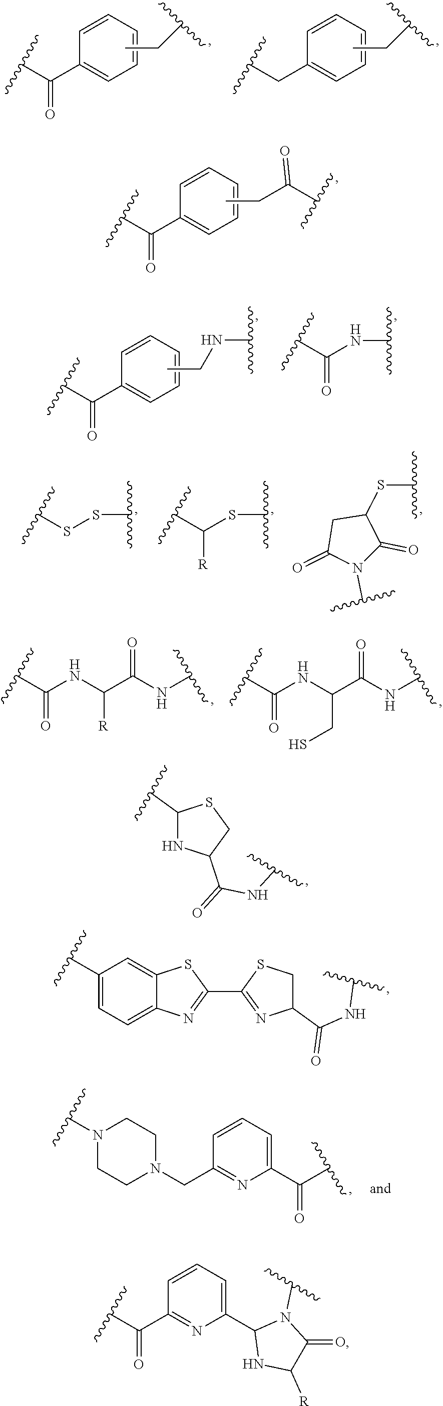

wherein R is alkyl, alkenyl, alkynyl, carbocyclyl, or heterocyclyl.

M can be selected from:

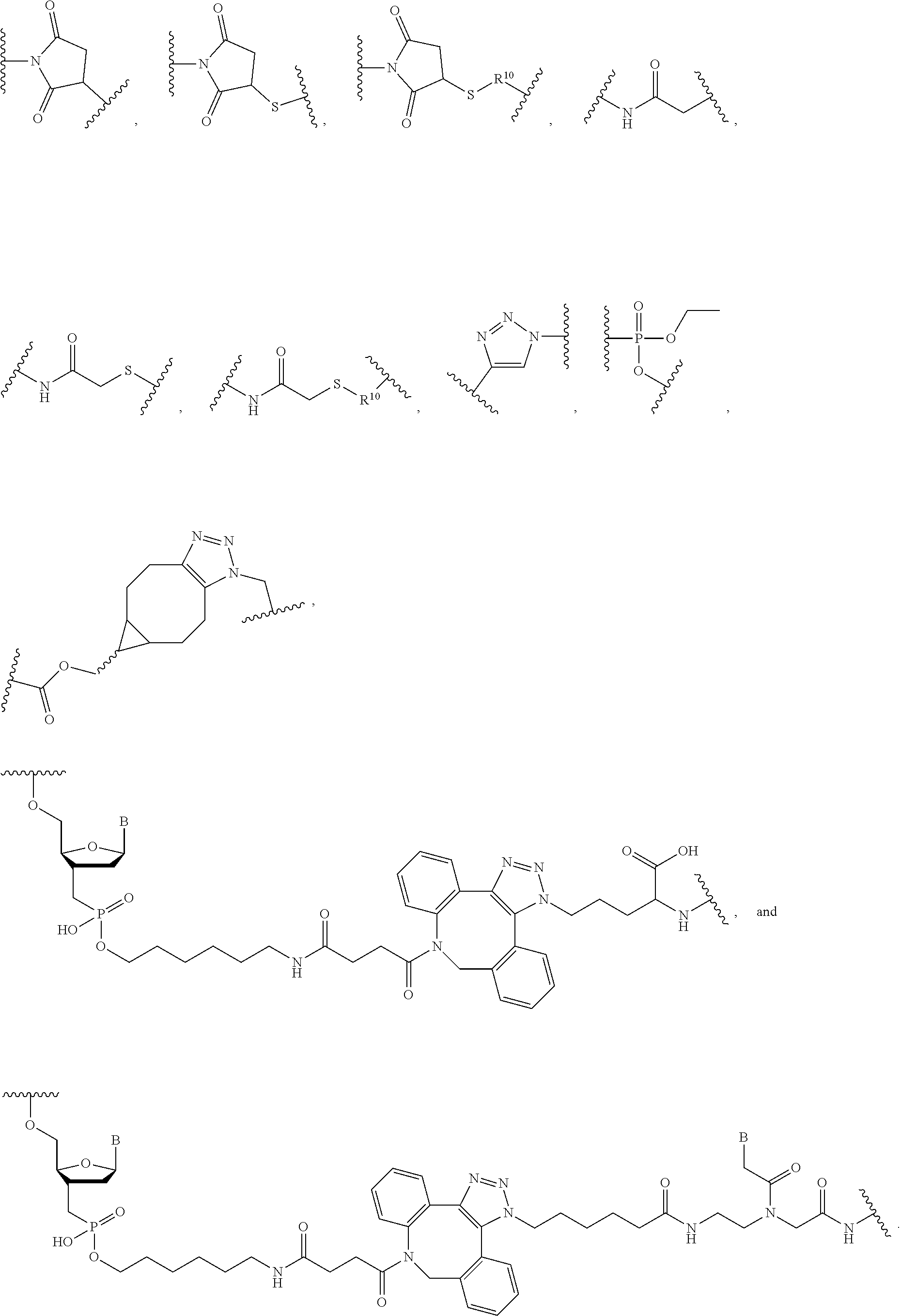

wherein: R¹⁰ is alkylene, cycloalkyl, or

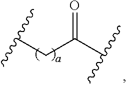

wherein a is 0 to 10.

M can be

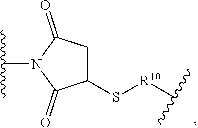

### R¹⁰ can be

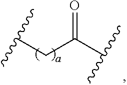

### and a is 0 to 10. M can be

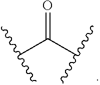 [SMILES: *C(*)=O]

M can be a heterobifunctional crosslinker, e.g.,

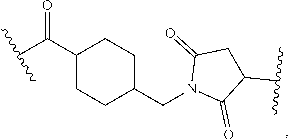 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2.o can be 3.

p can be 0 to 5, e.g., 0, 1, 2, 3, 4, or 5. p can be 0. p can be 1. p can be 2. p can be 3. p can be 4. p can be 5.

The linker can have the structure:

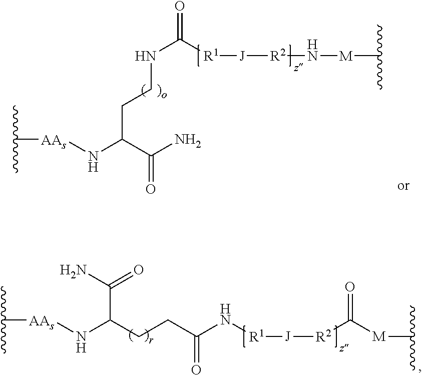

wherein M, AAₛ, each —(R¹-J-R²)z″-, o and z′ are defined herein; r can be 0 or 1.

r can be 0. r can be 1.

The linker can have the structure:

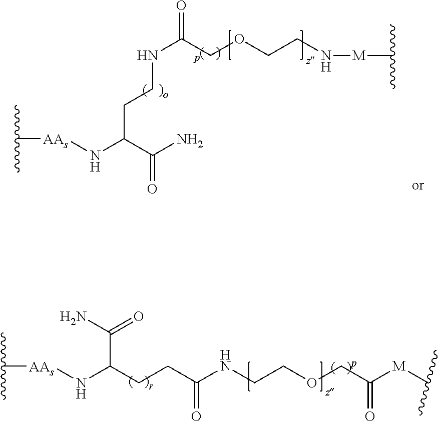

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

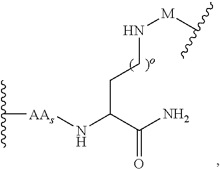

wherein:
M, AAₛ and o are as defined herein.

Other non-limiting examples of suitable linkers include:

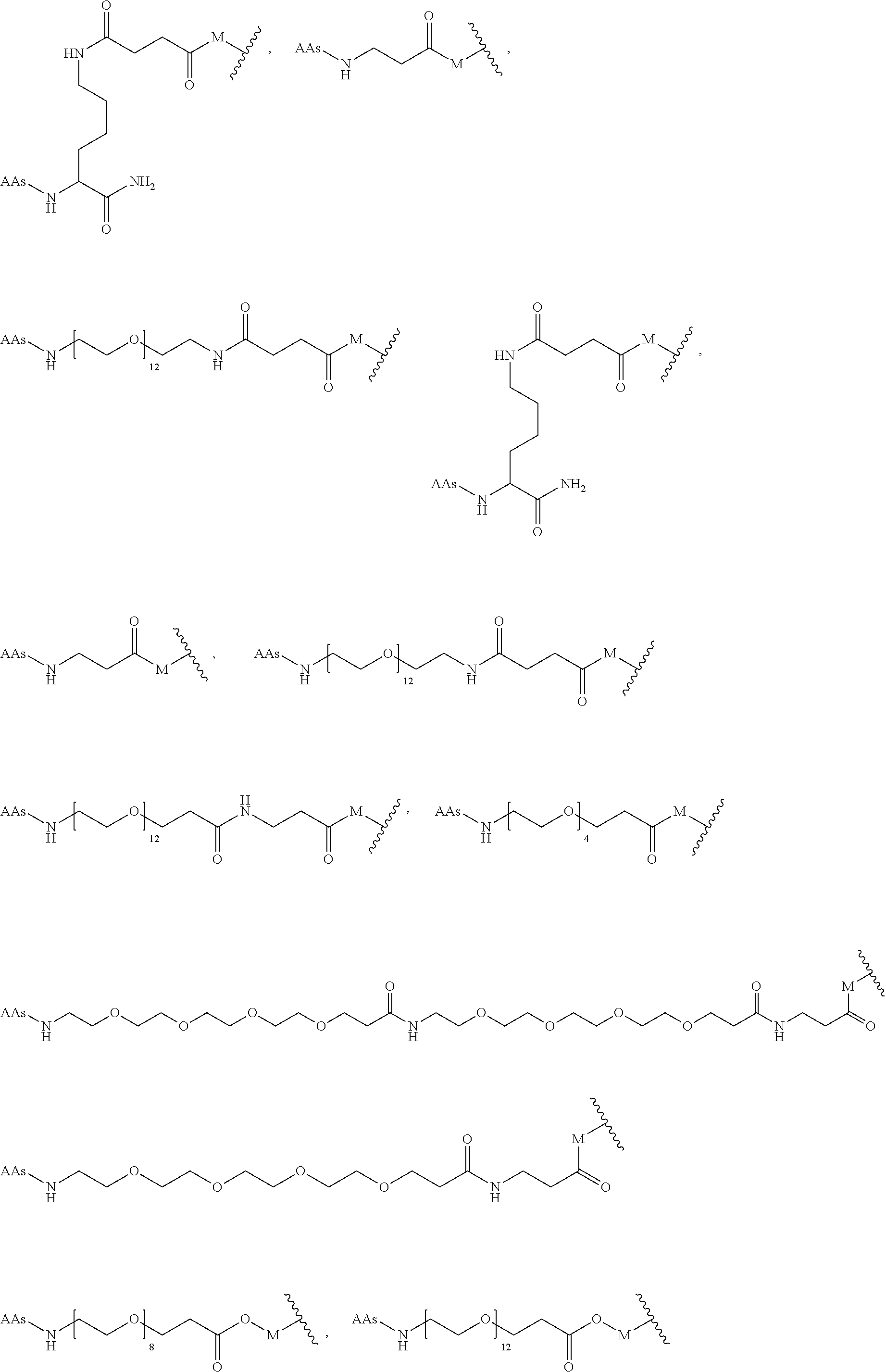

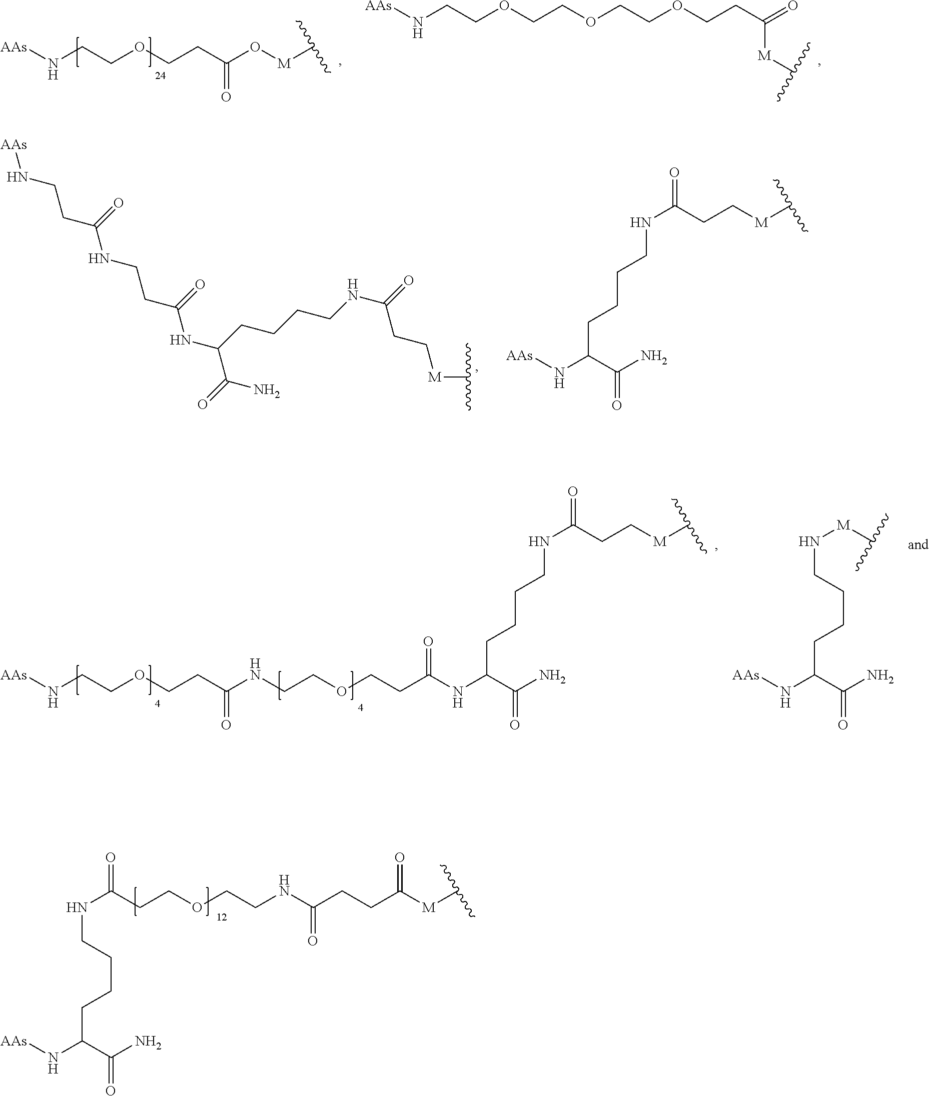

wherein M and AAₛ are as defined herein.

Other non-limiting examples of suitable L groups include:

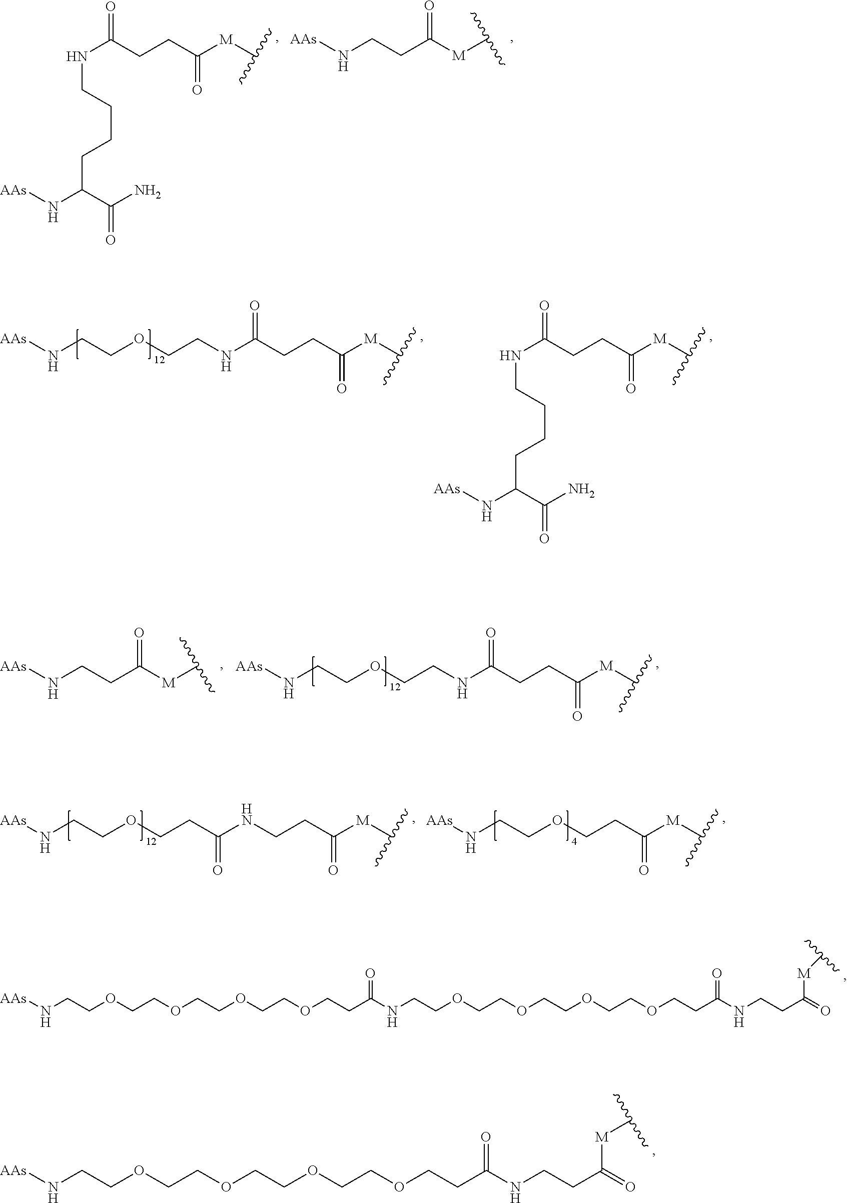

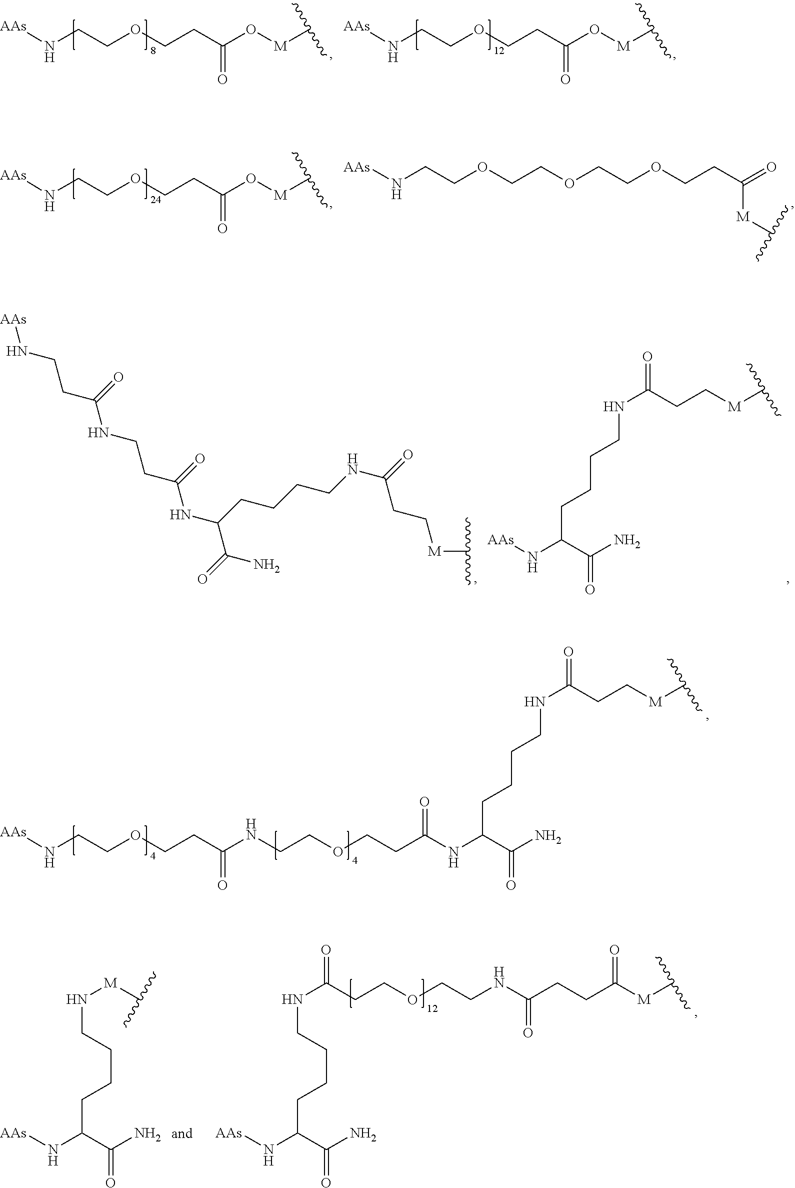

where AAₛ and M are as defined above.

Provided herein is a compound comprising a cCPP and an TO further comprising L, wherein the linker is conjugated to the TO through a bonding group (M), wherein M is

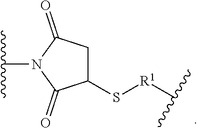

Provided herein is a compound comprising a cCPP and a TO, wherein the compound further comprises L, wherein the linker is conjugated to the TO through a bonding group (M), wherein M is selected from:

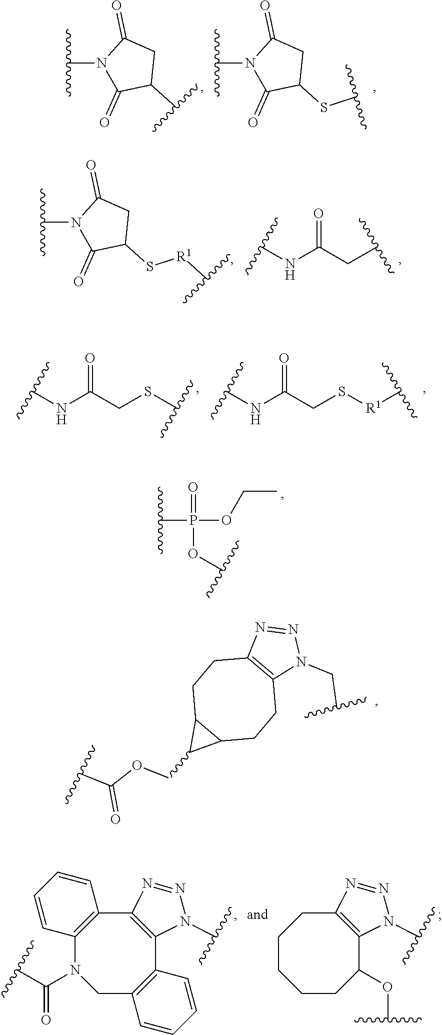

wherein: R¹ is alkylene, cycloalkyl, or

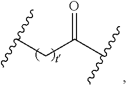

wherein t′ is 0 to 10 wherein each R is independently an alkyl, alkenyl, alkynyl, carbocyclyl, or heterocyclyl, wherein R¹ is

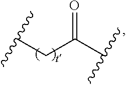

and t′ is 2.

The linker can have the structure:

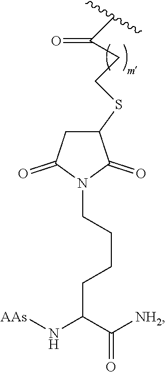

wherein AAₛ is as defined herein, and m′ is 0-10.

The linker can be of the formula:

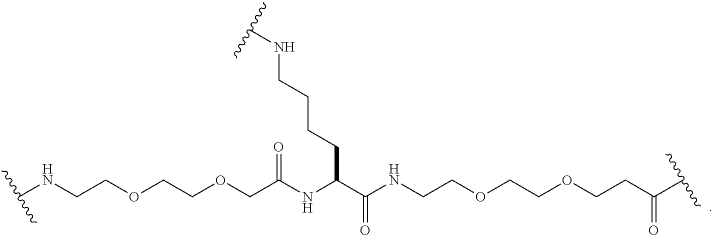 [SMILES: *NCCCC[C@H](NC(=O)COCCOCCN*)C(=O)NCCOCCOCCC(*)=O]

The linker can be of the formula:

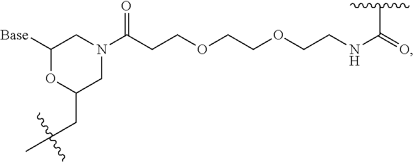

wherein “base” corresponds to a nucleobase at the 3′ end of a therapeutic moiety phosphorodiamidate morpholino oligomer.

The linker can be of the formula:

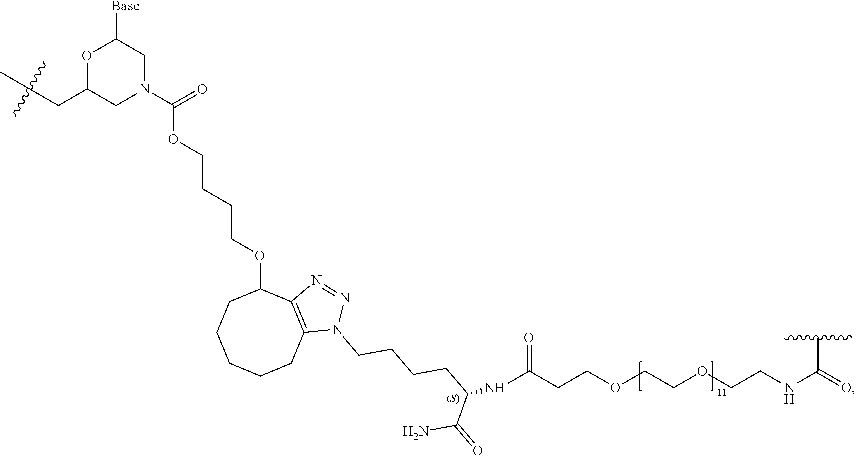

wherein “base” corresponds to a nucleobase at the 3′ end of a therapeutic moiety phosphorodiamidate morpholino oligomer.

The linker can be of the formula:

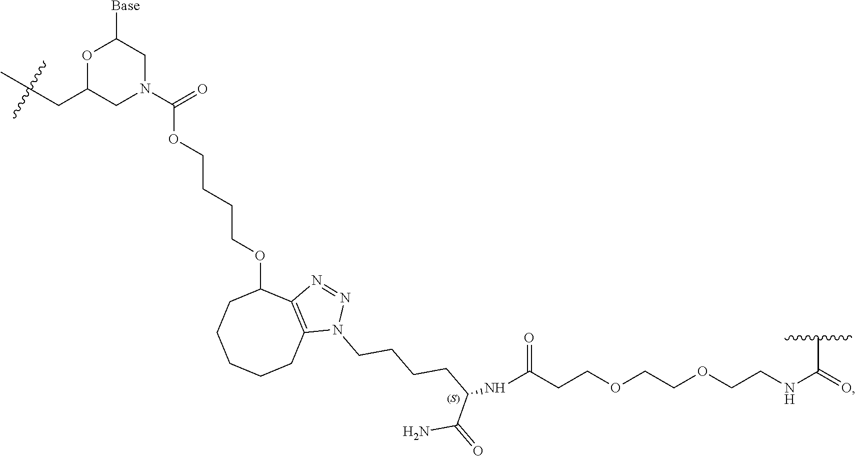

wherein “base” corresponds to a nucleobase at the 3′ end of a therapeutic moiety phosphorodiamidate morpholino oligomer.

The linker can be of the formula:

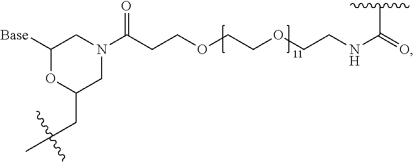

wherein “base” corresponds to a nucleobase at the 3′ end of a therapeutic moiety phosphorodiamidate morpholino oligomer.

The linker can be of the formula:

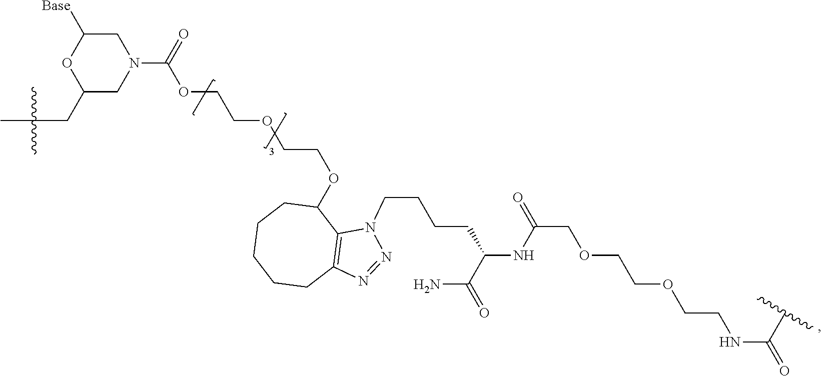

The linker can be covalently bound to a therapeutic moiety at any suitable location on the therapeutic moiety. The linker is covalently bound to the 3′ end of therapeutic moiety oligonucleotide or the 5′ end of an oligonucleotide therapeutic moiety. The linker can be covalently bound to the backbone of a therapeutic moiety oligonucleotide.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

In embodiments, the present disclosure provides a compound of Formula (IV) having the structure:

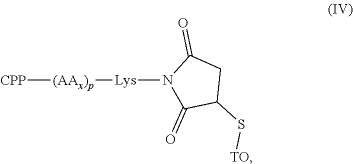

wherein CPP is a cell penetrating peptide, TO is a therapeutic oligonucleotide moiety as defined herein, and AAₓ and p are as defined above for Formula IX. A compound according to Formula XVI may be conjugated with one or more CTMs, optionally with one or more EP.

In embodiments, the present disclosure provides a compound of Formula (V) having the structure:

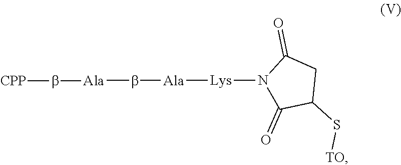

wherein CPP is a cell penetrating peptide and TO is a therapeutic oligonucleotide moiety as defined herein. A compound according to Formula XVII may be conjugated with one or more CTMs, optionally with one or more EP.

In embodiments, the present disclosure provides a compound of Formula (VI) having the structure:

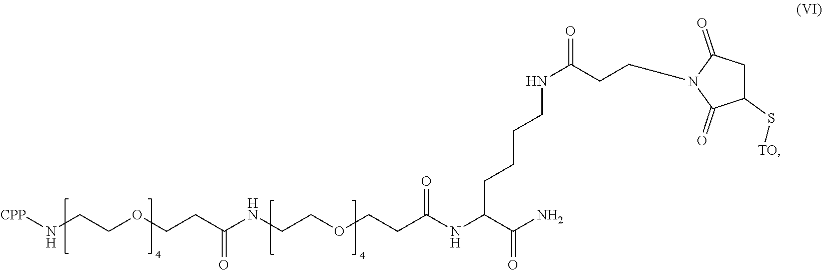

wherein CPP is a cell penetrating peptide and TO is a therapeutic oligonucleotide moiety as defined herein. A compound according to Formula XVIII may be conjugated with one or more CTMs, optionally with one or more EP.

In embodiments, the present disclosure provides a compound of Formula (VII) having the structure:

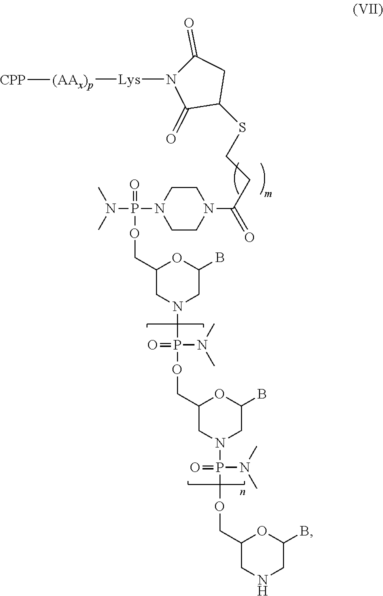

wherein m, n, p, AAₓ, and B are as defined above. A compound according to Formula XIX may be conjugated with one or more CTMs, optionally with one or more EP.

In embodiments, the present disclosure provides a compound of Formula (VIII) having the structure:

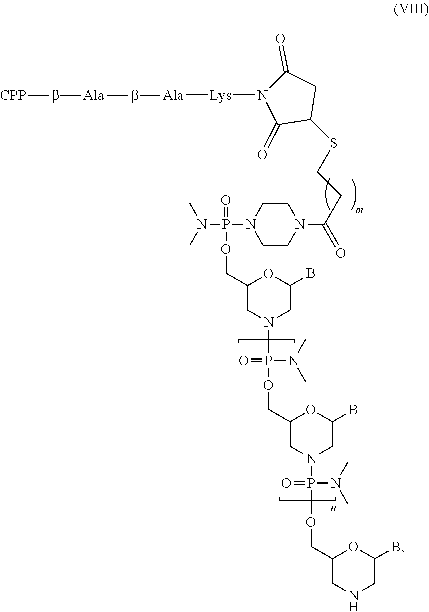

wherein CPP, m, n, and B are as defined above. A compound according to Formula (VIII) may be conjugated with one or more CTMs, optionally with one or more EP.

In embodiments, the present disclosure provides a compound of Formula (IX) having the structure:

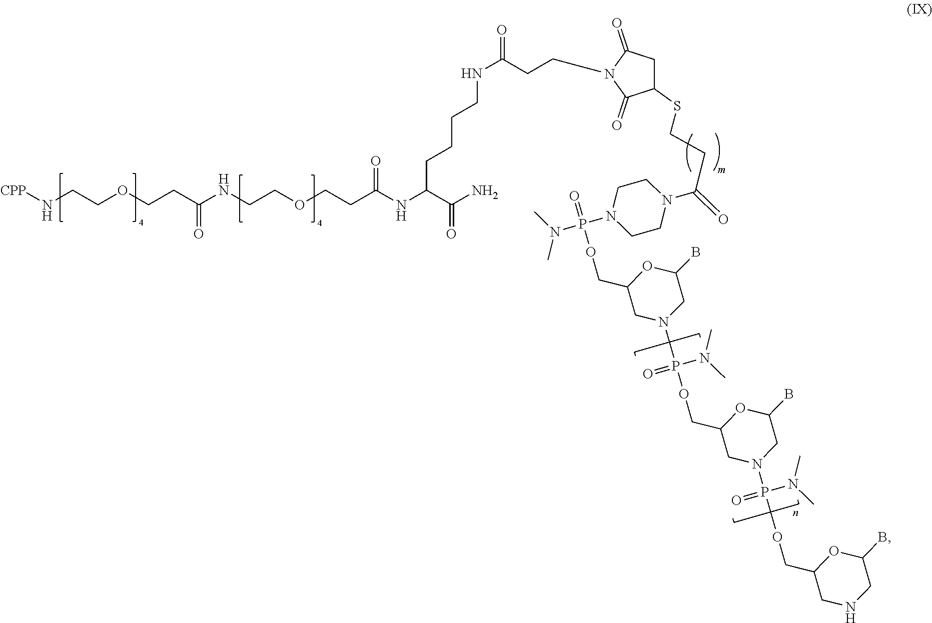

wherein CPP, m, n, and B are as defined above. A compound according to Formula (IX) may be conjugated with one or more CTMs, optionally with one or more EP.

In embodiments, the linker (L) contains a group which may be cleaved after cytosolic uptake of the compound to release the TO moiety. Non-limiting examples of physiologically cleavable linking groups include carbonate, thiocarbonate, thioester, disulfide, sulfoxide, hydrazine, protease-cleavable dipeptide linker, and the like.

In embodiments, a precursor to L also contains a thiol group, which forms a disulfide bond with the side chain of cysteine or cysteine in the CPP or TO moiety or that is attached to the 5′ end, 3′ end, or a nucleobase of the TO moiety.

Accordingly, in various embodiments, the compounds disclosed have the following structure of Formula (X):

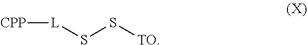

A compound according to Formula (X) may be conjugated with one or more CTMs, optionally with one or more EP.

In embodiments, the disulfide bond is formed between a thiol group on L, and the side chain of cysteine or an amino acid analog having a thiol group on CPP or attached to the 5′ end, the 3′ end, backbone, or a nucleobase of the TO moiety. Non-limiting examples of amino acid analogs having a thiol group which may be used with the compounds disclosed herein include:

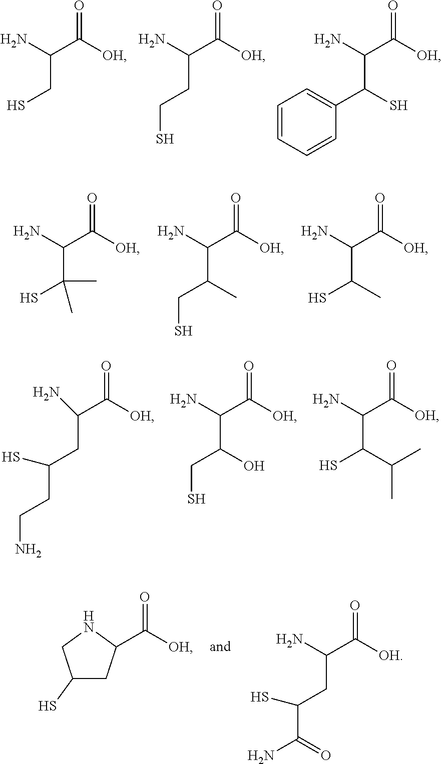

One skilled in the art will recognize that the amino acid analogs depicted above are shown as precursors, i.e., prior to incorporation into the compounds. When incorporated in the compounds of the present disclosure, the N- and C-termini are independently substituted to form peptide bonds, and the hydrogen on the thiol group is replaced with a bond to another sulfur atom to thereby form a disulfide.

Non-limiting examples of unconjugated TO structures (i.e., prior to conjugation to the CPP are provided below. In the structures below G is guanosine.

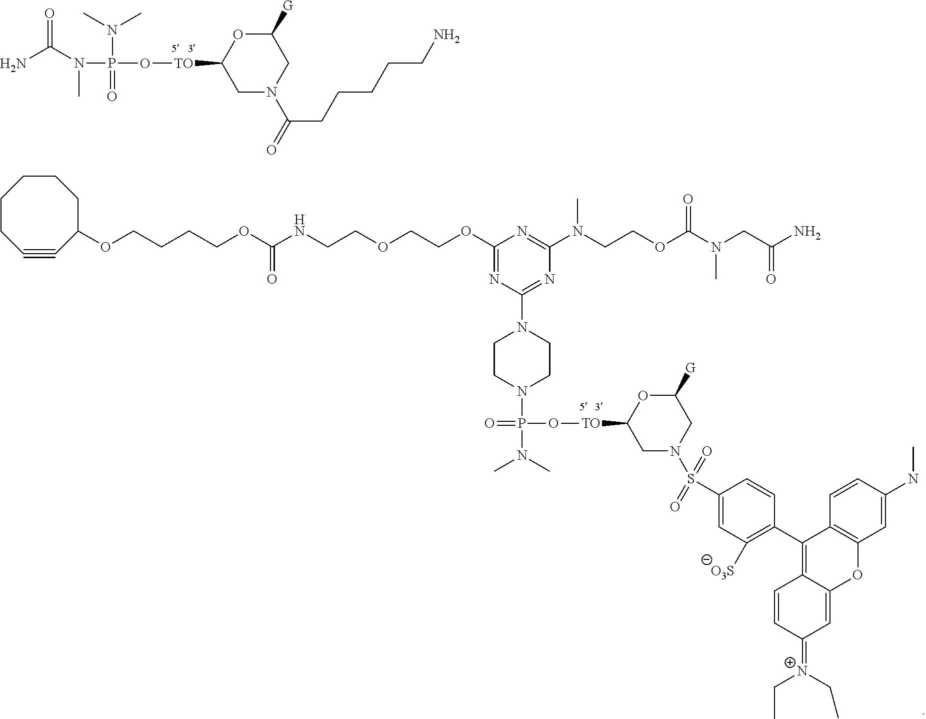

In embodiments, the TO, the linker, and M (along with a portion of the CPP) have the following structure:

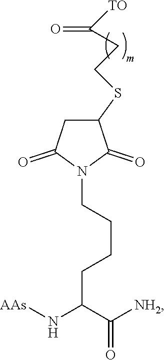

wherein TO, m and AAₛ are as defined above.

In embodiments, the present disclosure provides a compound comprising the following structure:

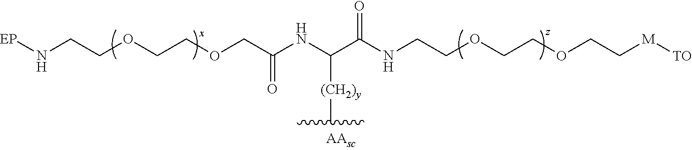

wherein:
EP is an exocyclic peptide and TO, M, AA_{SC}, x, y, and z are as defined above.

### Cyclic Peptide-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 5:

TABLE 5
cCPP-linker conjugates

cyclo(FfΦ-4gp-r-4gp-rQ)-PEG₄-K-NH₂
cyclo(FfΦ-Cit-r-Cit-rQ)-PEG₄-K-NH₂
cyclo(FfΦ-Pia-r-Pia-rQ)-PEG₄-K-NH₂
cyclo(FfΦ-Dml-r-Dml -rQ)-PEG₄-K-NH₂
cyclo(FfΦ-Cit-r-Cit-rQ)-PEG₁₂-OH
cyclo(fΦR-Cit-R-Cit-Q)-PEG₁₂-OH

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 6:

TABLE 6
cCPP-linker conjugate

Ac-PKKKRKV-Lys(cyclo[FfΦ-R-r-Cit-rQ])-PEG₁₂-K(N₃)-NH₂
Ac-PKKKRKV-Lys(cyclo[FfΦ-Cit-r-R-rQ])-PEG₁₂-K(N₃)-NH₂
Ac-PKKKRKV-K(cyclo(FfΦR-cit-R-cit-Q))-PEG₁₂-K(N₃)-NH₂
Ac-PKKKRKV-PEG2-Lys(cyclo[FfΦ-Cit-r-Cit-rQ])-B-k(N₃)-NH₂
Ac-PKKKRKV-PEG2-Lys(cyclo[FfΦ-Cit-r-Cit-rQ])-PEG2-k(N₃)-NH₂
Ac-PKKKRKV-PEG2-Lys(cyclo[FfΦ-Cit-r-Cit-rQ])-PEG4-k(N₃)-NH₂
Ac-PKKKRKV-Lys(cyclo[FfΦ-Cit-r-Cit-rQ])-PEG12-k(N₃)-NH₂
Ac-pkkkrkv-PEG2-Lys(cyclo[FfΦ-Cit-r-Cit-rQ])-PEG12-k(N₃)-NH₂
Ac-rrv-PEG2-Lys(cyclo[FfΦ-Cit-r-Cit-rQ])-PEG12-OH
Ac-PKKKRKV-PEG2-Lys(cyclo[FfΦ-Cit-r-Cit-r-Q])-PEG12-k(N₃)-NH₂
Ac-PKKK-Cit-KV-PEG2-Lys(cyclo[FfΦ-Cit-r-Cit-r-Q])-PEG12-
k(N₃)-NH₂
Ac-PKKKRKV-PEG2-Lys(cyclo[FfΦ-Cit-r-Cit-r-Q]-PEG12-K(N₃)-NH₂

EEVs comprising a cyclic cell penetrating peptide (cCPP), linker and exocyclic peptide (EP) are provided. An EEV can comprise the structure of Formula (S):

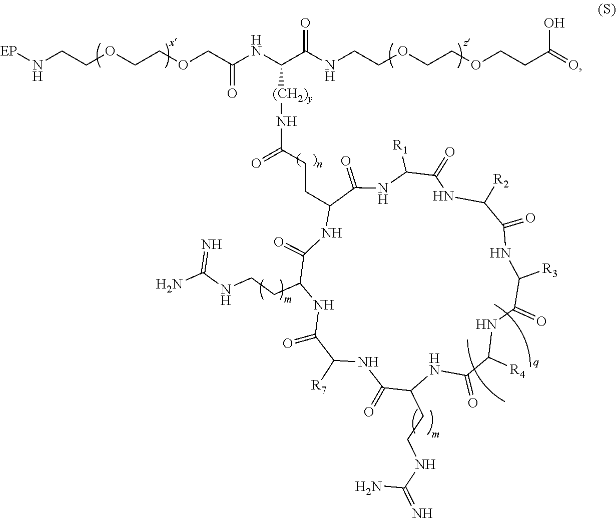

or a protonated form thereof,
wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.

R₁, R₂, R₃, R₄, R₇, EP, m, q, y, x′, z′ are as described herein.

n can be 0. n can be 1. n can be 2.

The EEV can comprise the structure of Formula (S-a) or (S-b):

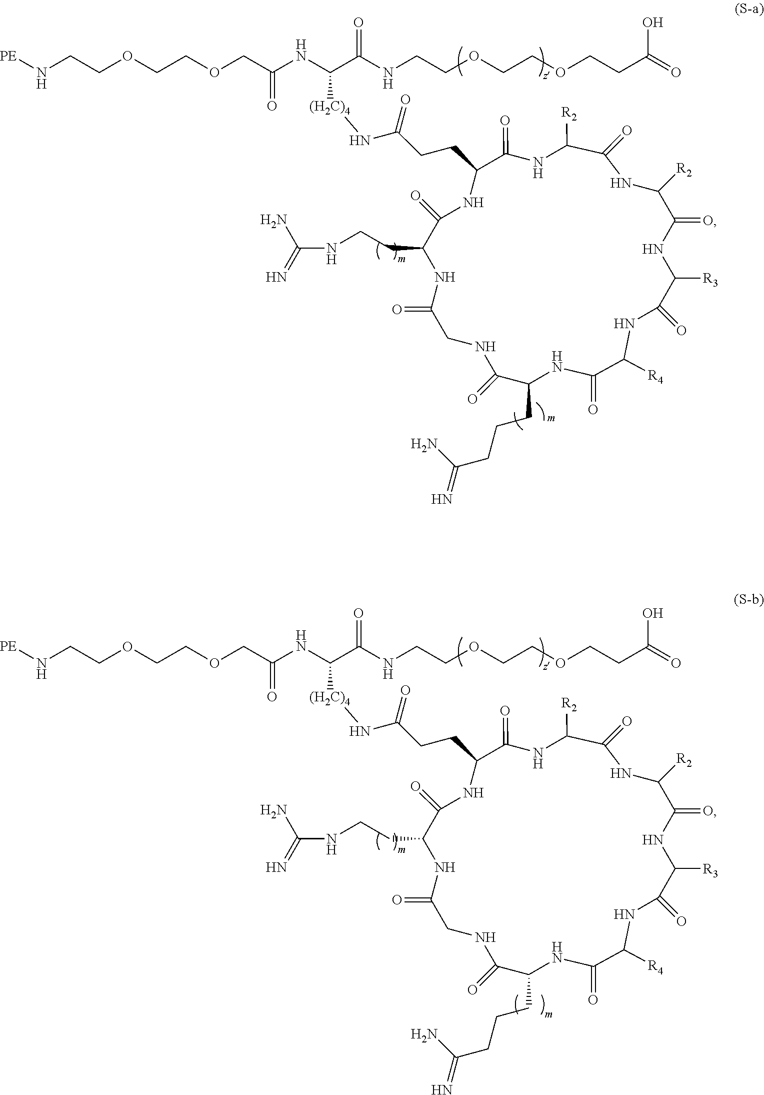

or a protonated form thereof, wherein EP (PE), R¹, R², R³, R⁴, m and z′ are as defined above in Formula (S).

The EEV can comprises the structure of Formula (S-c):

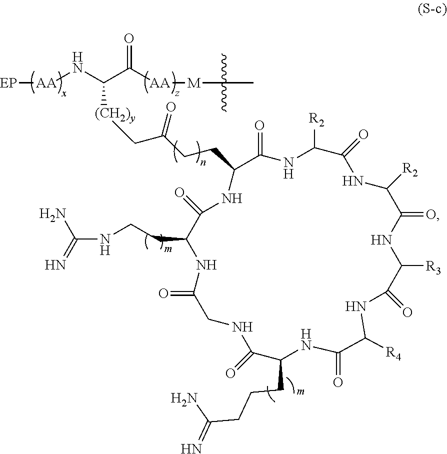

or a protonated form thereof, wherein EP, R¹, R², R³, R⁴, and m are as defined above in Formula (B); AA is an amino acid as defined herein. M is as defined herein; n is an integer from 0-2; x is an integer from 1-10; y is an integer from 1-5; and z is an integer from 1-10.

The EEV can have the structure of Formula (S-1), (S-2), (S-3), or (S-4):

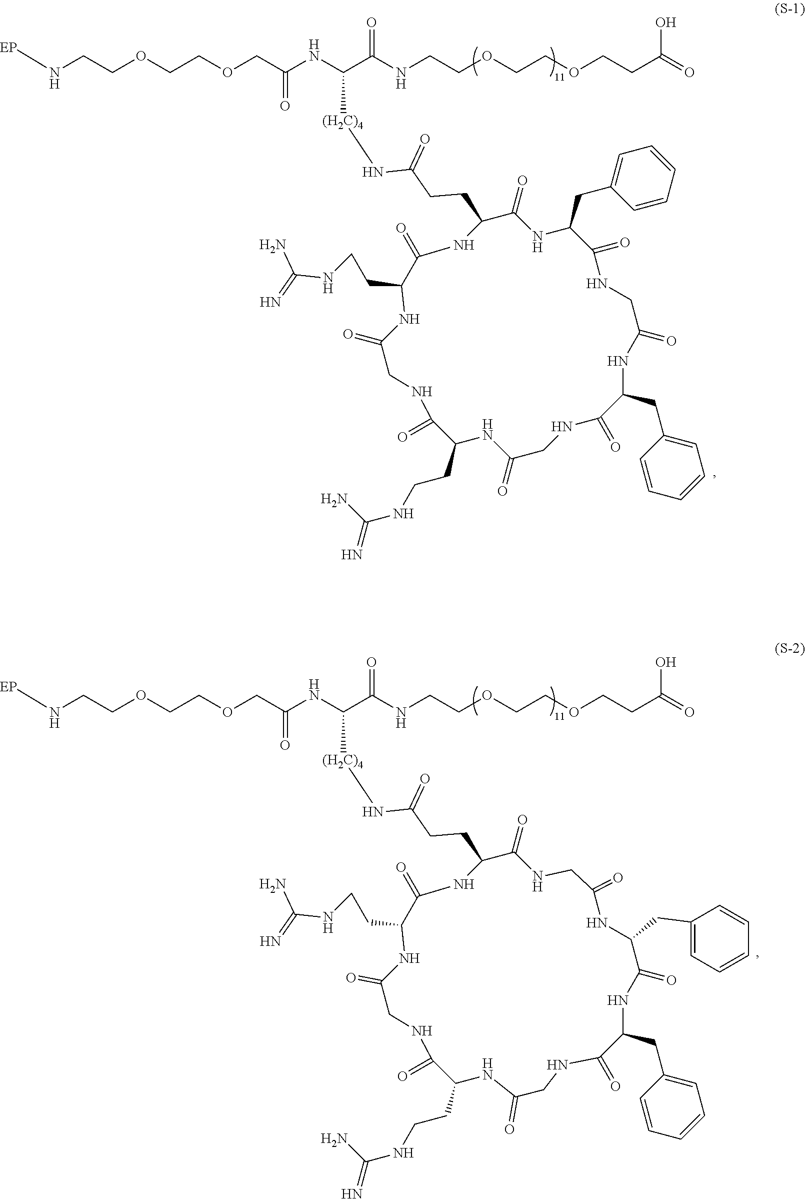

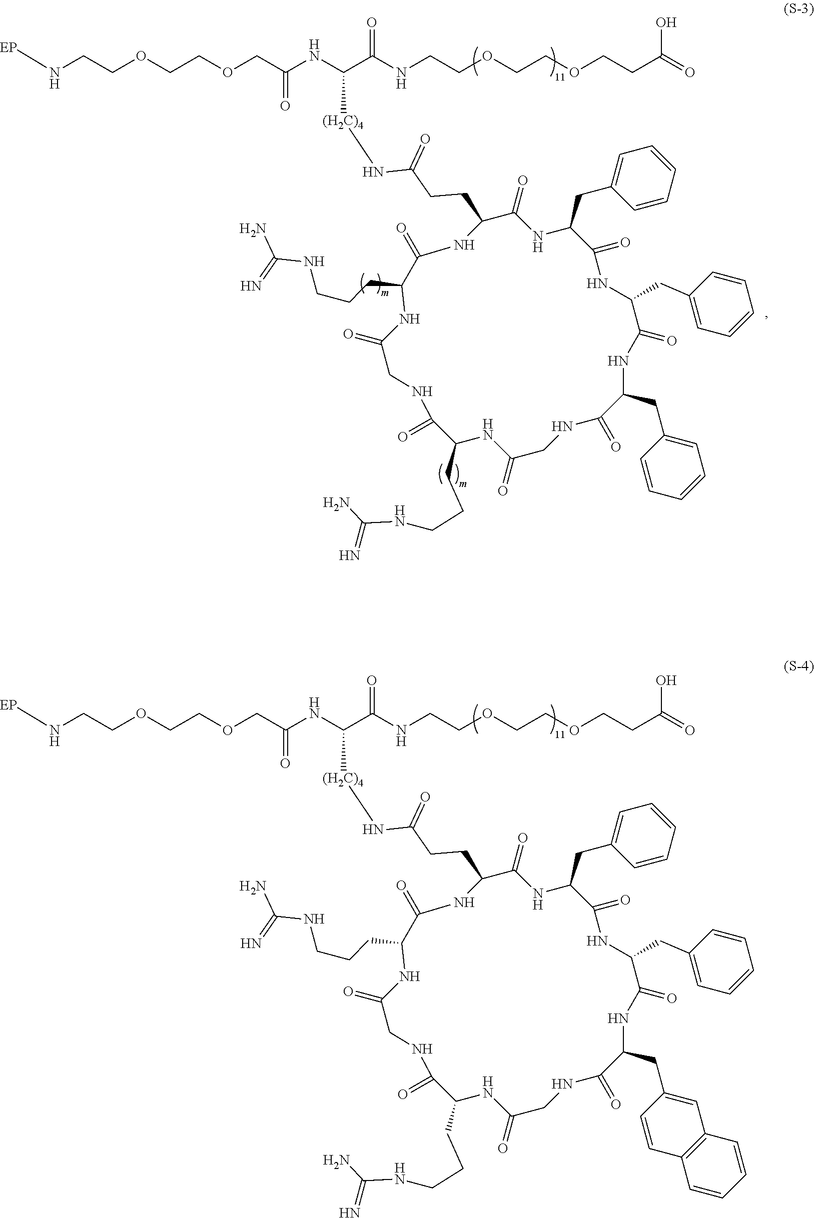

or a protonated form thereof, wherein EP is as defined above in Formula (S).

The EEV can comprise Formula (S) and can have the structure: Ac-PKKKRKVAEEA-K(cyclo[FGFGRGRQ])-PEG₁₂-OH or Ac-PK-KKR-KV-AEEA-K(cyclo[GfFGrGrQ])-PEG₁₂-OH.

The EEV can comprise a cCPP of formula:

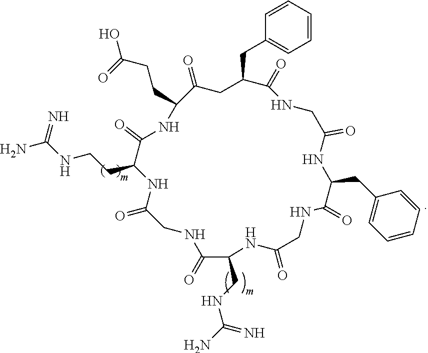

The EEV can comprise formula: Ac-PKKKRKV-miniPEG2-Lys(cyclo(FfFGRGRQ)-miniPEG2-K(N3).

The EEV can be Ac-P-K(Tfa)-K(Tfa)-K(Tfa)-R-K(Tfa)-V-AEEA-K-(cyclo[FGFGRGRQ])-PEG12-OH. The EEV can be:

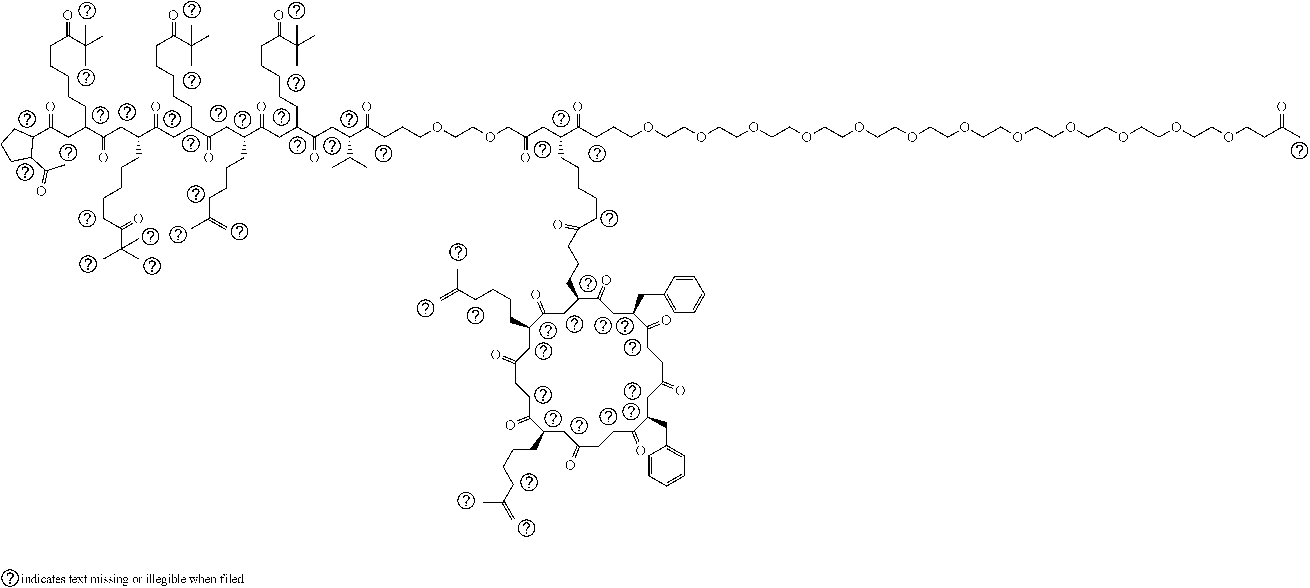 [SMILES: C=C(C)CCCC[C@@H]1CC(=O)CCC(=O)[C@H](Cc2ccccc2)CC(=O)CCC(=O)[C@H](Cc2ccccc2)CC(=O)[C@H](CCCC(=O)CCCCC[C@H](CC(=O)COCCOCCCC(=O)[C@@H](CC(=O)C(CCCCCC(=O)C(C)(C)C)CC(=O)[C@H](CCCCC(=C)C)CC(=O)C(CCCCCC(=O)C(C)(C)C)CC(=O)[C@H](CCCCCC(=O)C(C)(C)C)CC(=O)C(CCCCCC(=O)C(C)(C)C)CC(=O)C2CCCC2C(C)=O)C(C)C)C(=O)CCCOCCOCCOCCOCCOCCOCCOCCOCCOCCOCCOCCOCCC(C)=O)CC(=O)[C@H](CCCCC(=C)C)CC(=O)CCC1=O]

The EEV can be Ac-PKKKRKV-AEEA-Lys-(cyclo[FGFGRGRQ])-PEG12-OH. The EEV can be:

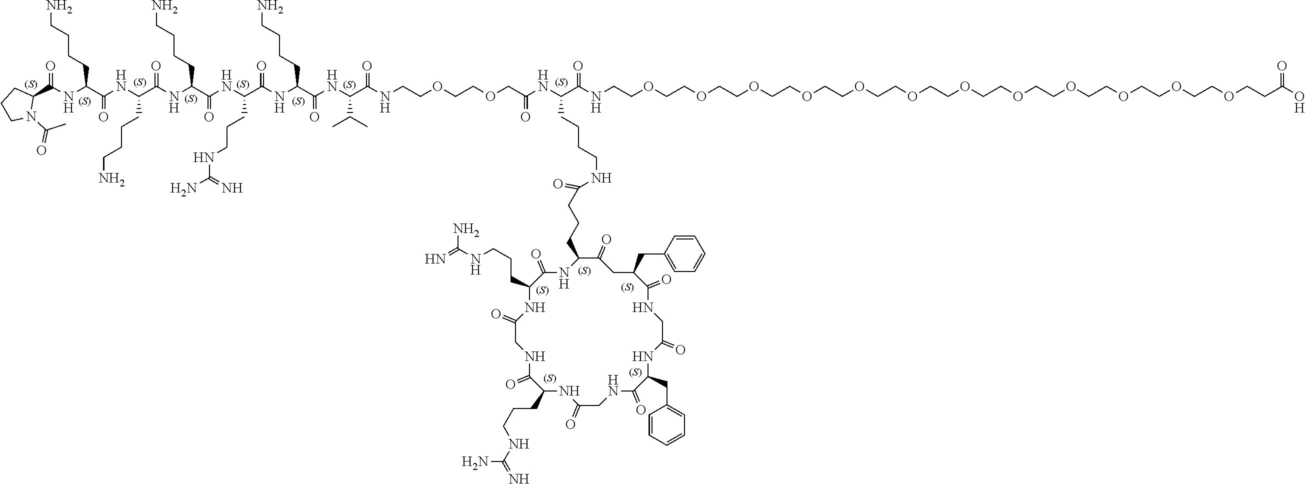

The EEV can be: Ac-PKKKRKV-miniPEG-K(cyclo(Ff-Nal-GrGrQ)-PEG₁₂-OH.

The EEV can be: Cyclo(FGFGRGRQ)-PEG₁₂-OH.

The EEV can be: Ac-PKKKRKV-miniPEG-K(cyclo(FGFGRRRQ)-PEG₁₂-OH.

The EEV can be: Ac-PKKKRKV-miniPEG-K(cyclo(FGFRRRRQ)-PEG₁₂-OH.

The EEV can be: Cyclo(FfΦGRGRQ)-PEG₁₂-OH.

The EEV can be: Cyclo(FGFGRRRQ)-PEG₁₂-OH.

### Exocyclic Peptides (EP) of Amino Acids

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring, amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise from 2 to 10 amino acid residues, wherein at least one amino acid residue is positively charged, at least one amino acid comprises a side chain comprising a guanidine group, or a protonated form thereof, or a combination thereof. The positively charged amino acid residue an comprise arginine.

The EP can comprise at least two lysine residues.

The EP can comprise KK, KR, RR, HH, HK, HR, RH, KKK, KGK, KBK, KBR, KRK, KRR, RKK, RRR, KKH, KHK. HKK, HRR, HRH, HHR, HBH, HHH, HHHH, KHKK, KKHK. KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine. The amino acids in the EP can have D or L stereochemistry.

The EP can comprise KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can comprise PKKKRKV, RR, RRR, RHR, RBR, RBRBR, RBHBR, or HBRBH, wherein B is beta-alanine. The amino acids in the EP can have D or L stereochemistry.

The EP can consist of KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can consist of PKKKRKV, RR, RRR, RHR, RBR, RBRBR, RBHBR, or HBRBH, wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV. The EP can consist of an NLS comprising the amino acid sequence PKKKRKV. The EP can comprise an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK. The EP can consist of an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK

All exocyclic sequences can also contain an N-terminal acetyl group. Hence, for example, the EP can have the structure: Ac-PKKKRKV.

### Cyclic Peptides Conjugated to a Cargo (TO) Moiety

In embodiments, the cyclic peptide of the present disclosure is conjugated to a cargo moiety defined herein. In embodiments, the cargo moiety comprises a TO moiety as defined herein.

In embodiments, an endosomal escape vehicle (EEV) is provided that comprises a cyclic peptide, an exocyclic peptide (EP) and linker, wherein the EEV is conjugated to a cargo and the EEV-conjugate comprises the structure of Formula (XI):

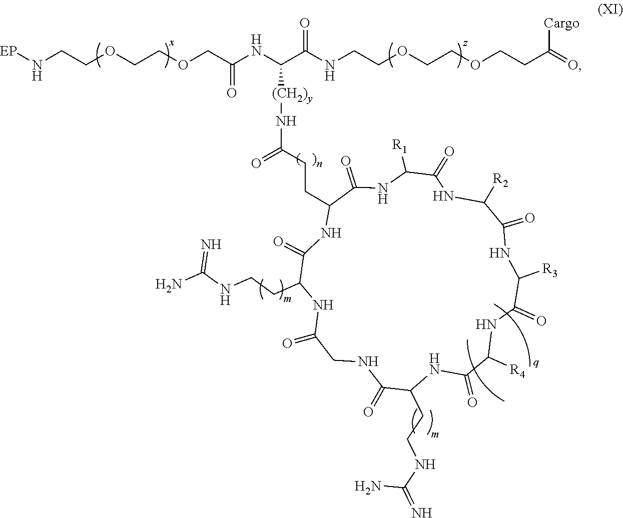

or a protonated form thereof,
wherein:
R₁, R₂, and R₃ are each independently H or an amino acid residue having a side chain comprising an aromatic group;
R₄ is H or an amino acid side chain;
EP is an exocyclic peptide as defined herein;
Cargo is a TO moiety as defined herein;
each m is independently an integer from 0-3;
n is an integer from 0 to 2;
x is an integer from 2 to 20;
y is an integer from 1 to 5;
q is an integer from 1 to 4; and
z is an integer from 2 to 20.

In embodiments, the compound, which may be further conjugated to a CTM, comprises the structure of Formula (XI-1A) or (XI-2A):

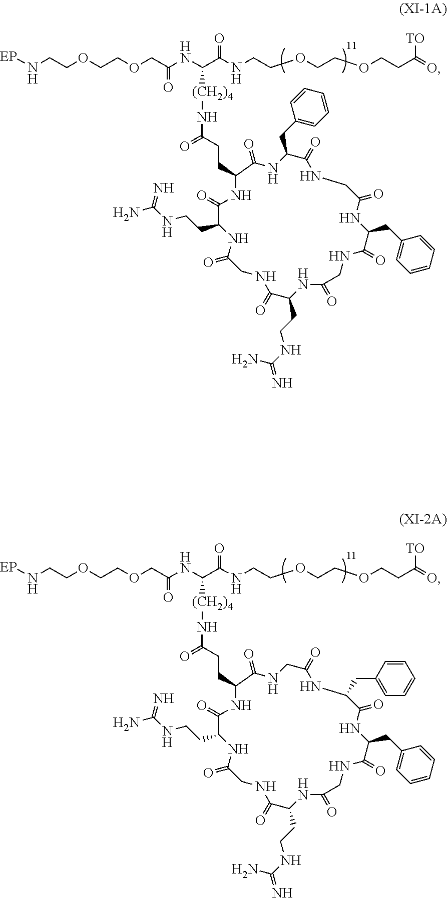

or a protonated form thereof, wherein EP is an exocyclic peptide as defined herein, and TO is as defined above.

In embodiments of the compound of Formula XXVI, R₁, R₂, and R₃ are each independently H, -alkylene-aryl, or -alkylene-heteroaryl. In embodiments, R₁, R₂, and R₃ are each independently H, —C₁₋₃alkylene-aryl, or —C₁₋₃alkylene-heteroaryl. In embodiments, R₁, R₂, and R₃ are each independently H or -alkylene-aryl. In embodiments, R₁, R₂, and R₃ are each independently H or —C₁₋₃alkylene-aryl. In embodiments, the C₁₋₃alkylene is a methylene. In embodiments, the aryl is a 6- to 14-membered aryl. In embodiments, the heteroaryl is a 6- to 14-membered heteroaryl having one or more heteroatoms selected from N, O, and S. In embodiments, the aryl is selected from phenyl, naphthyl, or anthracenyl. In embodiments, the aryl is phenyl or naphthyl. In embodiments, the aryl is phenyl. In embodiments, the heteroaryl is pyridyl, quinolyl, and isoquinolyl. In embodiments, R₁, R₂, and R₃ are each independently H, —C₁₋₃alkylene-Ph or —C₁₋₃alkylene-Naphthyl. In embodiments, R₁, R₂, and R₃ are each independently H, —CH₂Ph, or —CH₂Naphthyl. In embodiments, R₁, R₂, and R₃ are each independently H or —CH₂Ph.

In embodiments, R₄ is H, -alkylene-aryl, -alkylene-heteroaryl. In embodiments, R₄ is H, —C₁₋₃alkylene-aryl, or —C₁₋₃alkylene-heteroaryl. In embodiments, R₄ is H or -alkylene-aryl. In embodiments, R₄ is H or —C₁₋₃alkylene-aryl. In embodiments, the C₁₋₃alkylene is a methylene. In embodiments, the aryl is a 6- to 14-membered aryl. In embodiments, the heteroaryl is a 6- to 14-membered heteroaryl having one or more heteroatoms selected from N, O, and S. In embodiments, the aryl is selected from phenyl, naphthyl, or anthracenyl. In embodiments, the aryl is phenyl or naphthyl. In embodiments, the aryl is phenyl. In embodiments, the heteroaryl is pyridyl, quinolyl, and isoquinolyl. In embodiments, R₄ is H, —C₁₋₃alkylene-Ph or —C₁₋₃alkylene-Naphthyl. In embodiments, R₄ is H or the side chain of an amino acid in Table 1 or Table 2. In embodiments, R₄ is H or an amino acid residue having a side chain comprising an aromatic group. In embodiments, R₄ is H, —CH₂Ph, or —CH₂Naphthyl. In embodiments, R₄ is H or —CH₂Ph.

In embodiments, 1, 2, or 3 of R₁, R₂, R₃, and R₄ are —CH₂Ph. In embodiments, one of R₁, R₂, R₃, and R₄ is —CH₂Ph. In embodiments, two of R₁, R₂, R₃, and R₄ are —CH₂Ph. In embodiments, three of R₁, R₂, R₃, and R₄ are —CH₂Ph. In embodiments, at least one of R₁, R₂, R₃, and R₄ is —CH₂Ph.

In embodiments, 1, 2, or 3 of R, R₂, R₃, and R₄ are H. In embodiments, one of R, R₂, R₃, and R₄ is H. In embodiments, two of R₁, R₂, R₃, and R₄ are H. In embodiments, three of R₁, R₂, R₃, and R₄ are H. In embodiments, at least one of R₁, R₂, R₃, and R₄ is H.

In embodiments, q is 1, 2, or 3. In embodiments, q is 1 or 2. In embodiments, q is 1. In embodiments, q is 2. In embodiments, q is 3. In embodiments, q is 4.

In embodiments, m is 1-3. In embodiments, m is 1 or 2. In embodiments, m is 0. In embodiments, m is 1. In embodiments, m is 2. In embodiments, m is 3.

In embodiments, n is 0. In embodiments, n is 1. In embodiments, n is 2.

In embodiments, x is an integer from 2-20, e.g., 2, 3, 4, 5, 6, 7, 8, 9, 10, 11, 12, 13, 14, 15, 16, 17, 18, 19, or 20, inclusive of all ranges and subranges therebetween. In embodiments, x is an integer from 5-15. In embodiments, x is an integer from 9-13. In embodiments, x is 11.

In embodiments, y is an integer from 1-5, e.g., 1, 2, 3, 4, or 5, inclusive of all ranges and subranges therebetween. In embodiments, y is an integer from 2-5. In embodiments, y is an integer from 3-5. In embodiments, y is 3 or 4. In embodiments, y is 4 or 5. In embodiments, y is 3. In embodiments, y is 4. In embodiments, y is 5.

In embodiments, z is an integer from 2-20, e.g., 2, 3, 4, 5, 6, 7, 8, 9, 10, 11, 12, 13, 14, 15, 16, 17, 18, 19, or 20, inclusive of all ranges and subranges therebetween. In embodiments, z is an integer from 5-15. In embodiments, z is an integer from 9-13. In embodiments, z is 11.

In embodiments, the EEV is conjugated to a cargo and the EEV-conjugate comprises the structure of Formula (XI-A-1) or (XI-B-1):

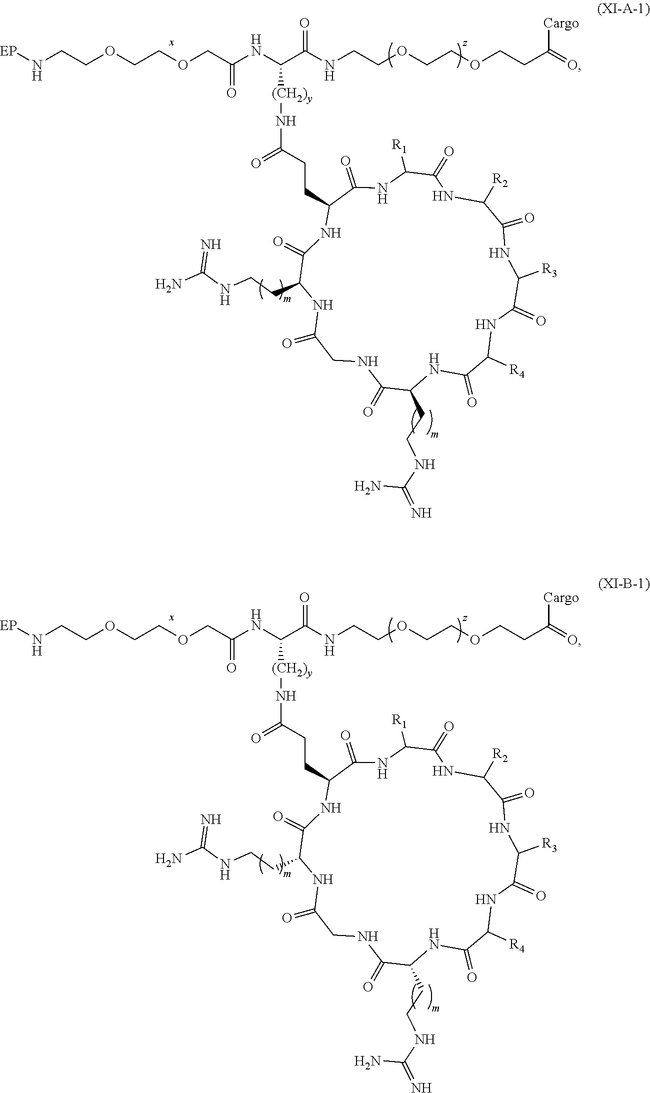

or a protonated form thereof, wherein EP, Cargo, m and z are as defined above in Formula (XI).

In embodiments, the EEV is conjugated to a cargo and the EEV-conjugate comprises the structure of Formula (XII-A):

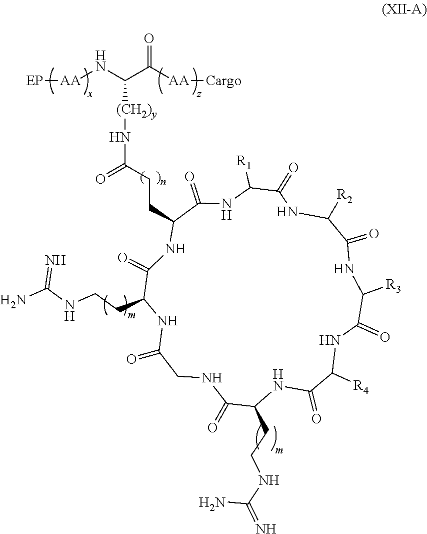

or a protonated form thereof,
wherein EP, R¹, R², R³, R⁴, Cargo, and m are as defined above in Formula (XI); AA is an amino acid as defined herein; n is an integer from 0 to 2; x is an integer from 1 to 10; y is an integer from 1 to 5; and z is an integer from 1 to 10.

The EEV can comprise formula: Ac-PKKKRKV-miniPEG2-Lys(cyclo(FfFGRGRQ)-miniPEG2-K(N3).

The EEV can be Ac-P-K(Tfa)-K(Tfa)-K(Tfa)-R-K(Tfa)-V-AEEA-K-(cyclo[FGFGRGRQ])-PEG12-OH. The EEV can be:

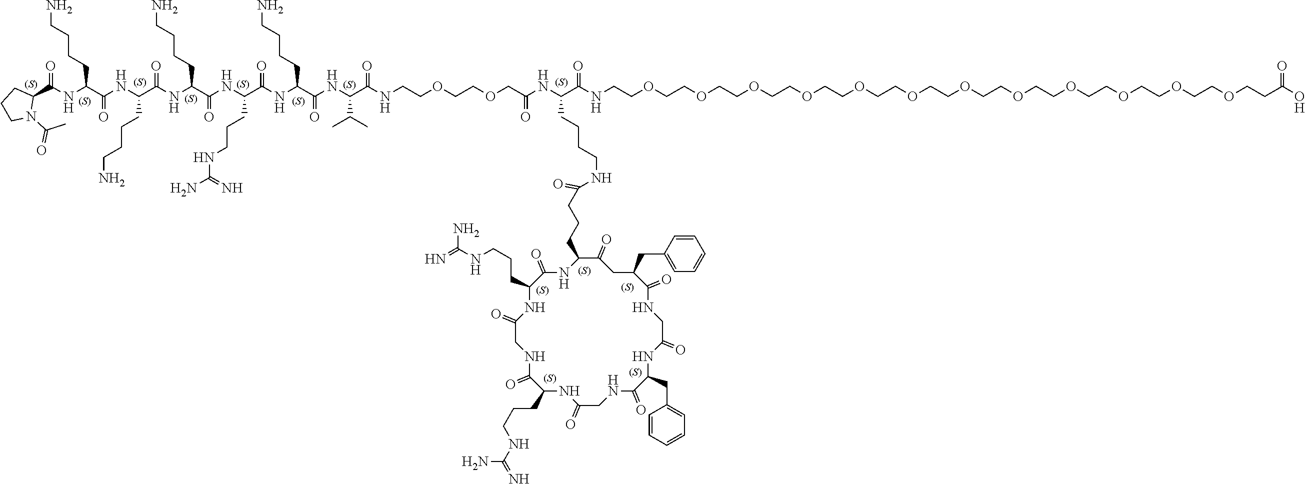

The EEV can be Ac-PKKKRKV-AEEA-Lys-(cyclo[FGFGRGRQ])-PEG12-OH. The EEV can be:

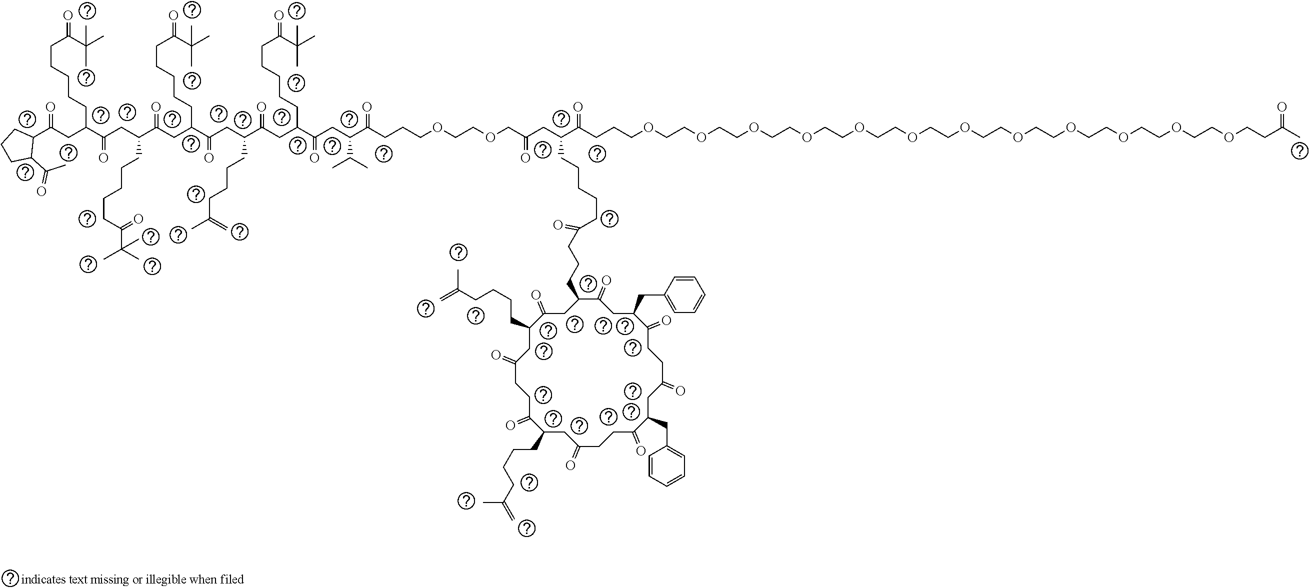 [SMILES: C=C(C)CCCC[C@@H]1CC(=O)CCC(=O)[C@H](Cc2ccccc2)CC(=O)CCC(=O)[C@H](Cc2ccccc2)CC(=O)[C@H](CCCC(=O)CCCCC[C@H](CC(=O)COCCOCCCC(=O)[C@@H](CC(=O)C(CCCCCC(=O)C(C)(C)C)CC(=O)[C@H](CCCCC(=C)C)CC(=O)C(CCCCCC(=O)C(C)(C)C)CC(=O)[C@H](CCCCCC(=O)C(C)(C)C)CC(=O)C(CCCCCC(=O)C(C)(C)C)CC(=O)C2CCCC2C(C)=O)C(C)C)C(=O)CCCOCCOCCOCCOCCOCCOCCOCCOCCOCCOCCOCCOCCC(C)=O)CC(=O)[C@H](CCCCC(=C)C)CC(=O)CCC1=O]

The EEV can be: Ac-PKKKRKV-miniPEG-K(cyclo(Ff-Nal-GrGrQ)-PEG₁₂-OH.

The EEV can be: Cyclo(FGFGRGRQ)-PEG₁₂-OH.

The EEV can be: Ac-PKKKRKV-miniPEG-K(cyclo(FGFGRRRQ)-PEG₁₂-OH.

The EEV can be: Ac-PKKKRKV-miniPEG-K(cyclo(FGFRRRRQ)-PEG₁₂-OH.

The EEV can be: Cyclo(FfΦGRGRQ)-PEG₁₂-OH.

The EEV can be. Cyclo(FGFGRRRQ)-PEG₁₂-OH.

The EEV can be: Cyclo(FGFRRRRQ)-PEG₁₂-OH.

The EEV can be selected from any EEV disclosed in WO 2022/213118 herein incorporated by reference.

### Carbohydrate Targeting Moiety (CTM)

The compounds described include a CTM, a CPP, and a therapeutic oligonucleotide. The compounds may further comprise an EP. The compounds may comprise any suitable CTM. The CTM may comprise a monosaccharide moiety or a polysaccharide moiety. In embodiments, the polysaccharide moiety comprises a disaccharide moiety or a trisaccharide moiety.

In embodiments, the CTM targets the compound to liver cells. In embodiments, the CTM targets the compound to hepatocytes. Liver cells may comprise receptors that recognize and bind carbohydrate moieties. For example, hepatic stellate cells comprise a mannose-6-phosphate receptor that may bind a mannose moiety or a mannose-6-phoshpate moiety. Hepatocytes comprise asialoglycoprotein receptors which may bind carbohydrate moieties such as galactoside moieties, galactosamine moieties, N-acetylgalactosamine (GalNAc) moieties, lactose moieties, lactobionic acid moieties, and sterylglucoside moieties. In embodiments, the CTM binds a mannose-6-phosphate receptor. In embodiments, the CTM binds a asialoglycoprotein receptor.

In embodiments, the CTM comprises a carbohydrate such as mannose, mannose-6-phosphate, galactosamine, N-acetylgalactosamine (GalNAc), lactose, lactobionic acid, galactose, galactosamine, galactoside, glucose, or steryl glucoside. In embodiments, the CTM comprises galactoside, galactosamine, GalNAc, lactose, lactobionic acid, or sterylglucoside. In embodiments, the CTM comprises galactosamine. In embodiments, the CTM comprises GalNAc, which may be alpha- or beta-GalNac. In embodiments, the CTM comprises beta-GalNAc. In embodiments, mannose is D-mannose. In embodiments, the CTM targets the compound to liver cells and comprises GalNAc and galactose. In embodiments, the CTM targets the compound to macrophages cells and comprises mannose and galactose. In embodiments, the CTM targets the compound to muscles and comprises glucose.

The compound may comprise a CTM moiety, for example, a GalNAc moiety, which can also be referred to as a GalNAc cluster, which are described in US Patent Application Publication No. US 2020/0361983 A1, which is hereby incorporated herein by reference in its entirety. As used herein, a CTM moiety can include one or more galactosamine moieties, for example, from one to four galactosamine moieties, one to nine galactosamine moieties or one, two, three, four, five, six, seven, eight or nine galactosamine moieties. Galactosamine, GalNac and GalNAc moiety are asialoglycoprotein receptor targeting moieties which may be used to target the compound to hepatotcytes, for example, to treat liver diseases. The asialoglycoprotein receptor is present at a high density on liver cells. Additionally, the turn-over rate of asialoglycoprotein receptors on liver cells is high. Due to the high concentration and rapid turnover of asialoglycoprotein receptors on liver cells, rapid accumulation of GalNAc or compounds comprising a GalNAc moiety into liver cells may occur through endocytosis.

In embodiments a CTM moiety comprises from one to four carbohydrate moieties, one to nine carbohydrate moieties or one, two, three, four, five, six, seven, eight or nine carbohydrate moieties. In embodiments, a CTM moiety comprises 3 or 4 carbohydrate moieties, such as 3 or 4 galactosamine moieties. In embodiments, a CTM moiety comprises 3 or 4 GalNAc moieties. In embodiments, a CTM moiety comprises 3 galactosamine moieties. In embodiments, a CTM moiety comprises 3 GalNAc moieties. In embodiments, a CTM moiety comprises more than one type of carbohydrate moiety, which may alter tissue distribution. For example, a CTM moiety may comprise at least one D-mannose moiety in addition to at least one GalNac moiety.

The galactosamine moieties of a CTM moiety may be conjugated to a branch point of a suitable linker. The linker may be of any suitable length. In embodiments, the linkers have length and other characteristics, such as hydrophilic-hydrophobic balance and spatial geometry, as described in Huang et al., Bioconjugate Chem. 2017, 28, 283-295, which is hereby incorporated herein by reference in its entirety.

In embodiments, the linker includes an alkylene linker or an ethylene glycol linker each of which contains one or more peptide functionalities (—CO—NH—) in the alkylene chain or the ethylene glycol chain. In embodiments, the linker contains one peptide functionality (—CO—NH—) in the alkylene or ethylene glycol chain. In embodiments, the linker comprises an arylene linker with —NHC(═S)-functionality. In embodiments, the linker comprises an alkylene linker or ethylene glycol linker each of which contains at least one functionality that can undergo click chemistry (e.g., an azide, —N₃, functionality). In embodiments, the linker comprises an ethylene glycol linker containing at least one functionality that can undergo click chemistry (e.g., an azide, —N₃, functionality).

Each galactosamine moiety of the CTM moiety may be bound to the linker via the same or different groups. In embodiments, each galactosamine moiety of the CTM moiety is bound to the linker via the same group. In embodiments, at least two of the galactosamine moieties of the CTM moiety are bound to the linker via a different group.

In embodiments, an alkylene linker comprises a C₂₋₁₂-alkylene bridge. In embodiments, the C₂₋₁₂-alkylene bridge comprises a bivalent linear or branched saturated hydrocarbon group of 2 to 12 carbon atoms. In embodiments, the alkylene linker comprises 4 to 8 carbon atoms. In embodiments, the alkylene linker comprises 6 carbon atoms. In embodiments, the alkylene linker comprises butylene, pentylene, hexylene, heptylene or octylene or their isomers. In embodiments, the alkylene linker comprises n-hexylene.

In embodiments, the linker comprises ethylene glycol. In embodiments, the linker comprises from 1 to 20 ethylene glycol, —(CH₂)₂—O—, units. In embodiments, the linker comprises 2 to 6, 2 to 10, 3 to 5, or 10 to 20 ethylene glycol units. In embodiments, the linker comprises 3 ethylene glycol units.

In embodiments, an arylene linker comprises a C₆₋₁₂-arylene bridge. In embodiments, the C₆₋₁₂-arylene bridge comprises a bivalent linear or branched aromatic group of 2 to 12 carbon atoms. In embodiments, the arylene linker comprises 6 to 10 carbon atoms. In embodiments, the aryelene linker comprises 6 carbon atoms. In embodiments, the arylene linker comprises phenylene, naphthylene and the like. In embodiments, the arylene linker comprises phenylene.

The linker may comprise a branch point. A “branch point” in this context typically means a small molecule which permits attachment of two or more, for example from one to four carbohydrate moieties (e.g., galactose or mannose derivatives), three or four carbohydrate moieties, one to nine carbohydrate moieties or one, two, three, four, five, six, seven, eight or nine carbohydrate moieties (e.g., galactose derivatives, such as galactosamine or GalNAc) and further permits attachment of the branch point to the oligomer (e.g., ethylene glycol) In embodiments, the branch point comprises di-lysine. Di-lysine contains three amine groups through which three galactose-linker-derivatives may be attached and a carboxyl group through which the CTM moiety may be attached to the oligonucleotide. In embodiments, the branch point comprises a polypeptide comprising from two to 20 peptides, such as from 2 to 10, 4 to 10, 6 to 12, 8 to 14 or 12 to 18 peptides or one, two, three, four, five, six, seven, eight, nine, ten, 11, 12, 13, 14, 15, 16, 17, 18, 19 or 20 peptides. In embodiments, the branch point may comprise any amino acid including, but not limited to, lysine. glycine. and combinations thereof.

In embodiments, a CTM moiety has a structure of Formula MC-A, as follows:

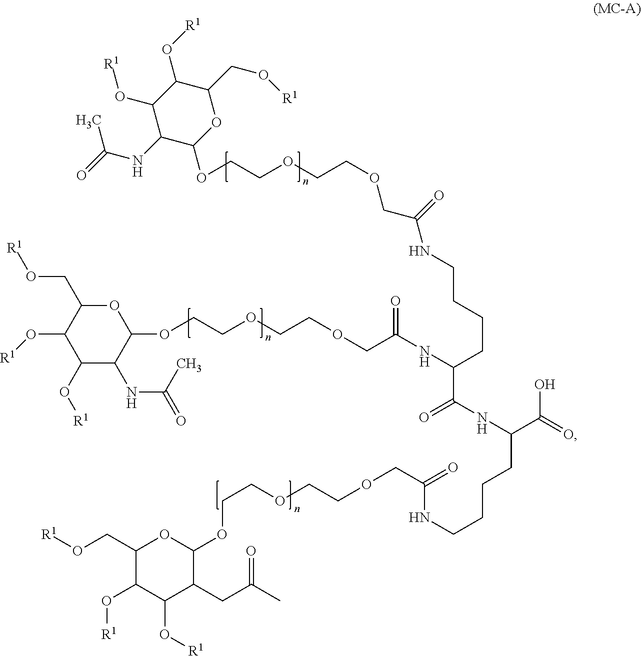

wherein
wherein R¹ is hydrogen or a hydroxy protecting group, and n is an integer from 0 to 10, and corresponding salts, enantiomers and/or a stereoisomer thereof.

In embodiments, R¹ is hydrogen or acetyl. In embodiments, R¹ is hydrogen.

In embodiments, n is 1 to 5. In embodiments, n is 2.

In embodiments, the CTM moiety is a GalNAc moiety having a structure of Formula MC-B, as follows:

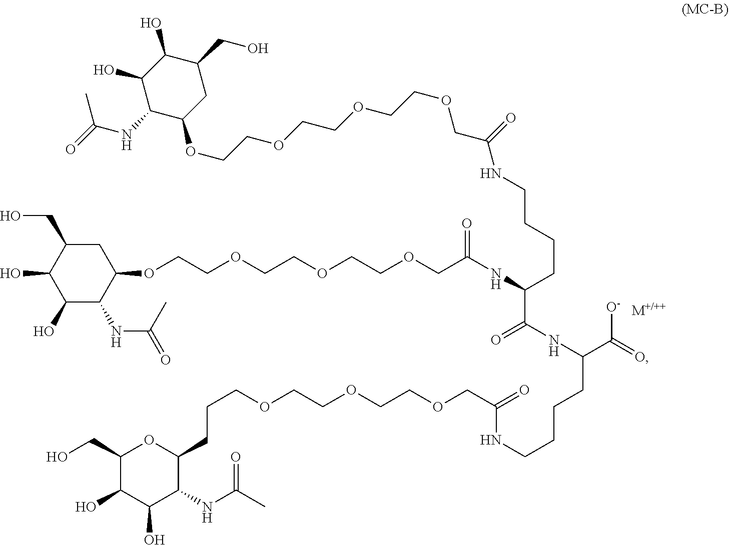 [SMILES: CC(=O)N[C@@H]1[C@@H](O)[C@@H](O)[C@@H](CO)O[C@H]1CCCOCCOCCOCC(=O)NCCCCC(NC(=O)[C@H](CCCCNC(=O)COCCOCCOCCO[C@@H]1C[C@H](CO)[C@H](O)[C@H](O)[C@H]1NC(C)=O)NC(=O)COCCOCCOCCO[C@@H]1C[C@H](CO)[C@H](O)[C@H](O)[C@H]1NC(C)=O)C(=O)[O-]]

wherein
wherein M^{+/++} is the cation of an alkali metal or of an earth alkali metal as defined above, preferably of an alkali metal and more preferably sodium.

In embodiments, a CTM moiety has a structure of Formula MC-C-MC-Q, as follows as follows:

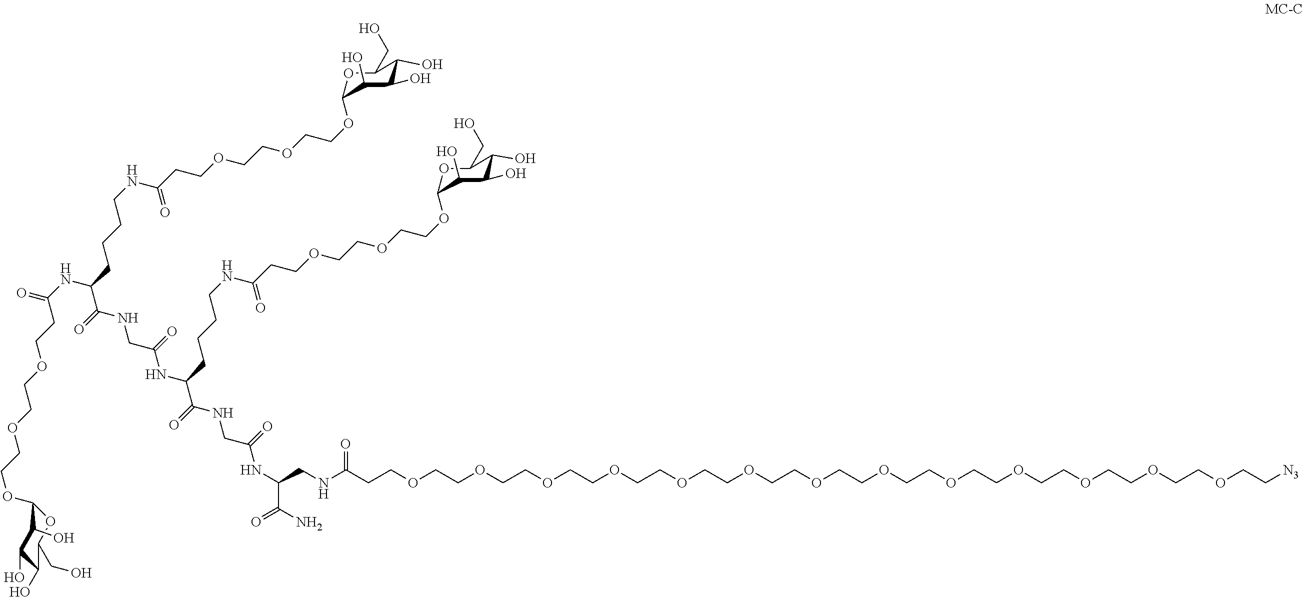

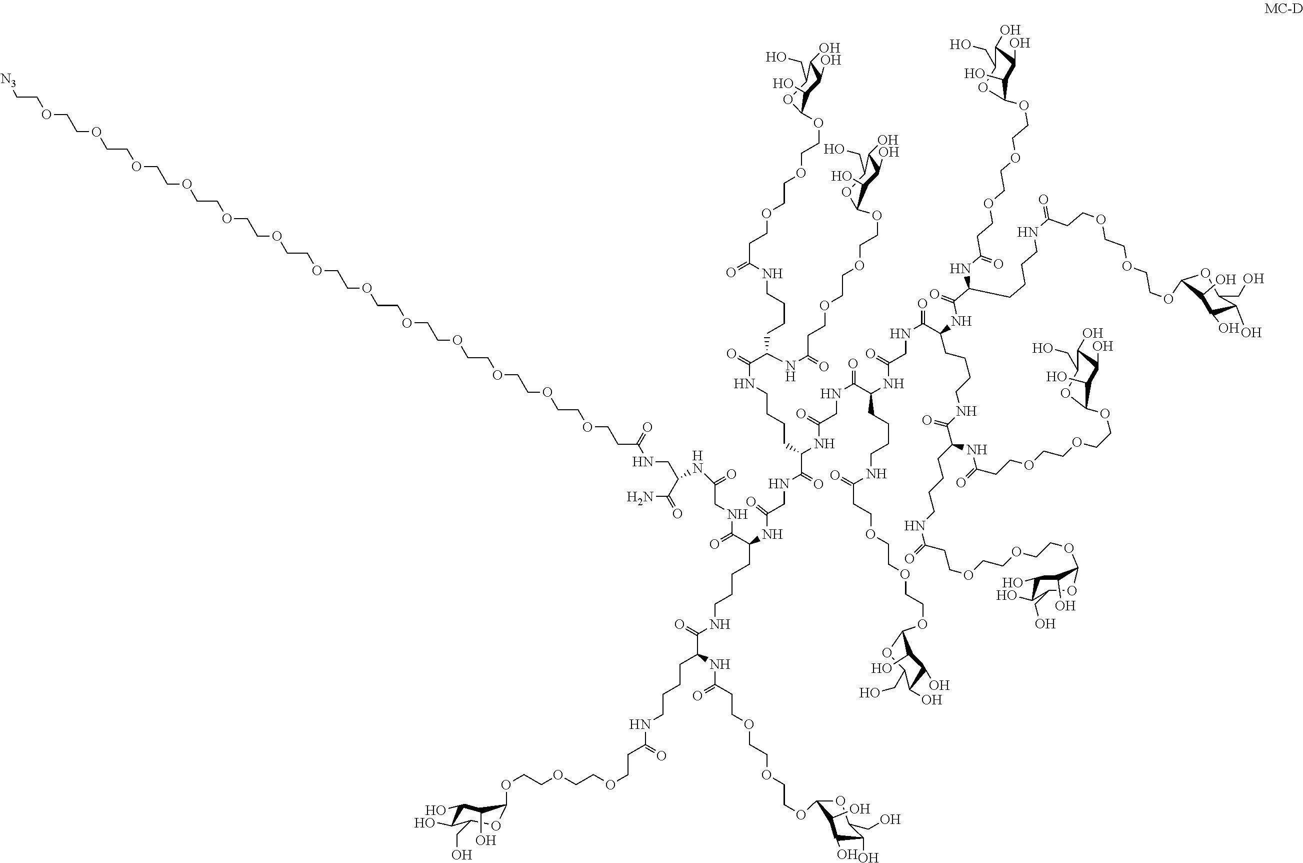

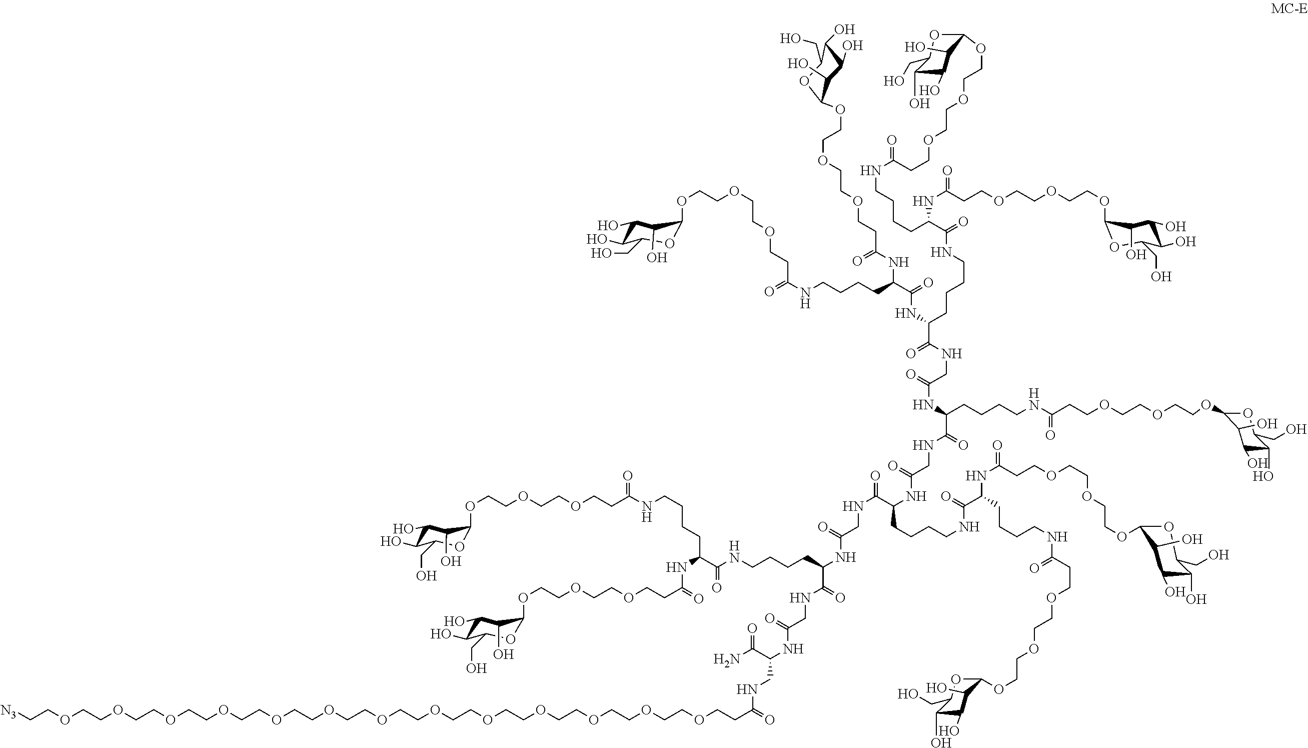

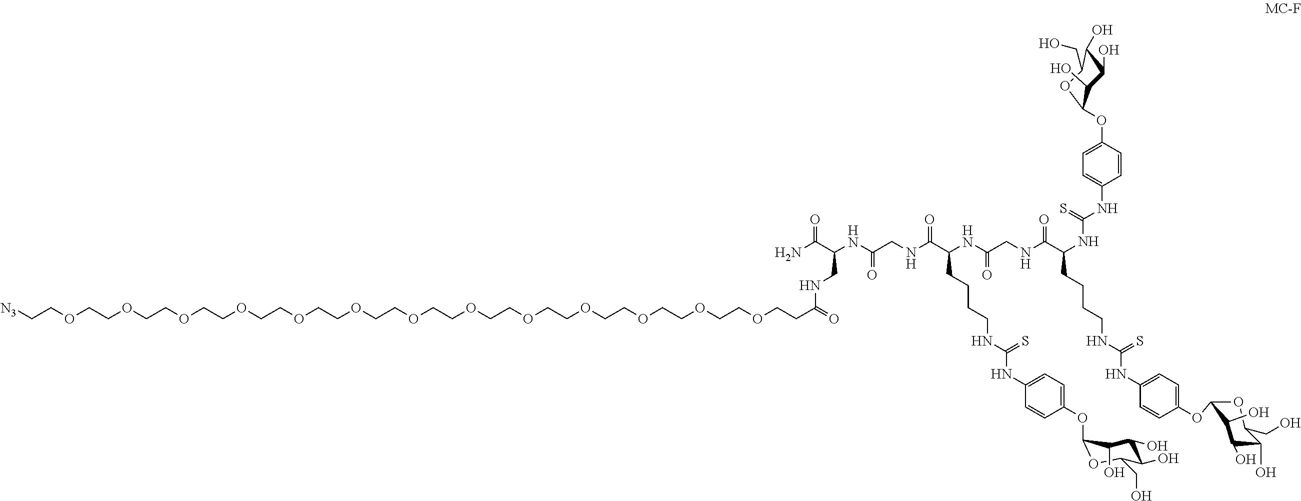

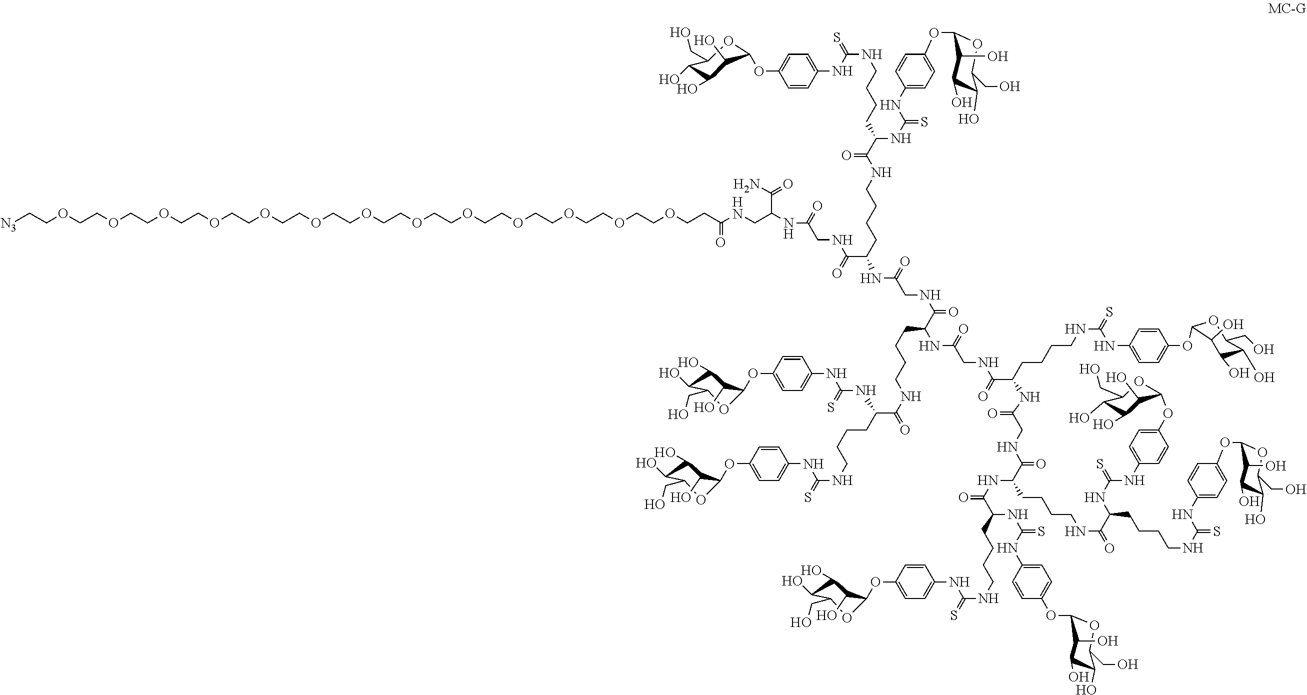

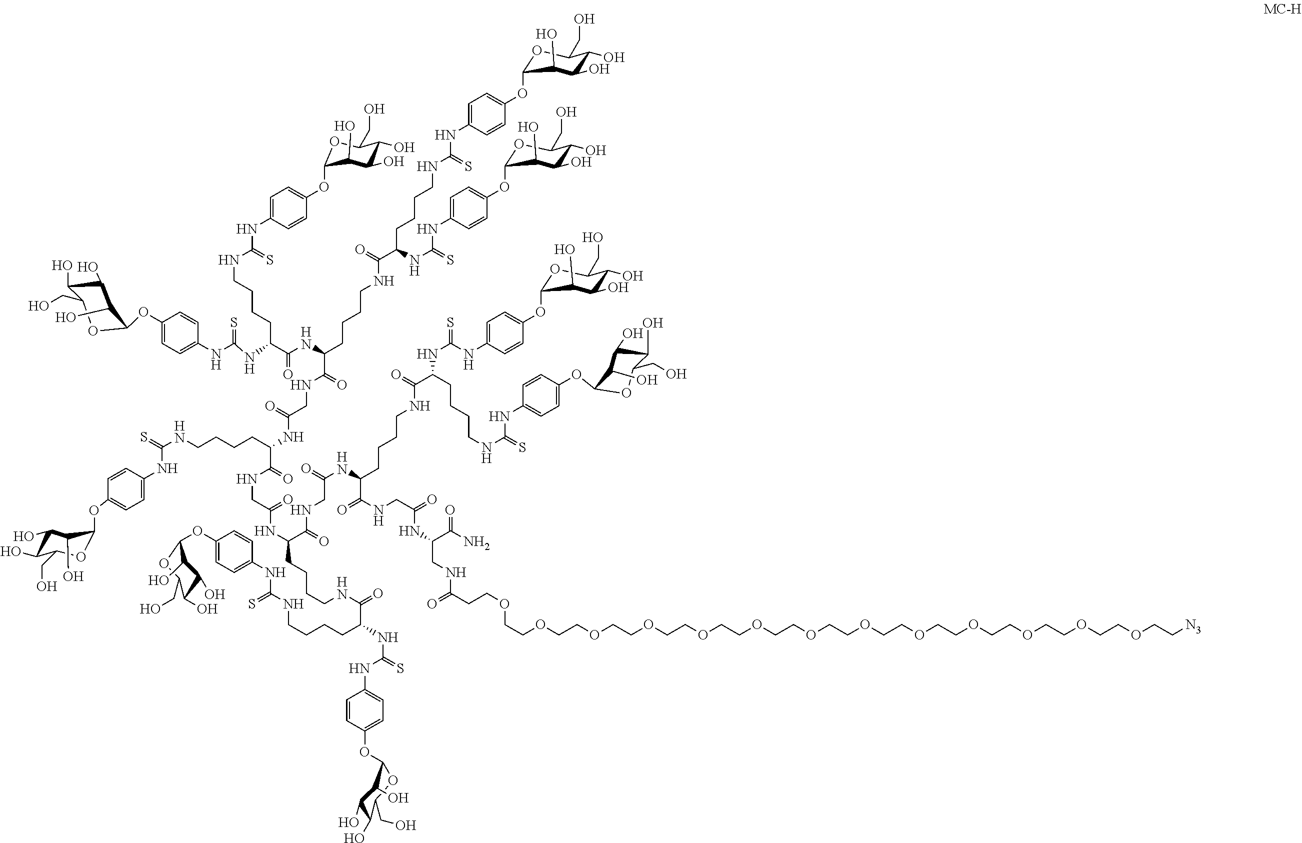

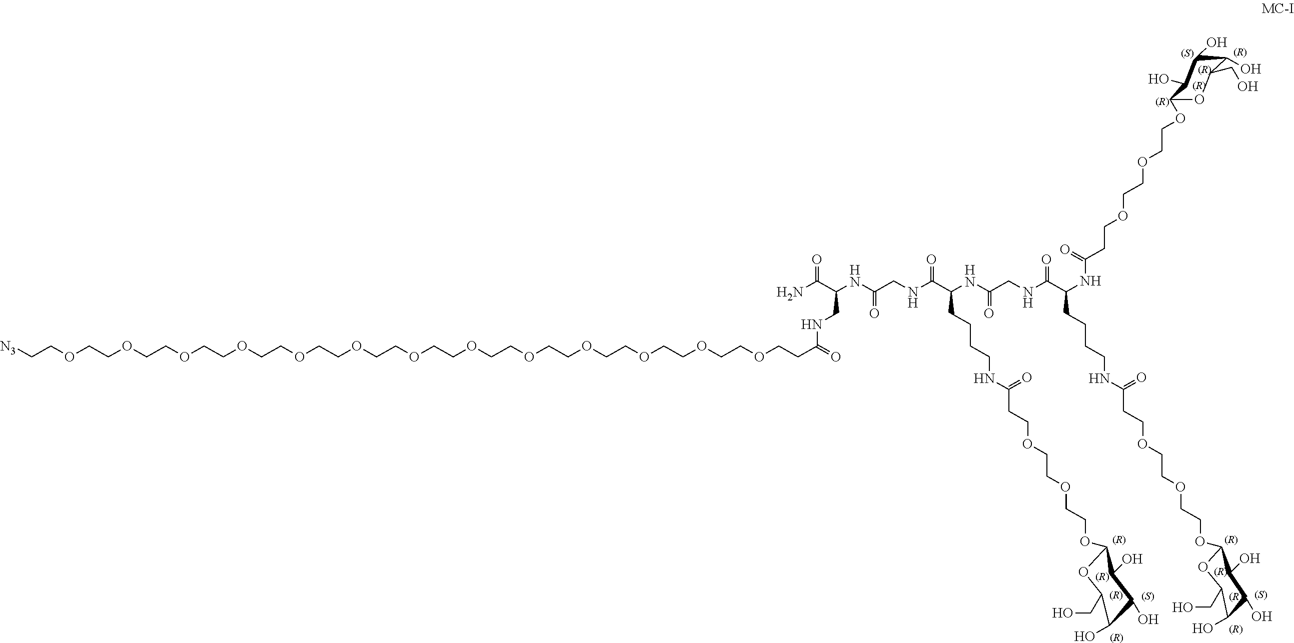

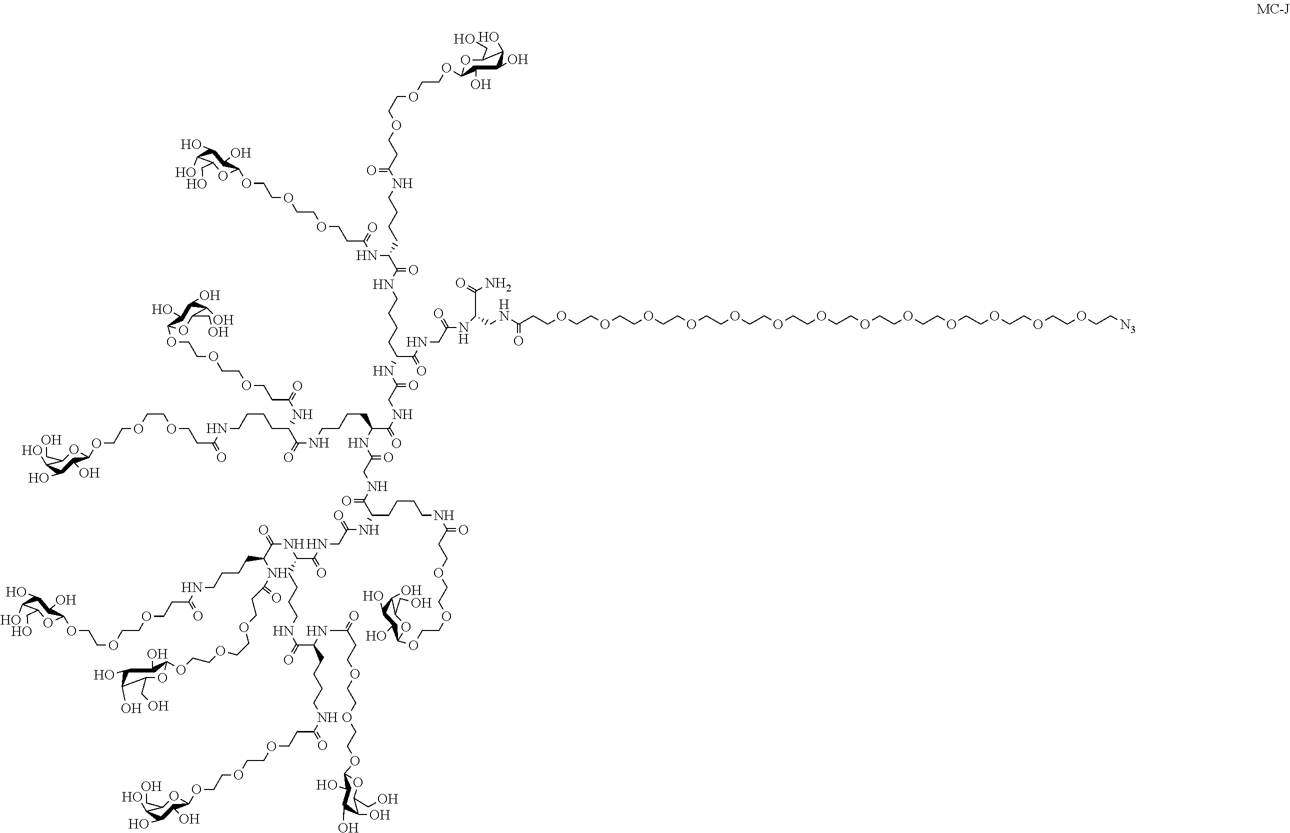

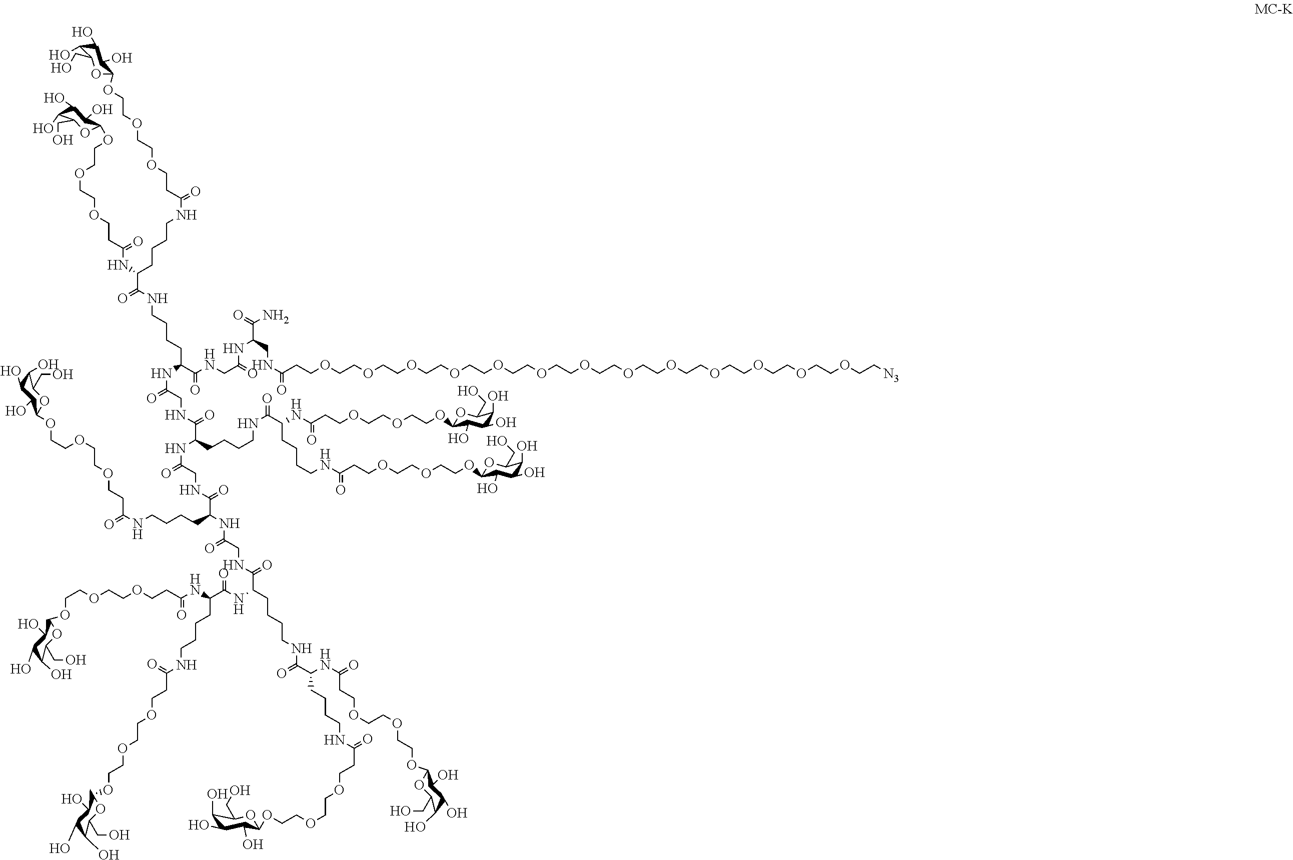

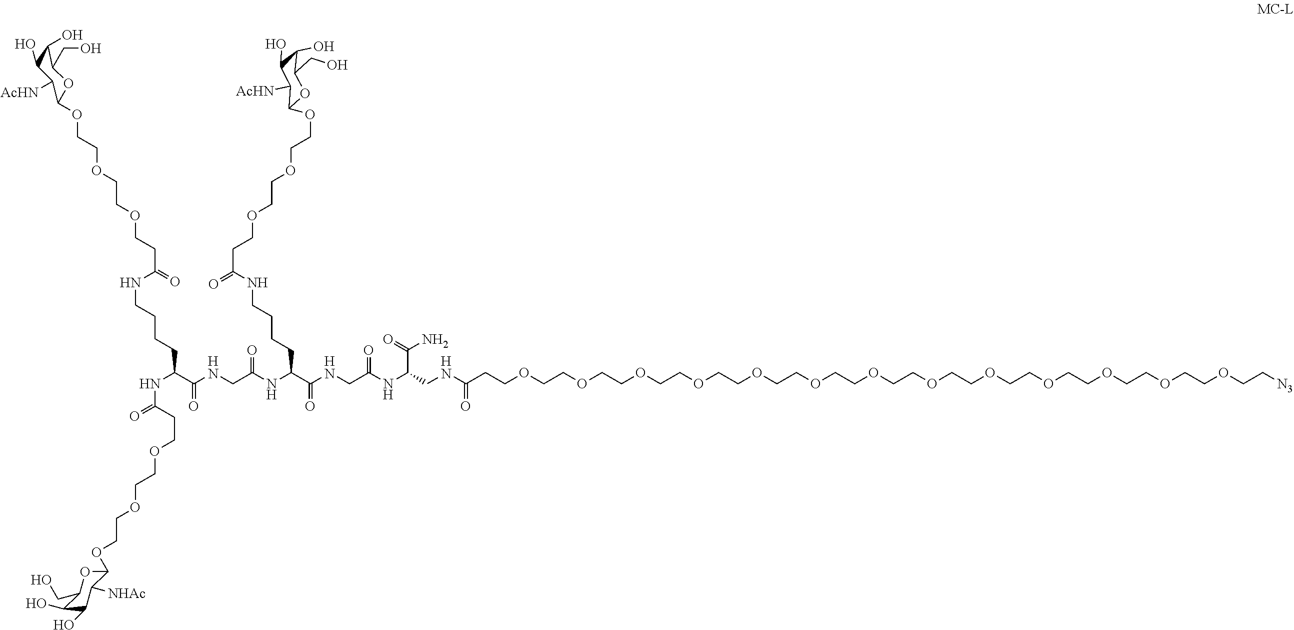

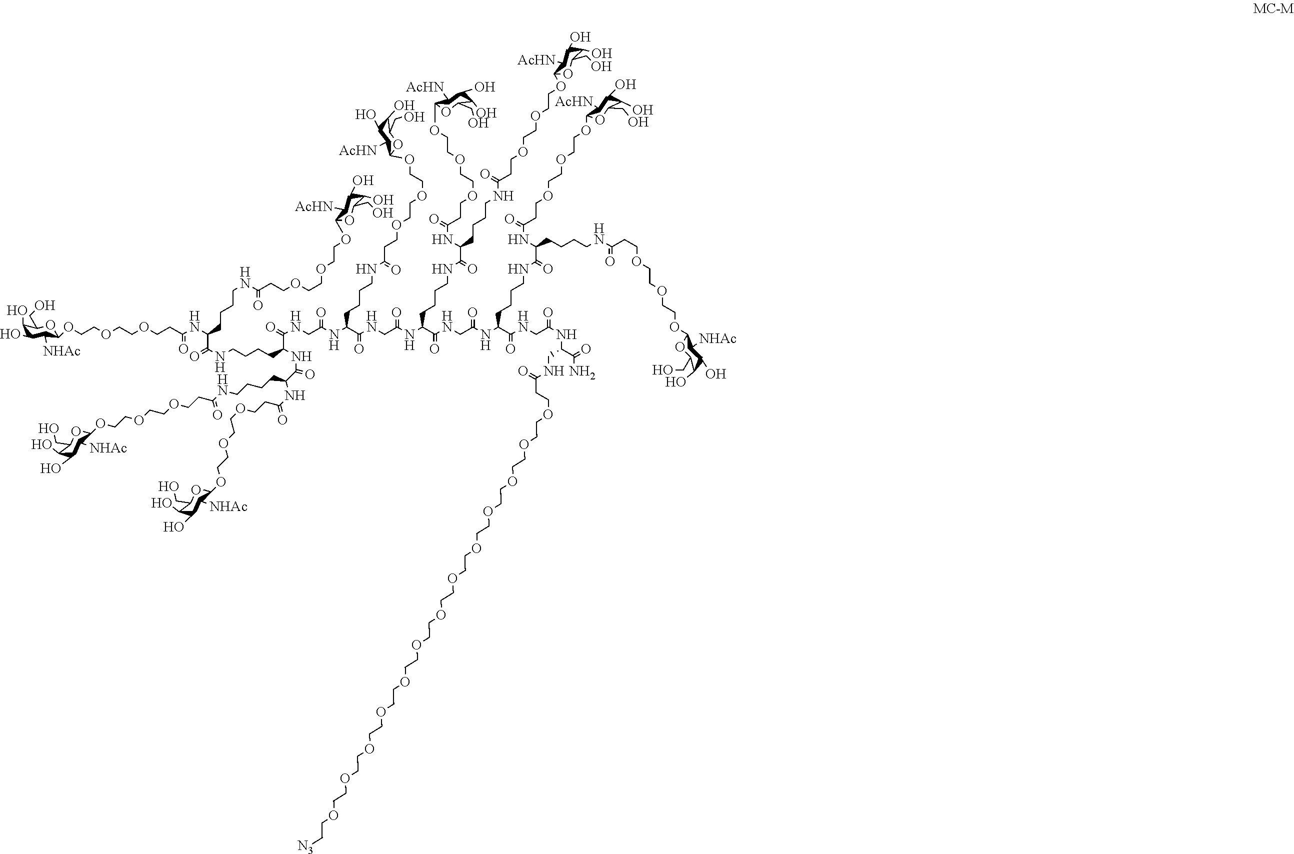

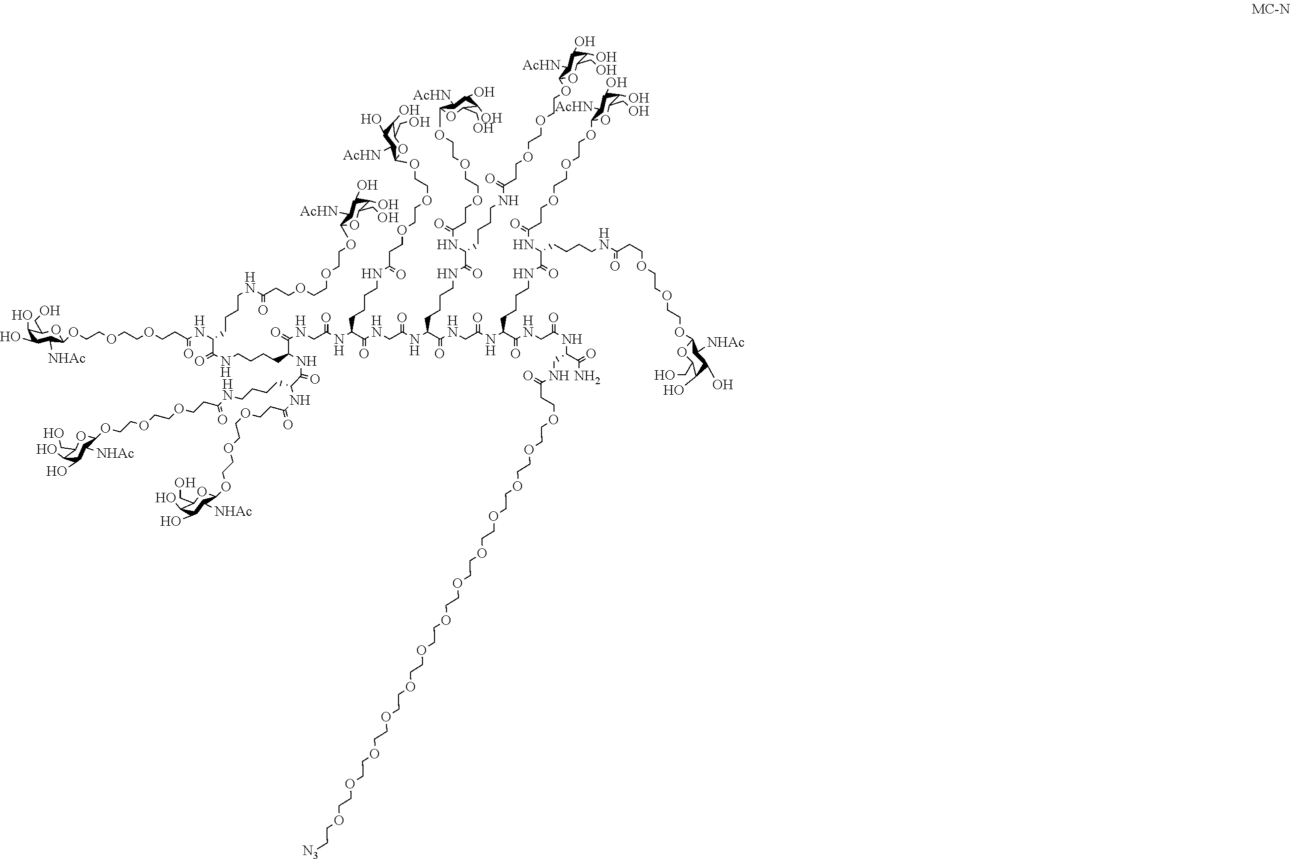

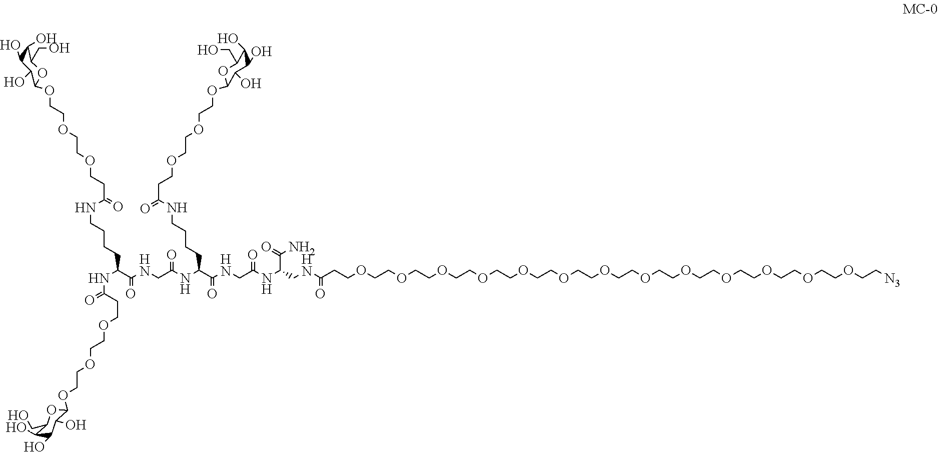

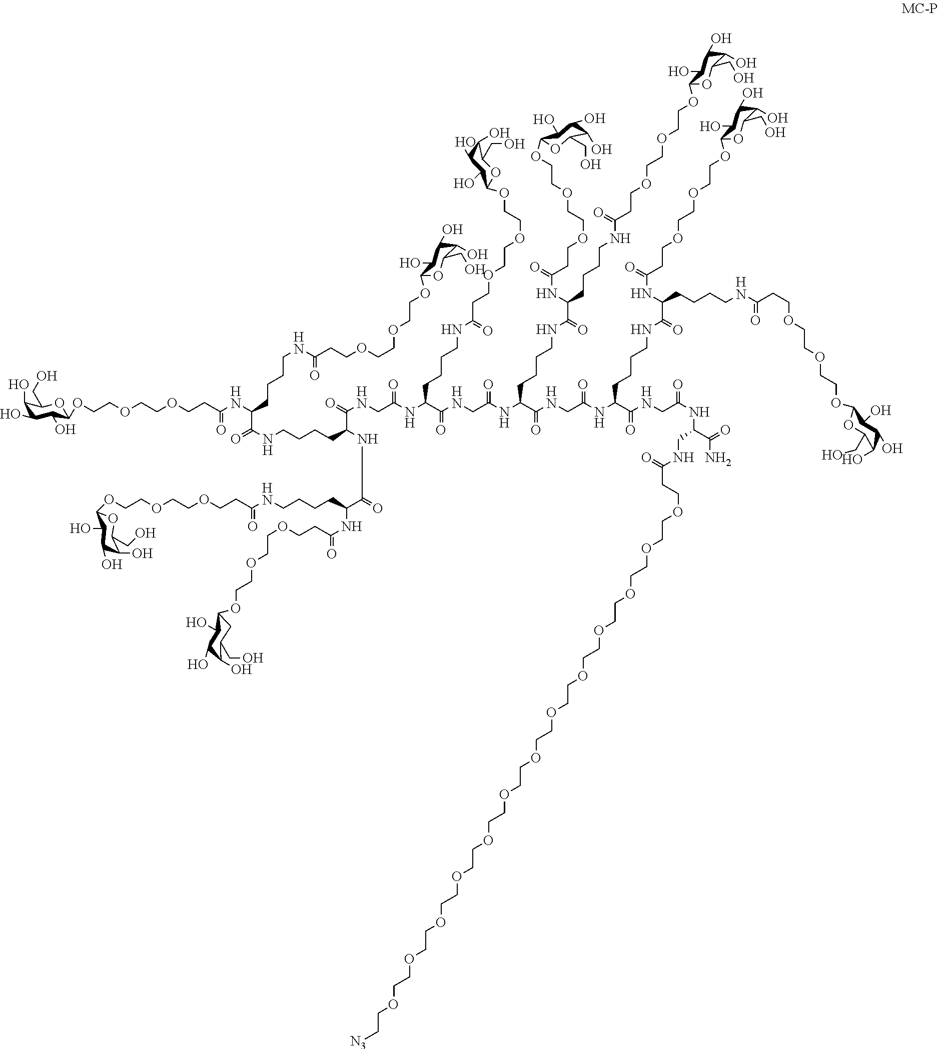

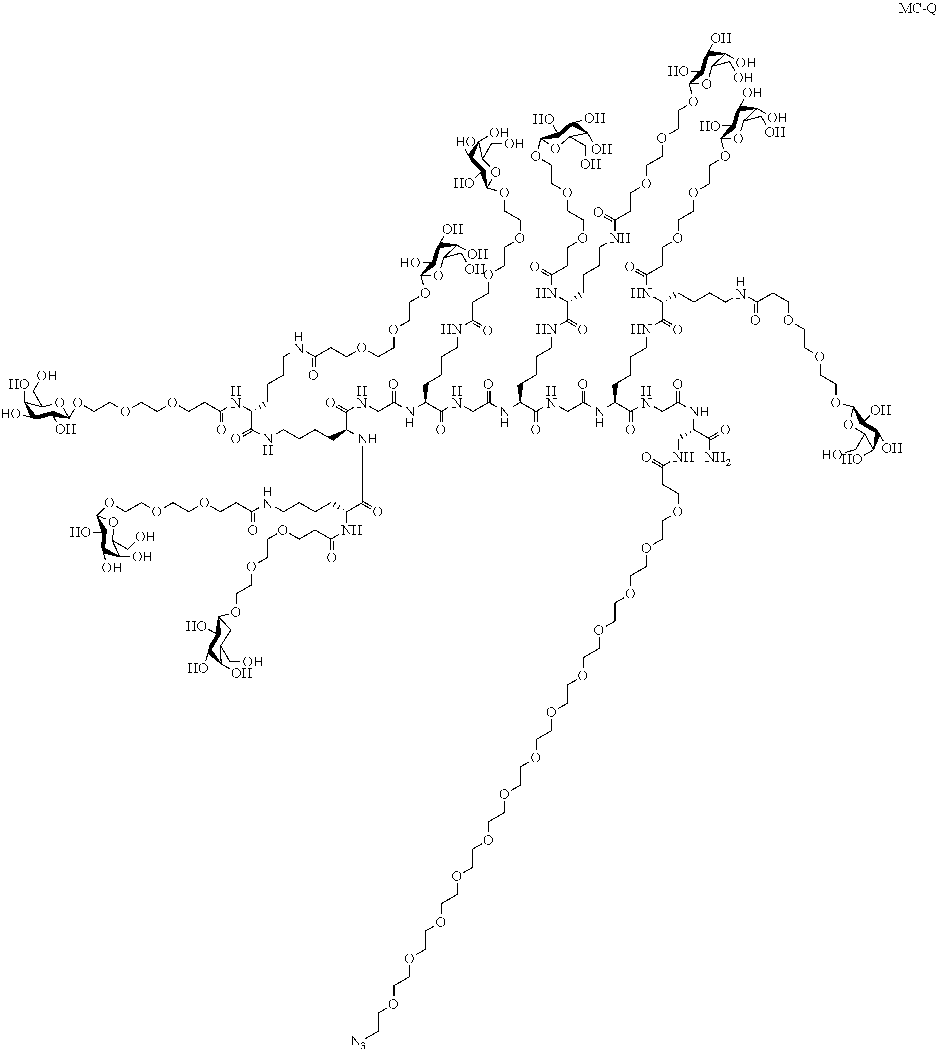

And even though the structures MC-C-MC-Q may comprise only mannose or only GalNac moieties, structures are contemplated herein wherein a mixture of mannose moieties and GalNac moieties are comprised with in the same CTM structure.

The CTM moiety may be prepared in any suitable manner. In embodiments, the CTM moiety is prepared according to the methods described in the PCT Publication WO2017/021385 (which is incorporated by reference as if fully set forth herein) and as shown in the scheme below.

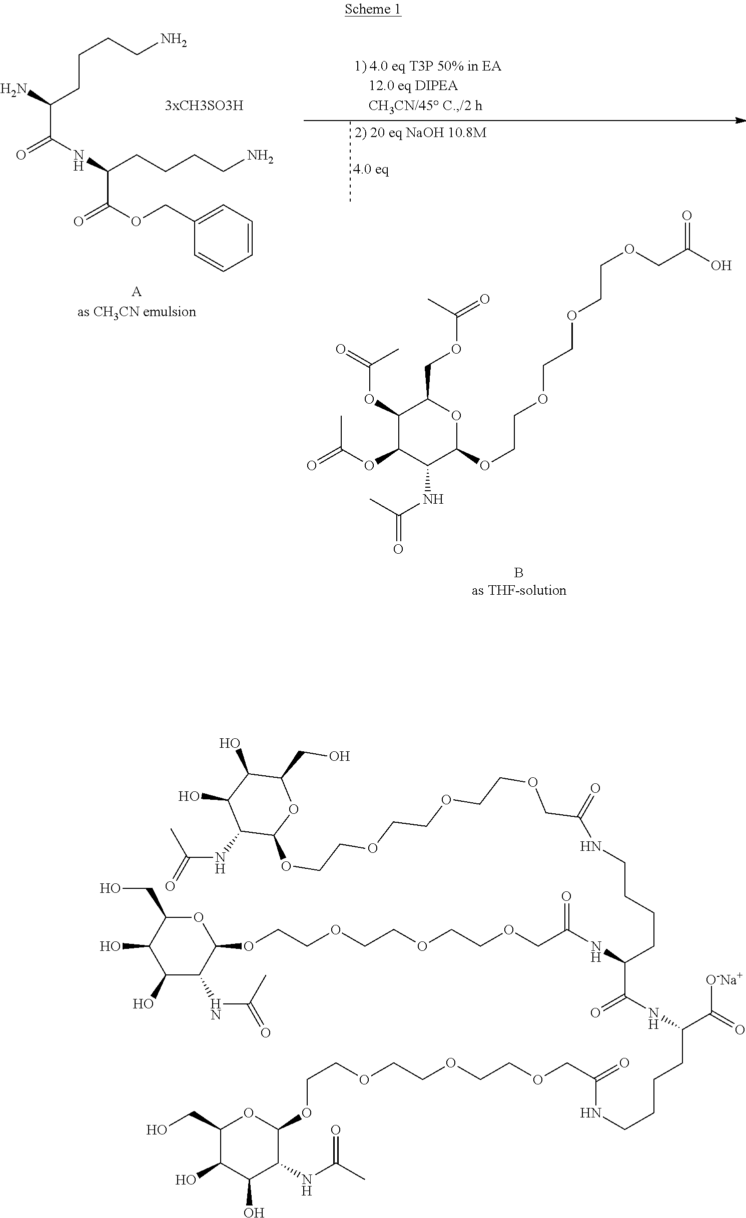

In embodiments, the therapeutic oligonucleotide (TO) is 5′ or 3′amino modified TO for reacting with the CTM moiety. The 5′ amino modified TO comprises a reactive amino group or azide covalently bound to a linker that is attached at the 5′ terminal group of an oligonucleotide. The 3′ amino modified TO comprises a reactive amino group or azide covalently bound to a linker that is attached at the 3′ terminal group of an oligonucleotide. In embodiments, the linker is an aliphatic alkyl group of 2 to 12 carbon atoms or an ethylene glycol linker containing 1 to 10 ethylene glycol units.

A 5′ modifier could comprise a cyclooctyne group (e.g., cyclooctyne, DBCO or BCN). The cyclooctyne group can further comprise a linker linking it to the TO, including a linker comprising a PEG group, aromatic group or a alkyl group. In embodiments, the 5′ amino-modifier is a C₂₋₁₂-alkyl linker, wherein the amino group is optionally protected. In embodiments, the 5′ amino-modifier is an C₄₋₈-alkyl linker, wherein the amino group is optionally protected. In embodiments, the 5′ amino-modifier is a C₆-alkyl linker.

A 5′ amino modified TO may comprise any suitable amino protecting group. In embodiments, the amino protecting group is trifluoroacetyl (TFA). In embodiments, the amino protecting group is monomethoxytrityl (MMT).

Accordingly, the 5′ amino modified TO can comprise: 5′-NR₂-linker1-X-TM-linker2-PMO, wherein NR₂ is a primary or secondary amino group optionally protected, X can be amide, carbamate, thioamide, or thiocarbamate, TM is a triazine moiety, linker1 and linker2 are independently PEG, aromatic or aliphatic linker of various length. Linker1 and linker2 can be the same or different.

A 3′ modifier could comprise a cyclooctyne group (e.g., cyclooctyne, DBCO or BCN). The cyclooctyne group can further comprise a linker linking it to the TO, including a linker comprising a PEG group, aromatic group or an alkyl group.

Accordingly, the 3′ amino modified TO can comprise: 3′-NR₂-linker-X′-PMO, wherein NR₂ is a primary or secondary amino group optionally protected, and the linker can be a PEG, aromatic or aliphatic linkage of various length, and X′ can be amide, carbamate, thioamide, or thiocarbamate,

A 5′ or 3′ modifier can comprise an amide, carbamate, or thiocarbamate between the cyclooctyne group the linkage between the cyclooctyne moiety linker linking it to the TO.

In embodiments, the 3′ amino-modifier can be any group comprising 2 amino groups and one carboxylic acid to install targeting moiety and/or cCPP. In some embodiment, the amino modifier comprises lysine, Dab (2,4-diaminobutyric acid), Dap (2,3-diaminopropanoic acid), and the like.

In embodiments, the amino linker may be introduced via a commercially available amino linker phosphoroamidite such as for instance via a TFA- or MMT-C₆-linker phosphoroamidite (e.g., from Sigma Aldrich) or via the 5′ amino modifier TEG (triethyleneglycol) CE phosphoroamidite from Glen Research.

In embodiments, a CTM-TO conjugate for producing compounds of the present disclosure may be as described in U.S. Pat. No. 8,450,467 B2, which is hereby incorporated by reference in its entirety.

The CTM may be coupled to a modified nucleotide of the TO. For example, the sugar moiety of one or more nucleotides of the TO can be replaced with another moiety, e.g., a non-carbohydrate (e.g., cyclic) carrier to which is coupled the CTM. A nucleotide in which the sugar has been so replaced is referred to herein as a replacement modification subunit (RMS). A cyclic carrier may be a carbocyclic ring system. In embodiments, all ring atoms are carbon atoms. In embodiments, the ring system is a heterocyclic ring system. In embodiments, one or more ring atoms are a heteroatom. In embodiments, the heteroatom is nitrogen, oxygen, or sulfur. The cyclic carrier may be a monocyclic ring system, or may contain two or more rings, e.g. fused rings. The cyclic carrier may be a fully saturated ring system, or it may contain one or more double bonds.

In embodiments, the carrier may further include (i) at least one backbone attachment point and (ii) at least one tethering attachment point. In embodiments, the carrier comprises two backbone attachment points. A “backbone attachment point,” as used herein, refers to a functional group or a bond available for, and that is suitable for, incorporation of the carrier into the backbone, e.g., the phosphate, or modified phosphate, e.g., sulfur containing, backbone, of a nucleic acid. In embodiments, the functional group comprises a hydroxyl group.

In embodiments, the carrier comprises a tethering attachment point (TAP). As used herein, a “tethering attachment point” is a constituent ring atom of a cyclic carrier, e.g., a carbon atom or a heteroatom (distinct from an atom which provides a backbone attachment point), that connects a selected CTM moiety. The CTM moiety comprise a carbohydrate, e.g. monosaccharide or a polysaccharide (e.g., a disaccharide, a trisaccharide, a tetrasaccharide, and an oligosaccharide). Optionally, the selected moiety is connected by an intervening tether to the cyclic carrier. In embodiments, the cyclic carrier includes a functional group, e.g., an amino group, or generally, provides a bond, that is suitable for incorporation or tethering of another chemical entity, e.g., a CTM to the constituent ring.

In embodiments, a CTM-TO conjugate, or portion thereof, may include a structure according to Formula (CI), as follows:

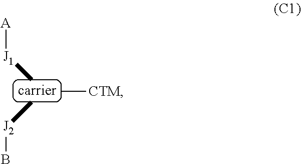

herein
A and B are independently for each occurrence hydrogen, protecting group, optionally substituted aliphatic, optionally substituted aryl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodiester, a phosphotriester, an activated phosphate group, an activated phosphite group, a phosphoramidite, a solid support, —P(Z¹)(Z²)—O-nucleoside, or —P(Z¹)(Z²)—O-oligonucleotide; wherein Z¹ and Z² are each independently for each occurrence O, S, N(alkyl) or optionally substituted alkyl; J₁ and J₂ are independently O, S, NRN, optionally substituted alkyl, OC(O)NH, NHC(O)O, C(O)NH, NHC(O), OC(O), C(O)O, OC(O)O, NHC(O)NH, NHC(S)NH, OC(S)NH, OP(N(RP)₂)O, or OP(N(RP)₂); [symbol]
is cyclic group or acyclic group; and
CTM is a carbohydrate targeting moiety as described herein.

In embodiments, the cyclic group of the carrier is pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl or decalin. In embodiments, the acyclic group is serinol backbone or diethanolamine backbone.

In embodiments, CTM comprises a monosaccharide. In embodiments, the CTM comprises a polysaccharide. In embodiments, the CTM comprises a disaccharide. In embodiments, the CTM comprises a trisaccharide. In embodiments, the CTM comprise a tetrasaccharide.

In embodiments, the CTM-TO conjugate, or a portion thereof, includes a pyrrolidine ring system as shown in Formula (CII)

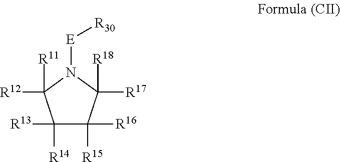

wherein E is absent or C(O), C(O)O, C(O)NH, C(S), C(S)NH, SO, SO₂, or SO₂NH;
R¹¹, R¹², R¹³, R¹⁴, R¹⁵, R¹⁶, R¹⁷, and R¹⁸ are each independently for each occurrence H, —CH₂OR^{a}, or OR^{b},
R^{a} and R^{b} are each independently for each occurrence hydrogen, hydroxyl protecting group, optionally substituted alkyl, optionally substituted aryl, optionally substituted cycloalkyl, optionally substituted aralkyl, optionally substituted alkenyl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodiester, a phosphotriester, an activated phosphate group, an activated phosphite group, a phosphoramidite, a solid support, —P(Z¹)(Z²)—O-nucleoside, —P(Z¹)(Z²)—O-oligonucleotide, —P(Z¹)(O— coupler —R^{L})—O-nucleoside, or —P(Z¹)(O— coupler —R^{L})—O-oligonucleotide;
R³⁰ is independently for each occurrence -coupler-R^{L} or R³¹;
R^{L} is hydrogen or a CTM;
R³¹ is —C(O)CH(N(R³²)₂)(CH₂)ₕN(R³²)₂;
R³² is independency for each occurrence H, —R^{L}, -coupler-R^{L} or R³¹;
Z¹ is independently for each occurrence O or S;
Z² is independently for each occurrence O, S, N(alkyl) or optionally substituted alkyl; and
h is independently for each occurrence 1-20.

For the pyrroline-based click-carriers, R¹¹ is —CH₂OR^{a} and R³ is OR^{b}; or R¹¹ is —CH₂OR^{a} and R⁹ is OR^{b}; or R¹¹ is —CH₂OR^{a} and R¹⁷ is OR^{b}; or R¹³ is —CH₂OR^{a} and R¹¹ is OR^{b}; or R¹³ is —CH₂OR^{a} and R¹⁵ is OR^{b}; or R¹³ is —CH₂OR^{a} and R¹⁷ is OR^{b}. In embodiments, CH₂OR^{a} and OR^{b} may be geminally substituted. For the 4-hydroxyproline-based carriers, R¹¹ is —CH₂OR^{a} and R¹⁷ is OR^{b}. In embodiments, the pyrroline- and 4-hydroxyproline-based carriers contain linkages (e.g., carbon-carbon bonds) wherein bond rotation is restricted about that particular linkage, e.g. restriction resulting from the presence of a ring. In embodiments, CH₂OR^{a} and OR^{b} may be cis or trans with respect to one another in any of the pairings delineated above. Accordingly, all cis/trans isomers are expressly included. The carriers may also contain one or more asymmetric centers and thus occur as racemates and racemic mixtures, single enantiomers, individual diastereomers and diastereomeric mixtures. All such isomeric forms of the carriers are expressly included (e.g., the centers bearing CH₂OR^{a} and OR^{b} can both have the R configuration; or both have the S configuration; or one center can have the R configuration and the other center can have the S configuration and vice versa).

In embodiments, R¹¹ is CH₂OR^{a} and R⁹ is OR^{b}.

In embodiments, R^{b} is a solid support.

In embodiments, carrier of Formula (CH) is a phosphoramidite, i.e., one of R^{a} or R^{b} is —P(O-alkyl)N(alkyl)₂, e.g., —P(OCH₂CH₂CN)N(i-propyl)₂. In embodiments, R^{b} is —P(O-alkyl)N(alkyl)₂.

In embodiments, the carrier comprises a ring system as shown in Formula (CIII).

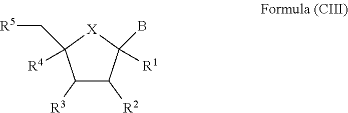

wherein:
X is O, S, NR^{N} or CR^{P}₂;
B is independently for each occurrence hydrogen, optionally substituted natural or non-natural nucleobase, optionally substituted natural nucleobase conjugated with -coupler —R^{L} or optionally substituted non-natural nucleobase conjugated with -coupler-R^{L};
R¹, R², R³, R⁴ and R⁵ are each independently for each occurrence H, OR⁶, F, N(R^{N}) or -J-coupler-R_{L},
J is absent, O, S, NR^{N}, OC(O)NH, NHC(O)O, C(O)NH, NHC(O), NHSO, NHSO₂, NHSO₂NH, OC(O), ((O)O, OC(O)O, NHC(O)NH, NHC(S)NH, OC(S)NH, OP(N(R^{N})₂)O, or OP(N(R^{P})₂);
R⁶ is independently for each occurrence hydrogen, hydroxyl protecting group, optionally substituted alkyl, optionally substituted aryl, optionally substituted cycloalkyl, optionally substituted aralkyl, optionally substituted alkenyl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodiester, a phosphotriester, an activated phosphate group, an activated phosphite group, a phosphoramidite, a solid support, —P(Z¹)(Z²)—O-nucleoside, —P(Z¹)(Z²)—O-oligonucleotide, —P(Z¹)(Z²)-formula (CIII), —P(Z¹)(O-coupler-R^{L})—O-nucleoside, —P(Z¹)(O-coupler-R^{L})—O-oligonucleotide, or —P(Z¹)(O— coupler —R^{L})—O-formula (CIII);
R^{N} is independently for each occurrence H, optionally substituted alkyl, optionally substituted alkenyl, optionally substituted alkynyl, optionally substituted aryl, optionally substituted cycloalkyl, optionally substituted aralkyl, optionally substituted heteroaryl or an amino protecting group
R^{P} is independently for each occurrence H, optionally substituted alkyl, optionally substituted alkenyl, optionally substituted alkynyl, optionally substituted aryl, optionally substituted cycloalkyl or optionally substituted heteroaryl;
R^{L} is hydrogen or a CTM;
Z¹ and Z² are each independently for each occurrence O, S N(alkyl) or optionally substituted alkyl; and
provided that R^{L} is present at least once and further provided that R^{L} is a CTM at least once.

In embodiments, the carrier of formula (CI) is an acyclic group and is termed an “acyclic carrier”. In embodiments, acyclic carriers have the structure shown in formula (CIV) or formula (CV) below.

In embodiments, the CTM-TO conjugate, or portion thereof, includes an acyclic carrier having the structure shown in Formula (CIV).

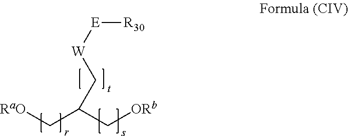

wherein.
W is absent, O, S or N(R^{N}), where R^{N} is independently for each occurrence H, optionally substituted alkyl, optionally substituted alkenyl, optionally substituted alkynyl, optionally substituted aryl, optionally substituted cycloalkyl, optionally substituted aralkyl, optionally substituted heteroaryl or an amino protecting group,
E is absent or C(O), C(O)O, C(O)NH, C(S), C(S)NH, SO, SO₂, or SO₂NH;
R^{a} and R^{b} are each independently for each occurrence hydrogen, hydroxyl protecting group, optionally substituted alkyl, optionally substituted aryl, optionally substituted cycloalkyl, optionally substituted aralkyl, optionally substituted alkenyl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodiester, a phosphotriester, an activated phosphate group, an activated phosphite group. a phosphoramidite, a solid support, —P(Z¹)(Z²)—O-nucleoside, —P(Z¹)(Z²)—O-oligonucleotide, —P(Z¹)(O— coupler —R^{L})—O— nucleoside, or —P(Z¹)(O— coupler —R^{L})—O-oligonucleotide;
R³⁰ is independently for each occurrence -coupler —R^{L} or R³¹;
R^{L} is hydrogen or a CTM;
R³¹ is —C(O)CH(N(R³²)₂(CH₂)ₕN(R³²)₂;
R³² is independently for each occurrence H, —R^{L}, -coupler —R^{L} or R³¹;
Z¹ is independently for each occurrence O or S;
Z² is independently for each occurrence O, S, N(alkyl) or optionally substituted alkyl,
h is independently for each occurrence 1-20; and
r, s and t are each independently for each occurrence 0, 1, 2 or 3.

When r and s are different, then the tertiary carbon can be either the R or S configuration. In embodiments, x and y are one and z is zero (e.g. carrier is based on serinol). The acyclic carriers can optionally be substituted, e.g. with hydroxy, alkoxy, perhaloalky.

In one embodiment, the CTM-TO conjugate includes an acyclic carrier having the structure shown in Formula (CV)

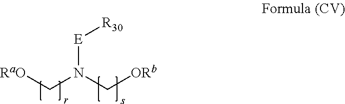

wherein E is absent or C(O), C(O)O, C(O)NH, C(S), C(S)NIH, SO, SO₂, or SO₂NH;
R^{a} and R^{b} are each independently for each occurrence hydrogen, hydroxyl protecting group, optionally substituted alkyl, optionally substituted aryl, optionally substituted cycloalkyl, optionally substituted aralkyl, optionally substituted alkenyl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodiester, a phosphotriester, an activated phosphate group, an activated phosphite group, a phosphoramidite, a solid support, —P(Z¹)(Z²)—O-nucleoside, —P(Z¹)(Z²)—O-oligonucleotide, —P(Z¹)(Z²)-formula (I), —P(Z¹)(O— coupler —R^{L})—O-nucleoside, or —P(Z¹)(O— coupler —R^{L})—O-oligonucleotide;
R³⁰ is independently for each occurrence -coupler —R^{L} or R³¹;
R^{L} is hydrogen or a CTM,
R³¹ is —C(O)CH(N(R³²)₂)(CH₂)ₕN(R₃₂)₂;
R³² is independently for each occurrence H, —R^{L}, -coupler —R^{L} or R³¹;
Z¹ is independently for each occurrence O or S;
Z² independently for each occurrence O, S, N(alkyl) or optionally substituted alkyl; and
h is independently for each occurrence 1-20; and
r and s are each independently for each occurrence 0, 1, 2 or 3.

In addition to the cyclic carriers described herein, RMS can include cyclic and acyclic carriers described in U.S. application Ser. No. 10/916,185 filed Aug. 10, 2004, now U.S. Pat. No. 7,745,608: U.S. application Ser. No. 10/946,873 filed Sep. 21, 2004; U.S. application Ser. No. 10/985,426, filed Nov. 9, 2004, now U.S. Pat. No. 7,723,509; U.S. application Ser. No. 10/833,934, filed Aug. 3, 2007, now U.S. Pat. No. 7,021,394; U.S. application Ser. No. 11/115,989, filed Apr. 27, 2005, now U.S. Pat. No. 7,626,014; and U.S. application Ser. No. 11/119,533, filed Apr. 29, 2005, now U.S. Pat. No. 7,674,778, each of which are hereby incorporated herein by reference in their respective entireties.

In embodiments, a CTM-TO conjugate, or a portion thereof, has the structure shown in Formula (D-I)

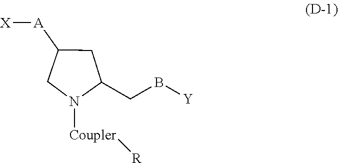

wherein
A and B are each independently for each occurrence O, N(R^{N}) or S;
R^{N} is independently for each occurrence H or C₁-C₆ alkyl;
X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)-oligonucleotide, a lipid, a PEG, a steroid, a polymer, a nucleotide, a nucleoside, —P(Z′)(Z″)O— coupler —OP(Z′″)(Z″″)O-oligonucleotide, an oligonucleotide, —P(Z′)(Z″)-formula (I), —P(Z′)(Z″)— or -coupler —R;
R is CTM or has the structure shown below.

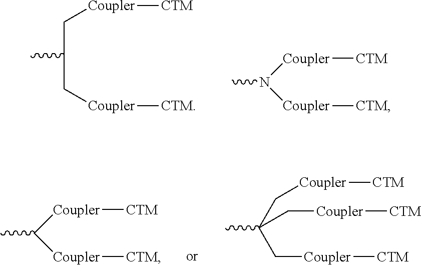

each CTM independently comprises a carbohydrate, and
Z′, Z″, Z′″, and Z′″ are each independently for each occurrence O or S.

The term “coupler” refers to an organic moiety that connects two parts of a compound. In embodiments, a coupler comprises a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkenyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one embodiment, the coupler is between 1-24 atoms, preferably 4-24 atoms, preferably 6-18 atoms, more preferably 8-18 atoms, and most preferably 8-16 atoms.

In embodiments, the coupler is —[(P-Q″-R)_{q}—X—(P′-Q′″—R′)_{q′}]-T-, wherein: P, R, T, P′, R′ and T are each independently for each occurrence absent, CO, NH, O, S, OC(O), NHC(O), CH₂, CH₂NH, CH₂O; NHCH(R^{a})C(O), —C(O)—CH(R)—NH—, CH═N—O,

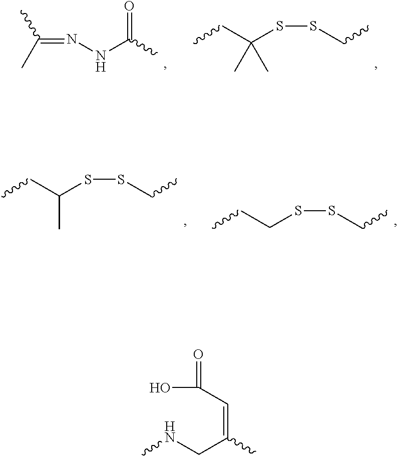

or heterocyclyl;
Q″ and Q′″ are each independently for each occurrence absent, —(CH₂)ₙ—, —C(R¹)(R²)(CH₂)ₙ—, —(CH₂)ₙC(R¹)(R²)—, —(CH₂CH₂O)ₘCH₂CH₂—, or —(CH₂CH₂O)ₘCH₂CH₂NH—;
X is absent or a cleavable coupling group:
R^{a} is H or an amino acid side chain;
R¹ and R² are each independently for each occurrence H, CH₃, OH, SH or N(R^{N})₂;
R^{N} is independently for each occurrence H, methyl, ethyl, propyl, isopropyl, butyl or benzyl;
q, q′ and q″ are each independently for each occurrence 0-20 and wherein the repeating unit can be the same or different; and
n is independently for each occurrence 1-20; and m is independently for each occurrence 0-50.

In embodiments, the coupler comprises at least one cleavable coupling group.

In embodiments, the coupler is a branched coupler. The branchpoint of the branched coupler may be at least trivalent, but may be a tetravalent, pentavalent or hexavalent atom, or a group presenting such multiple valencies. In embodiments, the branchpoint is, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In embodiments, the branchpoint is glycerol or glycerol derivative.

### Cleavable Coupling Groups

As used herein, a “cleavable coupling group” refers to a coupling group that is stable outside a cell, but which upon entry into a target cell is cleaved to release the two parts the coupler is holding together. In embodiments, the cleavable coupling group is cleaved at least 10 times or more in the target cell or under a first reference condition (which can, e.g., be selected to mimic or represent intracellular conditions) than in the blood of a subject, or under a second reference condition (which can, e.g., be selected to mimic or represent conditions found in the blood or serum). In embodiments, the cleavable coupling group is cleaved at least 100 times or more in the target cell or under a first reference condition (which can, e.g., be selected to mimic or represent intracellular conditions) than in the blood of a subject, or under a second reference condition (which can, e.g., be selected to mimic or represent conditions found in the blood or serum).

In embodiments, cleavable coupling groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable coupling group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable coupling group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some couplers will have a cleavable linking group that is cleaved at a preferred pH, thereby releasing the cationic lipid from the CTM inside the cell, or into the desired compartment of the cell.

In embodiments, a coupler includes a cleavable coupling group that is cleavable by an enzyme. The type of cleavable coupling group incorporated into a coupler can depend on the cell to be targeted. For example, liver targeting CTMs can be coupled to the cationic lipids through a coupler that includes an ester group. Liver cells are rich in esterases, and therefore the coupler will be cleaved more efficiently in liver cells than in cell types that are not esterase-rich. Other cell-types rich in esterases include cells of the lung, renal cortex, and testis.

Couplers that contain peptide bonds can be used when targeting cell types rich in peptidases, such as liver cells and synoviocytes.

In general, the suitability of a candidate cleavable coupling group can be evaluated by testing the ability of a degradative agent (or condition) to cleave the candidate coupling group. It will also be desirable to also test the candidate cleavable coupling group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Coupling Groups

In embodiments, a coupler includes a redox cleavable coupling group that is cleaved upon reduction or oxidation. In embodiments, the redox cleavable coupling group is a disulfide coupling group (—S—S—). To determine if a candidate cleavable coupling group is a suitable “reductively cleavable linking group,” or for example is suitable for use with a particular TO moiety and particular targeting agent, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In embodiments, candidate compounds are cleaved by at most 10% in the blood. In embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Coupling Groups

In embodiments, a coupler includes a phosphate-based cleavable coupling group. Phosphate-based cleavable coupling groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Coupling Groups

In embodiments, a coupler includes an acid cleavable coupling group. Acid cleavable coupling groups are coupling groups that are cleaved under acidic conditions. In embodiments, acid cleavable coupling groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable coupling groups. Examples of acid cleavable coupling groups include but are not limited to hydrazones, esters, and esters of amino acids. Acid cleavable groups can have the general formula —C═NN—, C(O)O, or —OC(O). In embodiments, the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Coupling Groups

In embodiments, a coupler includes an ester-based cleavable coupling group. Ester-based cleavable coupling groups are cleaved by enzymes such as esterases and amidases in cells. Examples of ester-based cleavable coupling groups include but are not limited to esters of alkylene, alkenylene and alkynylene groups. Ester cleavable coupling groups have the general formula —C(O)O—, or —OC(O)—. These candidates can be evaluated using methods analogous to those described above.

### Peptide-Based Cleaving Groups

In embodiments, a coupler includes a peptide-based cleavable coupling group. Peptide-based cleavable coupling groups are cleaved by enzymes such as peptidases and proteases in cells. Peptide-based cleavable coupling groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable coupling groups have the general formula —NHCHR^{A}C(O)NHCHR^{B}C(O)—, where R^{A} and R^{B} are the R groups of the two adjacent amino acids. These candidates can be evaluated using methods analogous to those described above.

In embodiments, a CTM-TO conjugate, or portion thereof, includes the structure shown in Formula (D-I′):

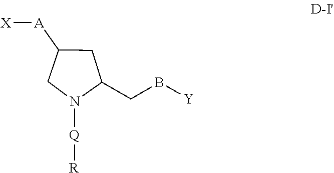

wherein:
A and B are each independently for each occurrence O, N(R^{N}) or S,
X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, —P(Z′)(Z″)O—R¹-Q′-R²—OP(Z″)(Z″″)O-oligonucleotide, or an oligonucleotide, —P(Z′)(Z″)-formula (I), —P(Z′)(Z″)— or -Q-R;
R is L¹ or has the structure shown in formula (D-II), (D-III), (D-IV), or (D-V):

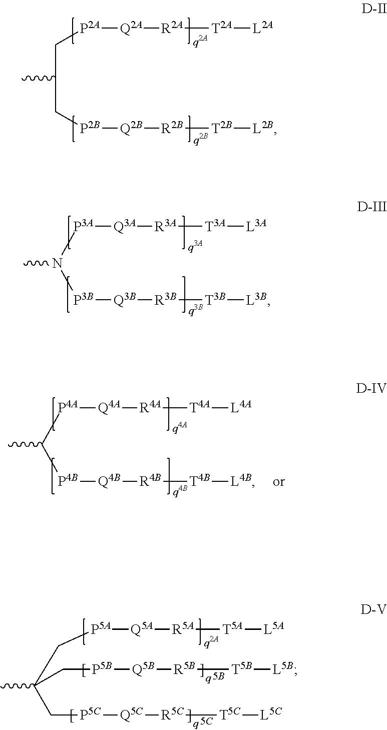

wherein
q^{2A}, q^{2B}, q^{3A}, q^{3B}, q^{4A}, q^{5B} and q^{5V} represent independently for each occurrence 0-20 and wherein the repeating unit can be the same or different;
Q and Q′ are independently for each occurrence is absent, —(P⁷-Q⁷-R⁷_ₚ-T⁷- or -T⁷-Q⁷-T⁷-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, P^{2B}, T^{3A}, P^{3B}, T^{4A}, P^{4B}, T^{4A}, P^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH—N(B^{L})—CH₂;
B^{L} is -T^{B}-Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸, and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);
T^{B} and T^{B′} are each independently for each occurrence absent, CO, NH, O, S, OC(O), OC(O)O, NHC(O), NHC(O) NH, NHC(O) O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O (hexadecyl) glycerol, geranyloxyhexyl group, hexadecylglycerol, borneol, menthol, 1,3-propanediol, heptadecyl group, palmitic acid, myristic acid, O3-(oleoyl) lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO, CH═N—O,

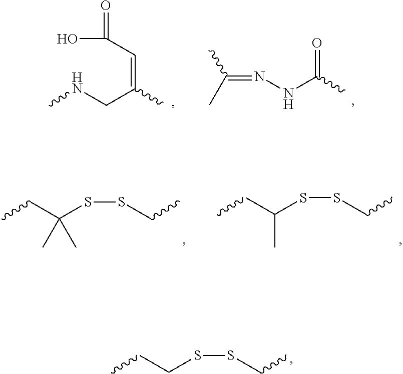

or heterocyclyl,
L¹, L^{2A}, L^{2B}, L^{3A}, L^{3B}, L^{4A}, L^{4B}, L^{5A}, L^{5B} and, L^{5C} are each independently for each occurrence a CTM;
R′ and R″ are each independently H, C¹-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z′″ are each independently for each occurrence O or S,
p represent independently for each occurrence 0-20.

In embodiments, a CTM-TO conjugate, or a portion thereof, includes a structure of Formula (D-I′)

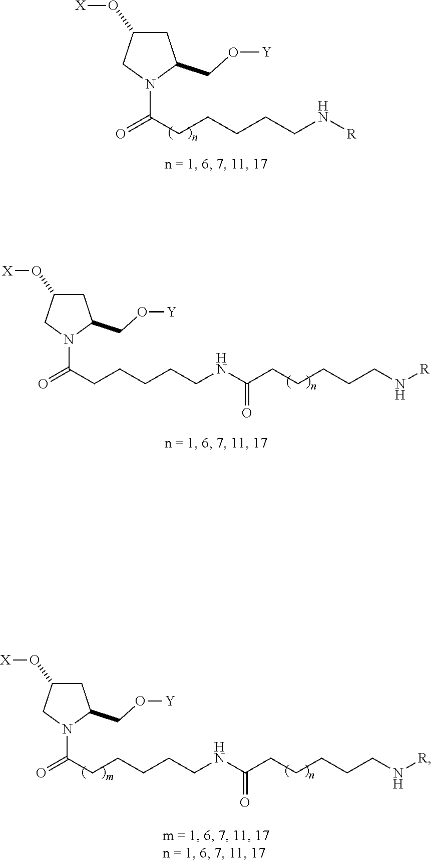

wherein X, Y, and R are as defined above regarding Formlula D-I′.

In embodiments, a compound of Formula (D-I′) has the structure

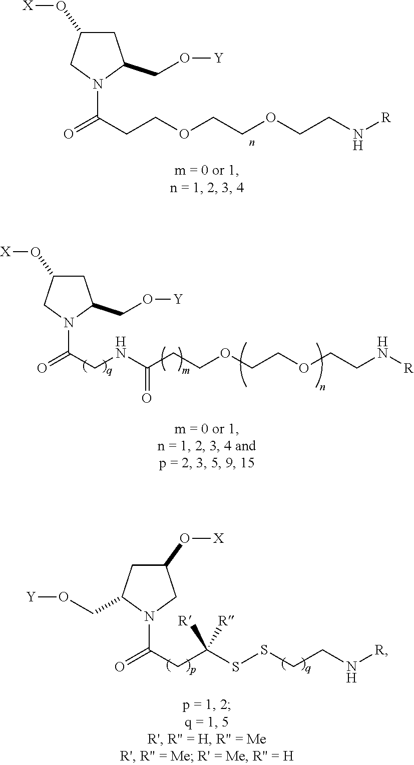

wherein X, Y, and R are as defined above regarding Formula D-I′.

In embodiments, a compound of the Formula (D-I′) has the structure

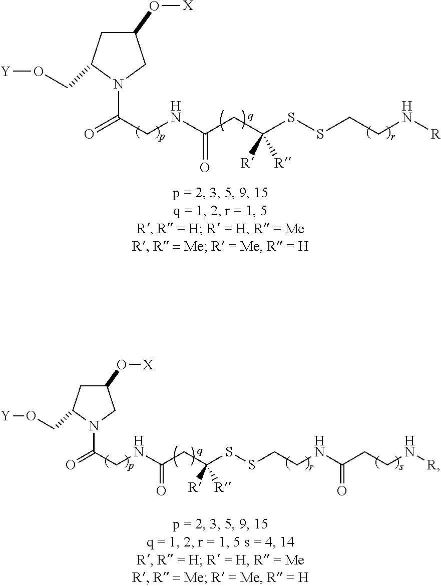

wherein X, Y, and R are as defined above regarding Formula D-I′.

In embodiments, a compound of the Formula (D-I′) has the structure

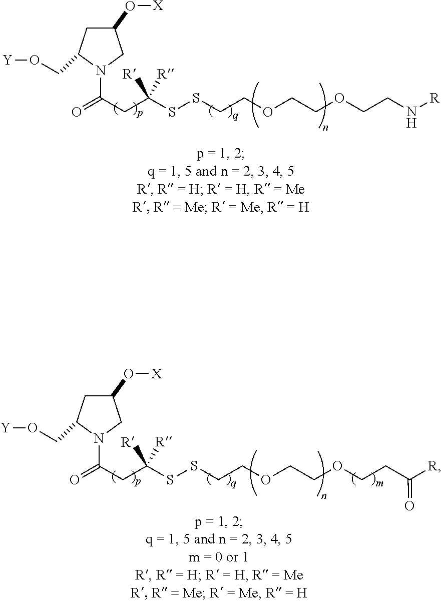

wherein X, Y, and R are as defined above regarding Formula D-I′.

In embodiments, a compound of the Formula (D-I′) has the structure

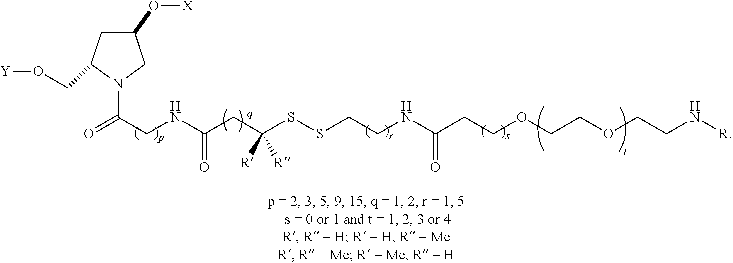

In embodiments, R is

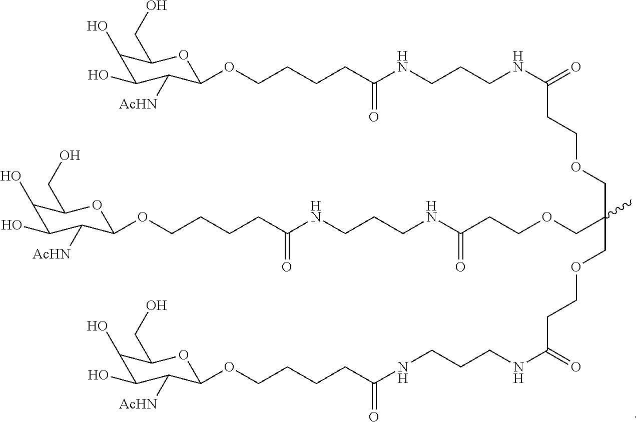 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In embodiments, R is

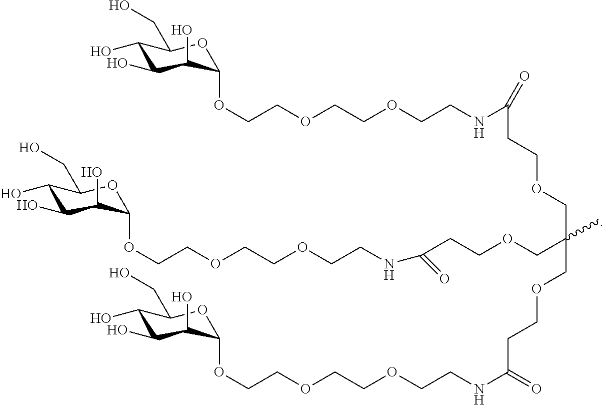

In embodiments, R is

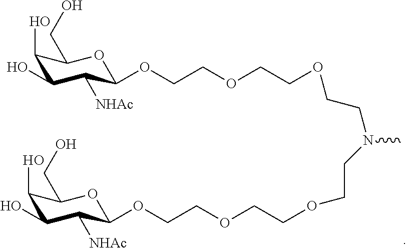

In embodiments, R is

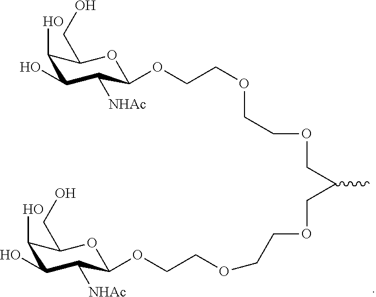

In embodiments, R is

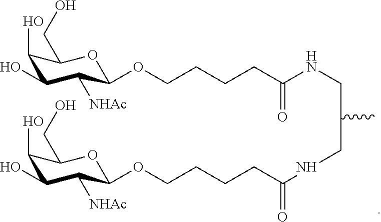

In embodiments, R is

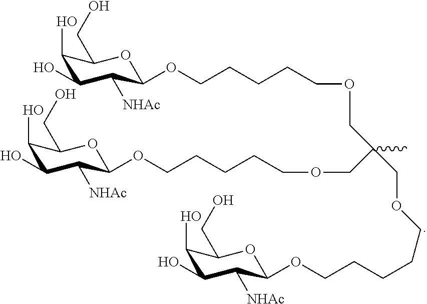

In embodiments, R is

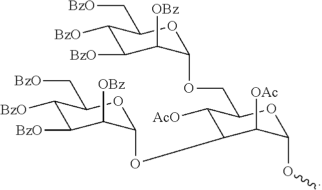

In embodiments, R is

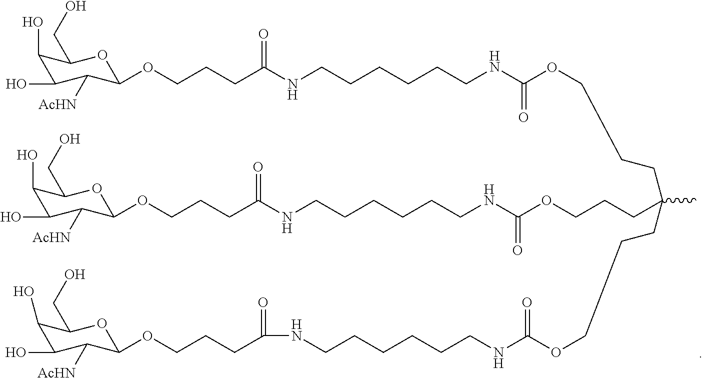 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In embodiments, R is

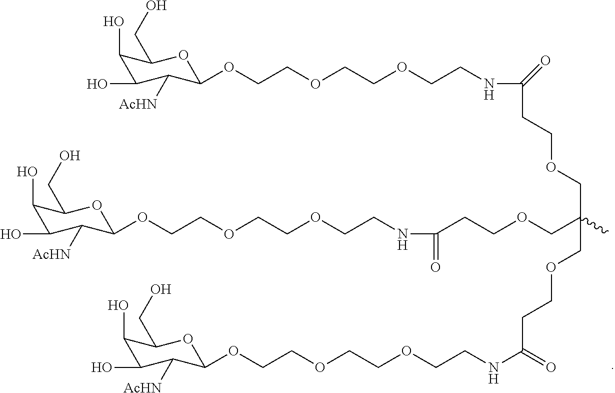 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In embodiments, R is

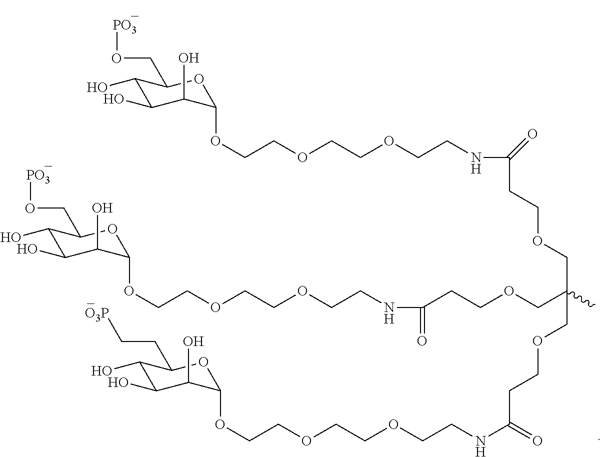

In embodiments, R is

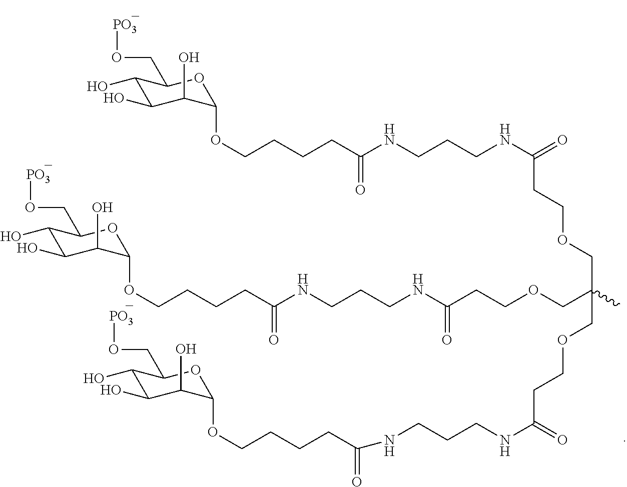

In embodiments, a compound of the Formula (D-I′) has the structure

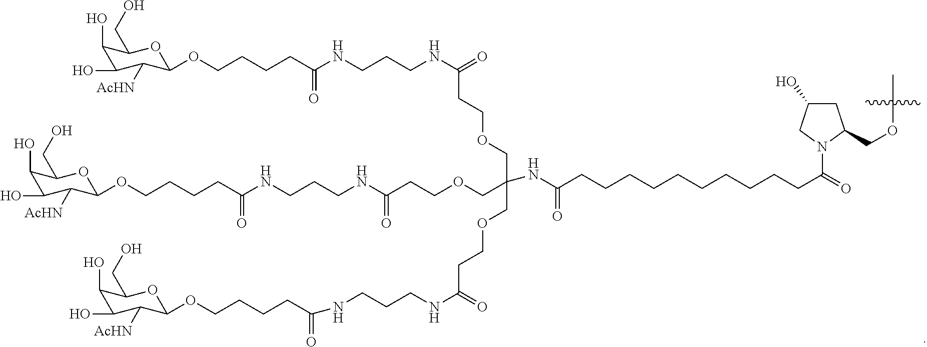 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In embodiments, R is

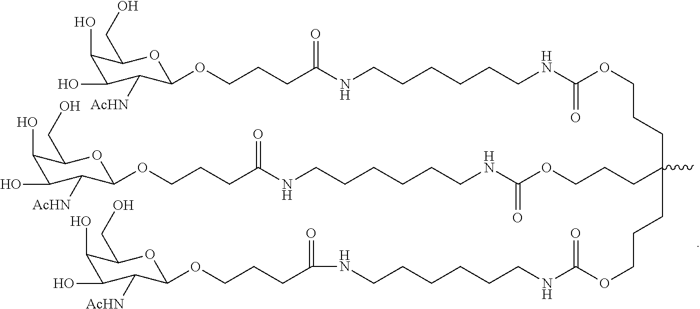 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In embodiments, a compound of the Formula (D-I′) has the structure

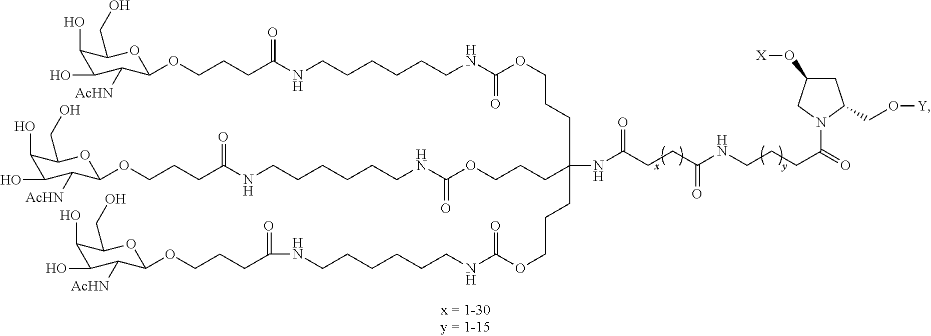

wherein X and Y are as defined above regarding Formula D-I′.

In embodiments, a compound of the Formula (D-I) has the structure

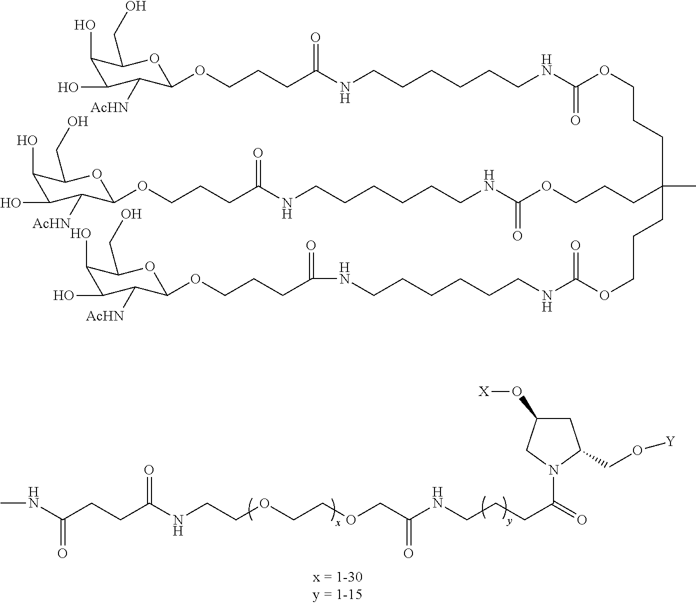

In embodiments, a compound of the Formula (D-I) has the structure

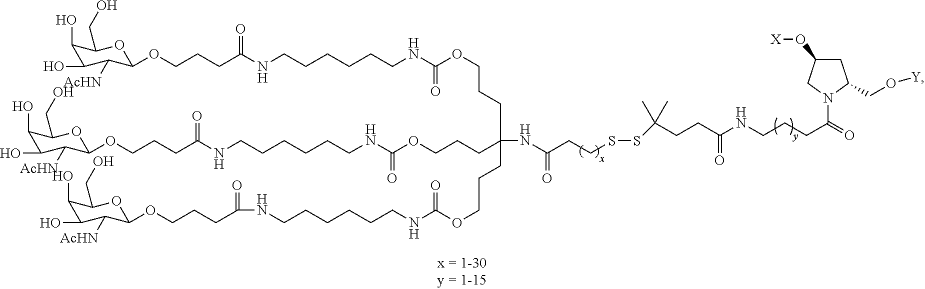

wherein X and Y are as defined above regarding Formula D-I.

In embodiments, a compound of the Formula (D-I) has the structure

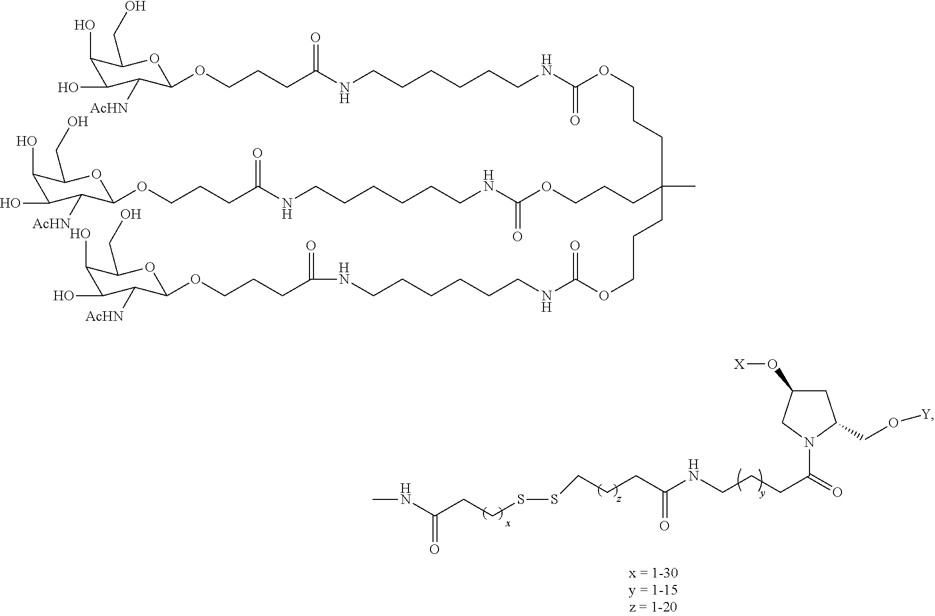

wherein X and Y are as defined above regarding Formula D-I.

In embodiments, a compound of the Formula (D-I) has the structure

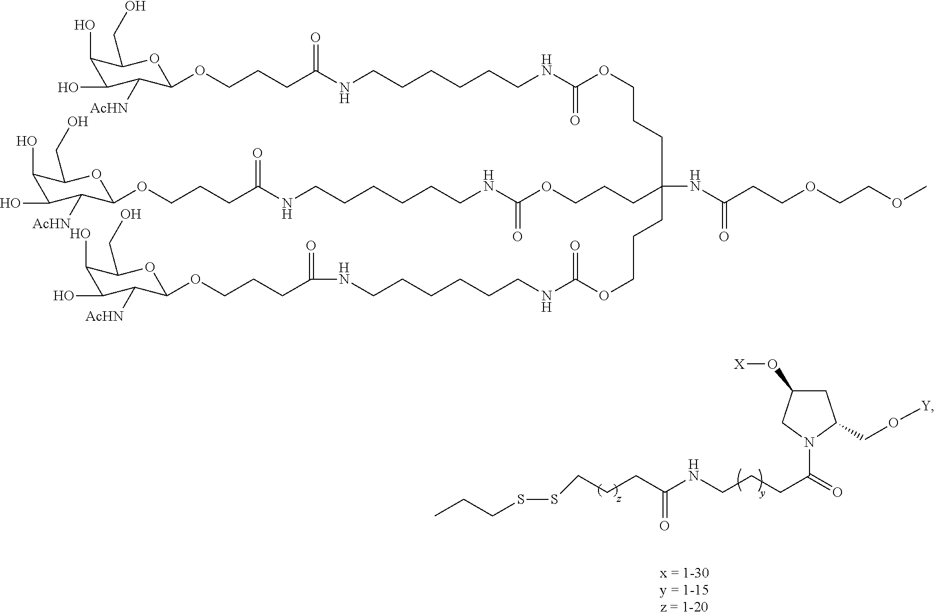

wherein X and Y are as defined above regarding Formula D-I.

In embodiments, a compound of the Formula (D-I) has the structure

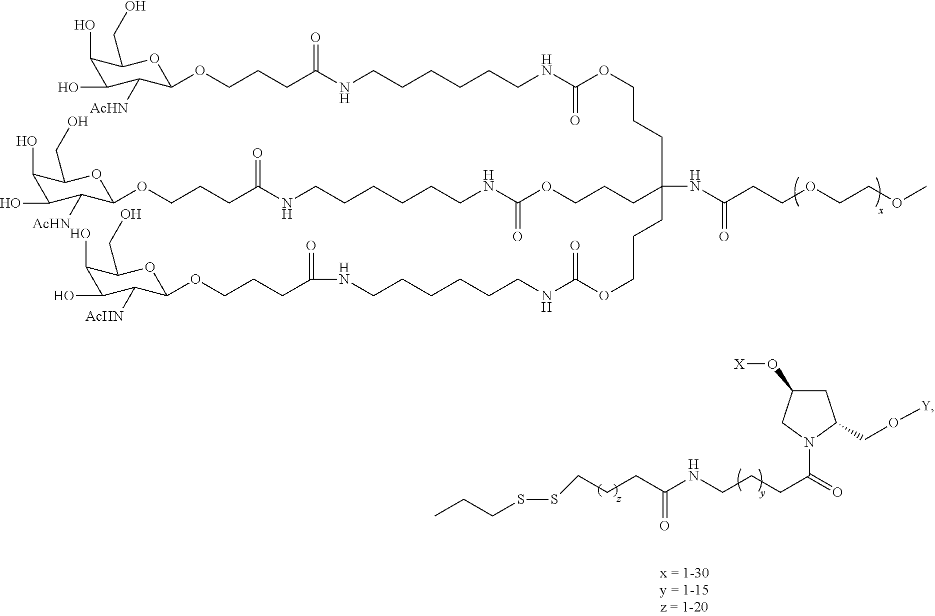

wherein X and Y are as defined above regarding Formula D-I.

In embodiments, R is

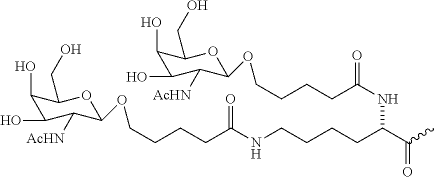

In embodiments, R is

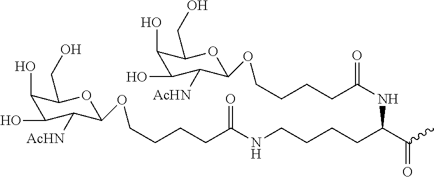

In embodiments, R is

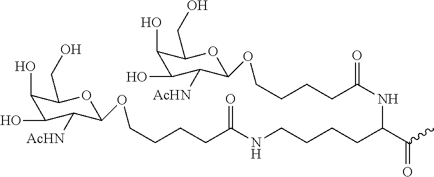

In embodiments, R is

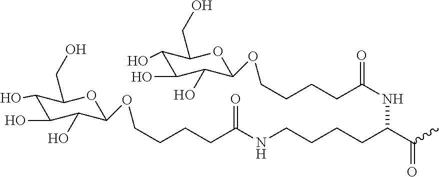

In embodiments, R is

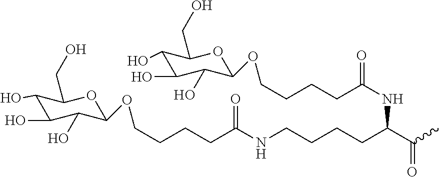

In embodiments, R is

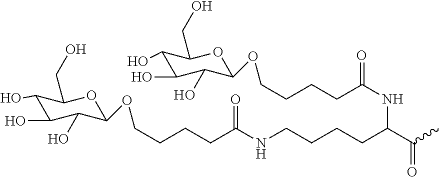

In embodiments, R is

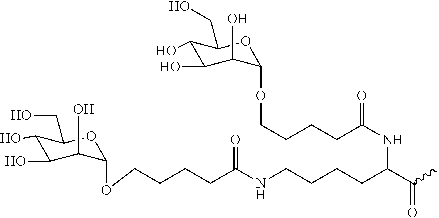

In embodiments, R is

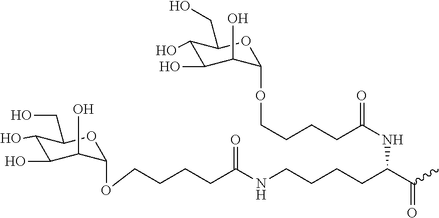

In embodiments, R is

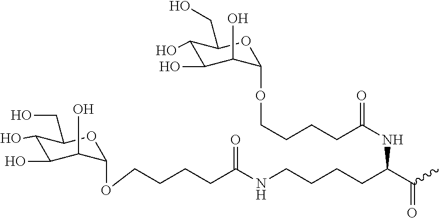

In embodiments, a compound of the Formula (D-I) has the structure

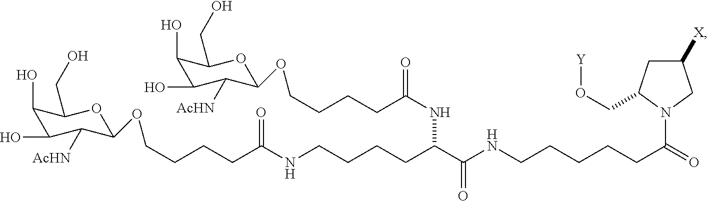

wherein X and Y are as defined above regarding Formula D-I.

In embodiments, a compound of the Formula (D-I) has the structure

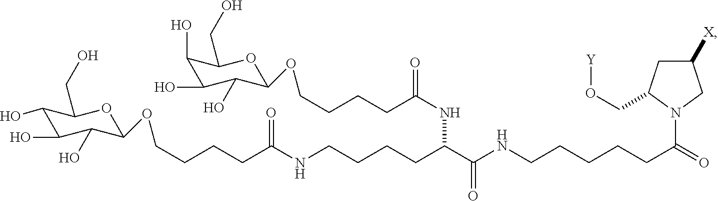

wherein X and Y are as defined above regarding Formula D-I.

In embodiments, a compound of the Formula (D-I) has the structure

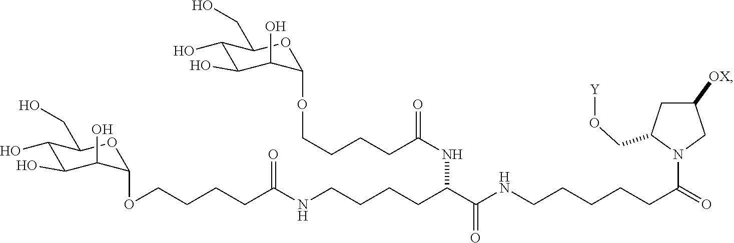

wherein X and Y are as defined above regarding Formula D-I.

In embodiments, a compound of the Formula (D-I) has the structure

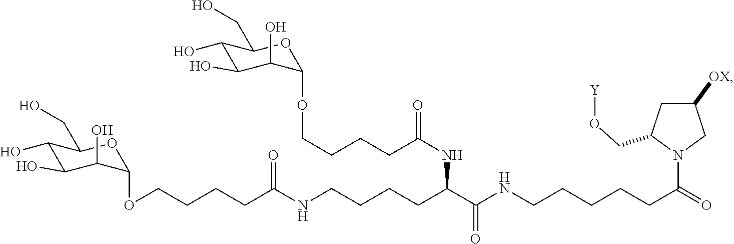

wherein X and Y are as defined above regarding Formula D-I.

In embodiments, a compound of the Formula (D-I) has the structure

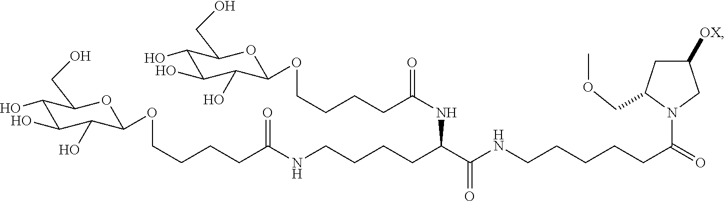

wherein X is as defined above regarding Formula D-I.

In embodiments, a compound of the Formula (D-I) has the structure

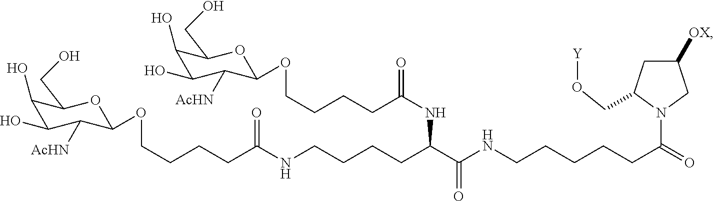

wherein X is as defined above regarding Formula D-I.

In embodiments, both L^{2A} and L^{2B} are the same. In embodiments, both L^{2A} and L^{2B} are different. In embodiments, both L^{3A} and L^{3B} are the same. In embodiments, both L^{3A} and L^{3B} are different. In embodiments, both L^{4A} and L^{4B} are the same. In embodiments, both L^{4A} and L^{4B} are different. In embodiments, all of L^{5A}, L^{5B} and L^{5C} are the same. In embodiments, two of L^{5A}, L^{5B} and L^{5C} are the same. In embodiments, L^{5A} and L^{5B} are the same. In embodiments, L^{5A} and L^{5C} are the same. In embodiments, L^{5B} and L^{5C} are the same.

In embodiments, a CTM-TO conjugate comprises at least nucleotide modified as indicated in Formula (D-I). In embodiments, the CTM-TO conjugate comprises 1, 2, 3, 4 or 5 modified nucleotides as indicated in Formula (D-I). In embodiments, the CTM-TO conjugate comprises 1, 2 or 3 modified nucleotides as indicated in Formula (D-I). In embodiments, the CTM-TO conjugate comprises 1 or 2 modified nucleotides as indicated in Formula (D-I). In embodiments, the CTM-TO conjugate comprises only one modified nucleotides as indicated in Formula (D-I).

In embodiments, all the modified nucleotides according to Formula (D-I) are on the same strand of a single stranded TO moiety.

In embodiments, all the modified nucleotides as indicated in Formula (D-I) are on the same strand of a double stranded TO moiety.

In embodiments, the modified nucleotides as indicated in Formula (D-I) are on separate strands of a double strand of a TO moiety.

In embodiments, all modified nucleotides as indicated in Formula (D-I) in a CTM-TO conjugate are the same.

In embodiments, two or more of the modified nucleotides as indicated in Formula (D-I) in a CTM-TO conjugate are different.

In embodiments, the modified nucleotides as indicated in Formula (D-I) in CTM-TO conjugate are all different.

In embodiments, only some modified nucleotides as indicated in Formula (D-I) in a CTM-TO conjugate are the same.

In embodiments, the modified nucleotides as indicated in Formula (D-I) will be next to each other in the CTM-TO conjugate.

In embodiments, the modified nucleotides as indicated in Formula (D-I) will be on the 5′-end, 3′-end, at an internal position, both the 3′- and the 5′-end, both 5′-end and an internal position, both 3′-end and internal position, and at all three positions (5′-end, 3′-end and an internal position) of CTM-TO conjugate.

In embodiments, R^{x} is cholesterol. In embodiments, R^{x} is lithocholic. In embodiments, IV is oleyl lithocholic.

In embodiments, IV has the structure

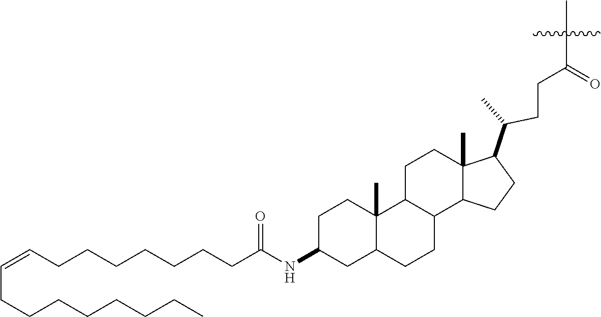

In embodiments, B^{L} has the structure

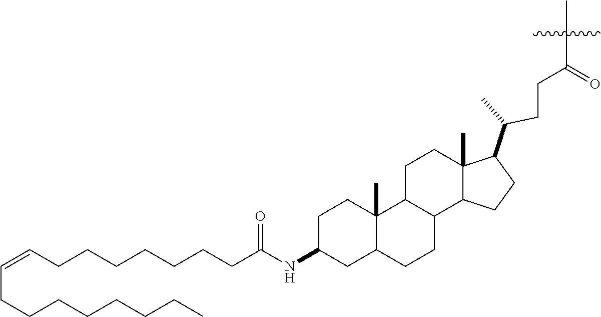

In embodiments, formula (I) has the structure

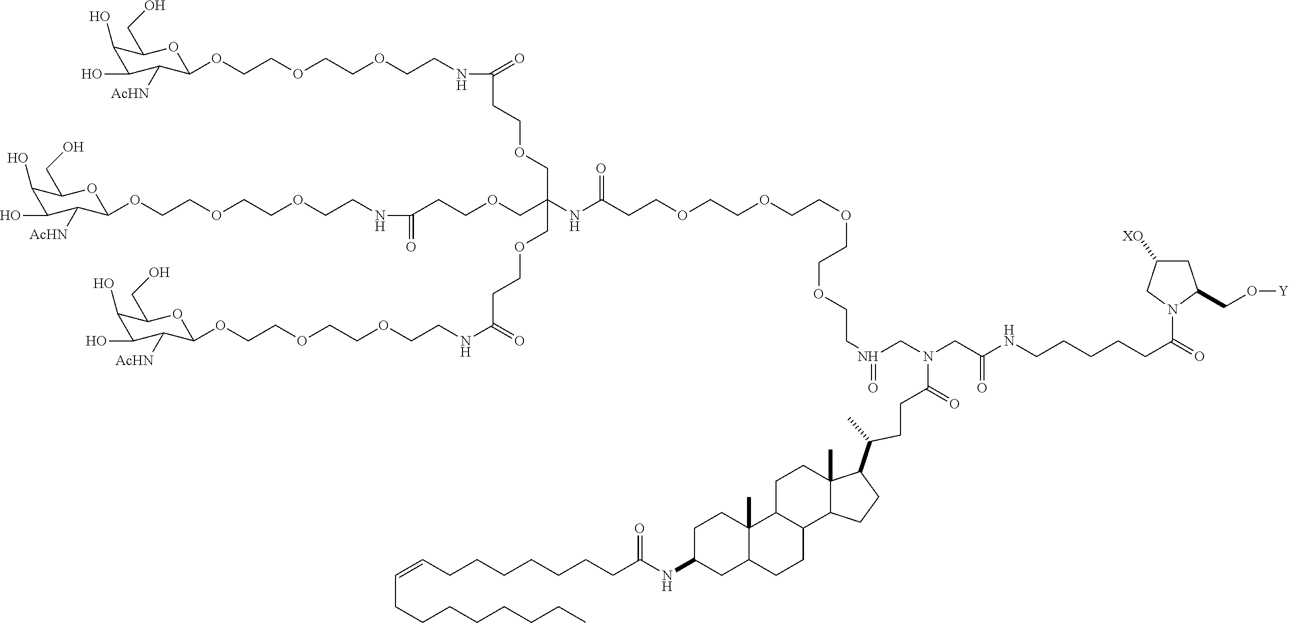

In embodiments, Formula (D-I) has the structure

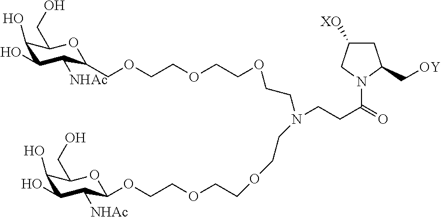

In embodiments, Formula (D-I) has the structure

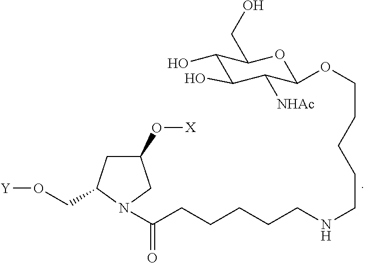

In embodiments, Formula (D-I) has the structure

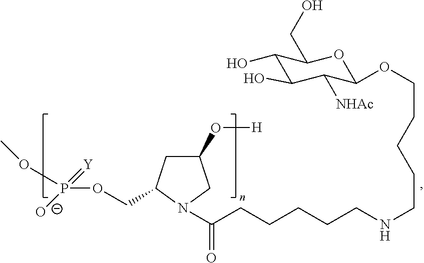

wherein Y is O or S and n is 3-6.

In embodiments, Formula (D-I) has the structure

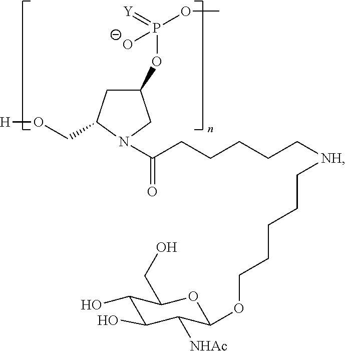

wherein Y is O or S and n is 3-6.

In embodiments, Formula (D-I) has the structure

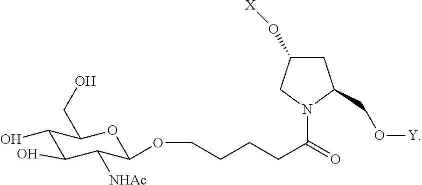

In embodiments, Formula (D-I) has the structure

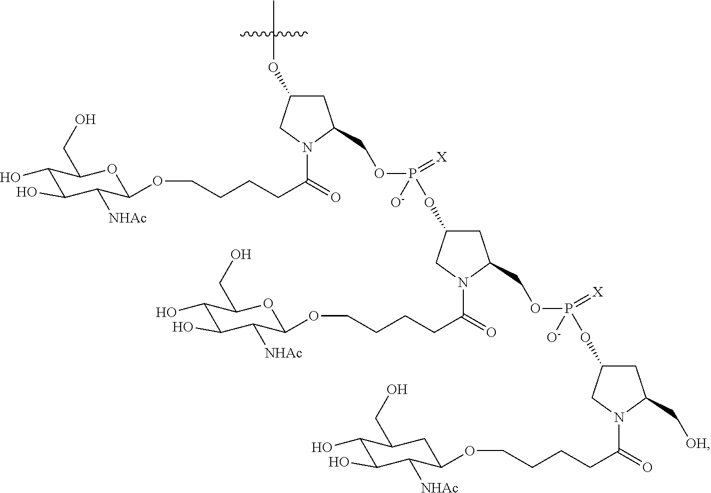

wherein X is O or S

In embodiments, Formula (D-I) has the structure

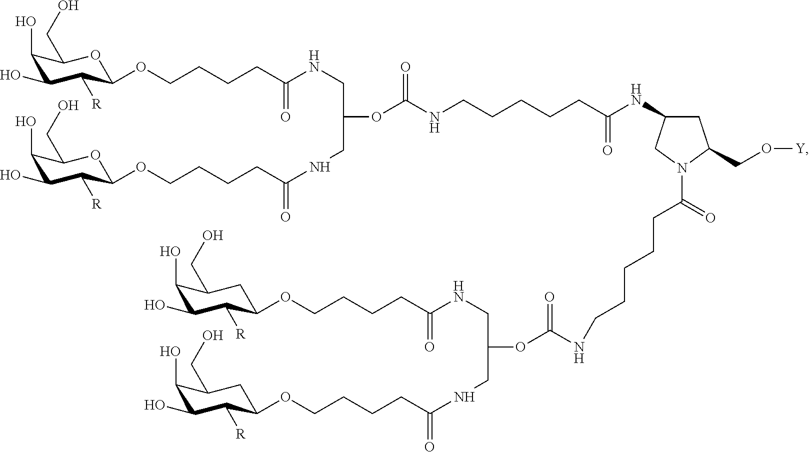

wherein R is OH or NHCOOH.

In embodiments, Formula (D-I) has the structure

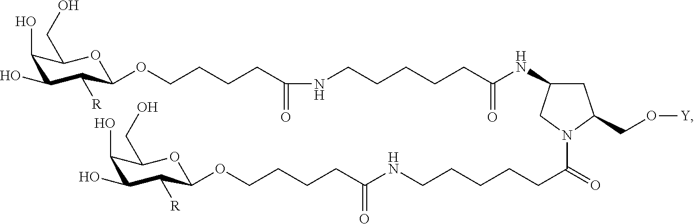

wherein R is OH or NHCOOH.

In embodiments, a modified nucleotides as indicated in Formula (D-I) is linked to the TO moiety through a coupler of formula (D-VI)

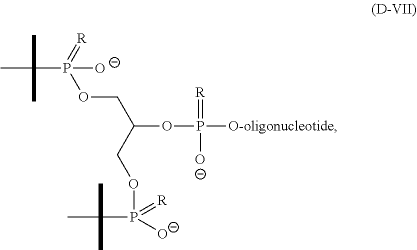

wherein R is O or S.

In embodiments, Formula (D-I) has the structure

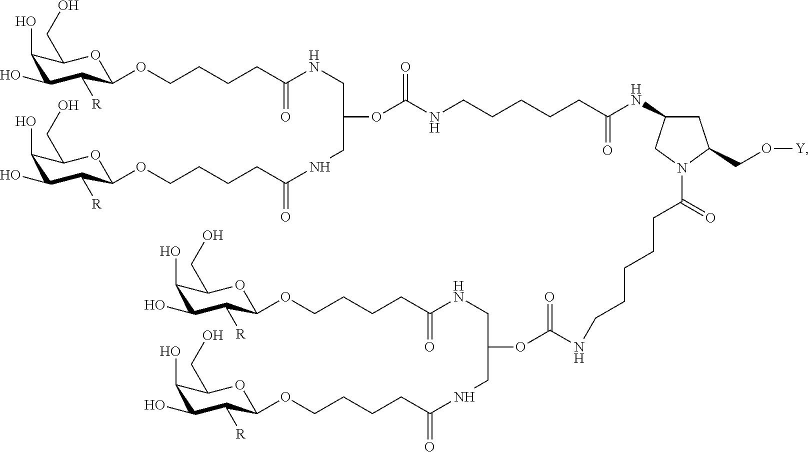

wherein R is OH or NHCOOH.

In embodiments, Formula (D-I) has the structure

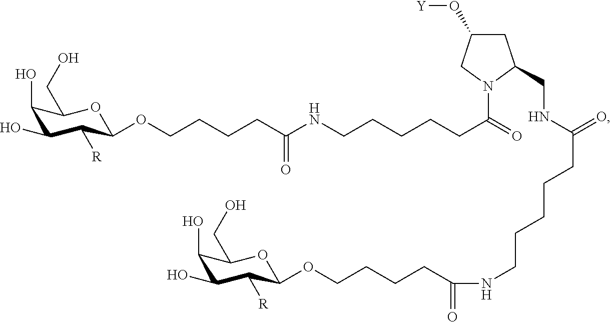

In embodiments, Formula (D-I) has the structure

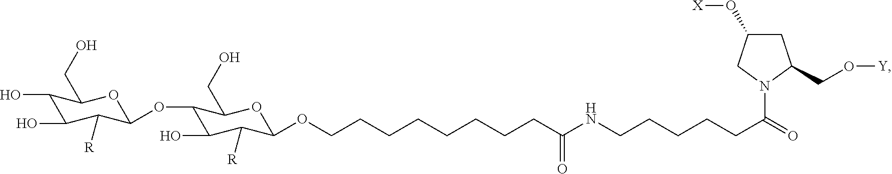

where in R is OH or NHCOOH.

In embodiments, Formula (D-I) has the structure

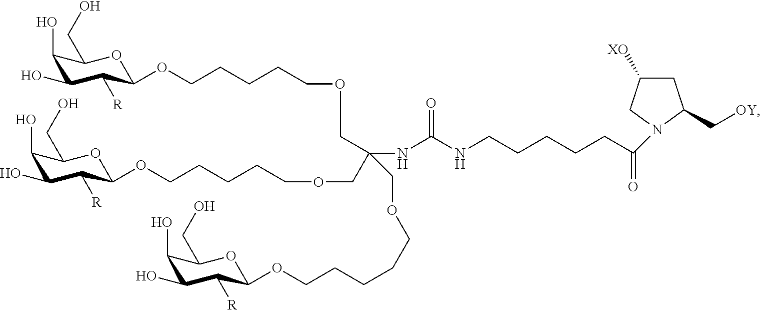

wherein R is OH or NHCOOH

In embodiments, Formula (D-I) has the structure

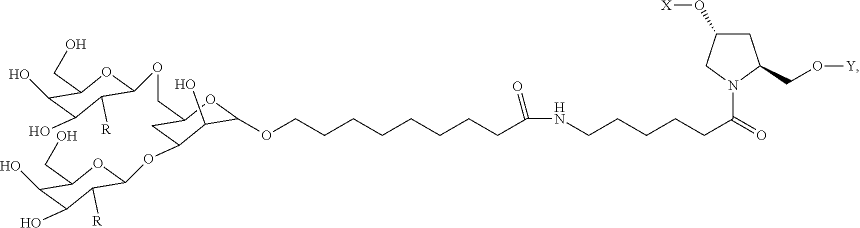

wherein R is OH or NHCOOH.

In embodiments, Formula (D-I) has the structure

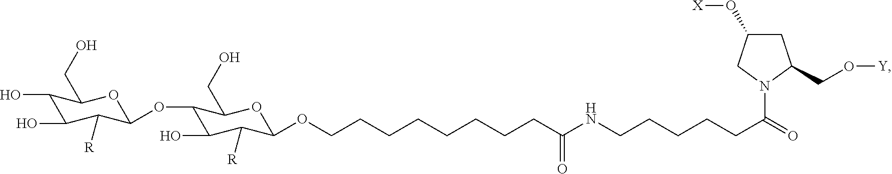

wherein R is OH or NHCOOH.

In embodiments, TO moiety has a modified nucleotide including the structure shown in formula (D-VI) in addition to modified nucleotide shown in Formula (D-I)

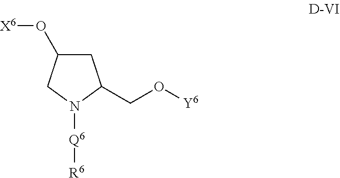

wherein X⁶ and Y⁶ are each independently H, OH, a hydroxyl protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, —P(Z′)(Z″)O—R¹-Q′-R²—OP(Z′″)(Z′″)O-oligonucleotide, a nucleotide, or an oligonucleotide, —P(Z′)(Z″)-formula (I) or —P(Z′)(Z″)—;
Q⁶ is absent or —(P⁶-Q⁶-R⁶)ᵥ-T⁶-;
P⁶ and T⁶ are each independently for each occurrence absent, CO, NH, O, S, OC(O), NHC(O), CH₂, CH₂NH or CHO;
Q⁶ is independently for each occurrence absent, substituted alkylene wherein one or more methylenes can be interrupted or terminated by one or more of O, S, S(O), SO₂, N(R^{N}), C(R′)═C(R′), C≡C or C(O);
R⁶ is independently for each occurrence absent, NH, O, S, CH₂, C(O)O, C(O)NH, NHCH(R^{a})C(O), —C(O)—CH(R^{a})—NH—, CO, CH═N—O,

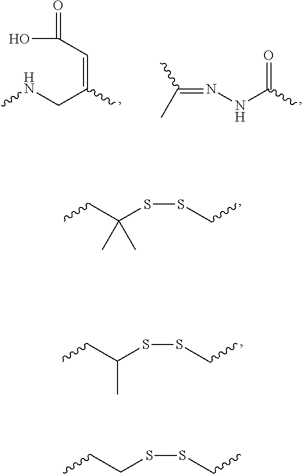

or heterocyclyl;
R′ and R″ are each independently H, C₁-C₆ alkyl OH, SH, N(R^{N})₂;
R^{N} is independently for each occurrence methyl, ethyl, propyl, isopropyl, butyl or benzyl;
R^{a} is H or amino acid side chain,
Z′, Z″, Z′″ and Z″″ are each independently for each occurrence O or S;
v represent independently for each occurrence 0-20,
R^{L} is a lipophile (e.g., cholesterol, cholic acid, adamantane acetic acid, 1-pyrene butyric acid, dihydrotestosterone, 1,3-Bis-O (hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid.

In embodiments, one or more, e.g., 1, 2, 3, 4 or 5, modified nucleotides, or portions thereof, of Formula (D-VI) in addition to one or more, e.g. 1, 2, 3, 4, or 5, modified nucleotides, or portions thereof, of Formula (D-I) are present in CTM-TO conjugate.

In embodiments, only 1 modified nucleotides, or portions thereof, of Formula (D-I) and 1 modified nucleotides, or portions thereof, of Formula (D-VI) are present in CTM-TO conjugate.

In embodiments, R^{L} is cholesterol. In embodiments, R^{L} is lithocholic. In embodiments, R^{L} is oleyl lithocholic.

In embodiments, a modified nucleotide, or portions thereof, of Formula (D-I) is covalently linked with the modified nucleotides, or portions thereof, of Formula (D-VI).

In embodiments, a modified nucleotide, or portions thereof, of Formula (D-I) is linked with the modified nucleotides, or portions thereof, of Formula (D-VI) through a phosphate linkage, e.g. a phosphodiester linkage, a phosphorothioate linkage, a phosphorodithioate linkage.

In embodiments, a modified nucleotide, or portions thereof, of Formula (D-I) is linked to the TO moiety through the modified nucleotides, or portions thereof, of Formula (D-VI).

In embodiments, a modified nucleotides or a portion thereof, of Formula (D-I) intervenes between the TO moiety and the modified nucleotides or a portion thereof, of formula (D-VI).

In embodiments, a modified nucleotides or a portion thereof, of Formula (D-I) and modified nucleotides or a portion thereof, of Formula (D-II) are directly linked to each other. In embodiments, a modified nucleotides or a portion thereof, of Formula (D-I) and a modified nucleotides or a portion thereof, of Formula (D-II) are not directly linked to each other.

In embodiments, a modified nucleotides or a portion thereof, of Formula (D-I) and modified nucleotides or a portion thereof, of Formula (D-VI) are on separate strands of a double stranded TO moiety.

In embodiments, a modified nucleotides or a portion thereof, of Formula (D-I) and a modified nucleotides or a portion thereof, of formula (D-VI) are on opposite terminal ends of the TO moiety.

In embodiments, a modified nucleotides or a portion thereof, of Formula (D-I) and a modified nucleotides or a portion thereof, of Formula (D-VI) are on the same terminal end of the TO.

In embodiments, one of modified nucleotides or a portion thereof, of Formula (D-I) or modified nucleotides or a portion thereof, of Formula (D-VI) is at an internal position while the other is at a terminal position of a TO moiety.

In some embodiments, a modified nucleotides or a portion thereof, of formula (D-I) and a modified nucleotides or a portion thereof, of Formula (D-VI) are both at an internal position of the TO moiety.

In embodiments, a modified nucleotides or a portion thereof, of Formula (D-VI) has the structure

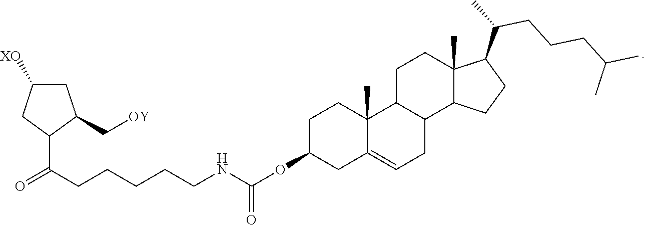

In embodiments, a CTM-TO conjugate is one of:

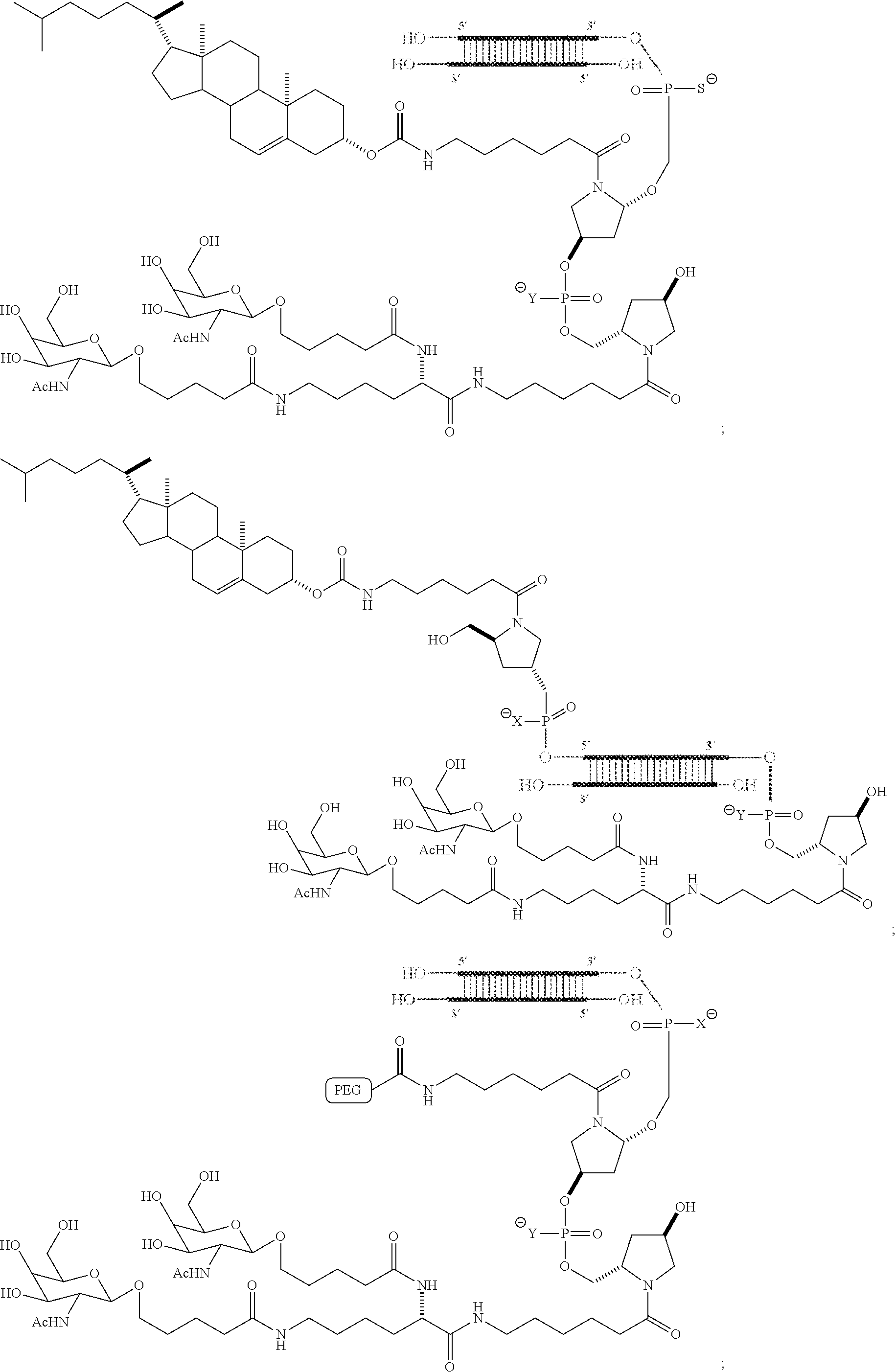

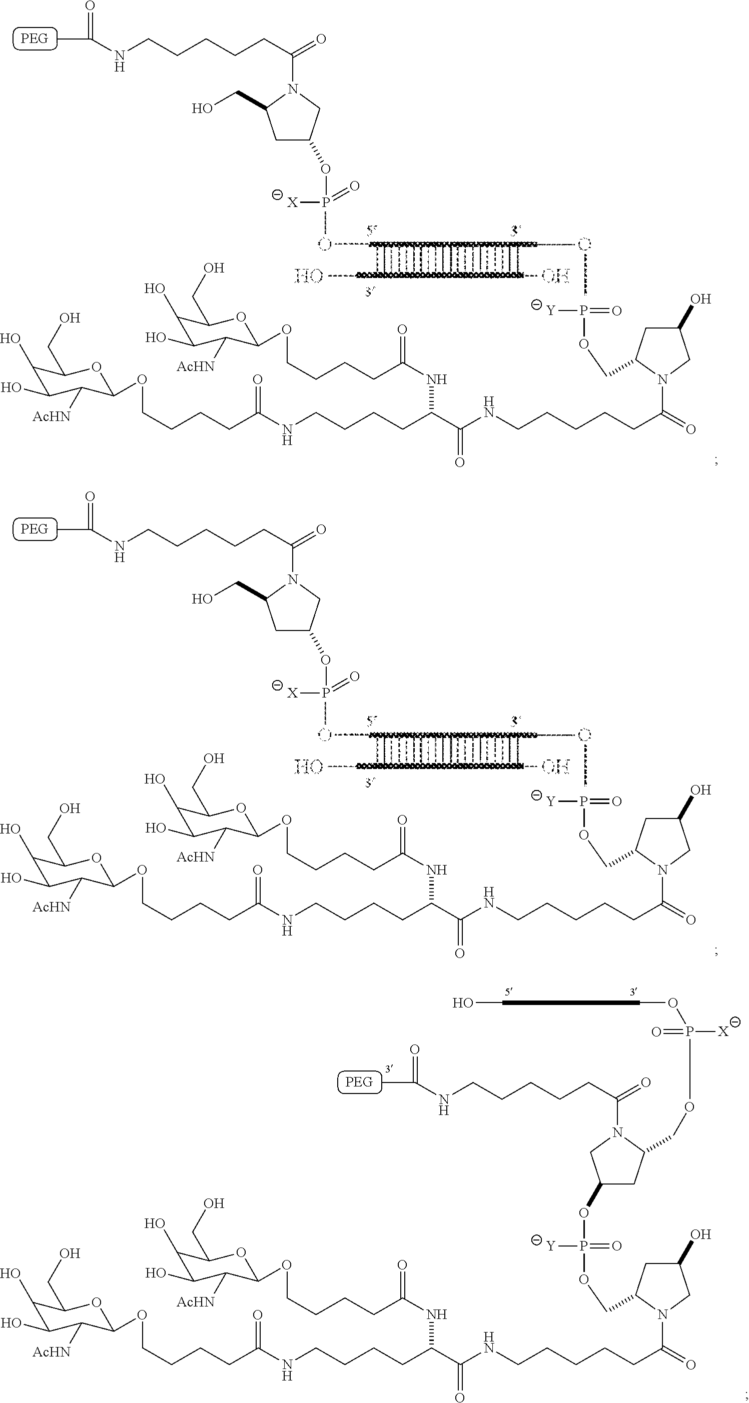

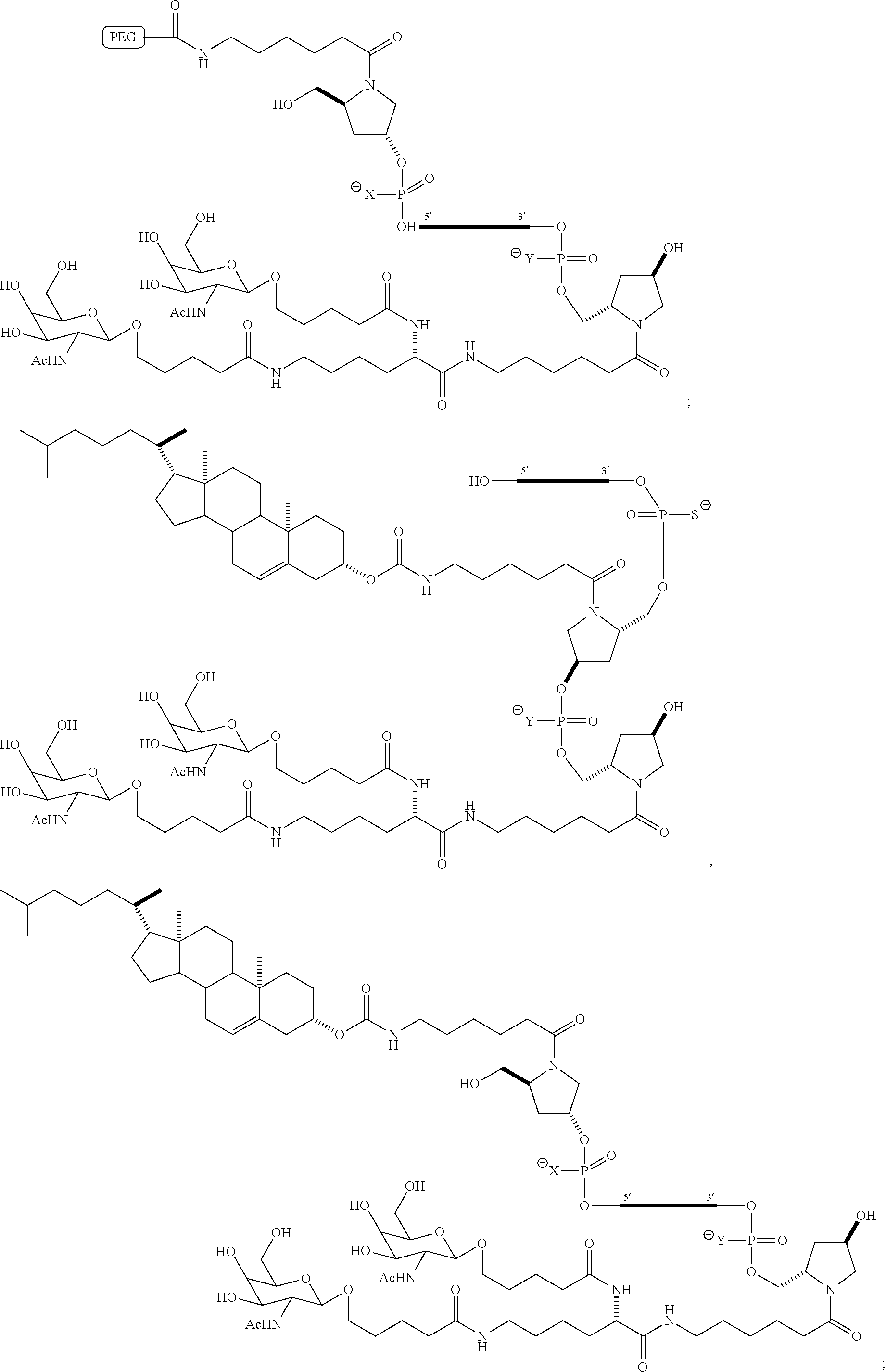

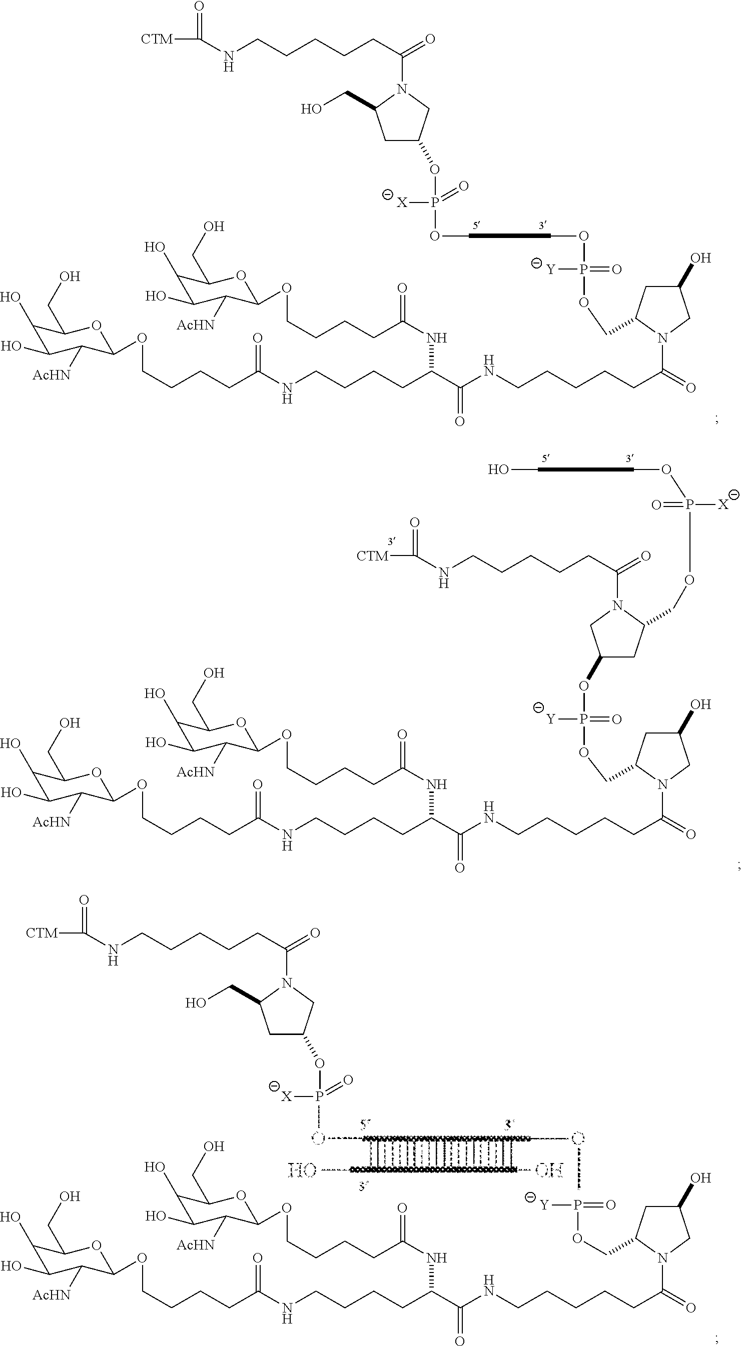

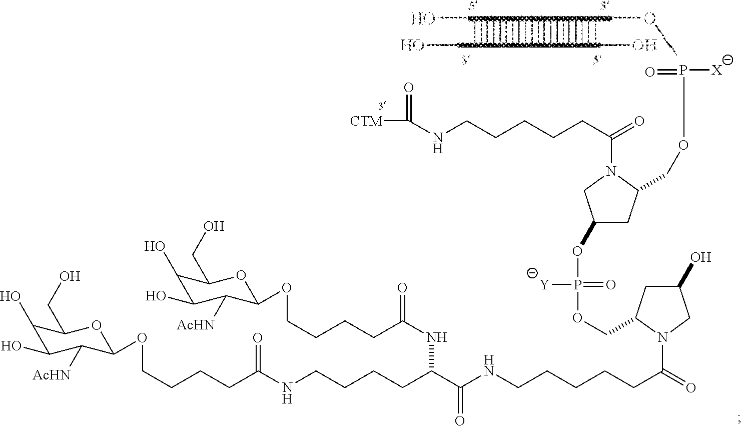

wherein the CTM is a carbohydrate: X═O or S; Y═O or S; PEG stands for ω—OH, ω-amino, ω-methoxy, ω—SH, ω-propargyl, ω-azido and ω-CTM PEGS with MW between 200 and 100,000.

### Therapeutic Oligonucleotide (TO)

The compounds and compositions provided herein comprise a therapeutic moiety suitable for treating a disease of the eye. Any suitable therapeutic agent known or proposed for treating a disease of the eye may be conjugated to a CPP or an EEV.

In embodiments, the therapeutic moiety comprises a oligonucleotide. In embodiments, the therapeutic moiety comprises a polypeptide. In embodiments, the therapeutic moiety comprises a small molecule.

### Oligonucleotides

In embodiments, the therapeutic moiety comprises a therapeutic oligonucleotide. In embodiments, the therapeutic oligonucleotide comprises an antisense oligonucleotide. In embodiments, the therapeutic oligonucleotide comprises siRNA, RNAi, microRNA, antagomir, an aptamer, a ribozyme, an immunostimulatory oligonucleotide, a decoy oligonucleotide, a supermir, a miRNA mimic, a miRNA inhibitor, or a combination thereof. See, for example, Chery, J., “RNA therapeutics: RNAi and antisense mechanisms and clinical applications,” Postdoc J, July 2016, 4(7): 35-50, and Zhu, et al., “RNA-based therapeutics: an overview and prospectus: Cell Death & Disease, 23 Jul. 2022, 12(644) (https://doi.org/10.1038/s41419-022-05075-2).

In embodiments, therapeutic oligonucleotides are provided that include from about 5 to about 100 nucleic acids in length. In embodiments, the therapeutic oligonucleotide is from about 5 to about 50, about 8 to about 40, about 10 to about 30, about 15 to about 30, or about 20 to about 30 nucleotides in length. In embodiments, the antisense compounds include one or more modified nucleosides, one or more modified internucleoside linkages, one or more conjugate groups, or combinations thereof.

### Antisense Oligonucleotides

In embodiments, the therapeutic oligonucleotide is an antisense oligonucleotide directed to a target polynucleotide. In embodiments, the target polynucleotide is a polynucleotide involved in a disease of the eye. In embodiments, the target polynucleotide is a gene or gene transcript for which modulation of expression in a cell of the eye may treat a disease of the eye. In embodiments, the target polynucleotide is a DNA polynucleotide. In embodiments, the DNA polynucleotide is a gene or portion thereof. In embodiments, the target polynucleotide is a RNA polynucleotide. In embodiments, the RNA polynucleotide is a pre-mRNA or portion thereof. In embodiments, the RNA polynucleotide is a mature mRNA polynucleotide or a portion thereof.

The “term “antisense oligonucleotide” or simply “antisense” is meant to include oligonucleotides that are complementary to a target polynucleotide sequence. Antisense oligonucleotides are single stranded molecules that contain DNA, RNA, or combinations or modifications thereof that are complementary to a chosen sequence, e.g. a target gene mRNA. The term “antisense compound” (AC) may be interchangeably used herein with “antisense oligonucleotide” or “antisense.”

The compounds described herein may contain one or more asymmetric centers and thus give rise to enantiomers, diastereomers, and other stereoisomeric configurations that may be defined, in terms of absolute stercochemistry, as (R) or (S), α or β, or as (D) or (L). Included in the antisense compounds provided herein are all such possible isomers, as well as their racemic and optically pure forms.

The antisense oligonucleotides may modulate one or more aspects of protein transcription, translation, and expression and functions via hybridization of the antisense oligonucleotide with a target nucleic acid. In embodiments, the antisense oligonucleotide modulates transcription, translation, or protein expression through steric blocking. The following review article describes the mechanisms of steric blocking and applications thereof and is incorporated by reference herein in its entirety: Roberts et al. Nature Reviews Drug Discovery (2020) 19:673-694.

In embodiments, hybridization of the antisense oligonucleotide to its target polynucleotide suppresses expression of a protein expressed from a gene or transcript thereof. In embodiments, hybridization of the antisense oligonucleotide to its target polynucleotide suppresses expression of one or more protein isoforms. In embodiments, hybridization of the antisense oligonucleotide to its target polynucleotide upregulates expression of the protein. In embodiments, hybridization of the antisense oligonucleotide to its target polynucleotide downregulates expression of the protein.

In embodiments, the antisense compound can inhibit gene expression by binding to a complementary mRNA. Binding to the target mRNA can lead to inhibition of gene expression either by preventing translation of complementary mRNA strands by binding to it or by leading to degradation of the target mRNA. Antisense DNA can be used to target a specific, complementary (coding or non-coding) RNA. If binding takes places this DNA/RNA hybrid can be degraded by the enzyme RNase H. In embodiments, the antisense oligonucleotide contains from about 10 to about 50 nucleotides, or about 15 to about 30 nucleotides. The term also encompasses antisense oligonucleotides that may not be fully complementary to the desired target gene. Thus, compounds disclosed herein can be utilized in instances where non-target specific-activities are found with antisense, or where an antisense sequence containing one or more mismatches with the target sequence is desired.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established.

In embodiments, the antisense oligonucleotide alters processing of mRNA. In embodiments, the antisense oligonucleotide binds to pre-mRNA to alter the structure of mature mRNA during mRNA processing. In embodiments, the antisense oligonucleotide causes alternative splicing of the pre-mRNA. In embodiments, the alternative splicing of the pre-mRNA results in exon skipping.

In embodiments, the antisense oligonucleotides modulates one or more aspects of protein transcription, translation, and expression. In embodiments, the antisense oligonucleotide is directed to a target sequence within a target pre-mRNA modulates one or more aspects of pre-mRNA splicing. As used herein, modulation of splicing refers to altering the processing of a pre-mRNA transcript such that the spliced mRNA molecule contains either a different combination of exons as a result of exon skipping or exon inclusion, a deletion in one or more exons, or the deletion or addition of a sequence not normally found in the spliced mRNA (e.g., an intron sequence). In embodiments, antisense oligonucleotides hybridization to a target sequence in a pre-mRNA molecule restores native splicing to a mutated pre-mRNA sequence. In embodiments, antisense oligonucleotides hybridization results in alternative splicing of the target pre-mRNA. In embodiments, antisense oligonucleotides hybridization results in exon inclusion or exon skipping of one or more exons. In embodiments, the skipped exon sequence comprises a frameshift mutation, a nonsense mutation, or a missense mutation. In embodiments, the skipped exon sequence comprises a nucleic acid deletion, substitution, or insertion. In embodiments, the skipped exon itself does not comprise a sequence mutation, but a neighboring exon comprises a mutation leading to a frameshift mutation or a nonsense mutation. In embodiments, antisense oligonucleotides hybridization to a target sequence within a target pre-mRNA prevents inclusion of an exon sequence in the mature mRNA molecule. In embodiments, antisense oligonucleotides hybridization to a target sequence within a target pre-mRNA results in preferential expression of a wild type target protein isomer. In embodiments, antisense oligonucleotides hybridization to a target sequence within a target pre-mRNA results in expression of a re-spliced target protein comprising an active fragment of a wild type target protein.

Pre-mRNA molecules are made in the nucleus and are processed before or during transport to the cytoplasm for translation. Processing of the pre-mRNAs includes addition of a 5′ methylated cap and an approximately 200-250 base poly(A) tail to the 3′ end of the transcript. The next step in mRNA processing is splicing of the pre-mRNA, which occurs in the maturation of 90-95% of mammalian mRNAs. Introns (or intervening sequences) are regions of a primary transcript (or the DNA encoding it) that are not included in the coding sequence of the mature mRNA. Exons are regions of a primary transcript that remain in the mature mRNA when it reaches the cytoplasm. The exons are spliced together to form the mature mRNA sequence. Splice junctions are also referred to as splice sites with the 5′ side of the junction often called the “5′ splice site,” or “splice donor site” and the 3′ side called the “3′ splice site” or “splice acceptor site.” In splicing, the 3′ end of an upstream exon is joined to the 5′ end of the downstream exon. Thus, the unspliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Cryptic splice sites are those which are less often used but may be used when the usual splice site is blocked or unavailable. Alternative splicing, defined as the splicing together of different combinations of exons, often results in multiple mRNA transcripts from a single gene.

In embodiments, the antisense oligonucleotide hybridizes with a sequence in a splice site. In embodiments, the antisense oligonucleotide hybridizes with a sequence comprising part of a splice site. In embodiments, the antisense oligonucleotide hybridizes with a sequence comprising part or all of a splice site. In embodiments, the antisense oligonucleotide hybridizes with a sequence comprising part or all of a splice donor site. In embodiments, the antisense oligonucleotide hybridizes with a sequence comprising part or all of a splice acceptor site. In embodiments, the antisense oligonucleotide hybridizes with a sequence comprising part or all of a cryptic splice site. In embodiments, the antisense oligonucleotide hybridizes with a sequence comprising an exon/intron junction.

Exon skipping using antisense oligonucleotides conjugated to cyclic peptides is described in International Patent Application No. PCT/US22/28357, filed on 9 May 2022, and entitled COMPOSITIONS AND METHODS FOR MODULATING mRNA SPLICING, which application is hereby incorporated herein by reference in its entirety. In embodiments, the antisense oligonucleotide interferes with the ability to add a polyA tail to mRNA. In embodiments, the antisense oligonucleotide binds to pre-mRNA at or near a polyadenylation site to prevent addition of the polyA tail. Antisense compounds conjugated to cyclic peptides for modulating polyadenylation of mRNA is disclose in International Patent Application No. PCT/US22/28354, filed on 9 May 2022, and entitled COMPOSITIONS AND METHODS FOR MODULATING GENE EXPRESSION, which application is hereby incorporated herein by reference in its entirety.

Methods of producing antisense oligonucleotides are known in the art and can be readily adapted to produce an antisense oligonucleotide that targets any polynucleotide sequence, including, for example, a protein involved in a disease of the eye. Methods for designing, synthesizing and screening antisense compounds for antisense activity against a preselected target nucleic acid can be found, for example in “Antisense Drug Technology, Principles, Strategies, and Applications” Edited by Stanley T. Crooke, CRC Press, Boca Raton, Florida, which is incorporated by reference in its entirety for any purpose.

Antisense mechanisms rely on hybridization of the antisense compound to the target nucleic acid. In embodiments, the therapeutic moiety includes an antisense compound that is complementary to an nucleic acid associated with a disease of the eye.

In embodiments, the AC hybridizes with a target nucleic acid having sequence from about 5 to about 50 nucleic acids in length, which can also be referred to as the length of the AC. In embodiments, the AC is from about 5 to about 50, about 8 to about 40, about 10 to about 30, about 15 to about 30, or about 20 to about 30 nucleic acids in length. In embodiments, the AC is at least about 5, about 6, about 7, about 8, about 9, about 10, about 11, about 12, about 13, about 14, or about 15, and up to about about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleic acids in length. In embodiments, the AC is about 15 nucleic acids in length. In embodiments, the AC is about 16 nucleic acids in length. In embodiments, the AC is about 17 nucleic acids in length. In embodiments, the AC is about 18 nucleic acids in length. In embodiments, the AC is about 19 nucleic acids in length. In embodiments, the AC is about 20 nucleic acids in length. In embodiments, the AC is about 21 nucleic acids in length. In embodiments, the AC is about 22 nucleic acids in length. In embodiments, the AC is about 23 nucleic acids in length. In embodiments, the AC is about 24 nucleic acids in length. In embodiments, the AC is about 25 nucleic acids in length. In embodiments, the AC is about 26 nucleic acids in length. In embodiments, the AC is about 27 nucleic acids in length. In embodiments, the AC is about 28 nucleic acids in length. In embodiments, the AC is about 29 nucleic acids in length. In embodiments, the AC is about 30 nucleic acids in length.

In embodiments, the AC may be less than about 100 percent complementary to a target nucleic acid sequence. As used herein, the term “percent complementarity” refers to the number of nucleobases of an AC that have nucleobase complementarity with a corresponding nucleobase of an oligomeric compound or nucleic acid divided by the total length (number of nucleobases) of the AC. One skilled in the art recognizes that the inclusion of mismatches is possible without eliminating the activity of the antisense compound. In embodiments, the ACs contain no more than about 15%, no more than about 10%, no more than 5%, or no mismatches. In embodiments, the ACs are at least about 80%, at least about 85%, at least about 90%, at least about 95%, at least about 96%, at least about 97%, at least about 98%, at least about 99% or about 100% complementary to a target nucleic acid. Percent complementarity of an oligonucleotide is calculated by dividing the number of complementary nucleobases by the total number of nucleobases of the oligonucleotide. Percent complementarity of a region of an oligonucleotide is calculated by dividing the number of complementary nucleobases in the region by the total number of nucleobases region.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or change in Tm (ΔTm) can be calculated by techniques that are familiar to one of ordinary skill in the art. For example, techniques described in Freier et al. (Nucleic Acids Research, 1997, 25, 22:4429-4443) allow one of ordinary skill in the art to evaluate nucleotide modifications for their ability to increase the melting temperature of an RNA: DNA duplex.

The efficacy of the ACs of the present disclosure may be assessed by evaluating the antisense activity effected by their administration. As used herein, the term “antisense activity” refers to any detectable and/or measurable activity attributable to the hybridization of an antisense compound to its target nucleic acid. Such detection and or measuring may be direct or indirect. In embodiments, antisense activity is assessed by detecting and or measuring the amount of target protein. In embodiments, antisense activity is assessed by detecting and/or measuring the amount of target nucleic acids.

### Illustrative Nucleosides

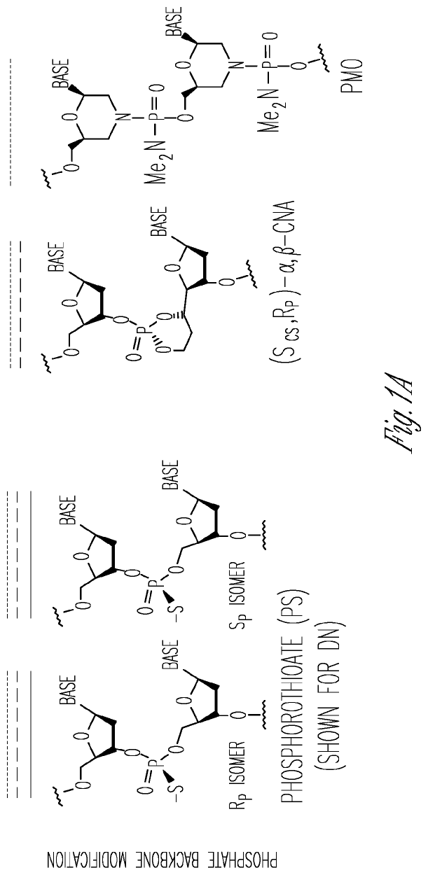

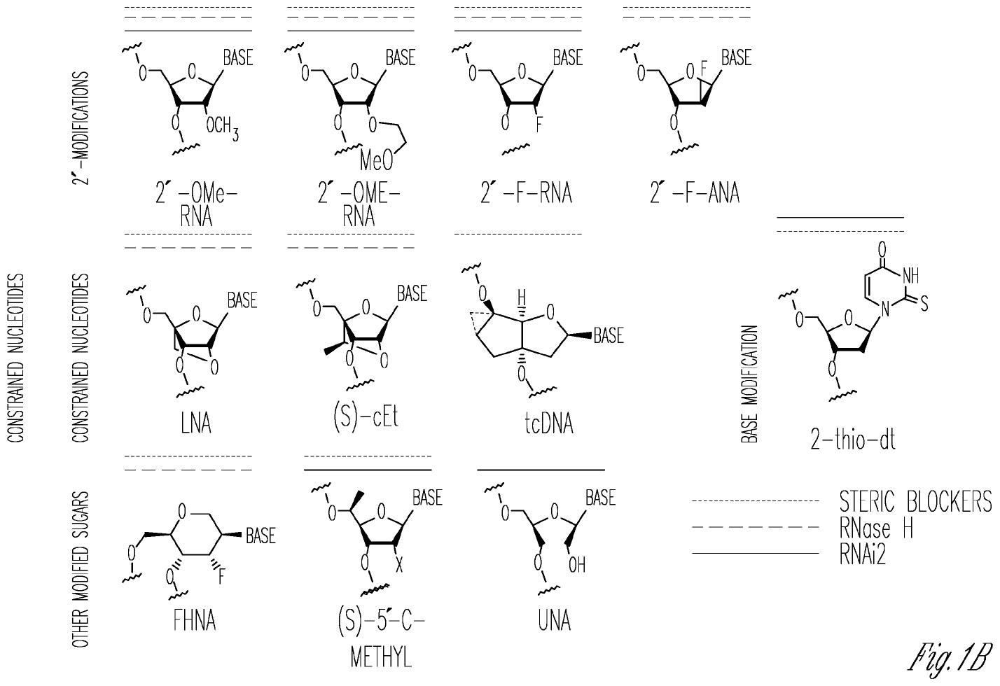

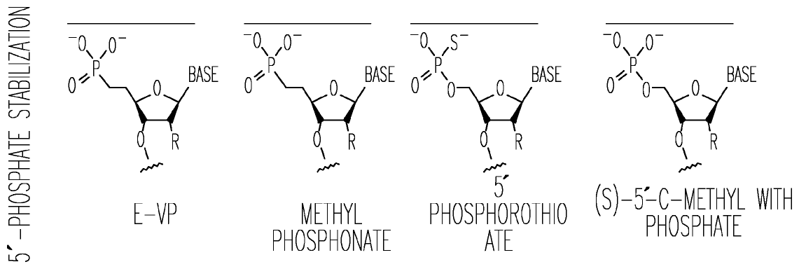

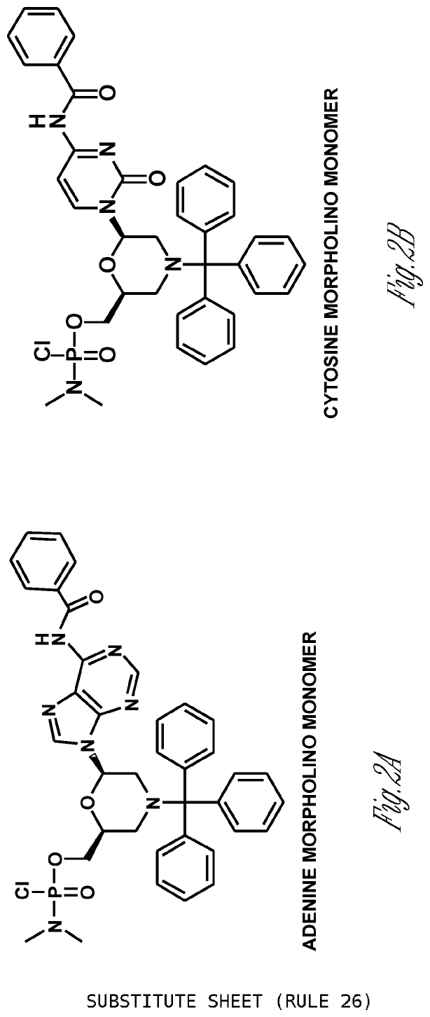

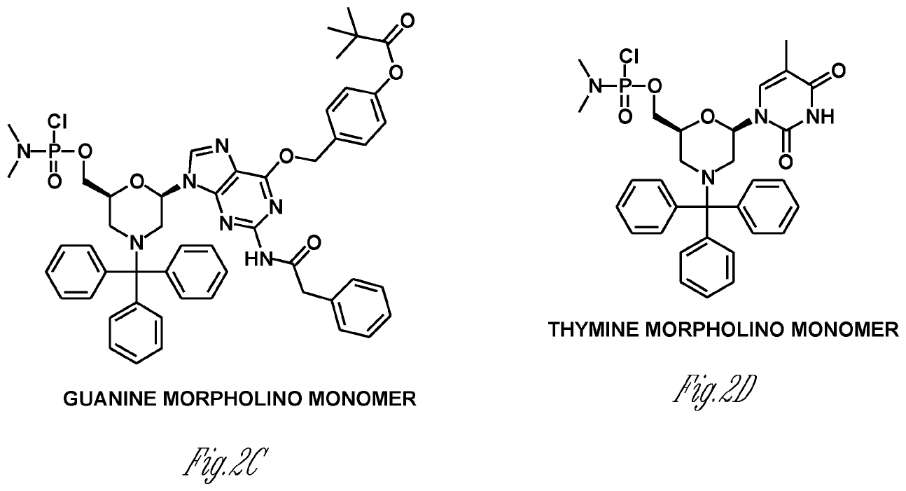

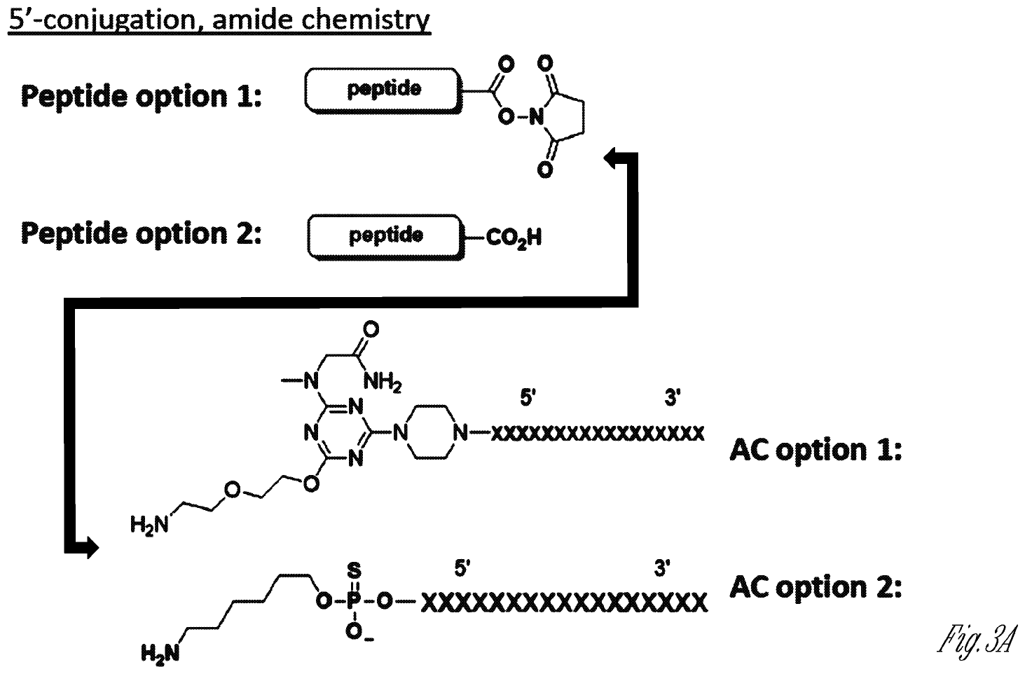

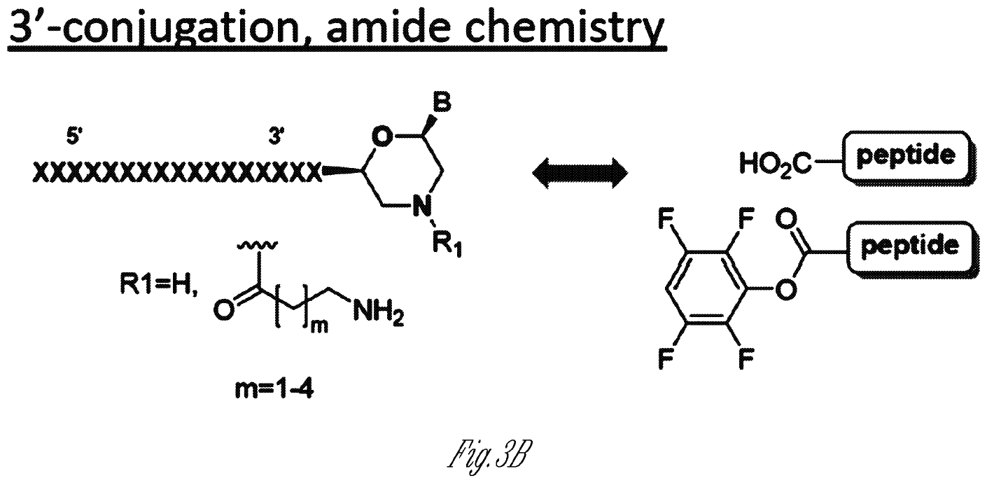

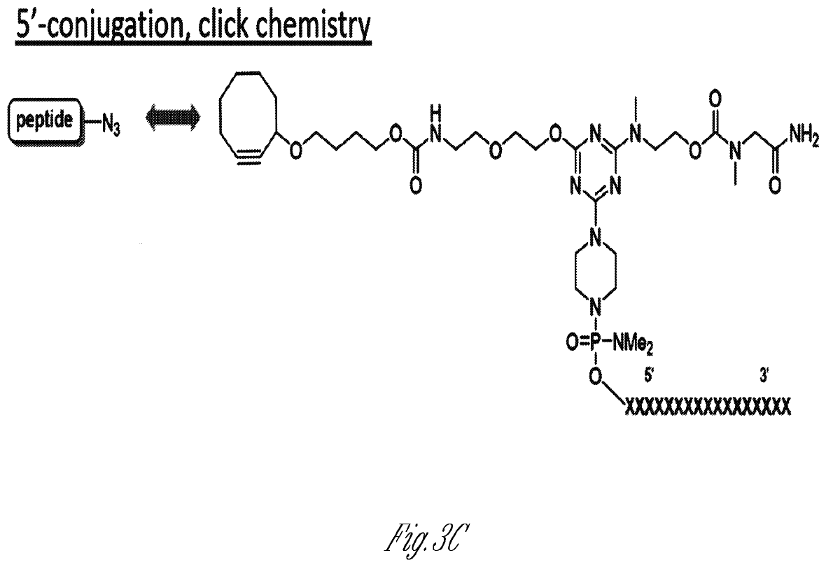

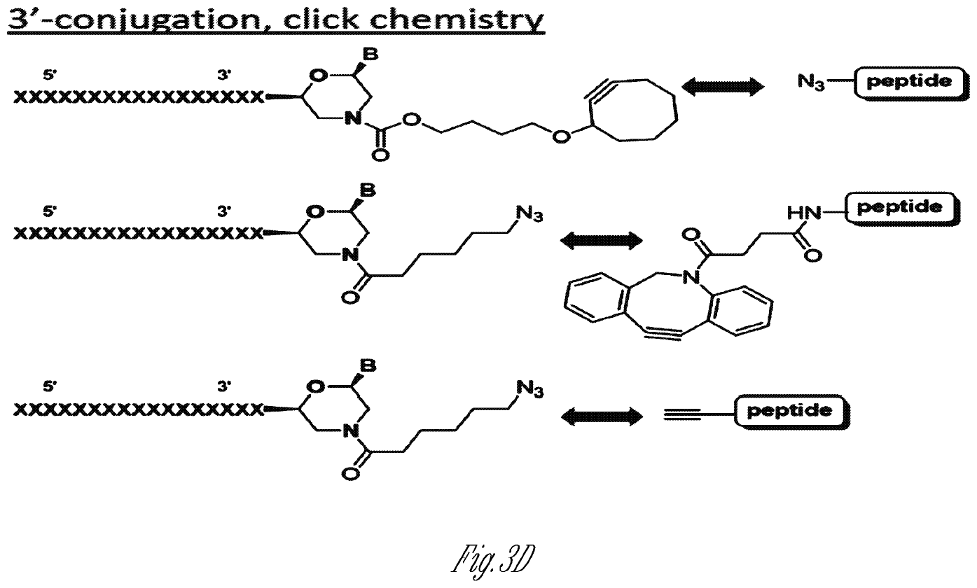

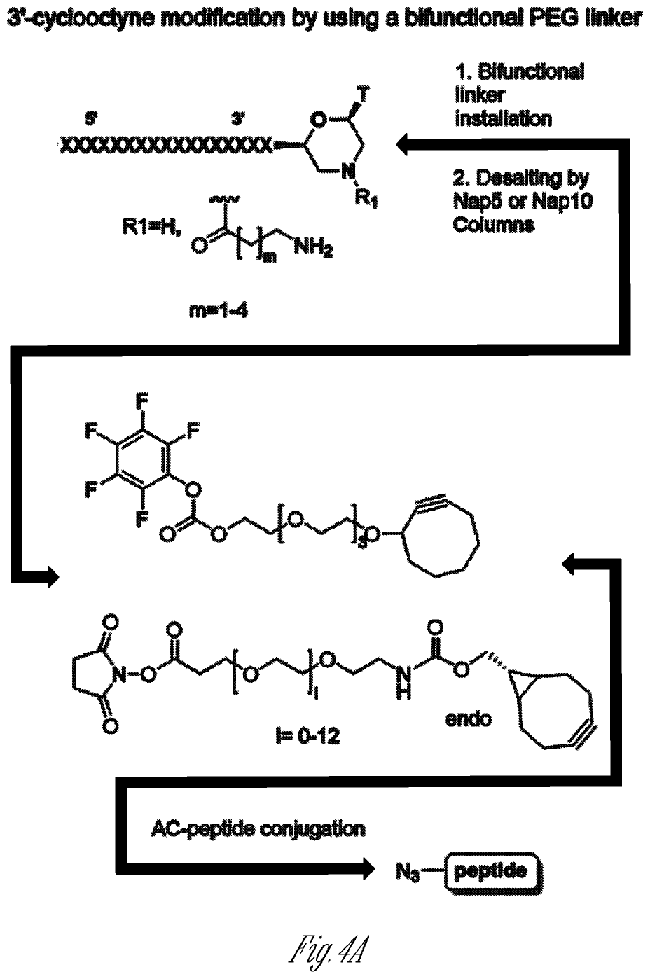

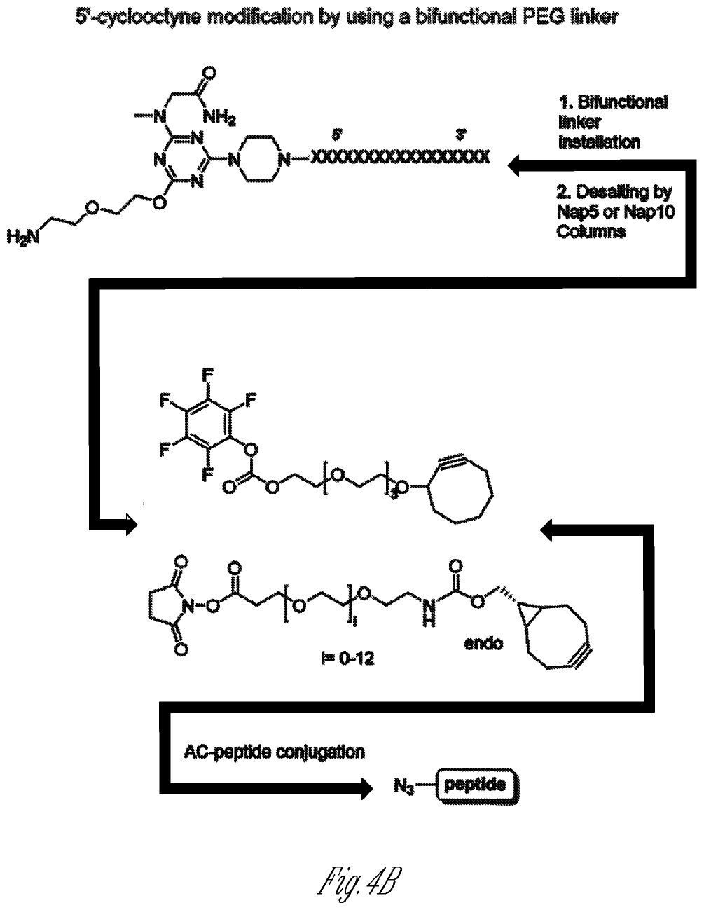

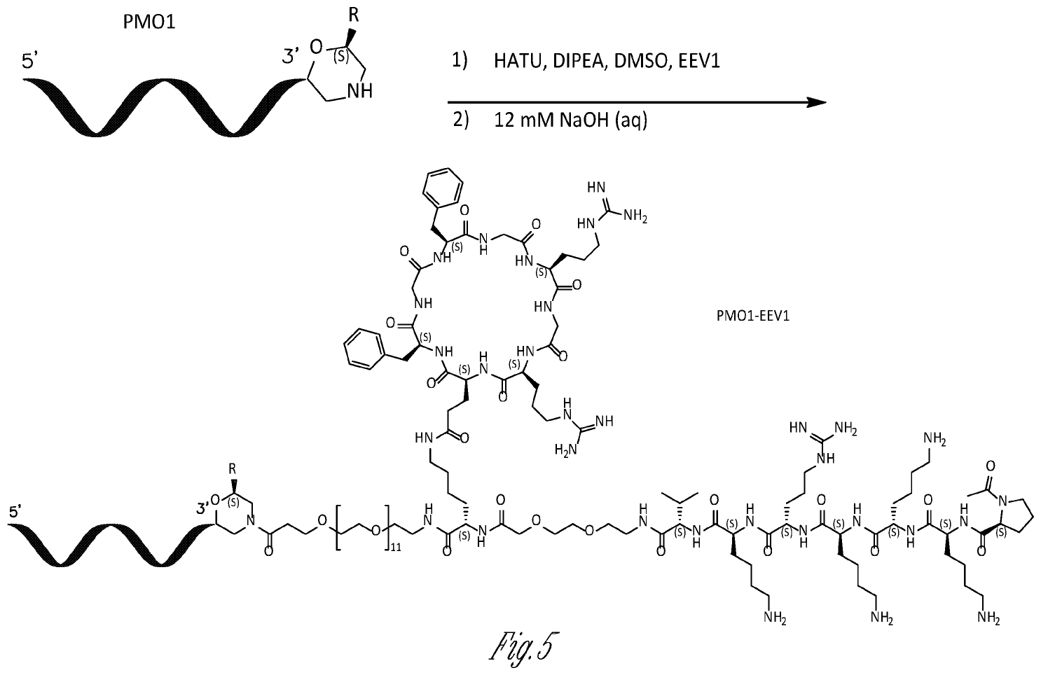

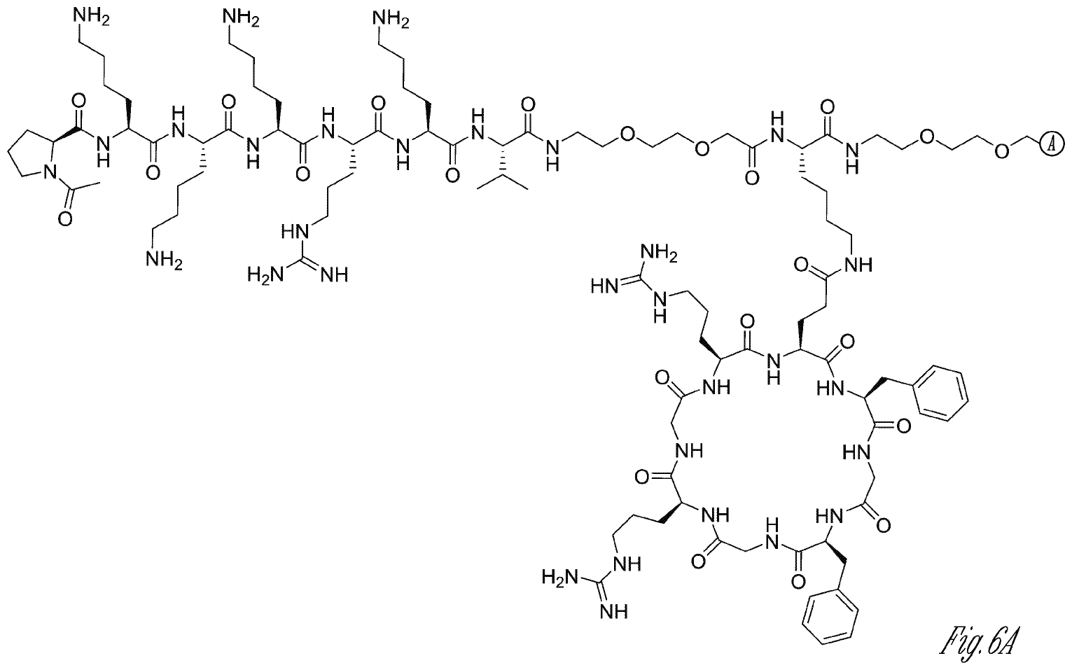

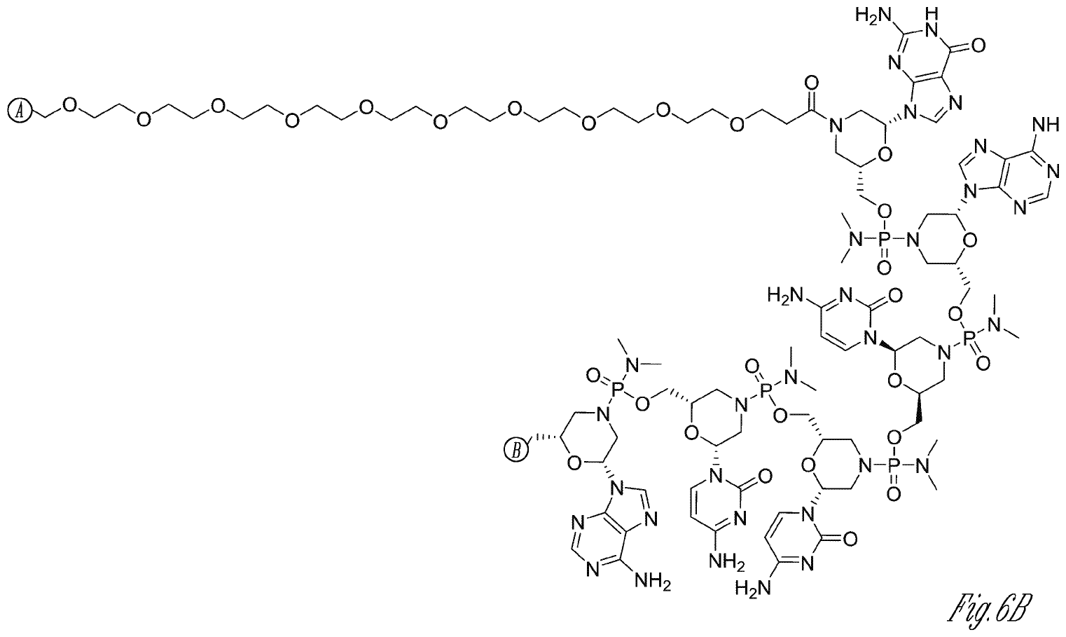

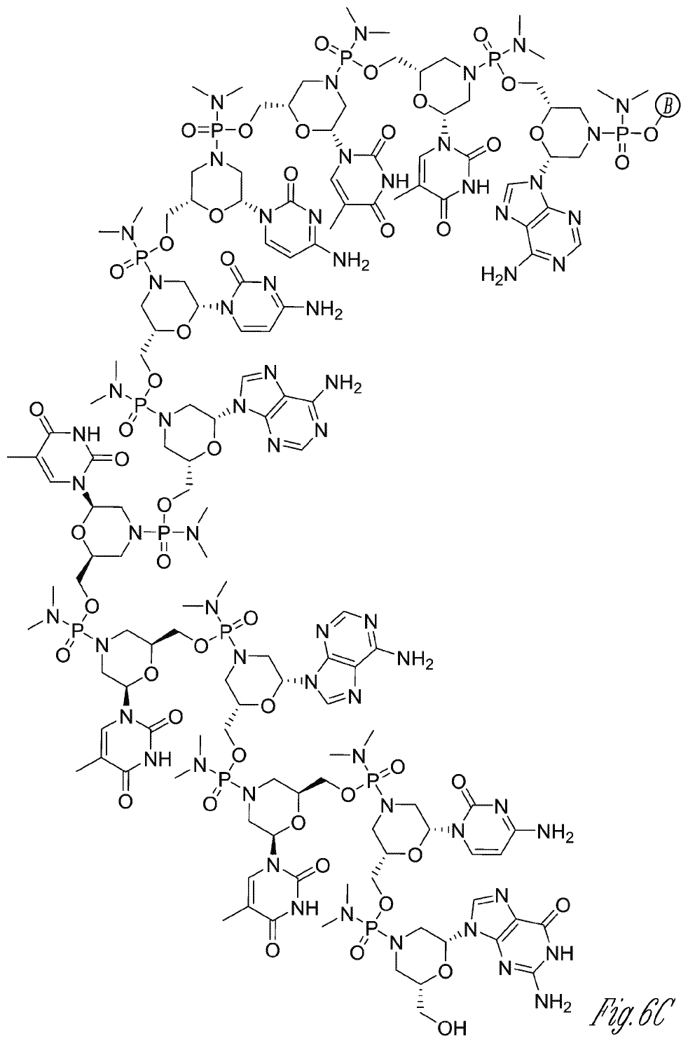

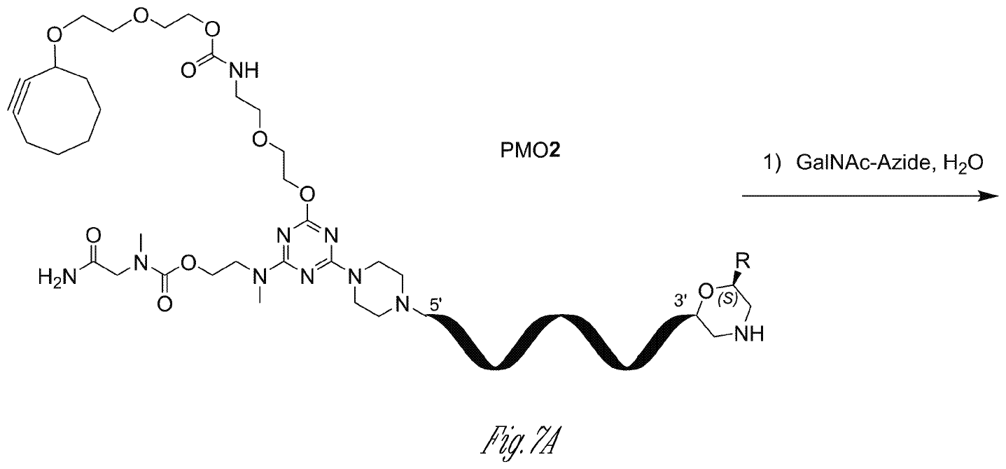

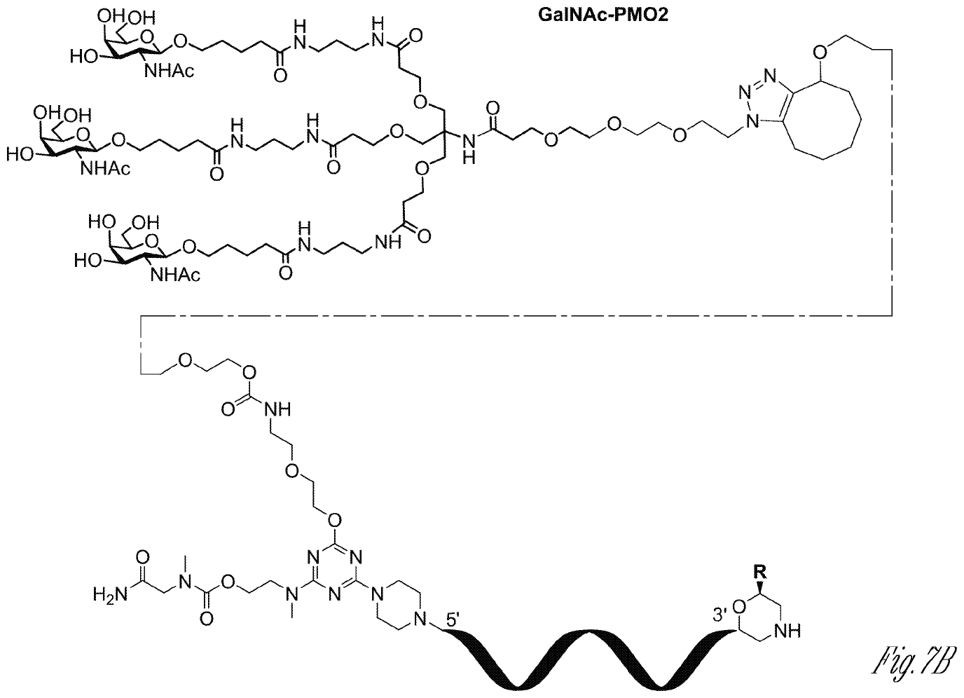

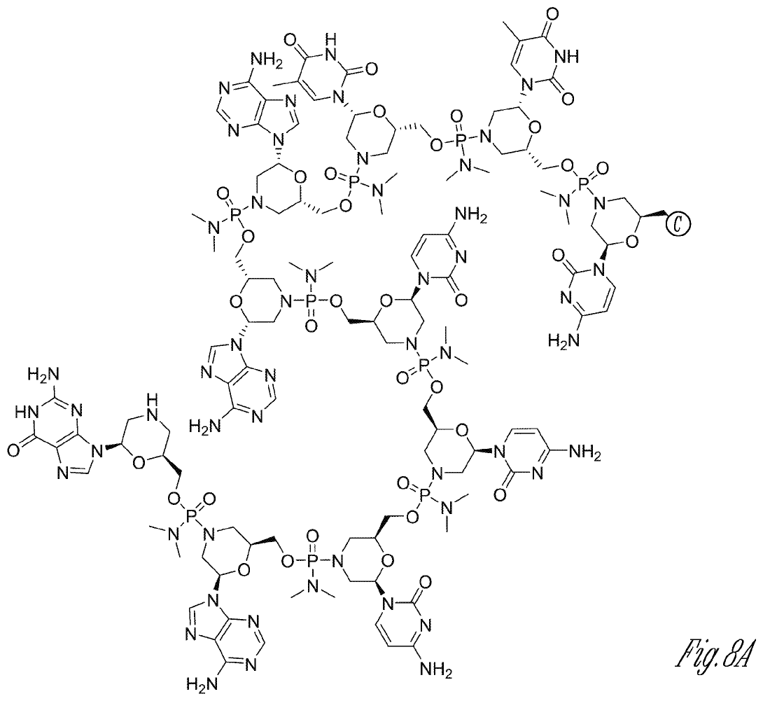

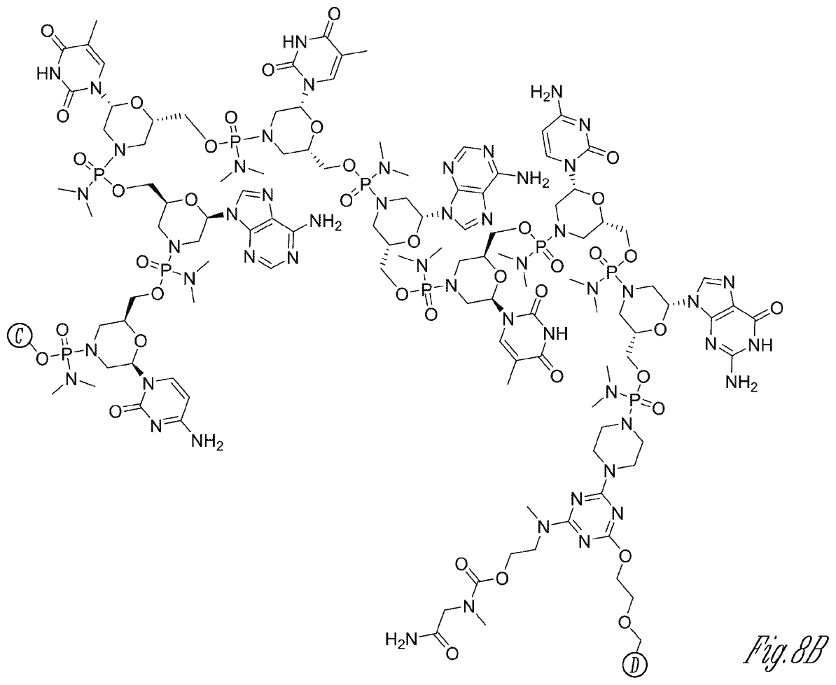

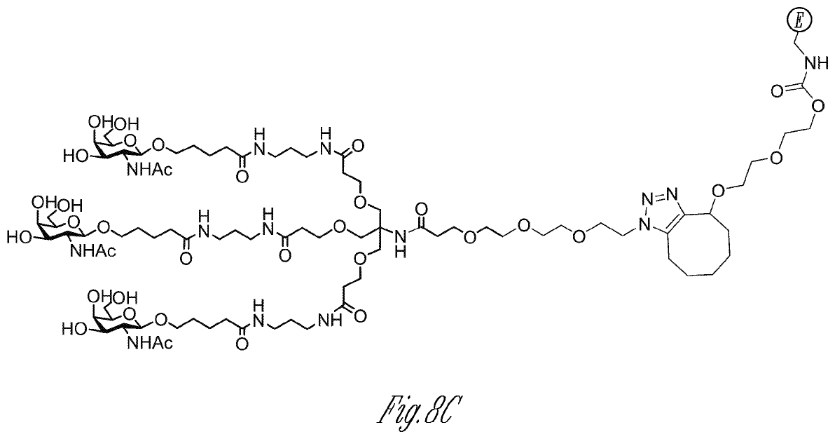

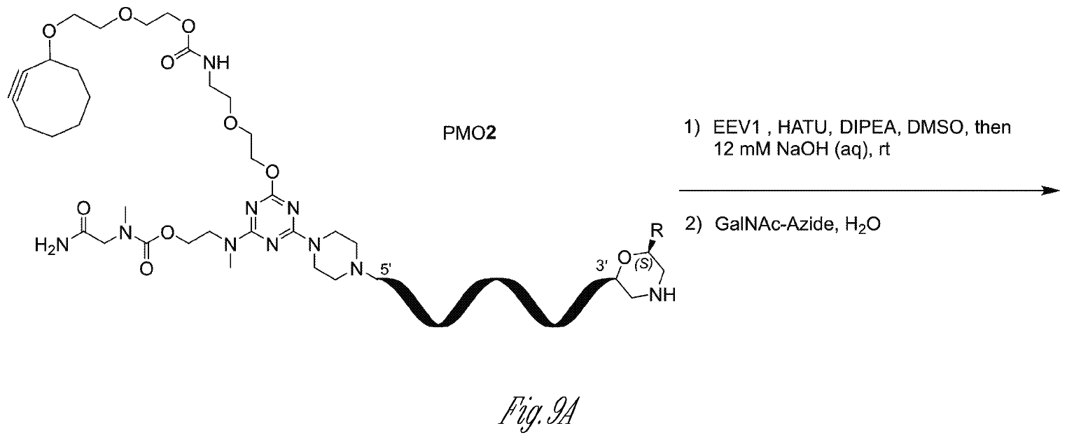

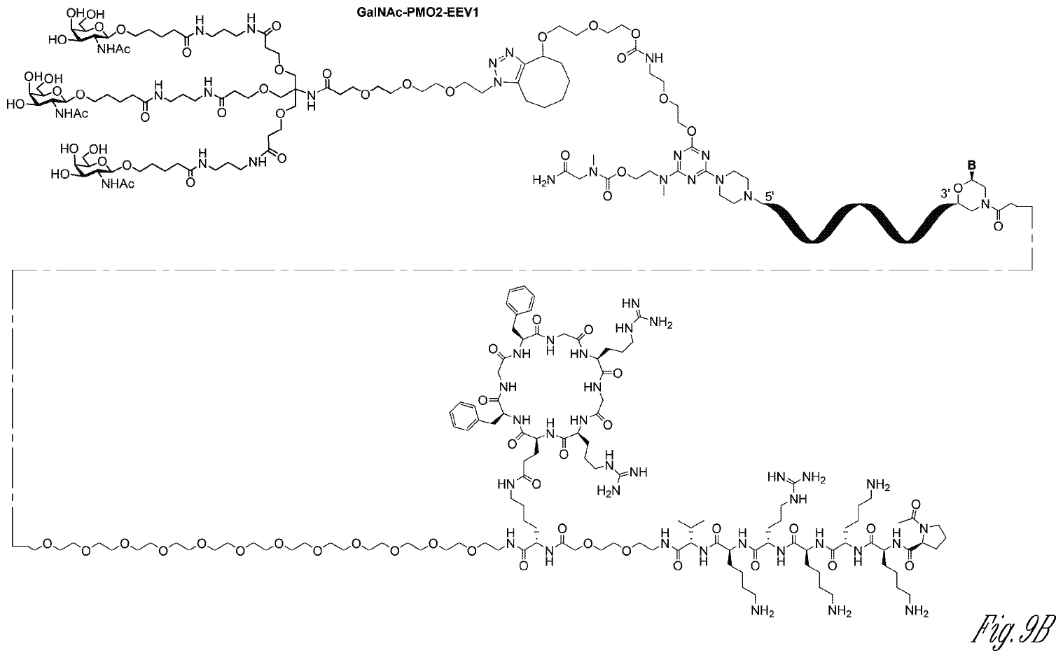

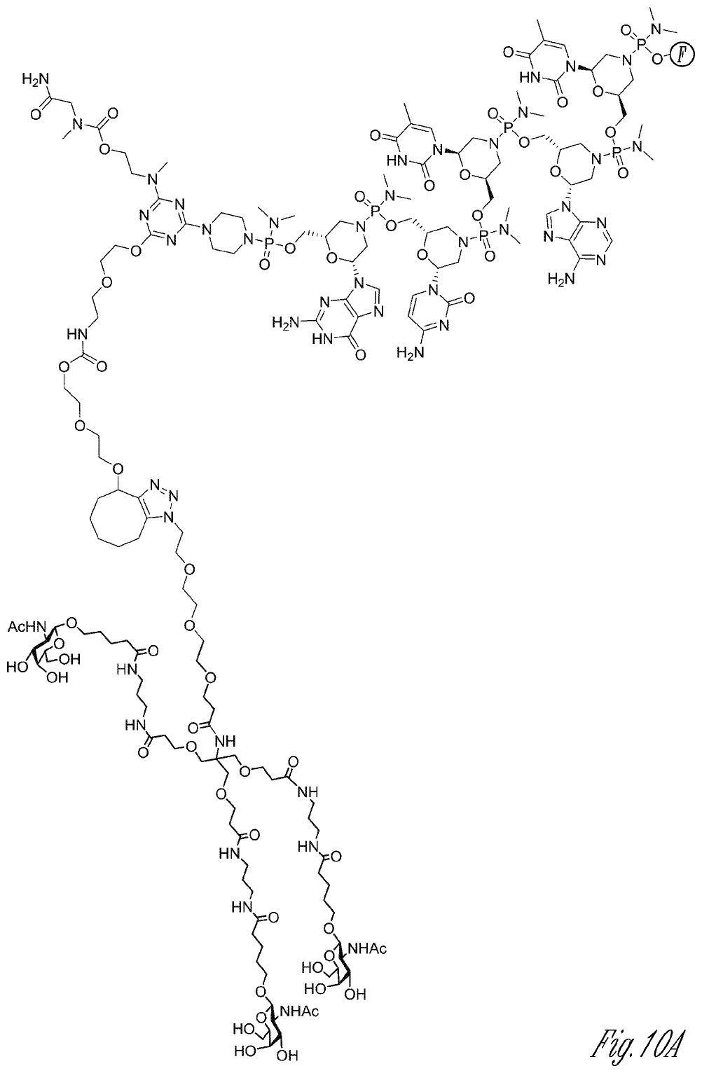

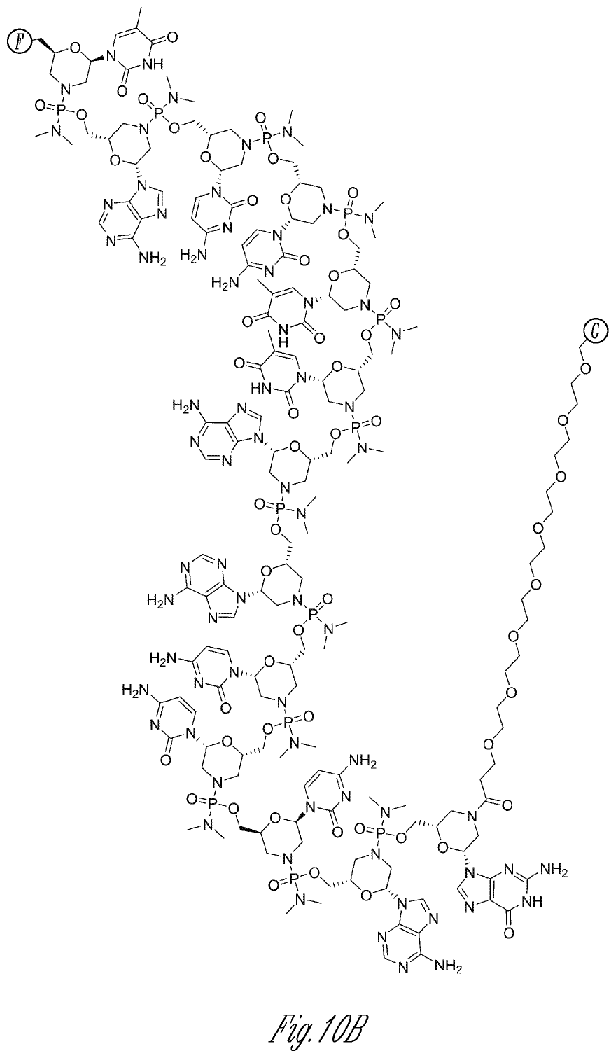

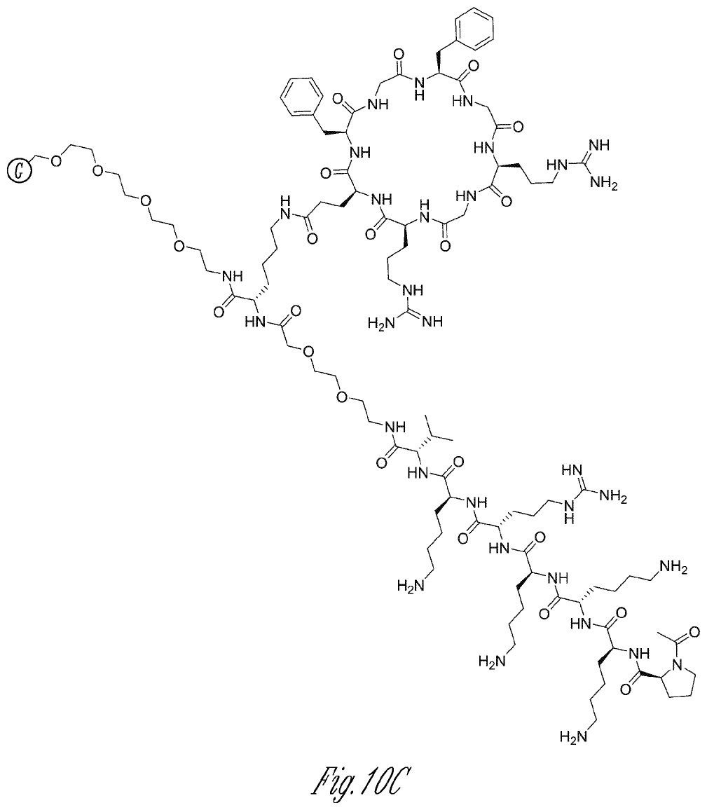

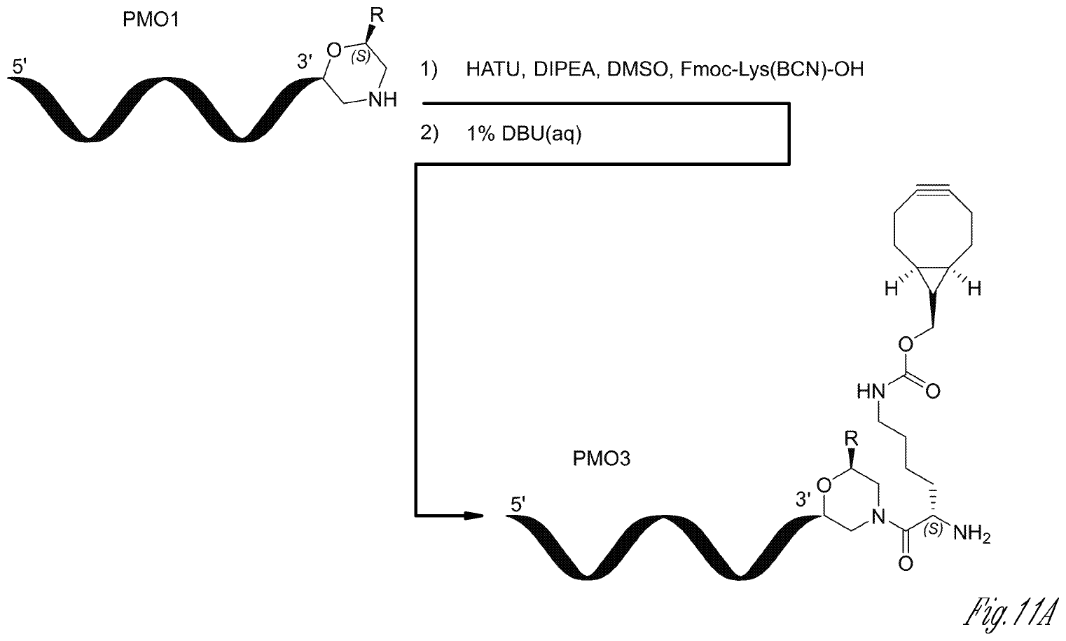

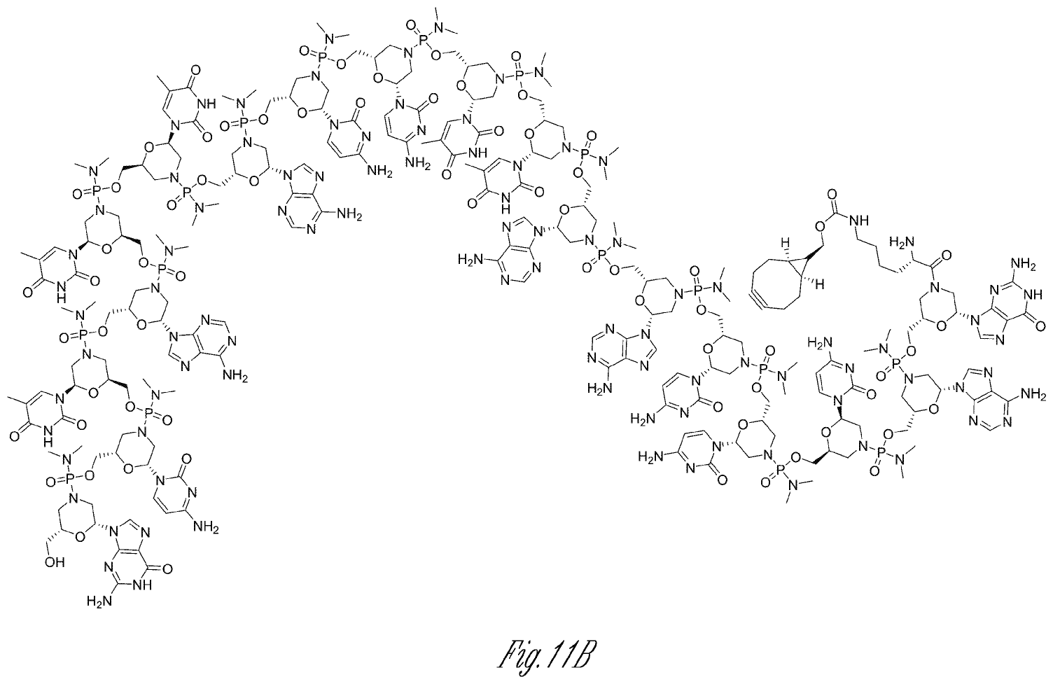

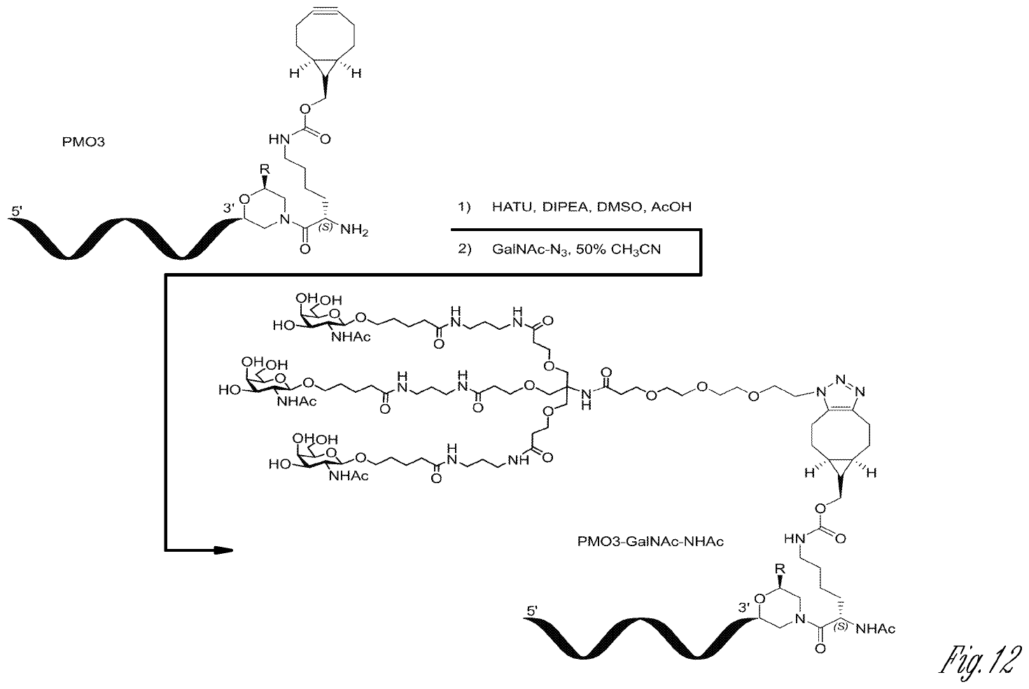

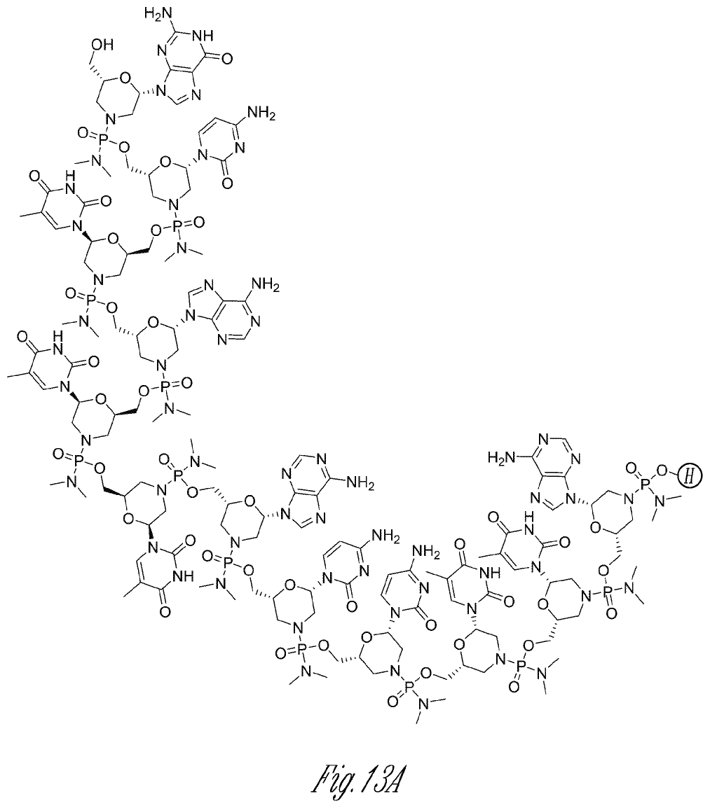

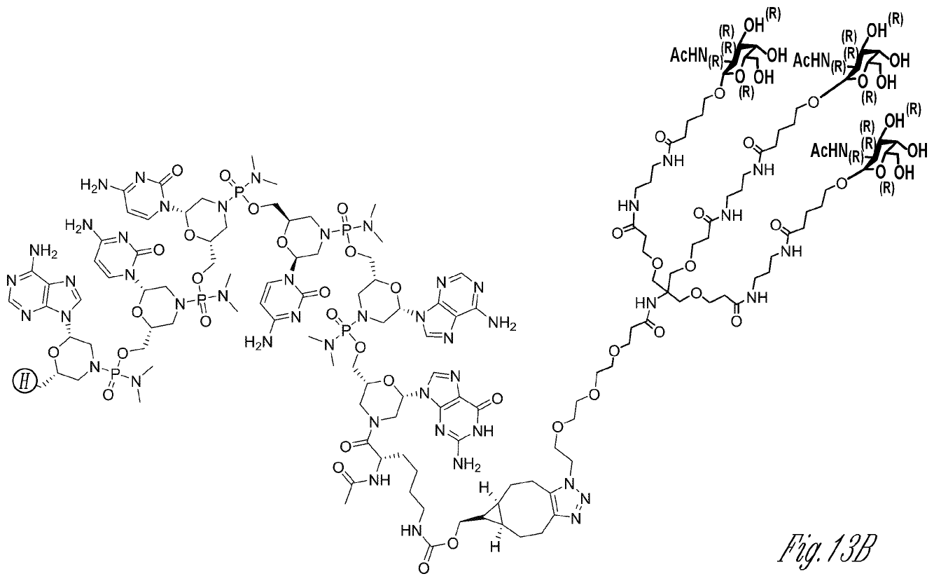

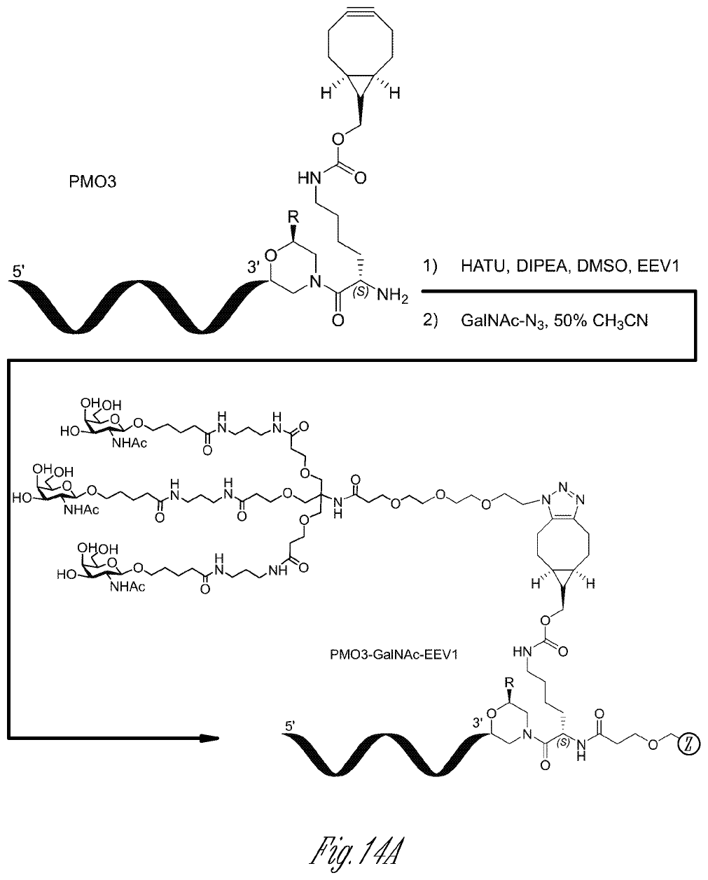

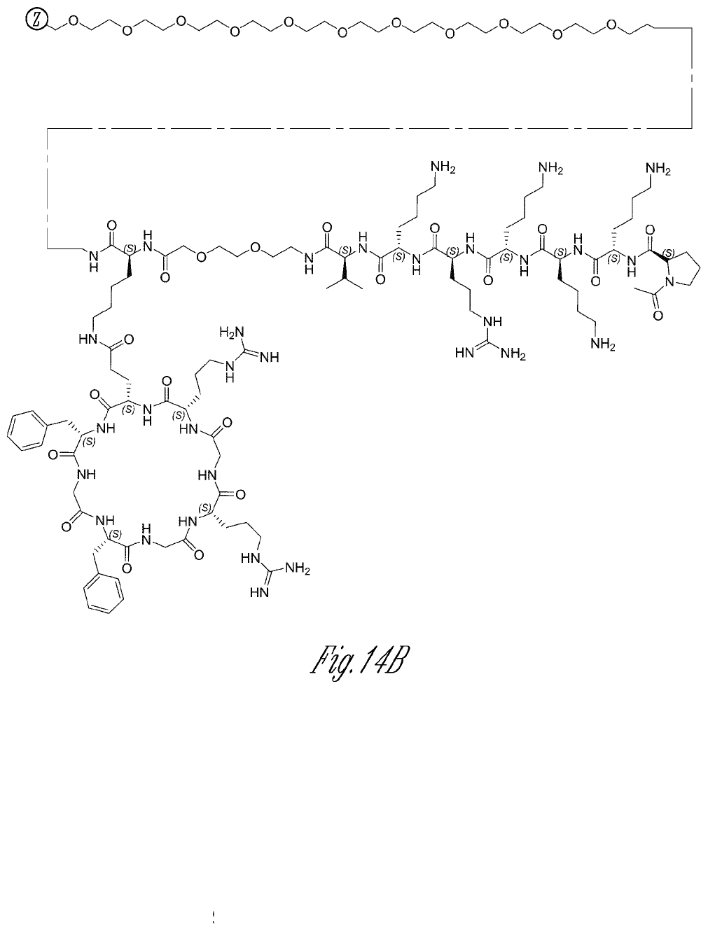

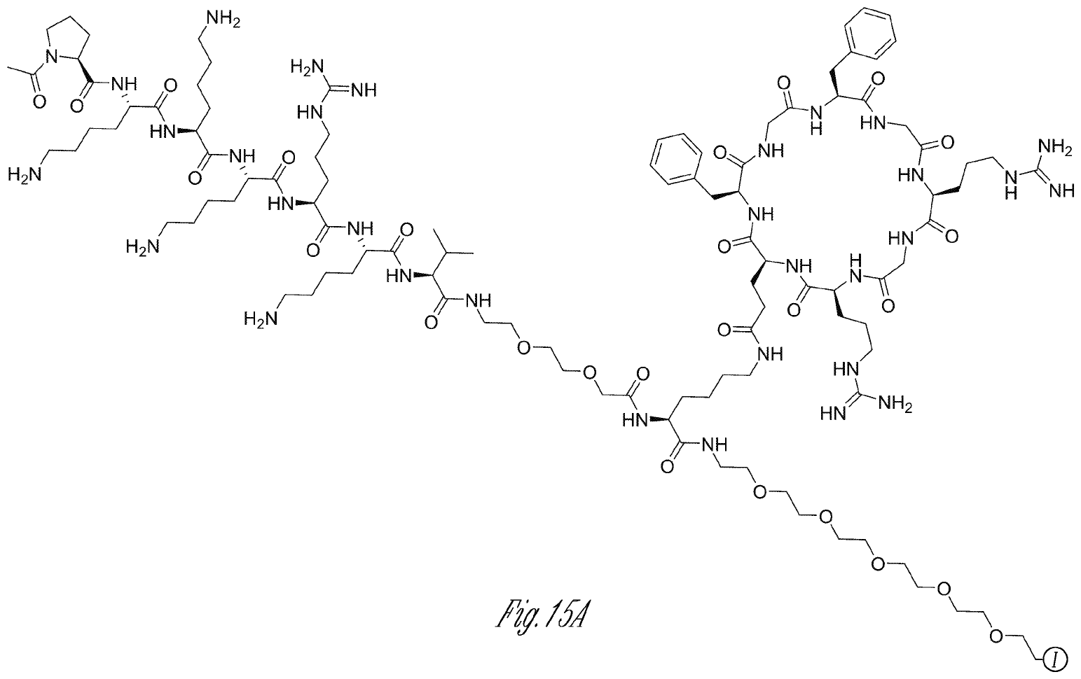

In embodiments, some or all of the nucleosides are modified nucleosides. In embodiments, one or more nucleosides include a modified nucleobase. In embodiments, one or more nucleosides include a modified sugar. Chemically modified nucleosides are routinely used for incorporation into antisense compounds to enhance one or more properties, such as nuclease resistance, pharmacokinetics or affinity for a target RNA. Non-limiting examples of nucleosides are provided in FIG. 1 and in Khvorova et al. Nature Biotechnology (2017) 35:238-248, which is incorporated by reference herein in its entirety.

In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The terms modified nucleobase and nucleobase mimetic can overlap but generally a modified nucleobase refers to a nucleobase that is fairly similar in structure to the parent nucleobase, such as for example a 7-deaza purine, a 5-methyl cytosine, or a G-clamp, whereas a nucleobase mimetic would include more complicated structures, such as for example a tricyclic phenoxazine nucleobase mimetic. Methods for preparation of the above noted modified nucleobases are well known to those skilled in the art.

In embodiments, therapeutic ologonucleotides provided herein include one or more nucleosides having a modified sugar moiety. In embodiments, the furanosyl sugar ring of a natural nucleoside can be modified in a number of ways including, but not limited to, addition of a substituent group, bridging of two non-geminal ring atoms to form a bicyclic nucleic acid (BNA) and substitution of an atom or group such as —S—, —N(R)— or —C(R1)(R2) for the ring oxygen at the 4′-position. Modified sugar moieties are well known and can be used to alter, typically increase, the affinity of the antisense compound for its target and/or increase nuclease resistance. A representative list of modified sugars includes but is not limited to non-bicyclic substituted sugars, especially non-bicyclic 2′-substituted sugars having a 2′-F, 2′-OCH3 or a 2′-O(CH2)2-OCH3 substituent group; and 4′-thio modified sugars. Sugars can also be replaced with a sugar mimetic group, for example, a methylenemorpholine ring, among others.

In embodiments, nucleosides include bicyclic modified sugars (BNA's), including LNA (4′-(CH2)-O-2′ bridge), 2′-thio-LNA (4′-(CH2)-S-2′ bridge), 2′-amino-LNA (4′-(CH2)-NR-2′ bridge), ENA (4′-(CH2)2-O-2′ bridge), 4′-(CH2)3-2′ bridged BNA, 4′-(CH2CH(CH3))-2′ bridged BNA″ cEt (4′-(CH(CH3)-O-2′ bridge), and cMOE BNAs (4′-(CH(CH2OCH3)-O-2′ bridge).

Also provided herein are “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Illustrative Internucleoside Linkages

Internucleoside linking groups link the nucleosides or otherwise modified monomer units of an oligonucleotide together. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, phosphorodiamidate, and phosphorothioates. Representative non-phosphorus containing internucleoside linking groups include, but are not limited to, methylenemethylimino (—CH2-N(CH3)-O-CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, a phosphate group can be linked to the 2′, 3′ or 5′ (or 6′, for a 6 membered ring, such as a methylenemorpholine ring) hydroxyl moiety of the sugar (or sugar mimetic). In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage. In embodiments, the oligonucleotide is a Phosphorodiamidate Morphoino Oligomer (PMO) comprising a backbone of methylenemorpholine rings linked through phosphorodiamidate internucleotide linkages.

Antisense PMOs are uncharged nucleic acid analogs bind to target nucleic acid through base paring. Antisense PMOs that bind to mRNA may block interaction of proteins to the mRNA through steric blockade. See, e.g., Nan and Zhang, Front. Microbiol. 20 Apr. 2019 (doi.org/10.3389/fmicb.2018.00750). As uncharged, or net neutral charged, oligonucleotides, PMOs are particularly effective for intracellular delivery with the endosomal escape vehicles (EEV) described herein.

### Conjugate Groups

In embodiments, therapeutic ologonucleotides are modified by covalent attachment of one or more conjugate groups. In general, conjugate groups modify one or more properties of the attached therapeutic ologonucleotides including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an therapeutic ologonucleotides. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the therapeutic ologonucleotide, a linker, an EP, or the cyclic peptide.

### (CRISPR) Gene-Editing Machinery

In embodiments, the therapeutic moiety comprises one or more component of CRISPR gene-editing machinery. As used herein, “CRISPR gene-editing machinery” refers to protein, nucleic acids, or combinations thereof, which may be used to edit a genome. Non-limiting examples of gene-editing machinery include guide RNAs (gRNAs), nucleases, nuclease inhibitors, and combinations and complexes thereof.

The CRISPR gene editing machinery may be used to repair a mutated gene or to introduce a mutation into a gene. The gene may be a gene associated with a disease of the eye.

In embodiments, a linker conjugates the cyclic peptide to the CRISPR gene-editing machinery. Any linker described in this disclosure or that is known to a person of skill in the art may be utilized.

gRNA

In embodiments, the compounds include a cyclic peptide conjugated to a gRNA. A gRNA targets a genomic loci in a prokaryotic or eukaryotic cell.

In embodiments, the gRNA is a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In embodiments, the spacer may be about 17-24 bases in length, such as about 20 bases in length.

In embodiments, the spacer targets a site that immediately precedes a 5′ protospacer adjacent motif (PAM). The PAM sequence may be selected based on the desired nuclease. For example, the PAM sequence may be any one of the PAM sequences shown in Table 7 below, wherein N refers to any nucleic acid, R refers to A or G, Y refers to C or T, W refers to A or T, and V refers to A or C or G.

TABLE 7
Nucleases and PAM sequences

| PAM sequence (5′ to 3′) | Nuclease | Isolated from |
|---|---|---|
| NGG | SpCas9 | Streptococcus pyogenes |
| NGRRT or NGRRN | SaCas9 | Staphylococcus aureus |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

In embodiments, a spacer may target a sequence of a mammalian gene, such as a human gene. In embodiments, the spacer may target a mutant gene. In embodiments, the spacer may target a coding sequence. In embodiments, the spacer may target an exonic sequence. In embodiments, the spacer may target a polyadenylation site (PS). In embodiments, the spacer may target a sequence element of a PS. In embodiments, the spacer may target a polyadenylation signal (PAS), an intervening sequence (IS), a cleavage site (CS), a downstream element (DES), or a portion or combination thereof. In embodiments, a spacer may target a splicing element (SE) or a cis-splicing regulatory element (SRE).

The scaffold sequence is the sequence within the sgRNA that is responsible for nuclease (e.g., Cas9) binding. The scaffold sequence does not include the spacer/targeting sequence. In embodiments, the scaffold may be about 10 to about 150 nucleotides in length, or about 50 to about 100 nucleotides in length.

In embodiments, the gRNA is a dual-molecule guide RNA, e.g, crRNA and tracrRNA. In embodiments, the gRNA may further include a poly(A) tail.

In embodiments, a compound that includes a CPP is conjugated to a nucleic acid that includes a gRNA. In embodiments, the nucleic acid includes about 1, about 2, about 3, about 4, about 5, about 6, about 7, about 8, about 9, about 10, about 11, about 12, about 13, about 14, about 15, about 16, about 17, about 18, about 19, or about 20 gRNAs. In embodiments, the gRNAs recognize the same target. In embodiments, the gRNAs recognize different targets. In embodiments, the nucleic acid that includes a gRNA includes a sequence encoding a promoter, wherein the promoter drives expression of the gRNA.

### Nuclease

In embodiments, the compounds include a cyclic peptide conjugated to a nuclease. In embodiments, the nuclease is a Type II, Type V-A, Type V-B, Type VC, Type V-U, Type VI-B nuclease. In embodiments, the nuclease is a transcription, activator-like effector nuclease (TALEN), a meganuclease, or a zinc-finger nuclease or a modified form or varient thereof. In embodiments, the nuclease is a Cas9, Cas12a (Cpf1), Cas12b, Cas12c, Tnp-B like, Cas13a (C2c2), Cas13b, or Cas14 nuclease or a modified form or varient thereof. For example, in some embodiments, the nuclease is a Cas9 nuclease or a Cpf1 nuclease.

In embodiments, a compound that includes a cyclic peptide is conjugated to a nuclease. In embodiments, the nuclease is a soluble protein.

In embodiments, a compound that includes a cyclic peptide is conjugated to a nucleic acid encoding a nuclease. In embodiments, the nucleic acid encoding a nuclease includes a sequence encoding a promoter, wherein the promoter drives expression of the nuclease.

gRNA and Nuclease Combinations

In embodiments, the compounds include one or more CPP (or cCPP) conjugated to a gRNA and a nuclease. In embodiments, the one or more CPP (or cCPP) are conjugated to a nucleic acid encoding a gRNA and/or a nuclease. In embodiments, the nucleic acid encoding a nuclease and a gRNA includes a sequence encoding a promoter, wherein the promoter drives expression of the nuclease and the gRNA. In embodiments, the nucleic acid encoding a nuclease and a gRNA includes two promoters, wherein a first promoter controls expression of the nuclease and a second promoter controls expression of the gRNA. In embodiments, the nucleic acid encoding a gRNA and a nuclease encodes from about 1 to about 20 gRNAs, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. In embodiments, the gRNAs recognize different targets. In embodiments, the gRNAs recognize the same target.

In embodiments, the compounds include a cell penetrating peptide (or cCPP) conjugated to a ribonucleoprotein (RNP) that includes a gRNA and a nuclease.

In embodiments, a composition that includes: (a) a cyclic peptide conjugated to a gRNA and (b) a nuclease is delivered to a cell. In embodiments, a composition that includes: (a) a cyclic peptide conjugated to a nuclease and (b) an gRNA is delivered to a cell.

In embodiments, a composition that includes. (a) a first cyclic peptide conjugated to a gRNA and (b) a second cyclic peptide conjugated to a nuclease is delivered to a cell. In embodiments, the first cyclic peptide and the second cyclic peptide are the same. In embodiments, the first cyclic peptide and the second second cyclic are different.

### Nuclease Inhibitors

In embodiments, the compounds disclosed herein include a cyclic peptide conjugated to an inhibitor of a nuclease (e.g., Cas9). A limitation of gene editing is potential off-target editing. The delivery of a nuclease inhibitor may limit off-target editing. In embodiments, the nuclease inhibitor is a polypeptide, polynucleotide, or small molecule.

### Conjugate Groups

In embodiments, TOs are modified by covalent attachment of one or more conjugate groups. In general, conjugate groups modify one or more properties of the attached TO including but not limited to pharmacodynamic, pharmacokinetic, binding, absorption, cellular distribution, cellular uptake, charge, and clearance. Conjugate groups are routinely used in the chemical arts and are linked directly or via an optional linking moiety or linking group to a parent compound such as a TO. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, and dyes. In embodiments, the conjugate group is a polyethylene glycol (PEG), and the PEG is conjugated to one or more of the TO, the EP, the CPP, and the CTM.

In embodiments, conjugate groups include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996,277,923).

Linking groups or bifunctional linking moieties such as those known in the art are amenable to the compounds provided herein. Linking groups are useful for attachment of chemical functional groups, conjugate groups, reporter groups and other groups to selective sites in a parent compound such as for example a TO. In general, a bifunctional linking moiety includes a hydrocarbyl moiety having two functional groups. One of the functional groups is selected to bind to a parent molecule or compound of interest and the other is selected to bind essentially any selected group such as chemical functional group or a conjugate group. Any of the linkers described here may be used. In embodiments, the linker includes a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of substituent groups includes hydroxyl, amino, alkoxy, carboxy, benzyl, phenyl, nitro, thiol, thioalkoxy, halogen, alkyl, aryl, alkenyl and alkynyl.

In embodiments, the TO may be an ASO. In embodiments, the ASO may be from about 5 to about 50 nucleotides in length. In embodiments, the ASO may be from about 5 to about 10 nucleotides in length. In embodiments, the ASO may be from about 10 to about 15 nucleotides in length. In embodiments, the ASO may be from about 15 to about 20 nucleotides in length. In embodiments, the ASO may be from about 20 to about 25 nucleotides in length. In embodiments, the ASO may be from about 25 to about 30 nucleotides in length. In embodiments, the ASO may be from about 30 to about 35 nucleotides in length. In embodiments, the ASO may be from about 35 to about 40 nucleotides in length. In embodiments, the ASO may be from about 40 to about 45 nucleotides in length. In embodiments, the ASO may be from about 45 to about 50 nucleotides in length.

### Detectable Moiety

In embodiments, the compound disclosed herein includes a detectable moiety. In embodiments, the detectible moiety is attached to any portion of the EEV. The detectable moiety can include any detectable label. The detectable moiety can contain a luminophore such as a fluorescent label or near-infrared label.

### Compositions

In embodiments, compositions are provided that include the compounds described herein.

In embodiments, pharmaceutically acceptable salts and/or prodrugs of the disclosed compounds are provided. Pharmaceutically acceptable salts include salts of the disclosed compounds that are prepared with acids or bases, depending on the substituents found on the compounds. Under conditions where the compounds disclosed herein are sufficiently basic or acidic to form stable nontoxic acid or base salts, administration of the compounds as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Mechanism of Modulation and Diseases

The present disclosure provides a method of treating disease in a patient in need thereof, that includes administering a compound disclosed herein. In embodiments, the disease is any of the diseases provided in the present disclosure. In embodiments, a method of treating a disease includes administering to the patient a compound disclosed herein, thereby treating the disease. The compound comprises a CTM, a CPP, and a TO. The compound may further comprise an EP.

In embodiments, the disease or disorder may include, but is not limited to, one or more of Pompe disease, Wilson disease, amyloidotic cardiomyopathy, hypercholesterolemia, hemophilia or rare bleeding disorders (including, for example, hemophilia A or hemophilia B), paroxysmal nocturnal hemoglobinuria, alpha-1-antitrypsin deficiency, primary hyperoxaluria type 1, hepatitis (including, for example, hepatitis A, hepatitis B, hepatitis C, hepatitis D, hepatitis E, hepatitis F, hepatitis G, or hepatitis H), hepatic porphyrias, beta-thalassemia or iron overload disorders, angioedema (including, for example, hereditary angioedema), thromboprophylaxis, hypertriglyceridemia, hyperlipidemia, hypertension (including, for example, treatment resistant hypertension), hereditary hemochromatosis (HH), pre-eclampsia, chronic liver infection, thrombosis, orphan genetic disease, cardiovascular disease, fibrotic liver diseases, Non-alcoholic Fatty Liver Disease (NAFLD) (including, for example, non-alcoholic steatohepatitis (NASH)), diabetes (including, for example, type 1 diabetes, type 2 diabetes, and pre-diabetes), high lipoprotein(a), dislipidemias, acromegaly, ornithine transcarbamylase deficiency, obesity, liver cancer (including, for example, hepatocellular carcinoma (HCC), fibrolamellar HCC, hepatoblastoma, chloangriocarcinoma, angiosarcoma, hemangiosarcoma, or liver metastasis, mucopolysaccharidosis type 1, mucopolysaccharidosis type 2, methylmalonic acidemia, autoimmune hepatitis, and phenylketonuria.

In embodiments, the disease or disorder to be treated includes liver diseases or disorders characterized by unwanted cell proliferation, genetic disorders, hematological disorders, metabolic disorders, and disorders characterized by inflammation. A proliferation disorder of the liver can be, for example, a benign or malignant disorder, e.g., a cancer, e.g., a hepatocellular carcinoma (HCC), hepatic metastasis, or hepatoblastoma. A hepatic hematology or inflammation disorder can be a disorder involving clotting factors, a complement-mediated inflammation or a fibrosis, for example. Metabolic diseases of the liver include dyslipidemias and irregularities in glucose regulation.

### Methods of Treatment

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have wild type target protein expression or activity levels that are less than about 1% to about 99% of normal protein expression or activity levels in an individual not afflicted with the disease. In embodiments, the range includes, but is not limited to less than about 80% to about 99%, less than about 65% to about 80%, less than about 50% to about 65%, less than about 30% to about 50%, less than about 25% to about 30%, less than about 20% to about 25%, less than about 15% to about 20%, less than about 10% to about 15%, less than about 5% to about 10%, less than about 1% to about 5% of normal thymidine phosphorylase expression or activity levels. In embodiments, the individual may have target protein expression or activity levels that are 1% to about 500% higher than normal wild type target protein expression or activity levels. In embodiments, the range includes, but is not limited to, greater than about 1% to about 10%, about 10% to about 50%, about 50% to about 100%, about 100% to about 200%, about 200% to about 300%, about 300% to about 400%, about 400% to about 500%, or about 500% to about 1000%.

In embodiments, the individual is a patient who has been recently diagnosed with the disease. Typically, early treatment (treatment commencing as soon as possible after diagnosis) reduces the effects of the disease and to increase the benefits of treatment.

### Methods of Making

The compounds described herein can be prepared in a variety of ways known to one skilled in the art of organic synthesis or variations thereon as appreciated by those skilled in the art. The compounds described herein can be prepared from readily available starting materials. Reaction conditions can vary with the reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety. 16691 The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminal is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group can be used for the synthesis of the disclosed compounds. Other side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopentyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl). In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, for example, piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminal in the amino acids of the growing peptide chain are protected with Fmoc. The removal of the Fmoc protecting group from the α-N-terminal side of the growing peptide is accomplished by treatment with a secondary amine, for example, piperidine. In embodiments, each protected amino acid is then introduced in about 3-fold molar excess, and the coupling is carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either in successively or in a single operation. Removal of the polypeptide and deprotection can be accomplished in a single operation by treating the resin-bound polypeptide with a cleavage reagent that includes thioanisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution: high performance liquid chromatography (HPLC), especially reverse-phase HPLC on octyl- or octadecylsilyl-silica bonded phase column packing.

The above polymers, such as PEG groups, can be attached to the TO moiety under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, (x-haloacetyl, maleimido or hydrazino group) to a reactive group on the TO (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., (x-iodo acetic acid, α-bromoacetic acid, α-chloroacetic acid). If attached to the TO by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

In order to direct covalently link the TO moiety to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making TO and conjugating TO to linear CPP are generally described in US Pub. No. 2018/0298383, which is herein incorporated by reference for all purposes. The methods may be applied to the cyclic CPPs disclosed herein.

The disclosure relates to a method of making a conjugate of the formula (X), (Y) or (Z):

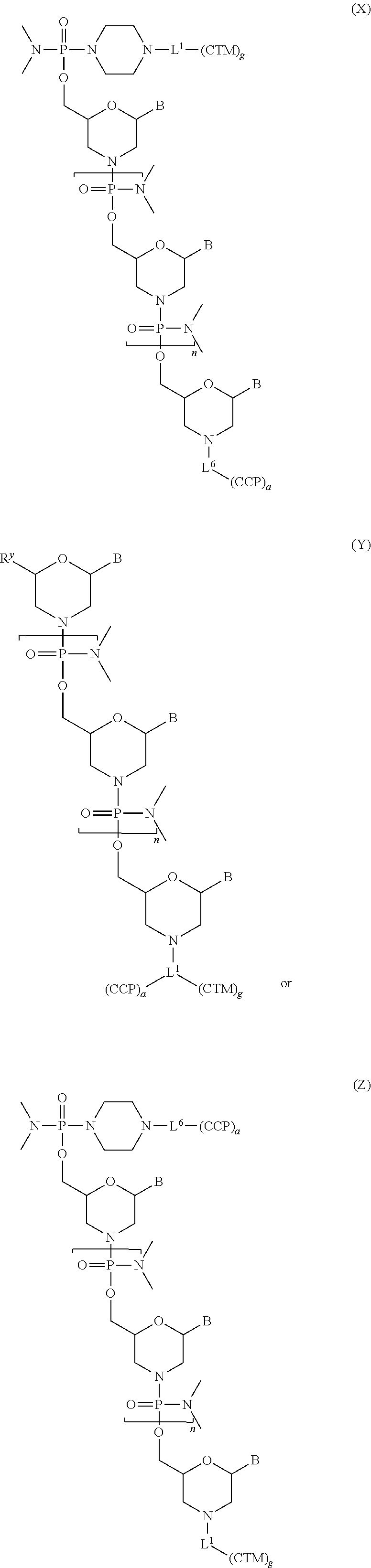

wherein:
CPP is a cell penetrating peptide;
a is an integer from 1 to 10;
the conjugates (X) and (Z) optionally further comprise an (EP), group, which is an exocyclic peptide, wherein c is an integer from 0 to 10;
L¹ and L⁶ are each, independently, a linker;
CTM is a carbohydrate targeting moiety;
g is an integer from 1 to 10;
R^{y} is H of —CH₂—OR;
R^{z} is a capping group;
B is each independently a nucleobase of the therapeutic oligonucleotide; and
n is an integer from 1 to 1000.
the method comprising (i) contacting a compound of the formula (X′) (Y′) or (Z′):

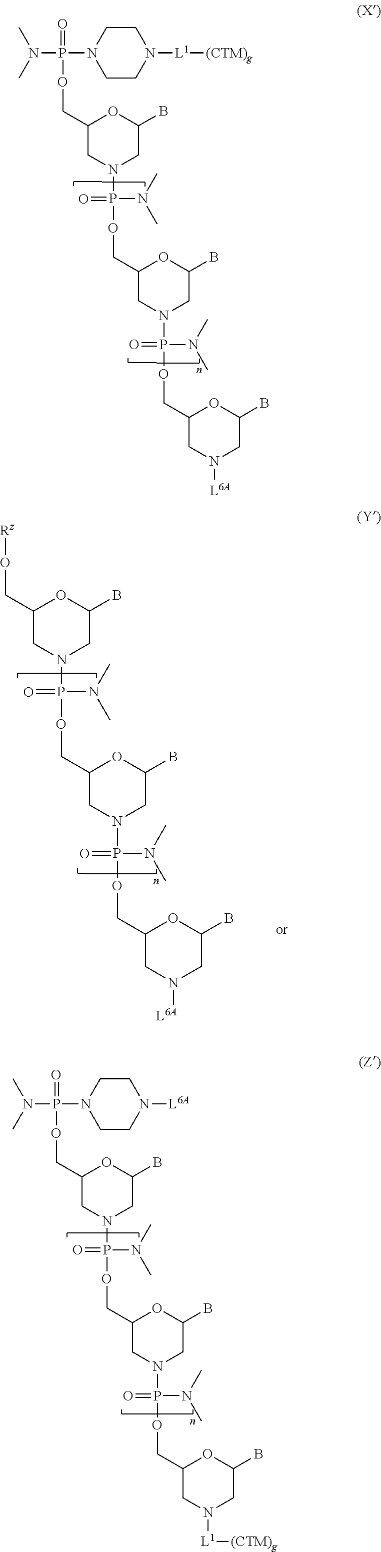

wherein:
(X′) and (Z′) optionally further comprise an (EP)_{c} group, which is an exocyclic peptide, wherein c is an integer from 0 to 10; and
L^{6A} is a linker comprising a nucleophilic group;
(ii) with a compound of the formula HO₂C-L⁷-(CPP)ₐ, wherein L⁷ is a linker, in the presence of a coupling reagent and a hindered base;
to give the conjugate of the formula (X), (Y), and (Z).

In embodiments, the nucleophilic group comprises an alkylamino group.

In embodiments, the coupling reagent is 7-azabenzotriazol-1-yloxy)tripyrrolidinophosphonium hexafluorophosphate.

In embodiments, wherein the hindered base is N,N-diisopropylethylamine.

The disclosure relates to a method of making a conjugate of the formula (X-1), (Y-1) or (Z-1):

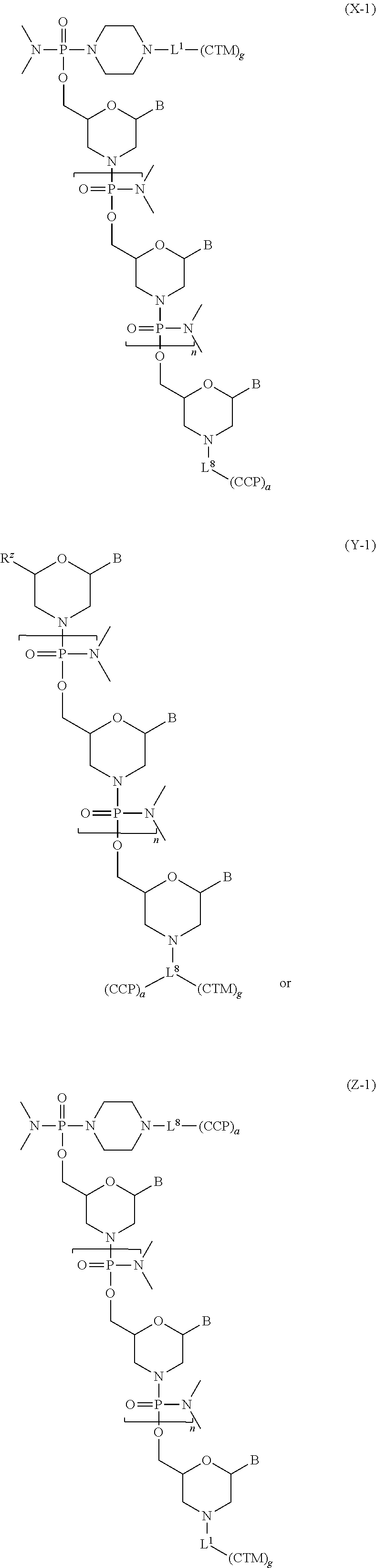

wherein:
CPP is a cell penetrating peptide;
a is an integer from 1 to 10;
the conjugate (Y) and (Q) optionally further comprise an (EP), group, which is an exocyclic peptide, wherein c is an integer from 0 to 10;
L¹ and L⁸ are each, independently, a linker;
CTM is a carbohydrate targeting moiety;
g is an integer from 1 to 10;
B is each independently a nucleobase of the therapeutic oligonucleotide; and
n is an integer from 1 to 1000; the method comprising contacting a compound of the formula (X′-1) (Y′-1) or (Z′-1):

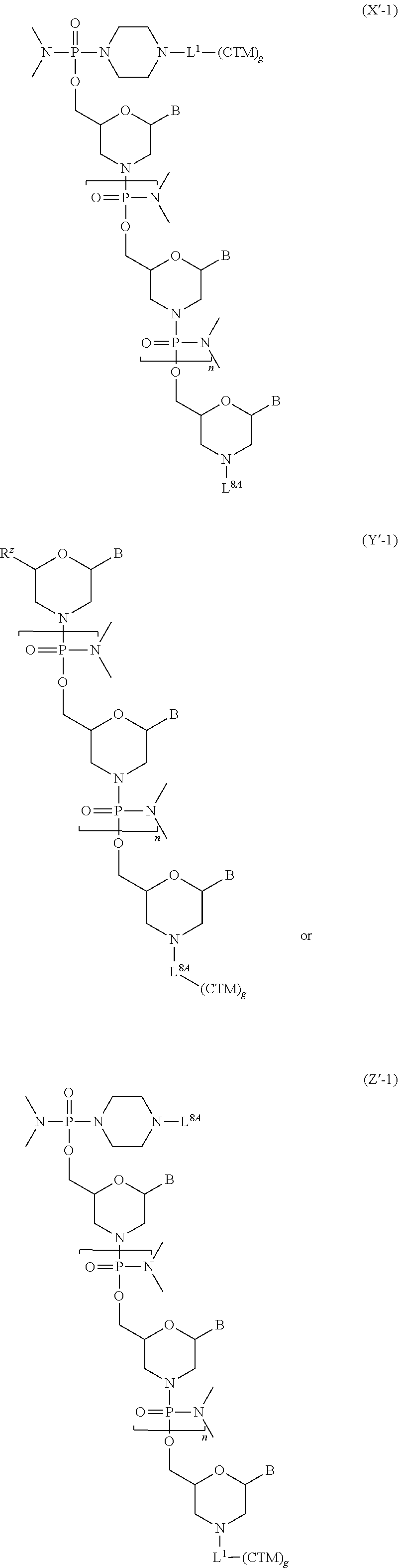

wherein L^{8A} is a linker comprising an alkyne;
with a compound of the formula N₃-L⁹-(CPP)ₐ under strain-promoted azide-alkyne cycloaddition conditions;
to give the compound of the formula (Y) and (Q).

In embodiments, L^{8A} comprises a —C(═O)—O-alkyl-O-group.

In embodiments, the alkyne is an alkyne of the formula:

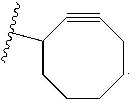 [SMILES: *C1C#CCCCCC1]

In embodiments, L⁸ comprises a group of the formula:

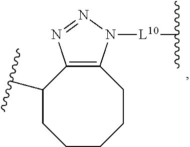

wherein L¹⁰ is a linker. In embodiments, L⁸ can comprise a group of the formula:

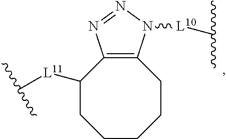

wherein L¹⁰ and L¹¹ are each, independently, linkers. In embodiments, L¹¹ can comprise a group of the formula —C(═O)—O-alkyl-O—. In embodiments, L¹¹ comprises a group of the formula
—C(═O)-alkyl-(OCH₂CH₂)_{q}O—, wherein q is an integer from 1 to 5.

In embodiments, the alkyne is an alkyne of the formula:

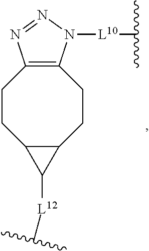

wherein L¹⁰ and L¹² are each, independently, linkers. In embodiments, L¹² comprises a group of the formula: —C(═O)-alkyl-(OCH₂CH₂)_{q}O—, wherein q is an integer from 1 to 5. In embodiments, L¹² comprises a group of the formula:
—C(═O)alkyl-NH—C(═O)-alkyl-(OCH₂CH₂)_{q}O—, wherein q is an integer from 1 to 5. In embodiments, L¹² comprises a group of the formula:
—C(═O)alkyl-NH—C(═O)-alkyl-(OCH₂CH₂)_{q}O-alkyl-NH—C(═O)O-alkyl, wherein q is an integer from 1 to 5.

The disclosure relates to a method of making a conjugate of the formula (G) or (D):

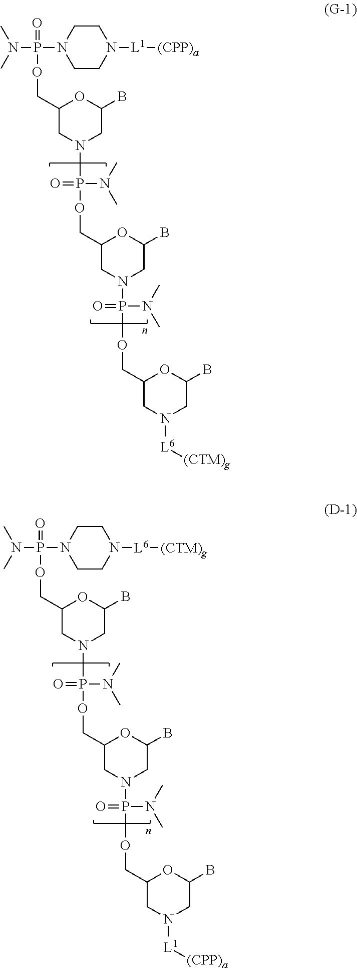

wherein:
CPP is a cell penetrating peptide;
a is an integer from 1 to 10;
the conjugates (G-1) and (D-1) optionally further comprise an (EP) group, which is an exocyclic peptide, wherein c is an integer from 0 to 10;
L¹ and L⁶ are each, independently, a linker;
CTM is a carbohydrate targeting moiety;
g is an integer from 1 to 10;
B is each independently a nucleobase of the therapeutic oligonucleotide; and
n is an integer from 1 to 1000.
the method comprising (i) contacting a compound of the formula (G′) or (D′):

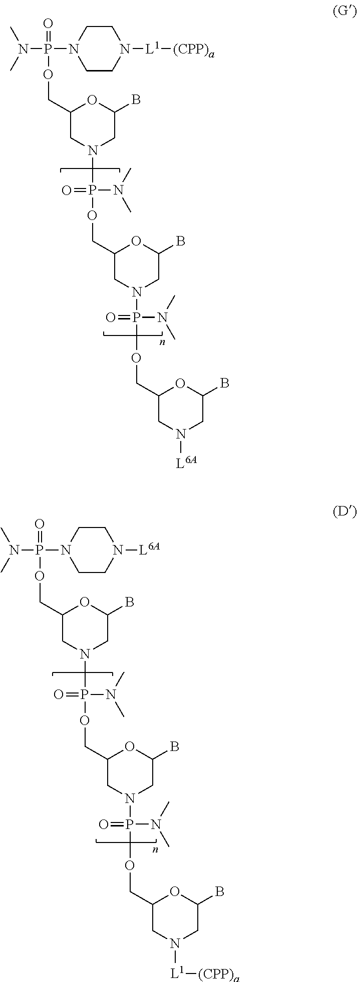

wherein:
(G′) and (D′) optionally further comprise an (EP)_{c} group, which is an exocyclic peptide, wherein c is an integer from 0 to 10; and
L^{6A} is a linker comprising a nucleophilic group;
(ii) with a compound of the formula HO₂C-L⁷-(CTM)_{g}, wherein L⁷ is a linker, in the presence of a coupling reagent and a hindered base;
to give the conjugate of the formula (G) or (D).

In embodiments, the nucleophilic group comprises an alkylamino group.

In embodiments, the coupling reagent is 7-azabenzotriazol-1-yloxy)tripyrrolidinophosphonium hexafluorophosphate.

In embodiments, the hindered base is N,N-diisopropylethylamine.

The disclosure also relates to a method of making a conjugate of the formula (S) (T) or (U):

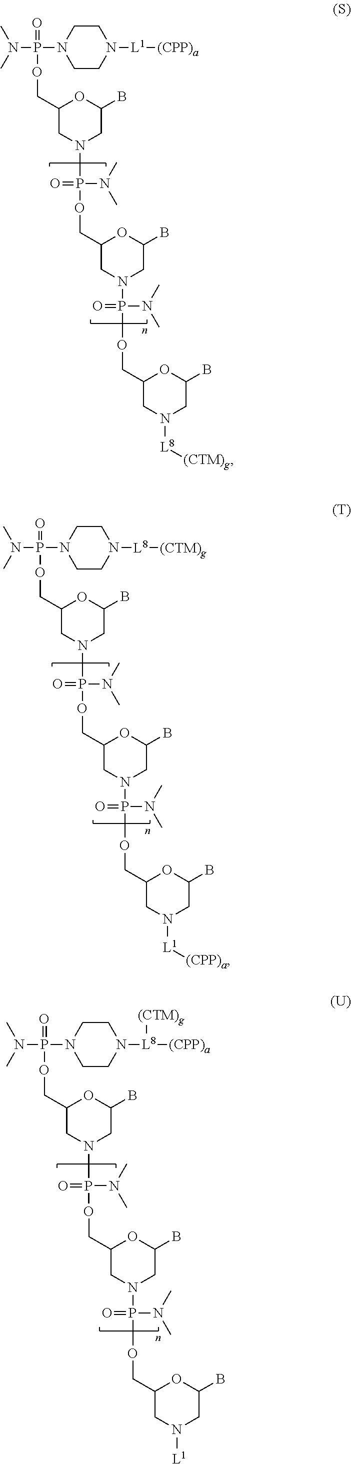

wherein:
CPP is a cell penetrating peptide;
a is an integer from 1 to 10;
the conjugate (S), (T) and (U) optionally further comprise an (EP), group, which is an exocyclic peptide, wherein c is an integer from 0 to 10;
L¹ and L⁸ are each, independently, a linker;
CTM is a carbohydrate targeting moiety;
g is an integer from 1 to 10;
B is each independently a nucleobase of the therapeutic oligonucleotide; and
n is an integer from 1 to 1000.
the method comprising (i) contacting a compound of the formula (S′) (T′) or (U′):

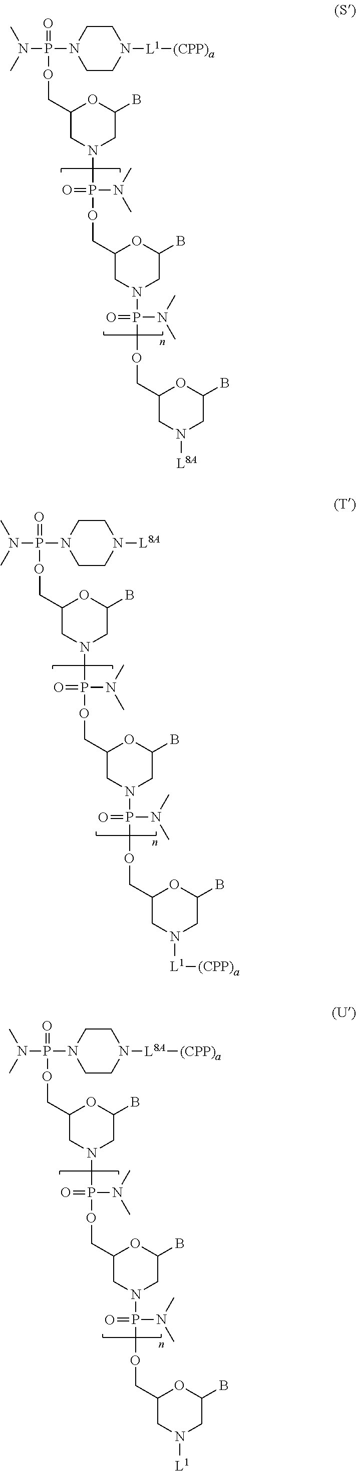

wherein L^{8A} is a linker comprising an alkyne;
with a compound of the formula N₃-L⁹-(CTM)_{g} under strain-promoted azide-alkyne cycloaddition conditions;
to give the compound of formula (S) (T) or (U).

In embodiments, wherein L^{8A} comprises a —C(═O)—O-alkyl-O-group.

In embodiments, the alkyne is an alkyne of the formula:

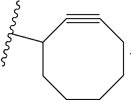 [SMILES: *C1C#CCCCCC1]

In embodiments, L⁸ comprises a group of the formula:

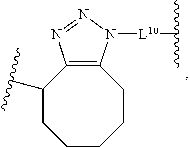

wherein L¹⁰ is a linker. In embodiments, L⁸ comprises a group of the formula:

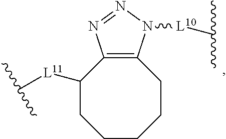

wherein L¹⁰ and L¹¹ are each, independently, linkers. In embodiments, L¹¹ comprises a group of the formula —C(═O)—O-alkyl-O—. In embodiments, L¹¹ comprises a group of the formula —C(═O)-alkyl-(OCH₂CH₂)_{q}O—, wherein q is an integer from 1 to 5.

In embodiments, the alkyne is an alkyne of the formula:

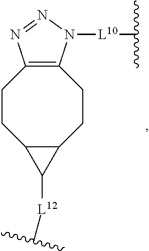

wherein L¹⁰ and L¹² are each, independently, linkers. In embodiments, L¹² comprises a group of the formula: —C(═O)-alkyl-(OCH₂CH₂)_{q}O—, wherein q is an integer from 1 to 5. In embodiments, L¹² comprises a group of the formula: —C(═O)alkyl-NH—C(═O)-alkyl-(OCH₂CH₂)_{q}O—, wherein q is an integer from 1 to 5. L¹² comprises a group of the formula:
—C(═O)alkyl-NH—C(═O)-alkyl-(OCH₂CH₂)_{q}O-alkyl-NH—C(═O)O-alkyl, wherein q is an integer from 1 to 5.

Synthetic schemes are provided in FIGS. 3A-3D, FIG. 4.

CPPs can contain reactive groups (e.g., TFP) for conjugation to a TO moiety.

The CPPs can have free carboxylic acid groups that may be utilized for conjugation to a TO moiety.

The CPPs can contain azide functional groups on the linker that may be utilized to facilitate addition of a TO moiety. In embodiments, the CPP may also include an EP conjugated to the side chain of an amino acid in the CPP.

The structure below is a 3′ cyclooctyne modified PMO used for a click reaction with CPPs and/or NLS containing an azide:

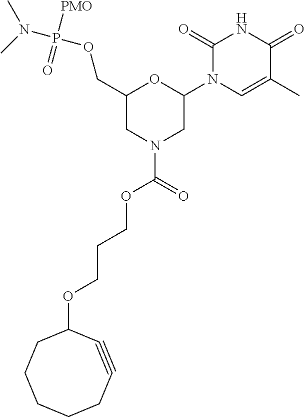

An example scheme of conjugation of a CPP and linker to the 3′ end of a TO moiety via an amide bond is shown below.

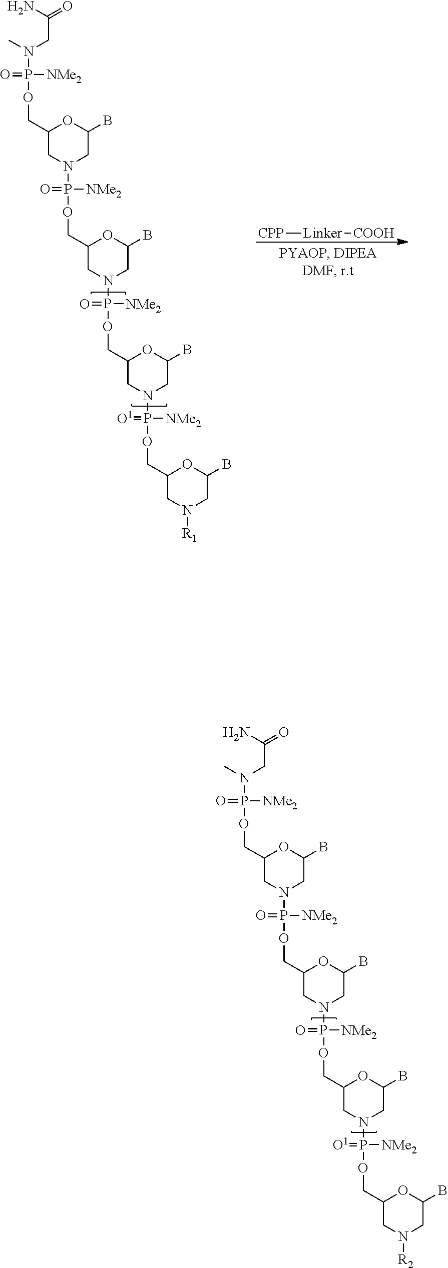

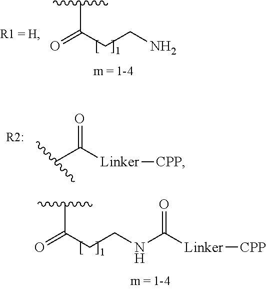

An example scheme of conjugation of a CPP and linker to a 3′-cyclooctyne modified PMO via strain-promoted azide-alkyne cycloaddition is shown below:

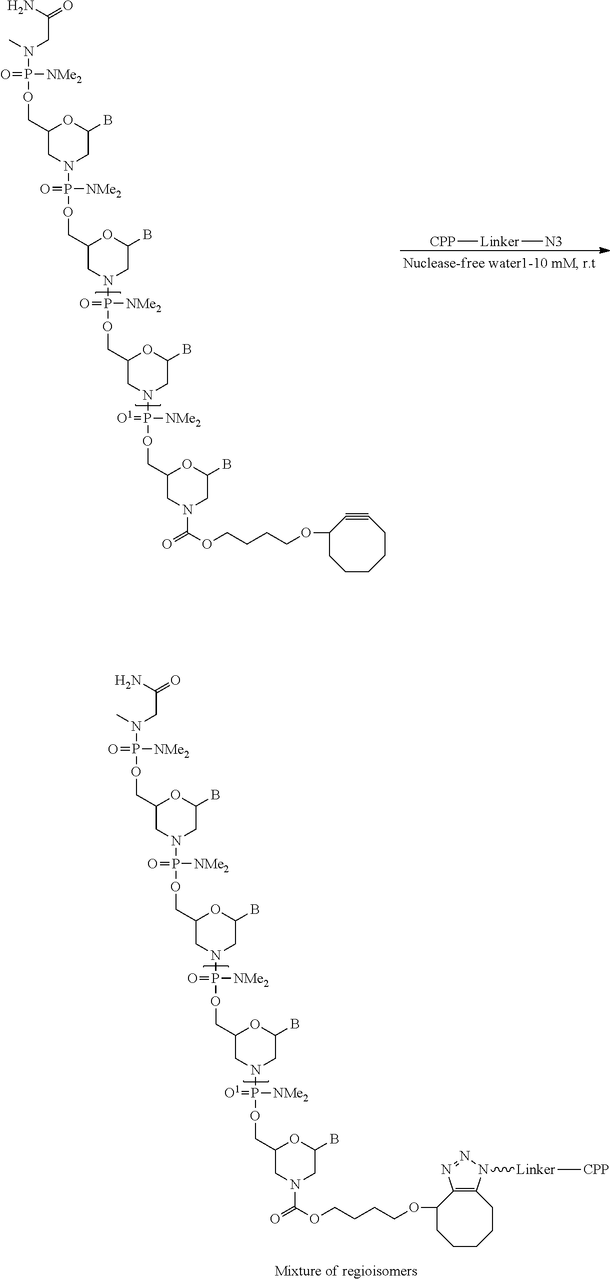

An example of the conjugation chemistry used to connect a TO moiety and CPP with an additional linker containing a polyethylene glycol moiety is shown below:

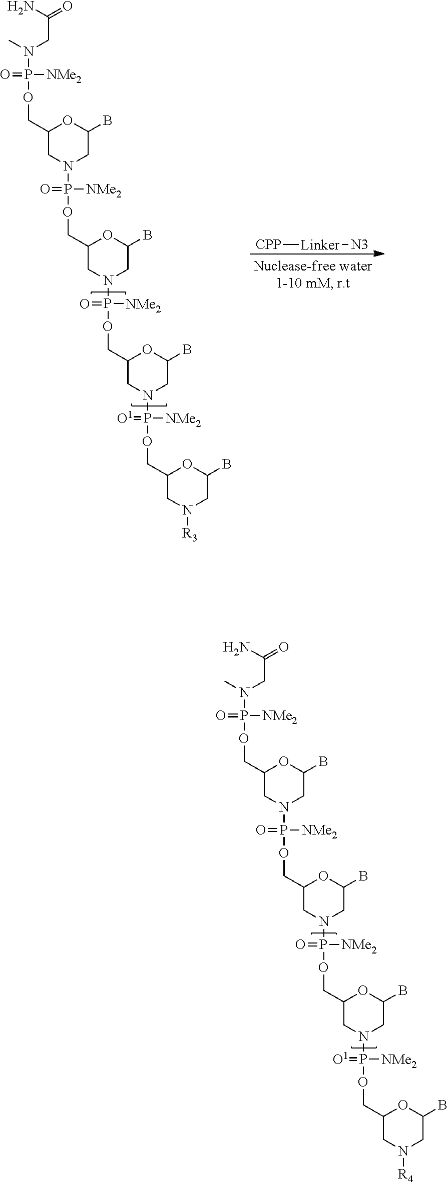

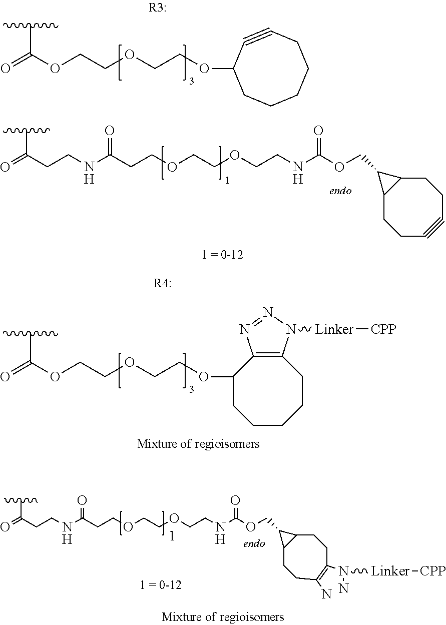

An example of conjugation of a CPP-linker to a 5′-cyclooctyne modified PMO via strain-promoted azide-alkyne cycloaddition (click chemistry) is shown below:

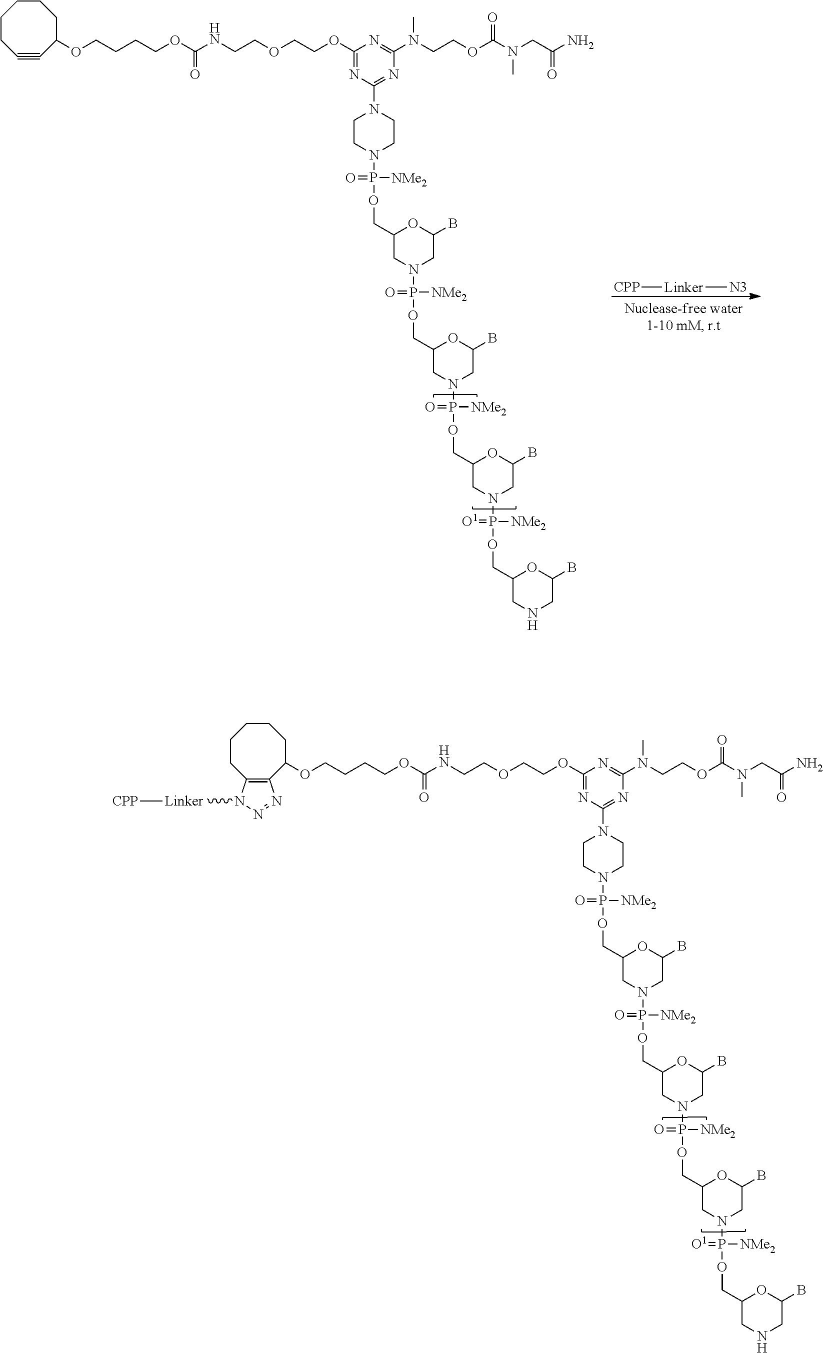

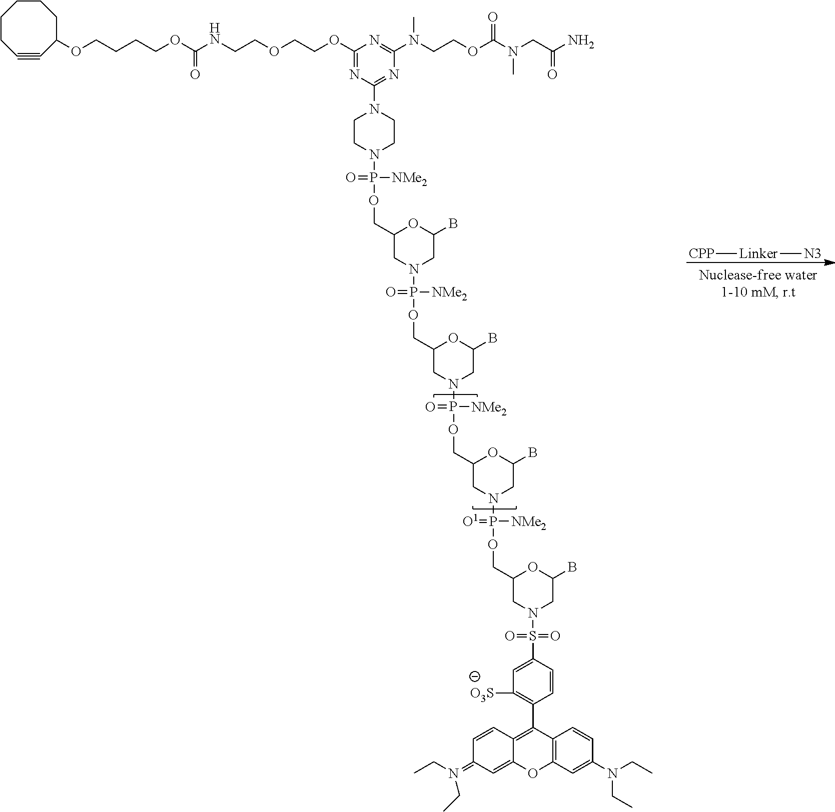

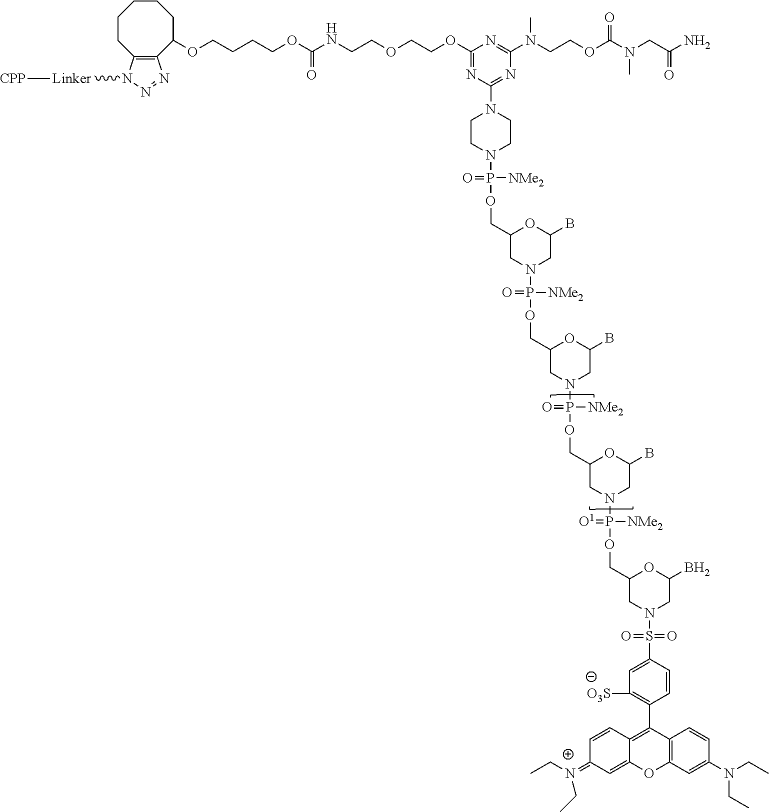

Methods of synthesizing oligomeric TO moieties compounds are known in the art. The present disclosure is not limited by the method of synthesizing the TO moiety. In embodiments, provided herein are compounds having reactive phosphorus groups useful for forming internucleoside linkages including for example phosphodiester and phosphorothioate internucleoside linkages. Methods of preparation and/or purification of precursors of DNA or RNA or TOS are not a limitation of the compositions or methods provided herein. Methods for synthesis and purification of DNA, RNA, and the TO moieties are well known to those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

TO moieties provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The invention is not limited by the method of antisense oligonucleotide synthesis.

Methods of oligonucleotide purification and analysis are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

In embodiments, the solid phase peptide synthesis method includes attaching the α-C-terminal amino acid to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, for example, piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, for example, piperidine. Each protected amino acid is then introduced in about 3-fold molar excess, and the coupling can be carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent that includes thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing. Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. In embodiments, the compounds and compositions containing them are administered intravenously. Administration of the disclosed compounds or compositions can be a single administration, or at continuous or distinct intervals as can be readily determined by a person skilled in the art.

The compounds disclosed herein, and compositions that include them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously include between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients mentioned above, the compositions disclosed herein can include other agents conventional in the art having regard to the type of formulation in question.

Compounds disclosed herein, and compositions that include them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell includes attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. In embodiments, compounds and compositions disclosed herein, including pharmaceutically acceptable salts or prodrugs thereof, are administered intravenously. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders that include the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium that includes, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the methods of preparation include vacuum drying and the freeze-drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a patient's skin to reduce the size (and can include complete removal) of malignant or benign growths, or to treat an infection site. Compounds and agents disclosed herein can be applied directly to the growth or infection site. In embodiments, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to improve the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that include a compound disclosed herein in combination with a pharmaceutically acceptable carrier. In embodiments, the pharmaceutical composition is adapted for oral, topical or parenteral administration. The dose administered to a patient, for example, a human, should be sufficient to achieve a therapeutic response in the patient over a reasonable time frame, without lethal toxicity, and without causing more than an acceptable level of side effects or morbidity. One skilled in the art will recognize that dosage will depend upon a variety of factors including the condition (health) of the patient, the body weight of the patient, kind of concurrent treatment, if any, frequency of treatment, therapeutic ratio, as well as the severity and stage of the pathological condition.

In embodiments, a compound of the disclosure is administered to a patient at a dose of between about 0.01 mg/kg and about 1000 mg/kg, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 210 mg/kg, about 220 mg/kg, about 230 mg/kg, about 240 mg/kg, about 250 mg/kg, about 260 mg/kg, about 270 mg/kg, about 280 mg/kg, about 290 mg/kg, about 300 mg/kg, about 310 mg/kg, about 320 mg/kg, about 330 mg/kg, about 340 mg/kg, about 350 mg/kg, about 360 mg/kg, about 370 mg/kg, about 380 mg/kg, about 390 mg/kg, about 400 mg/kg, about 410 mg/kg, about 420 mg/kg, about 430 mg/kg, about 440 mg/kg, about 450 mg/kg, about 460 mg/kg, about 470 mg/kg, about 480 mg/kg, about 490 mg/kg, about 500 mg/kg, about 510 mg/kg, about 520 mg/kg, about 530 mg/kg, about 540 mg/kg, about 550 mg/kg, about 560 mg/kg, about 570 mg/kg, about 580 mg/kg, about 590 mg/kg, about 600 mg/kg, about 610 mg/kg, about 620 mg/kg, about 630 mg/kg, about 640 mg/kg, about 650 mg/kg, about 660 mg/kg, about 670 mg/kg, about 680 mg/kg, about 690 mg/kg, about 700 mg/kg, about 710 mg/kg, about 720 mg/kg, about 730 mg/kg, about 740 mg/kg, about 750 mg/kg, about 760 mg/kg, about 770 mg/kg, about 780 mg/kg, about 790 mg/kg, about 800 mg/kg, about 810 mg/kg, about 820 mg/kg, about 830 mg/kg, about 840 mg/kg, about 850 mg/kg, about 860 mg/kg, about 870 mg/kg, about 880 mg/kg, about 890 mg/kg, about 900 mg/kg, about 910 mg/kg, about 920 mg/kg, about 930 mg/kg, about 940 mg/kg, about 950 mg/kg, about 960 mg/kg, about 970 mg/kg, about 980 mg/kg, about 990 mg/kg, or about 1000 mg/kg, including all values and ranges therein and in between.

Also disclosed are kits that include a compound disclosed herein in one or more containers. The disclosed kits can optionally include pharmaceutically acceptable carriers and/or diluents. In embodiments, a kit includes one or more other components, adjuncts, or adjuvants as described herein. In another embodiment, a kit includes one or more anti-cancer agents, such as those agents described herein. In embodiments, a kit includes instructions or packaging materials that describe how to administer a compound or composition of the kit. Containers of the kit can be of any suitable material, e.g., glass, plastic, metal, etc., and of any suitable size, shape, or configuration. In embodiments, a compound and/or agent disclosed herein is provided in the kit as a solid, such as a tablet, pill, or powder form. In another embodiment, a compound and/or agent disclosed herein is provided in the kit as a liquid or solution. In embodiments, the kit includes an ampoule or syringe containing a compound and/or agent disclosed herein in liquid or solution form.

A number of embodiments have been described herein. Nevertheless, it will be understood that various modifications may be made without departing from the spirit and scope of the invention. Accordingly, other embodiments are within the scope of the following claims.

### EXAMPLES

### Example 1. Synthesis of Compounds

PMO refers to a phosphorodiamidate morpholino oligomer. PMOs described herein were synthesized as described in WO 2021/127650 A1, entitled COMPOSITIONS FOR DELIVERY OF ANTISENSE COMPOUNDS, which application is hereby incorporated herein by reference in its entirety.

PMO1-EEV1 was synthesized as shown in FIG. 5. PMO1 is a PMO having the following sequence: 5′-GCT ATT ACC TTA ACC CAG-3′ (with 5′-OH and 3′ secondary amine morpholino; SEQ ID NO:2). EEV refers to an endosomal escape vehicle comprising a cCPP and an EP.

TFA-lysine protected EEV1 was reacted with PMO1 with the following sequence (5′-GCT ATT ACC TTA ACC CAG-3′) and subsequently deprotected to furnish the desired conjugate. Briefly, PMO1 (1.0 equiv), EEV1 (1.8 equiv), and DIPEA (6.0 equiv) were dissolved in DMSO (10 mM). HATU (2.0 equiv) in DMSO (300 mM) was then added at room temperature, causing the reaction to turn yellow. The reaction was incubated for 2 hours at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. Upon completion, in situ deprotection of TFA-protected lysines was initiated by adding a solution of 320 mM NaOH (aq) (40 equiv). The reaction was incubated for 1 hr and monitored by LCMS (Q-TOF), using the analysis method noted above. The crude mixture was loaded directly onto a C18 reverse-phase column. The crude product was then purified using an appropriate gradient using water with 0.1% FA and acetonitrile as solvents and a flow rate of 20 mL/min. Fractions containing the desired product were pooled, and the pH of the solution was adjusted to 7 using 0.5 M NaOH. The solution was frozen and lyophilized, affording white powder. The solid was dissolved in water. The material was then run through a 3-kD MW-cutoff amicon tube repeatedly (centrifuged at 3000 rpm for 20-40 min). This process was performed three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration. The purity and identity of each formulation was assessed by LCMS (QTOF); 99% c purity by RP-FA; 82% purity by CEX; MW calculated for C₃₃₀H₁₃₀N₁₃₀O₁₀₅P₁₇, 8530.27, found 8531.

The structure of the resulting compound (PMO1-EEV1) is shown in FIG. 6.

GalNAc-PMO2 was synthesized as shown in FIG. 7. GalNAc-N₃ was reacted with PMO2 with the following sequence (Cyclooctyne-5′-GCT ATT ACC TTA ACC CAG-3′). A stock solution of GalNAc-N₃ (100 mg/mL) was combined with a solution of PMO2 in H₂O and mixed thoroughly. The reaction was incubated for ˜12 hr at room temperature. The reaction was monitored by LCMS (Q-TOF), using BEH C18 column (130 Å, 1.7 μm, 2.1 mm-50 mm), buffer A: water (0.1% FA), buffer B: acetonitrile (0.1% FA), flow rate: 0.4 mL/min, starting with 2% buffer B and ramping up to 98% over 3.4 min. The crude mixture was loaded directly onto a C18 reverse-phase column. The crude product was then purified using an appropriate gradient using water with 0.1% FA and acetonitrile as solvents and a flow rate of 20 mL/min. Fractions containing the desired product were pooled, and the pH of the solution was adjusted to 7 using 0.5 M NaOH. The solution was frozen and lyophilized, affording white powder. The solid was dissolved in water. The material was then run through a 3-kD MW-cutoff amicon tube repeatedly (centrifuged at 3000 rpm for 20-40 min). This process was performed three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration. The purity and identity of each formulation was assessed by LCMS (QTOF); 99% purity by RP-FA; Calculated MW for C₄₃₃H₇₁₅N₁₅₃O₁₄₆P₁₈, 8288.72, found 8289.42.

The structure of the resulting compound (GalNAc-PMO2) is shown in FIG. 8.

GalNAc-PMO2-EEV1 was synthesized as shown in FIG. 9. GalNAc-PMO2-EEV1 was synthesized according to the following procedure. TFA-lysine protected EEV1 was reacted with PMO2 with the following sequence (Cyclooctyne-5′-GCT ATT ACC TTA ACC CAG-3′) and subsequently deprotected to furnish the desired EEV-PMO conjugate. Briefly, PMO2 (1.0 equiv), EEV1 (1.8 equiv), and DIPEA (6.0 equiv) were dissolved in DMSO (10 mM). HATU (2.0 equiv) in DMSO (300 mM) was then added at room temperature, causing the reaction to turn yellow. The reaction was incubated for 2 hours at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate; 0.4 mL/min, starting with 2% buffer B and ramping up to 98% over 3.4 min. Upon completion, in situ deprotection of TFA-protected lysines was initiated by adding a solution of 320 mM NaOH (aq) (40 equiv). The reaction was incubated for 1 hr and monitored by LCMS (Q-TOF), using the analysis method noted above. The crude mixture was loaded directly onto a C18 reverse-phase column. The crude product was then purified using an appropriate gradient using water with 0.1% HCl and acetonitrile as solvents and a flow rate of 20 mL/min. Fractions containing the desired product were pooled, neutralized with 0.5 M NaOH(aq), and lyophilized.

GalNAc-N₃ was then reacted with PMO2-EEV1. A stock solution of GalNAc-N₃ (100 mg/mL) was combined with a solution of PMO2-EEV1 in H₂O and mixed thoroughly. The reaction was incubated for ˜12 hr at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. The crude mixture was loaded directly onto a C18 reverse-phase column. The crude product was then purified using an appropriate gradient using water with 0.1% FA and acetonitrile as solvents and a flow rate of 20 mL/min. Fractions containing the desired product were pooled, and the pH of the solution was adjusted to 7 using 0.5 M NaOH. The solution was frozen and lyophilized, affording white powder. The solid was dissolved in water. The material was then run through a 3-kD MW-cutoff amicon tube repeatedly (centrifuged at 3000 rpm for 20-40 min). This process was performed three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration. Calculated MW for C₄₃₃H₇₁₅N₁₅₃O₁₄₆P₁₈, 10957.94, found 10958.88.

The structure of the resulting compound (GalNAc-PMO2-EEV1) is shown in FIG. 10.

PMO3 (PMO1-Lys(BCN) was synthesized as shown in FIG. 11A. Fmoc-Lys(BCN)—OH was reacted with compound PMO1 with the following sequence (5′-GCT ATT ACC TTA ACC CAG-3′) and subsequently deprotected to furnish the desired PMO conjugate. Briefly, PMO1 (1.0 equiv), Fmoc-Lys(BCN)—OH (2.5 equiv), and DIPEA (6.0 equiv) were dissolved in DMSO (10 mM). HATU (2.0 equiv) in DMSO (300 mM) was then added at room temperature, causing the reaction to turn yellow. The reaction was incubated for 2 hours at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. Upon completion, in situ deprotection of Fmoc was initiated by adding a solution of 1% DBU(aq) (20-fold dilution). White precipitate slowly formed in the reaction. The reaction was incubated for 2 hr and monitored by LCMS (Q-TOF), using the analysis method noted above. The crude mixture was filtered through a 0.2 um nylon syringe filter and loaded onto a C18 reverse-phase column. The crude product was then purified using an appropriate gradient using water with 0.1% FA and acetonitrile as solvents and a flow rate of 20 mL/min. Fractions containing the desired product were pooled, neutralized with 0.5 M NaOH(aq), and lyophilized. Calculated MW for C₂₂₅H₃₄₉N₁₀₀O₇₄P₁₇, 6165.45, found 6166.

The structure of the resulting compound PMO3 is shown in FIG. 11B

PMO3-GalNAc-NHAc was synthesized as shown in FIG. 12. AcOH was reacted with compound PMO1-Lys(BCN) with the following sequence (5′-GCT ATT ACC TTA ACC CAG-3′) to furnish the desired PMO conjugate. Briefly, PMO3 (1.0 equiv), AcOH (1.8 equiv), and DIPEA (6.0 equiv) were dissolved in DMSO (10 mM). HATU (2.0 equiv) in DMSO (300 mM) was then added at room temperature, causing the reaction to turn yellow. The reaction was incubated for 2 hours at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. The crude mixture was loaded directly onto a C18 reverse-phase column. The crude product was then purified using an appropriate gradient using water with 0.1% FA and acetonitrile as solvents and a flow rate of 20 mL/min. Fractions containing the desired product were pooled, neutralized with 0.5 M NaOH(aq), and lyophilized.

GalNAc-N₃ was then reacted with PMO1-Lys(BCN)—NHAc. A stock solution of GalNAc-N₃ (100 mg/mL) was combined with a solution of PMO1-Lys(BCN)—NHAc in 50% CH₃CN(aq) and mixed thoroughly. The reaction was incubated for ˜12 hr at room temperature. The reaction was monitored by LCMS (Q-TOF), using BEH C₁₈ column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. The crude mixture was loaded directly onto a C18 reverse-phase column. The crude product was then purified using an appropriate gradient using water with 0.1% FA and acetonitrile as solvents and a flow rate of 20 mL/min. Fractions containing the desired product were pooled, and the pH of the solution was adjusted to 7 using 0.5 M NaOH. The solid was dissolved in water. The material was then run through a 3-kD MW-cutoff amicon tube repeatedly (centrifuged at 3000 rpm for 20-40 min). This process was performed three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration. Calculated MW for C₂₉₇H₄₇₆N₁₁₃O₁₀₆P₁₇, 7852.31, found 7853.

The structure of the resulting compound PMO3-GalNAc-NHAc is shown in FIG. 13.

PMO3-GalNAc-EEV1 was synthesized as shown in FIG. 14. EEV1 was reacted with compound PMO1-Lys(BCN) with the following sequence (5′-GCT ATT ACC TTA ACC CAG-3′) to furnish the desired PMO conjugate. Briefly, PMO1-Lys(BCN) (1.0 equiv), EEV1 (1.8 equiv), and DIPEA (6.0 equiv) were dissolved in DMSO (10 mM). HATU (2.0 equiv) in DMSO (300 mM) was then added at room temperature, causing the reaction to turn yellow. The reaction was incubated for 2 hours at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. Upon completion, in situ deprotection of TFA-protected lysines was initiated by adding a solution of 320 mM NaOH (aq) (40 equiv). The reaction was incubated for 1 hr and monitored by LCMS (Q-TOF), using the analysis method noted above. The crude mixture was loaded directly onto a C18 reverse-phase column The crude product was then purified using an appropriate gradient using water with 0.05% HCl and acetonitrile as solvents and a flow rate of 20 mL/min. Fractions containing the desired product were pooled, neutralized with 0.5 M NaOH(aq), and lyophilized.

GalNAc-N₃ was then reacted with PMO1-Lys(BCN)-EEV1. A stock solution of GalNAc-N₃ (100 mg/mL) was combined with a solution of PMO3-EEV1 in 50% CH₃CN(aq) and mixed thoroughly. The reaction was incubated for ˜12 hr at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. The crude mixture was loaded directly onto a C18 reverse-phase column. The crude product was then purified using an appropriate gradient using water with 0.1% FA and acetonitrile as solvents and a flow rate of 20 mL/min. Fractions containing the desired product were pooled, and the pH of the solution was adjusted to 7 using 0.5 M NaOH. The solution was frozen and lyophilized, affording white powder. The solid was dissolved in water. The material was then run through a 3-kD MW-cutoff amicon tube repeatedly (centrifuged at 3000 rpm for 20-40 min). This process was performed three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration. Calculated MW for C₄₁₇H₆₈₄N₁₄₅O₁₃₉P₁₇, 10479.49, found 10480.

The structure of the resulting compound PMO3-GalNAc-1120 is shown in FIG. 15.

### Example 2. Dose Dependent Pharmacodynamic Study

Experiments were performed to determine if GalNAc conjugation to PMO or PMO-EEV could improve liver efficacy of PMO or PMO-EEV in EGFP-654 mice (Sazani et al., Nat Biotechnol. 2002 December; 20(12):1228-33. doi: 10.1038/nbt759.) in a single dosing schedule. Mice (EGFP-654, n=3/group; one female and two male) were injected intravenously (IV) with saline as control and 20 mpk of PMO1 and PMO1-EEV1; 0.2, 2, and 20 mpk (all normalized based on PMO dose) GalNAc-PMO2 and GalNAc-PMO2-EEV1 via both intravenous (TV) and subcutaneous (SC) route of administration. Both the PMO1 and the PMO2 have the same sequence and can be conjugated to EEV at the 3′ end, however, they have different functional handles at 5′ end. The PMO2 with sequence cyclooctyne-5′-GCT ATT ACC TTA ACC CAG-3′ has a cyclooctyne linker that can be conjugated to GalNAc while the PMO1 does not have a linker that allows for conjugation to GalNAc. Thus, PMO1 is used as a control to when exploring the GalNAc liver targeting effects of the PMO2 GalNAc conjugates. An overview of the study design is illustrated in FIG. 16.

No acute toxicity observed in animals after dosing all concentrations and they maintained their normal state and were sacrificed seven days post injections. Liver, kidney, diaphragm and heart were collected. Only liver was analyzed for splice correction and restoration of eGFP protein by RT-PCR and ELISA, respectively. The drug exposure in the tissues were analyzed by LC-MS.

eGFP-654 transgenic mouse. The eGFP-654 transgenic mouse line was generated previously for evaluating splice switching oligonucleotides (Sazani et al. 2002 Nat Biotech; JAX stock #027617). The eGFP-654 transgene was cloned under a hybrid promoter with a cytomegalovirus early enhancer element, chicken beta-actin, and rabbit beta-globin, yielding widespread expression throughout the body. A mutated intron 2 (at nucleotide 654) from the human beta-globin gene is introduced to interrupt the eGFP coding sequence (at nucleotide 105). This human beta-globin mutation at nucleotide 654 activates an aberrant splice site, leading to retention of the intron in the spliced mRNA, preventing proper eGFP translation. Blocking this aberrant splice site has been shown to restore proper splicing, allowing eGFP translation (Sazani et al. 2002 Nat Biotech). The mice used in the experiments are homozygous for the eGFP-654 transgene.

eGFP relative protein analysis by capillary electrophoresis (ELISA). Tissues were pulverized and a fraction of the pulverized powder was removed and placed in an Omni International homogenization tube with 1.4 mm ceramic beads (Omni International, #19-627). Lysis buffer containing RIPA with 1×HALT protease inhibitors (Thermo Fisher 78430) was added to each sample at 4 degrees C. Samples were homogenized for 30 seconds at 6 m/s using an Omni International bead mill homogenizer. Homogenized samples were centrifuged at 21,000×g for 3 minutes at 4 degrees C. and the supernatant was transferred to another tube. Samples were centrifuged at 21,000×g for 10 minutes at 4 degrees C. and the supernatant was collected. Concentration was measured using a bicinchoninic acid (BCA) assay according to the manufacturer's protocol (Pierce BCA Protein Assay Kit 23227), diluting with the lysis buffer as needed. Sample solutions were diluted to normalize protein concentration. Sample eGFP levels were analyzed using enzyme-linked immunosorbent assay (ELISA) analysis with Abcam GFP ELISA kit (ab171581) according to the manufacturer's protocol. Sample eGFP levels were interpolated using the standard curve with the ELISA kit and reported as mass of eGFP detected (as measured by ELISA) per mass of protein content in the tissue lysate (as measured by BCA assay). the pg/mL of protein detected in each sample divided by the total protein concentration of lysates in ug/ml.

Detection of splicing correction by RT-PCR The detection of splicing correction process was measured by RT-PCR 20-50 mg pulverized tissue sample was transferred to a soft tissue homogenization tube (SKU 19-627, Omni International) followed by addition of 1 ml Qiagen RLT lysis buffer (Catalog 79216, QIAGEN). The samples were homogenized on an Omni Bead Ruptor Elite (SKU 19-040E, Omni International) followed by centrifugation of the homogenate at 20,000×g for 10 min at 4° C. Supernatants were collected for RNA extraction using QIAGEN RNAeasy kits (Catalog 74004, QIAGEN) according to manufacturer's protocol. RT-PCR was performed with 200 ng of the extracted total RNA using a QIAGEN OneStep RT-PCR Kit (Catalog 210212, QIAGEN). A reaction solution was prepared in accordance with the protocol according to the kit using forward primer 5′-CGTAAACGGCCACAAGTTCAGCG-3′ (SEQ ID NO:4) and reverse primer 5′-GTGGTGCAGATGAACTTCAGGGTC-3′ (SEQ ID NO:5). 2 μl RT-PCR product was loaded for each tissue sample on an 2% E-gel (G401002, Thermo Fisher Scientific) and run on a E-Gel Power Snap Electrophoresis System (G8300, Thermo Fisher Scientific) for 12 min. The RT-PCR readout of tissues without splicing correction resulted in a 160 bp gene fragment and a new 87 bp gene fragment showed up after splicing correction. The intensities of exon-skipped and full-length bands were analyzed using ImageJ. The degree (percentage) of splicing correction detected by RT-PCR was calculated using the following equation: % correction=(intensity of 87 bp fragment band)/(intensity of 87 bp fragment band+intensity of 160 bp fragment band).

Bioanalytical Sample Analysis. Tissues were thawed, weighed, and homogenized (w/v, 1/5) with RIPA buffer spiked with 1× protease inhibitor cocktail (ThermoFisher Scientific, Ref #1860932). The homogenates were centrifuged at 5000 rpm for 5 minutes at 4° C. The supernatants were precipitated with a mixture of H₂O, acetonitrile and MeOH, and centrifuged at 15000 rpm for 15 minutes at 4° C. The supernatants were transferred to an injection plate for LC-MS/MS analysis using Shimadzu UPLC integrated with Triple Quad Sciex 4500 instrument. The dynamic range of the LC-MS/MS assay was 25 to 50,000 ng/g tissue. The details of the LC-MS/MS method are outlined here. Briefly, the UPLC was operated using Waters Acquity UPLC BEH C4, 300 Å, 1.7 um, 2.1×150 mm, buffer A: H₂O, 0.2% FA, buffer B. 95% acetonitrile in H₂O, 0.2% FA, flow rate (0.3 mL/min) and column temperature at 50° C. The 10 min run started with 2% buffer B and ramping up to 35% for 3.5 min followed by 90% for 1 min, staying at 90% gradient for 2.5 min and finally running at 2% gradient for 2 min. The major metabolites were identified for each compound, and total metabolite concentration was used for semi-quantitation of drug exposure for each compound.

### Dose Dependent Pharmacodynamics

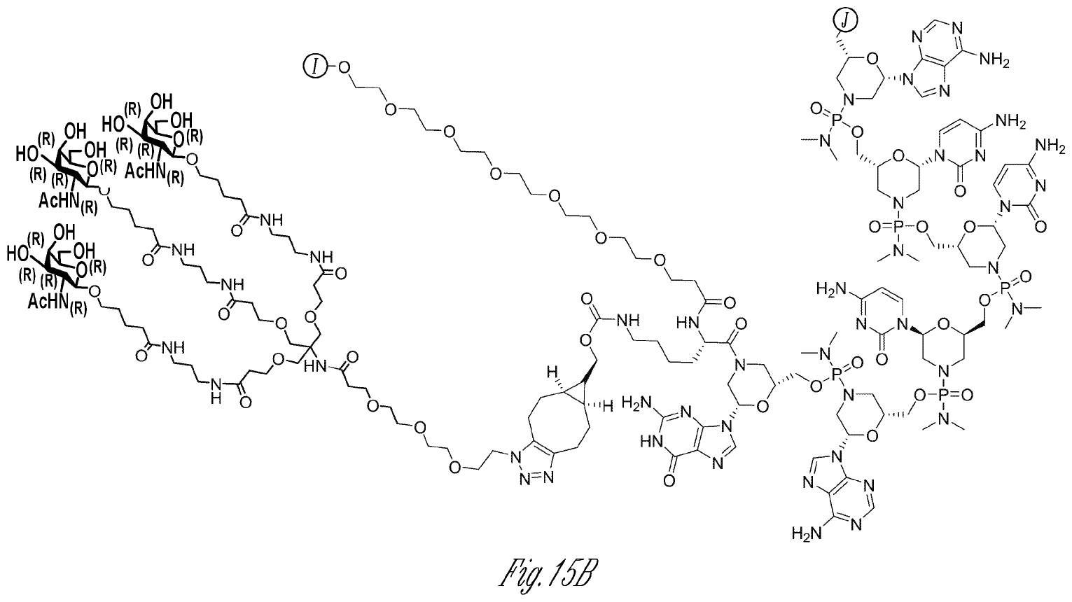

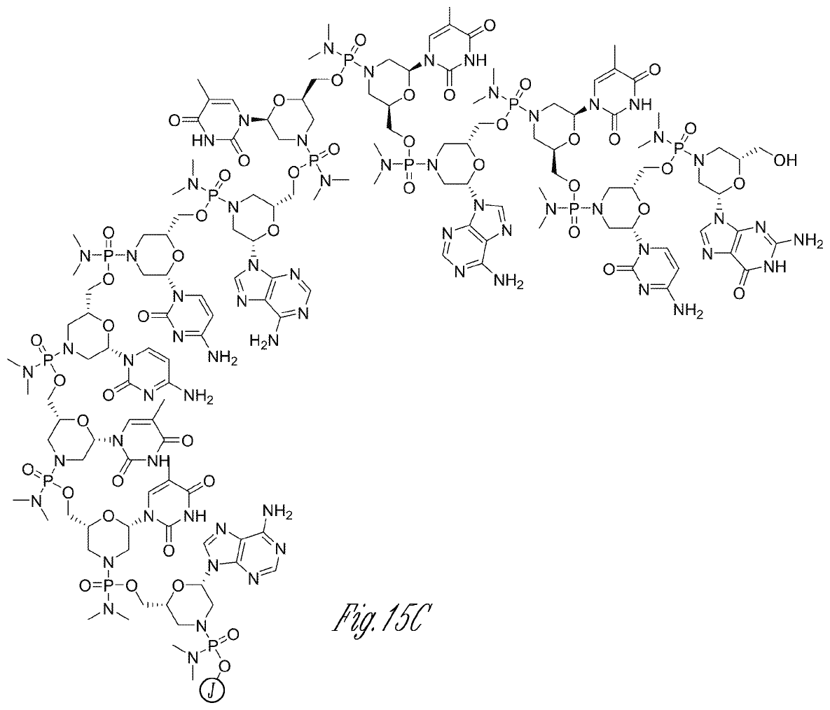

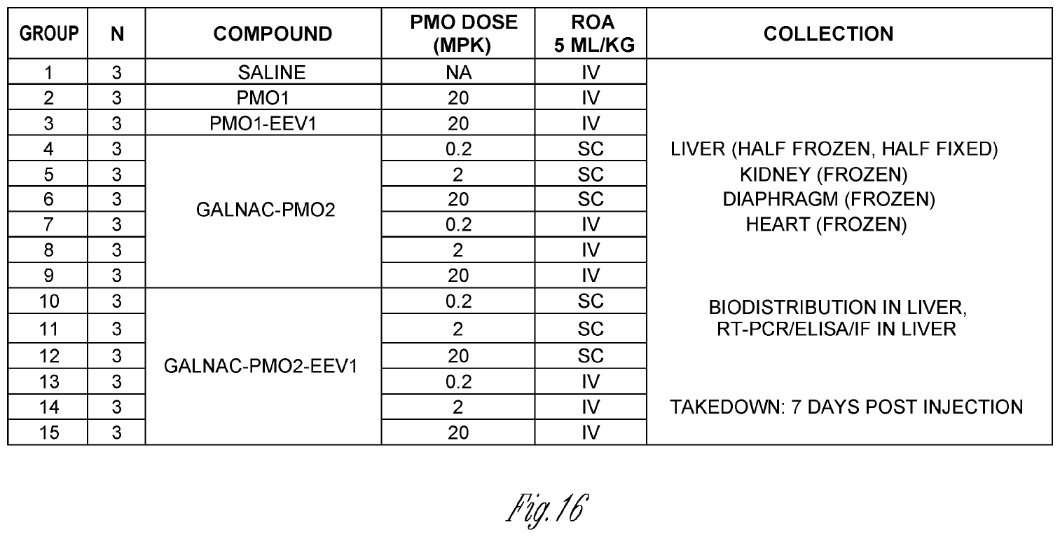

Results and Discussion. Results illustrating exon skipping percentage, eGFP (pg/μg) are shown in FIGS. 17A and 17B. The amount of compound concentration in liver tissue are shown in FIG. 18. Filled circle data for male mice and open circle data for female mice. In FIGS. 17A-17B and 18 “mpk” indicates dose in mg/kg.

The addition of the EEV to GalNAc-PMO2 in construct (GalNAc-PMO2-EEV1) resulted in substantially higher exon skipping (FIG. 17A) and eGFP protein level (FIG. 17B) and drug exposure (FIG. 18) relative to the GalNAc targeted PMO (GalNAc-PMO2) when administered both intravenously and subcutaneously.

EEV conjugation leads to synergistic improvement in efficacy; about (1.3-1.5)-fold higher exon skipping and (1.6-2.6) fold higher eGFP protein relative to GalNAc-PMO2 alone at similar 20 mpk dose via subcutaneous (SC) and intravenous (IV), respectively. GalNAc-PMO2-EEV1 demonstrates efficacy via both IV and SC route of administration GalNAc conjugation enables effective delivery in previously intractable hepatic cells at the doses above 2 mpk.

Similar level drug exposure in liver observed in female & male mice for each compound (FIG. 18); while eGFP protein level is much lower (FIG. 17B) and exon skipping percentage is generally higher for the female mice (FIG. 17A), suggesting the lower aberrant transcript level in female mice.

EEV conjugation to GalNAc-PMO2 enhanced the liver exposure by about 28-fold after SC and 37-fold by IV (FIG. 18); However, the eGFP protein level was only increased by about 1.6-fold after SC and 2.6-fold by IV, suggesting EEV mediated uptake to other cells beyond hepatocytes.

The lower efficacy of 20 mpk of GalNAc-PMO2 in IV route of administration compared to subcutaneous dose might be attributed to preferential uptake of PMO from blood circulation by kidney and subsequent faster clearance. In contrast, subcutaneous injection allows slow release of PMO to circulation and extends the distribution half-life of PMO resulting in more compound retention in liver. The higher biodistribution of SC route in GalNAc-PMO2 in FIG. 18 also supports this hypothesis. Additionally, EEV conjugation to GalNAc-PMO2 showed similar efficacy IV and SC suggesting that it might be contributing to plasma proteins binding by EEV which results in an increase in distribution half-life via IV route of administration.

For both 20 mpk GalNAc-PMO2 and GalNac-PMO2-EEV1, subcutaneous administration showed higher drug exposure compared to IV administration.

PMO1-EEV1 at 20 mpk showed no efficacy while having a comparable drug exposure in liver. This non-productive drug exposure may suggest the accumulation of PMO2-EEV1 in other cells in liver which have less or no eGFP transcript.

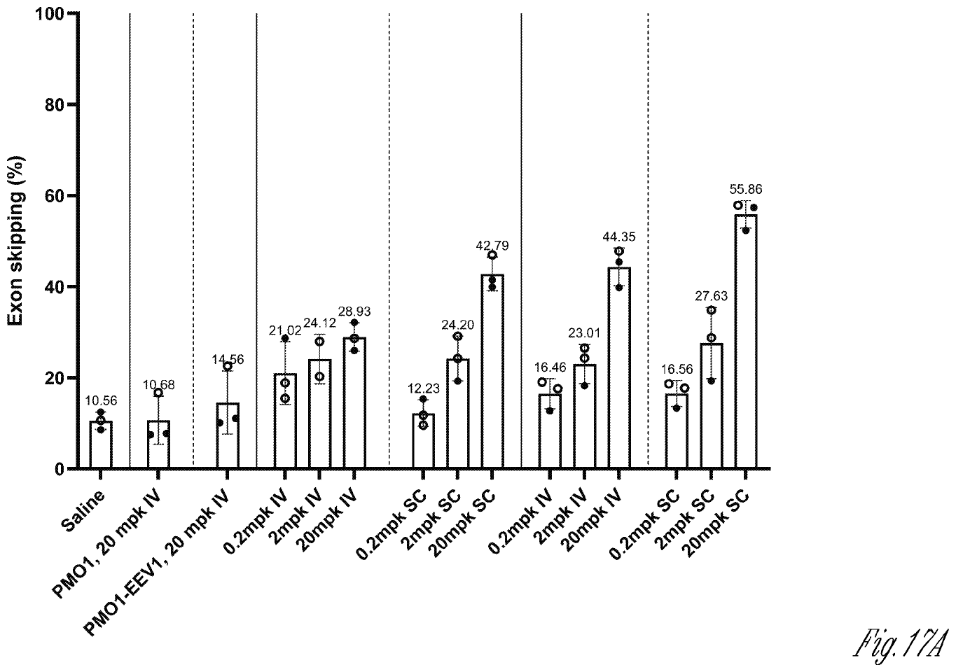

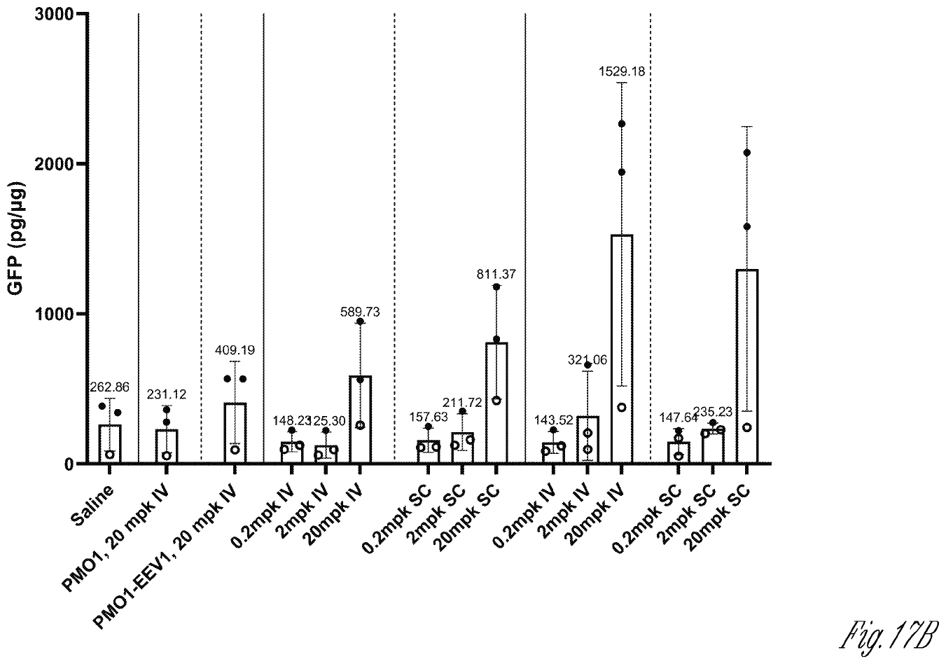

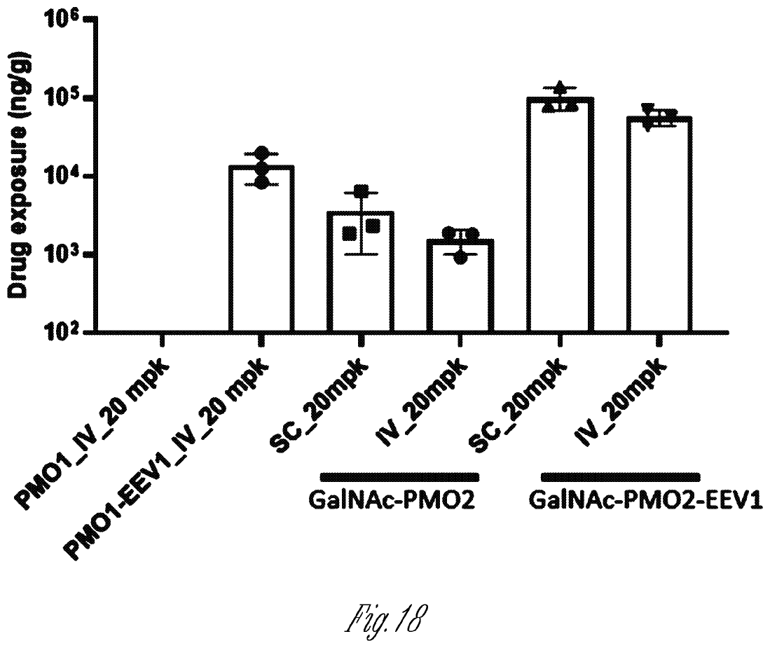

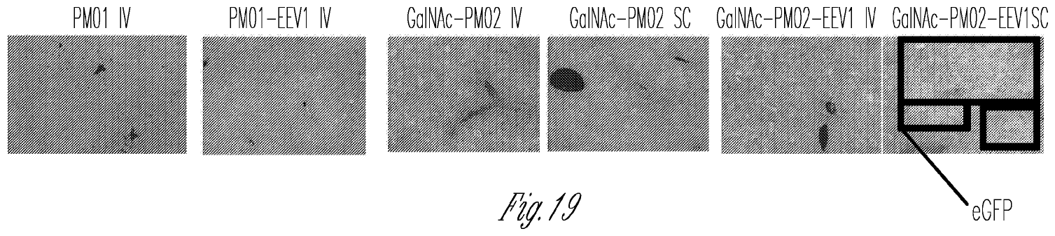

Half of liver tissues were harvested for cryosectioning and examination under a fluorescence microscope. Representative images of liver sections from all groups 7-days post injection are presented in FIG. 19.

GalNAc-PMO2-EEV1 construct with GalNAc and EEV conjugation had synergistic homing and target engagement via SC route which led to an enhanced eGFP fluorescence in liver, and it was consistent with ELISA protein expression data in FIG. 17B.

FIG. 20 shows strong eGFP for GalNAc-PMO2-EEV1 which is colocalized with arginase1 (hepatocyte marker) and is homogeneously distributed in the entire liver tissue. GalNAc-PMO2, showed very moderate eGFP signal but still colocalized with arginase1. No colocalization was observed for PMO1 or PMO1-EEV1.

FIG. 21 shows significant co-localization of eGFP and CD31 stain for PMO1-EEV1 and GalNAc-PMO2-EEV1 suggesting that EEV conjugation enables delivery to endothelial cells.

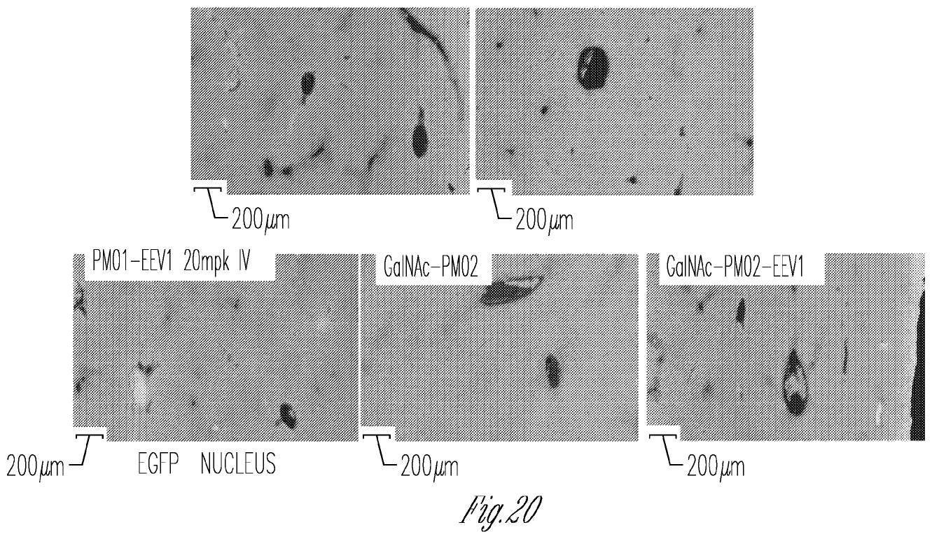

FIG. 22 shows more co-localization of eGFP and F4/80 stain for PMO1 and PMO1-EEV1 suggesting the preferential uptake to macrophages. GalNAc constructs showed minimal co-localization with F4/80, the marker for macrophages.

### Example 3. Duration of Action

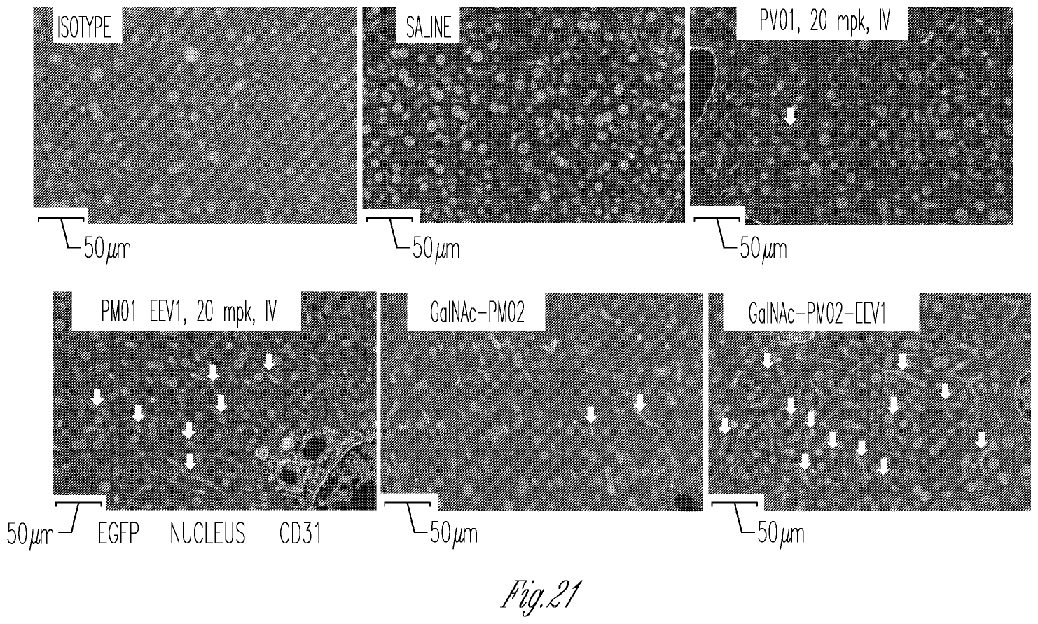

Mice were injected with 20 mpk of GalNAc-PMO2 and GalNAc-PMO2-EEV1 (Normalized based on PMO) via SC route and sacrificed after 2, 4 and 8 weeks. Data from 1 week was obtained from example 1. An overview of the study design is illustrated in FIG. 23.

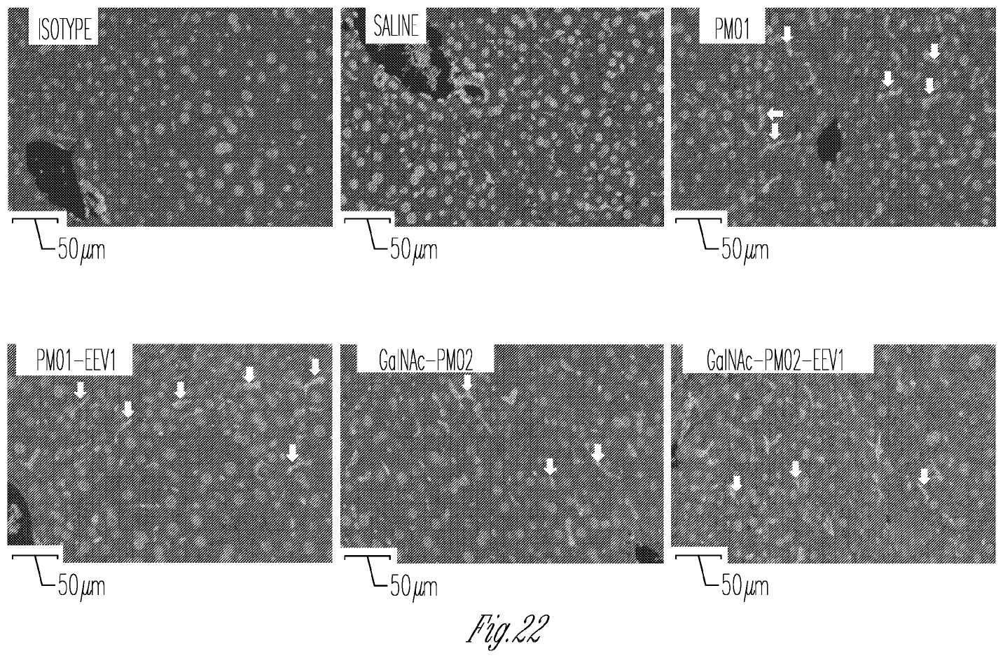

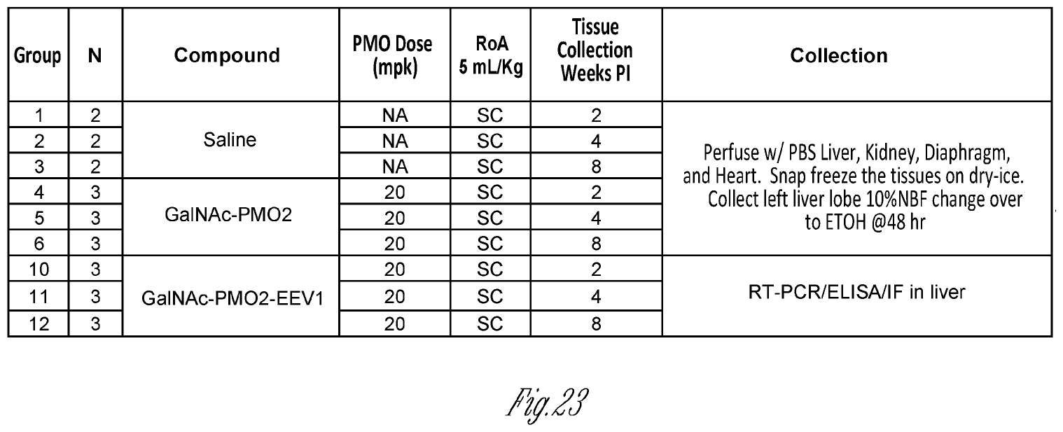

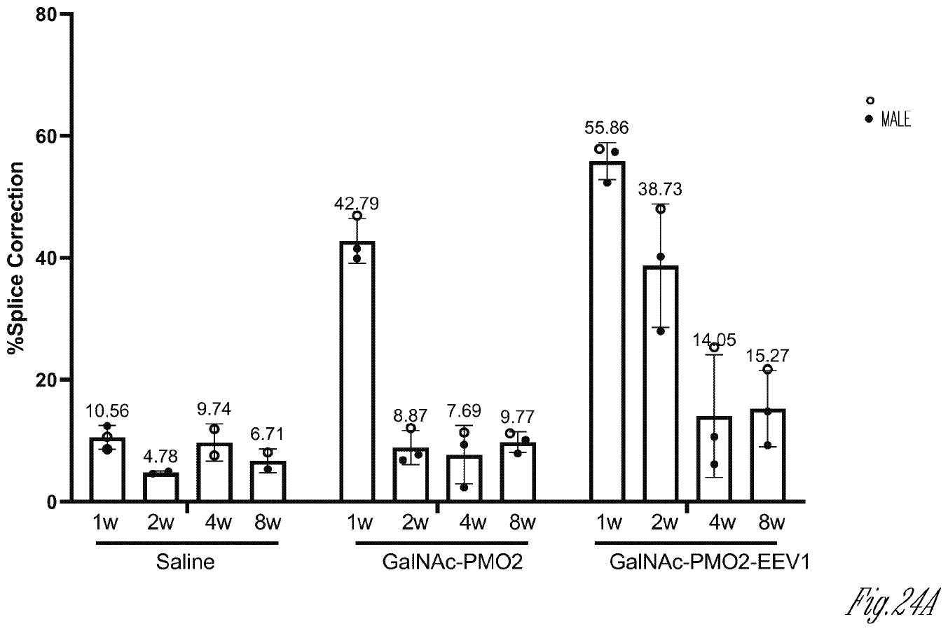

FIGS. 24A-24B, shows percent splice correction and eGFP (pg/μg), respectively, after 1 week, 2 weeks, 4 weeks and 8 weeks. Longer duration of action was obtained with GalNAc-PMO-EEV1 for both splice correction and functional protein level up to 8 weeks. GalNAc-PMO2-EEV1 showed about 4-fold higher efficacy after 4 weeks compared to GalNAc-PMO2. Effect of different EEVs on efficacy of GalNAc-PMO constructs

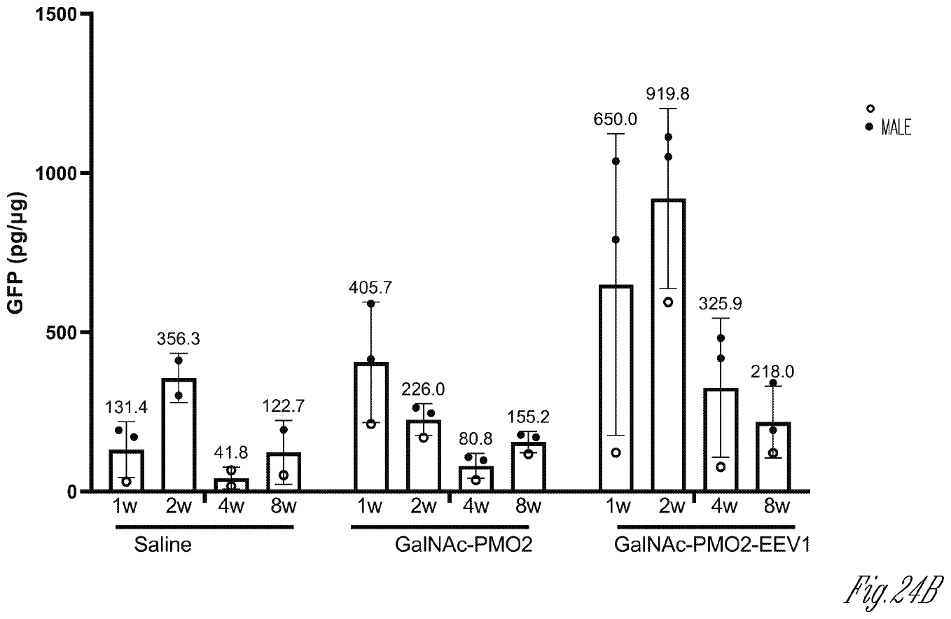

Results and Discussion. Mice (EGFP-654, n=3/group; all male) were injected intravenously (IV) with saline as control and 20 mpk PMO equivalent of different GalNAc-PMO2-EEVs. Each EEV is comprised of different charge and hydrophobicity. Study design was summarized in FIG. 25.

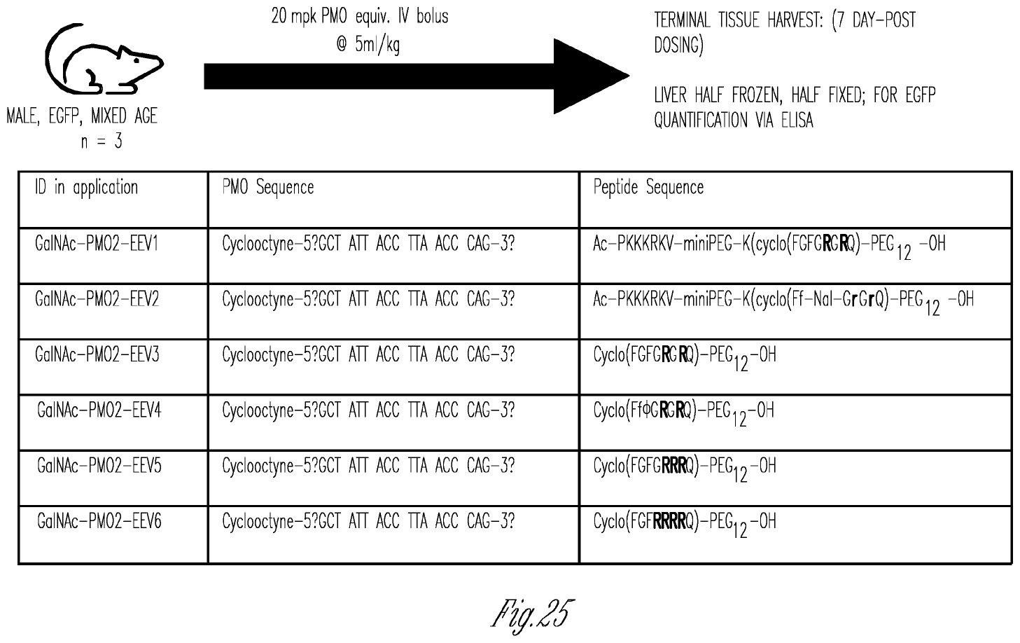

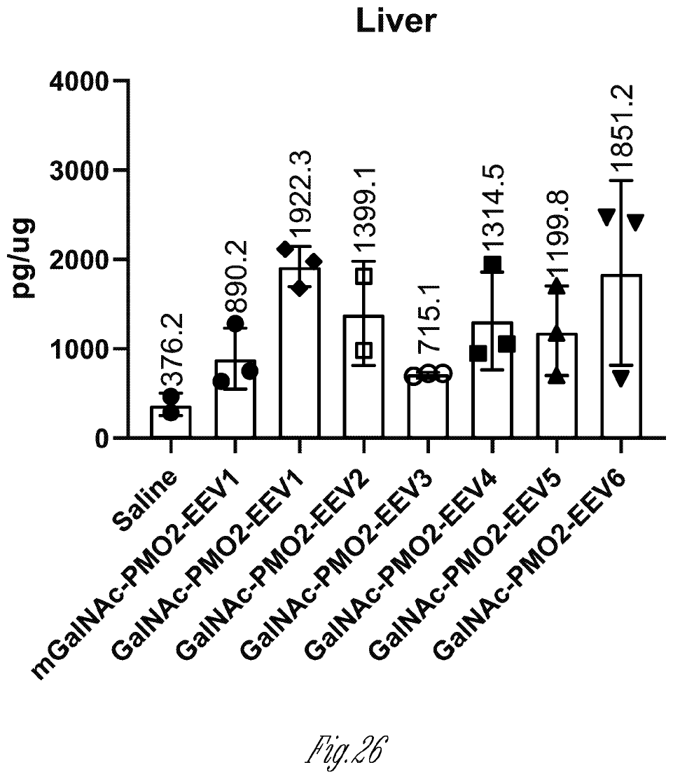

FIG. 26 shows an optimal EEV amino acid composition needed to act synergistically with GalNAc liver targeting. This suggests EEV mediated uptake by other tissues/cell types beyond liver/hepatocytes.

Both EEV1 with sequence (Ac-PKKKRKV-miniPEG-K(cyclo(FGFGRGRQ)-PEG₁₂-OH) and EEV6 with sequence (Cyclo(FGFRRRRQ)-PEG₁₂-OH) conjugations to GalNAc-PMO2 resulted in highest eGFP protein restoration.

Removing the NLS (PKKKRKV) exocyclic moiety led to dramatic decrease in efficacy in EEV conjugated construct with sequence (Cyclo(FGFGRGRQ)-PEG₁₂-OH).

Trimeric GalNAc is much more effective targeting ligand than the monomeric GalNAc (mGalNAc); Consistent with higher binding affinity of the trimeric ligand with the ASGPR receptor.
Effect of site of GalNAc conjugation; 3′ vs 5′

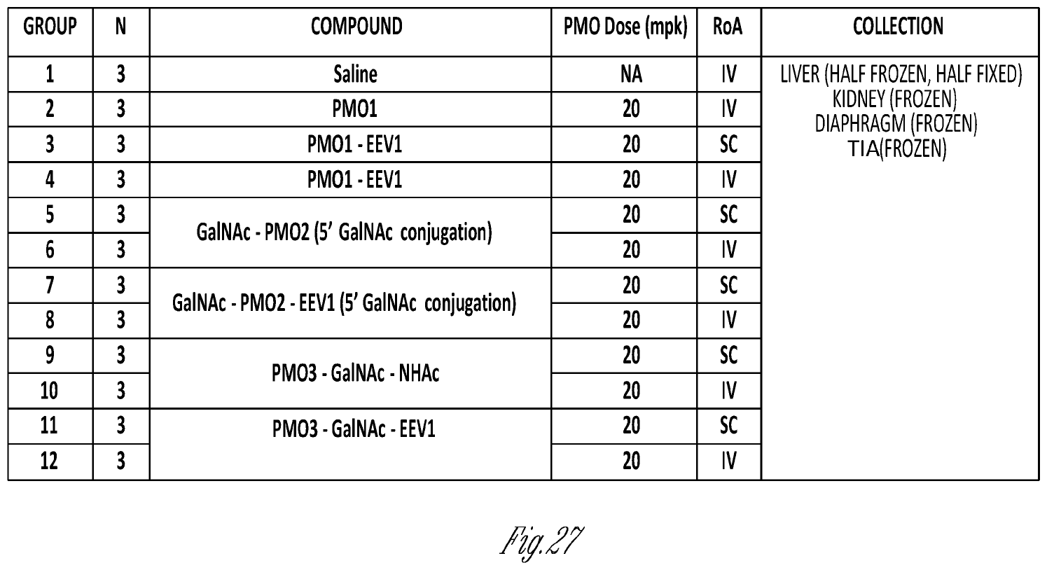

Mice were injected with 20 mpk of GalNAc-PMO2, GalNAc-PMO2-EEV1, PMO3-GalNAc-NHAc and PMO3-GalNAc-EEV1 (Normalized based on PMO) via both IV and SC route and sacrificed after one week. FIG. 26 illustrates eGFP protein level in liver after 1 week. Study design was summarized in FIG. 27.

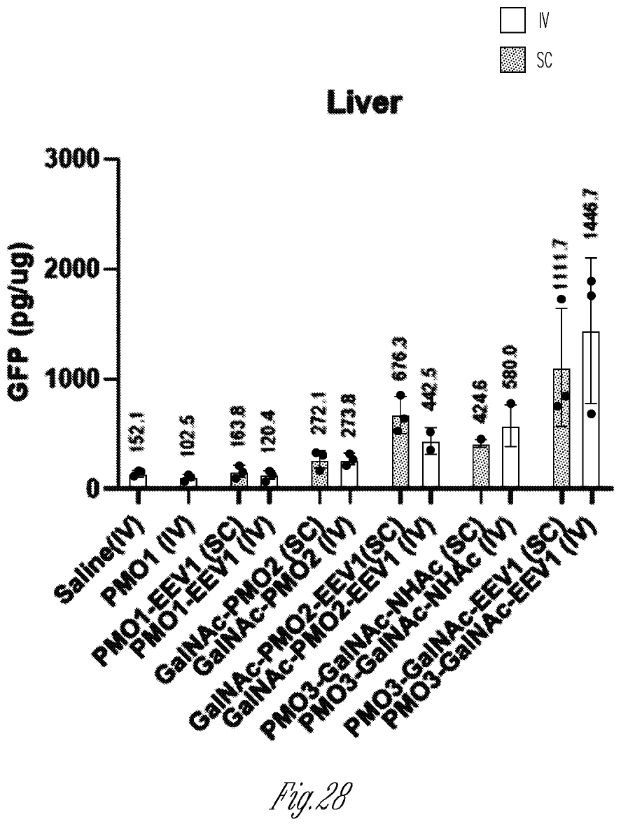

FIG. 28 illustrates eGFP (pg/μg) protein level in liver after 1 week.

Significant synergistic effect combining 3′-GalNAc and EEV conjugation was observed. PMO3-GalNAc-EEV1 enhanced the efficacy by 14-fold and 8.9-fold compared to PMO1 and PMO1-EEV1, respectively via IV route of administration.

In comparison to 5′-conjugation, the 3′ conjugation of GalNAc enhanced the efficacy by 1.6-fold and 2.1-fold for GalNAc-PMO constructs via SC and IV, respectively. Additionally, 3′-conjugation of GalNAc enhanced the liver efficacy by 1.6- and 3.2-fold for GalNAc-PMO-EEV constructs via SC and IV, respectively. 5′-GalNAc conjugation constructs showed similar or slightly higher efficacy using SC route but 3′-GalNAc conjugation, the IV route demonstrated slightly higher efficacy.

1-127. (canceled)

128. A compound comprising
(a) a cyclic cell penetrating peptide (CPP) comprising the structure of Formula (I): 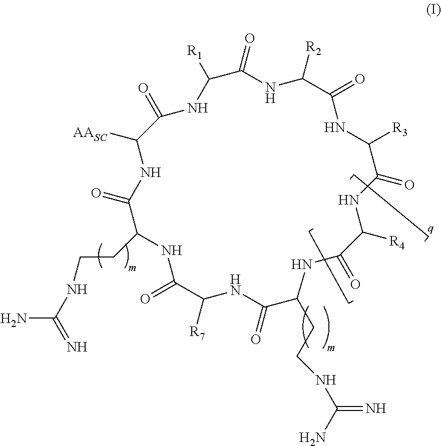
or a protonated form thereof,
wherein:
R₁, R₂, and R₃ can each independently be H or an amino acid residue having a side chain comprising an aromatic group;
two or three of R₁, R₂, and R₃ is an aromatic or heteroaromatic side chain of an amino acid;
two of R₁, R₂, and R₁ are a side chain of phenylalanine;
R₄ and R₇ are independently H or an amino acid side chain;
AA_{SC} is an amino acid side chain;
q is 1, 2, 3 or 4; and
each m is independently an integer 0, 1, 2, or 3.
(b) a therapeutic moiety (TM) selected from am oligonucleotide, a peptide or a small molecule;
(c) from 1 to 9 carbohydrate targeting moieties (CTM); and
wherein and one or more linkers link the CPP, the EP, the TO, and the CTM.

129. The compound of claim 128, having the Formula (N) 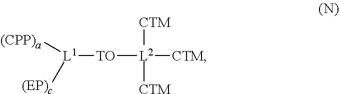
wherein
EP is an exocyclic peptide comprising from 2 to 10 amino acid residues, wherein at least one amino acid residue is arginine.
a is an integer from 1 to 3;
c is 0 or 1;
L¹ is a linker comprising the structure: 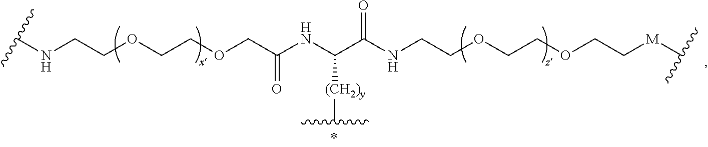
wherein: x′ is an integer from 1-23; y is an integer from 1-5; z′ is an integer from 1-23; * is he point of attachment to the AA_{SC}; and M is a bonding group;
L² is a linker comprising: 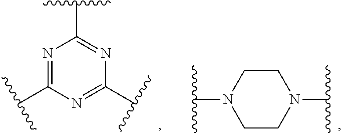 [SMILES: *c1nc(*)nc(*)n1 ; *N1CCN(*)CC1]
or a combination thereof;
TO is a therapeutic oligonucleotide.

130. The compound of claim 129, wherein L² is a linker comprising: 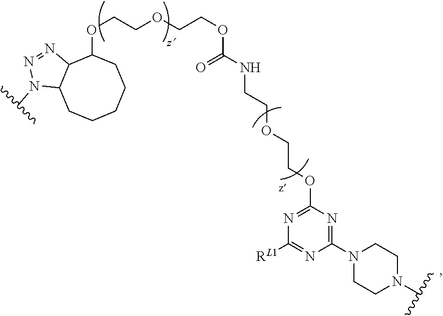
wherein, each z′ is, independently, an integer from 1 to 23, and R^{L1} is an optionally substituted amino group.

131. The compound of claim 128, wherein the comp und has a structure of Formula 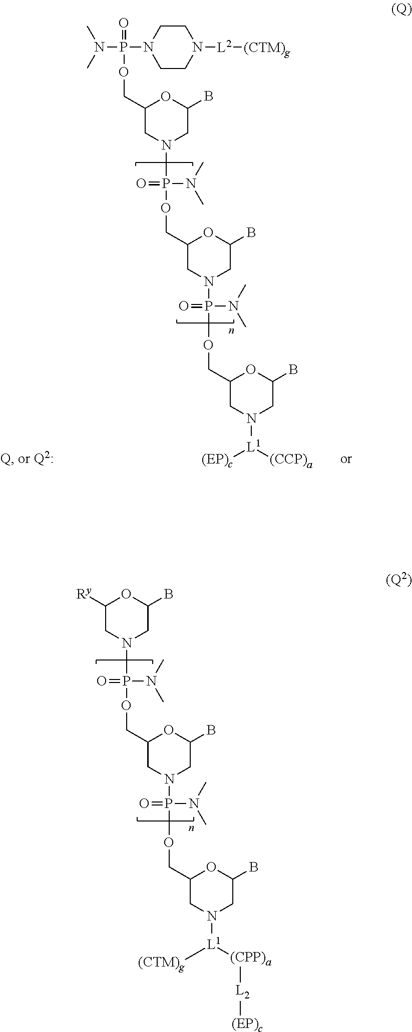
wherein:
a is an integer from 1 to 3;
c is 0 or 1;
g is an integer from 1 to 4;
L¹ is a linker of formula; 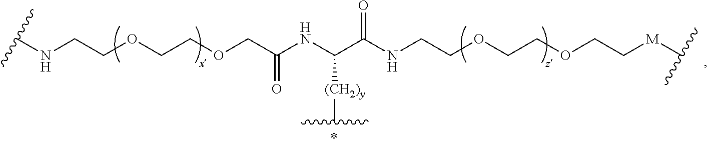
wherein:
x′ is an integer from 1-23; y is an integer from 1-5; z′ is an integer from 1-23; * is the point of attachment to the AA_{SC}; and M is a bonding group;
L² is a linker comprising: 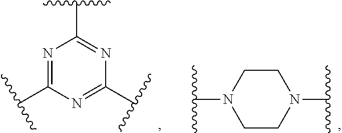 [SMILES: *c1nc(*)nc(*)n1 ; *N1CCN(*)CC1]
or a combination thereof;
B is each independently a nucleobase of the therapeutic oligonucleotide; and
n is an integer from 1 to 1000.

132. The compound of claim 131, wherein n is an integer from 5 to 50.

133. The compound of claim 128, wherein the compound is of the formula: 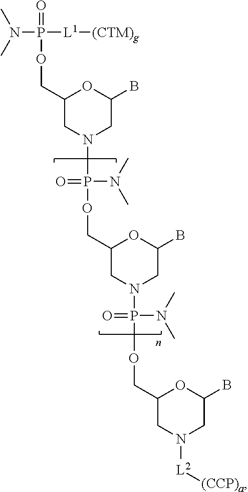
wherein:
n is an integer from 1 to 1000; and
L¹ or L² comprises:

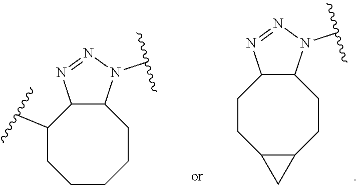 [SMILES: *C1CCCCCC2C1N=NN2* ; *N1N=NC2CCC3CC3CCC21]

134. The compound of claim 133, wherein n is an integer from 5 to 50.

135. The compound of claim 128, wherein the therapeutic moiety is a therapeutic oligonucleotide (TO) comprising at least one modified nucleotide or nucleic acid comprising a phosphorothioate (PS) nucleotide, a phosphorodiamidate morpholino nucleotide, a locked nucleic acid (LNA), a peptide nucleic acid (PNA), a nucleotide comprising a 2′-O-methyl (2′—OMe) modified backbone, a 2′O-methoxy-ethyl (2′-MOE) nucleotide, a 2′,4′ constrained ethyl (cEt) nucleotide, a 2′-deoxy-2′-fluoro-beta-D-arabinonucleic acid (2′F-ANA), or a combination thereof.

136. The compound of claim 135, wherein the TO comprises a small interfering RNA (siRNA), a microRNA (miRNA), a ribozyme, an immune stimulating nucleic acid, an antisense oligonucleotide (ASO), an antagomir, an antimir, a microRNA a mimic, a supermir, a UL adaptor, an aptamer, or a guide RNA.

137. The compound of claim 135, wherein the TO comprises a phosphorodiamidate morpholino (PMO) oligonucleotide.

138. The compound of claim 128, wherein the CTM comprises a monosaccharide selected from galactose, galactosamine, N-acetyl-galactosamine (GalNAc), and combinations thereof.

139. The compound of claim 128, wherein the CTM comprises GalNAc.

140. The compound of claim 128, wherein the cyclic CPP comprises:
Formula (I-1): 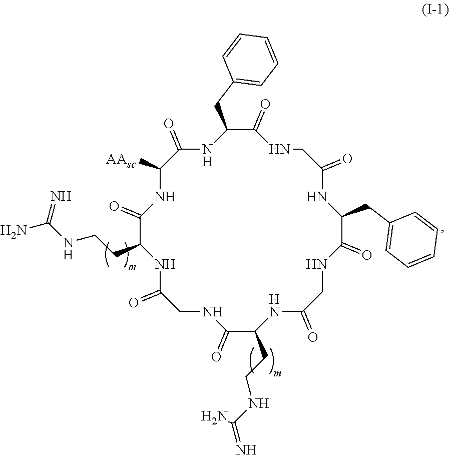
or a protonated form thereof;
Formula (I-2): 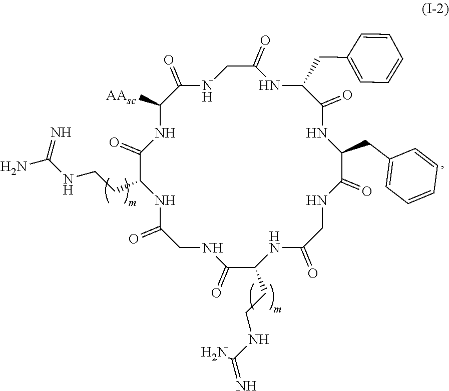
or a protonated form thereof;
Formula (I-3): 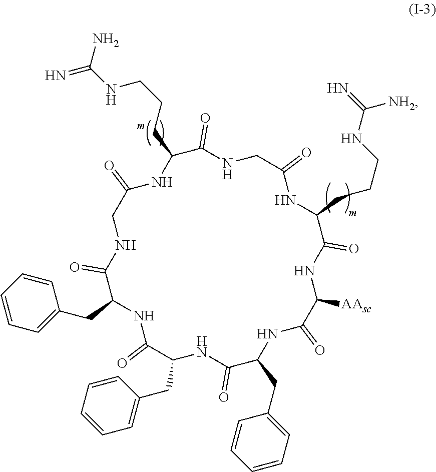
or a protonated form thereof;
Formula (I-4): 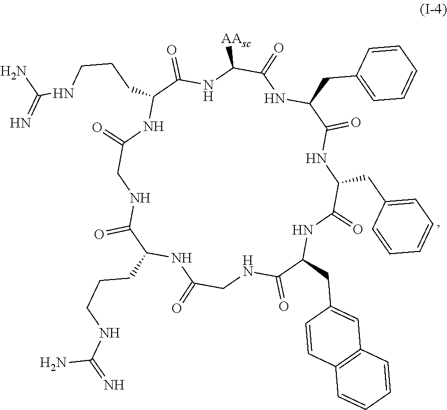
or a protonated form thereof;
Formula (I-5): 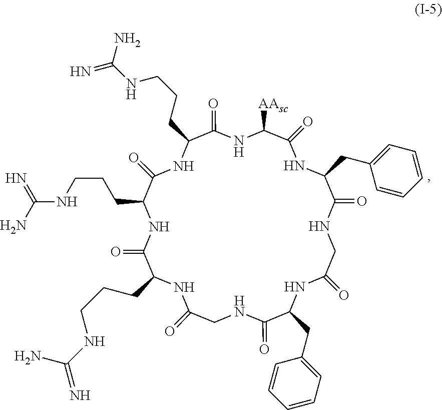
or a protonated form thereof;
or
Formula (I-6): 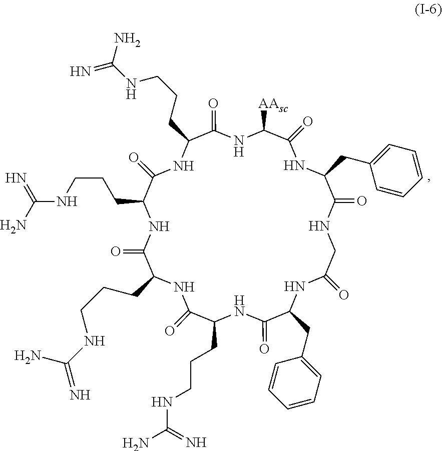
or a protonated form thereof.

141. The compound of claim 129, wherein M comprises

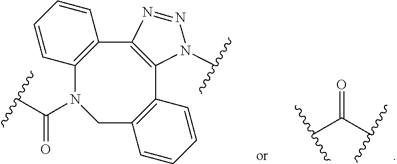

142. The compound of claim 129, wherein the exocyclic peptide comprises at least two lysine residues.

143. The compound of claim 129, wherein the exocyclic peptide (EP) comprises one of the following sequences: KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHHH RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG.

144. The compound of claim 129, wherein the EP comprises: PKKKRKV.

145. The compound of claim 128, wherein the compound has the formula:

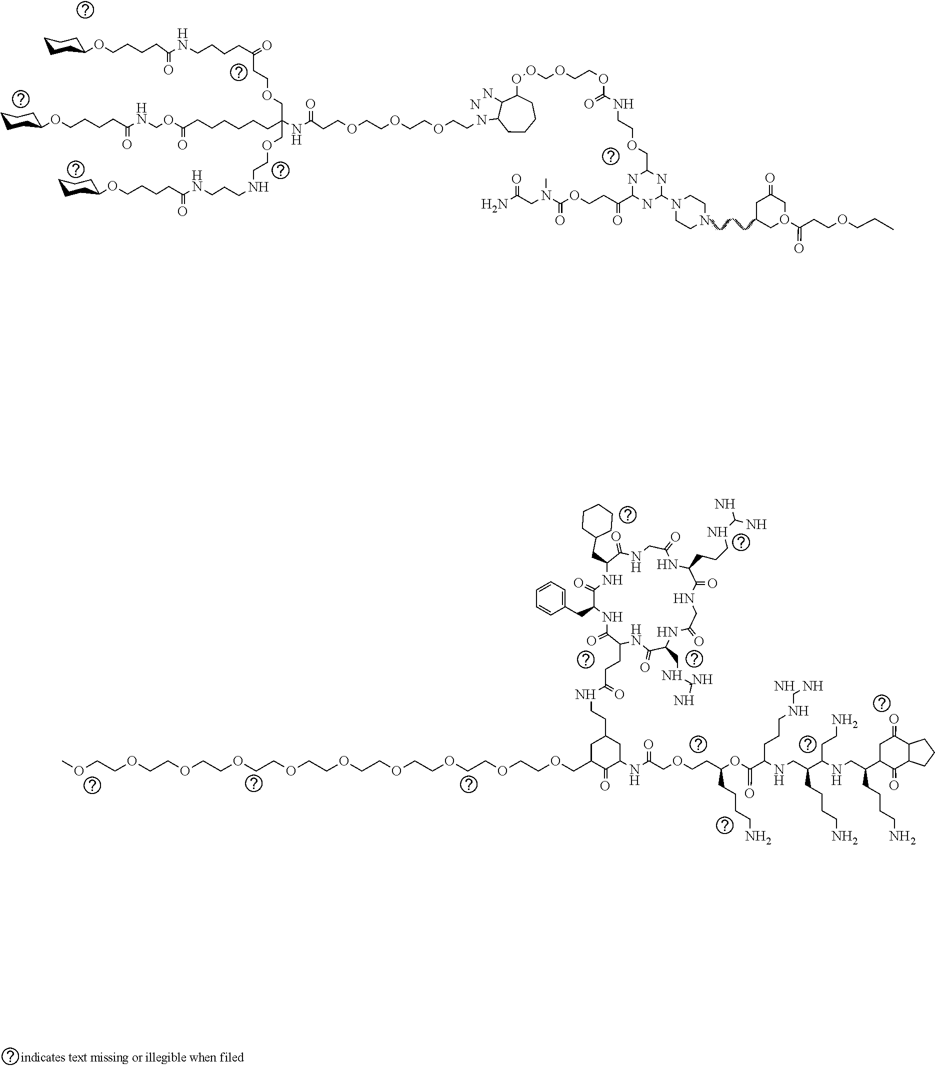

146. A pharmaceutical composition comprising a compound of claim 128 and a pharmaceutically acceptable carrier.

147. A method of treating a disease or disorder in a patient, comprising administering to the patient a therapeutically effective amount of a compound of claim 128.

148. The method of claim 147, wherein administration of the compound comprises parenteral administration.

149. The method of claim 148, wherein parenteral administration comprises subcutaneous, intramuscular, intravenous, interarticular, intrabronchial, intraabdominal, intracranial, intrathecal, intragastric, intrahepatic, intramyocardial, intrapleural, or intrapulmonary administration.
